(19) United States
(12) Patent Application Publication
(10) Pub. No.: US 2024/0100159 A1
(43) Pub. Date: Mar. 28, 2024

(54) USE OF IL-7R-ALPHA-GAMMA AGONIST PEPTIDES IN CELL MANUFACTURING

(71) Applicant: MEDIKINE, INC., MENLO PARK, CA (US)

(72) Inventors: WILLIAM J. DOWER, MENLO PARK, CA (US); MICHAEL C. NEEDELS, MENLO PARK, CA (US); RONALD W. BARRETT, SARATOGA, CA (US); ALICE V. BAKKER, CUPERTINO, CA (US); STEVEN E. CWIRLA, MENLO PARK, CA (US); SUNIL BAJAD, FREMONT, CA (US); INKYUNG PARK, MORGAN HILL, CA (US)

(21) Appl. No.: 18/462,276

(22) Filed: Sep. 6, 2023

Related U.S. Application Data
(60) Provisional application No. 63/404,062, filed on Sep. 6, 2022.

(51) Int. Cl. A61K 39/00; C07K 14/54; C12N 5/0783

(52) U.S. Cl. CPC A61K 39/4613; C07K 14/5418; C12N 5/0646; C12N 2501/2302; C12N 2501/2307

(57) ABSTRACT

The use of IL-7Rαγc agonist peptides in cell manufacturing is disclosed. The IL-7Rαγc agonist peptides can facilitate the selective growth of immune cell populations. Culture media comprising the IL-7Rαγc agonist peptides, methods of expanding a target immune cell population, and methods of manufacturing a target immune cell population are disclosed. The enriched immune cell populations can be used in immunotherapy.

This application claims the benefit under 35 U.S.C. § 119(e) of U.S. Provisional Application No. 63/404,062 filed on Sep. 6, 2022, which is incorporated by reference in its entirety.

### FIELD

The present disclosure relates to the use of IL-7Rαγc agonist peptides in cell manufacturing. The IL-7Rαγc peptide agonists can facilitate the selective growth of immune cell populations. The enriched immune cell populations are useful in immunotherapy.

### SEQUENCE LISTING

The Sequence Listing associated with this application is filed in electronic format by EFS-Web and is incorporated by reference in its entirety. The XML document in WIPO ST.26 format is named 62AJ-001610US-374593 9-6-2023 A_XML and is 464 kilobytes in size.

### BACKGROUND

Recombinant cytokines can be used to stimulate immune cell proliferation.

The use of recombinant cytokines to stimulate immune cell proliferation has a number of drawbacks. Recombinant cytokines can have a wide range of activity depending on the source and batch and therefore the activity must be measured at the time of use and custom dilutions made to accommodate accurate and reproducible cell manufacturing. Recombinant cytokines are unstable as a liquid formulation and therefore are typically provided as lyophilizates that must be reconstituted before use. The process for manufacturing GMP-grade recombinant cytokines is expensive. The purity of recombinant cytokines can be compromised by contamination with endotoxins and host cell proteins for bacterial systems and by adventitious agents for mammalian and recombinant insect systems. Also, quality control procedures required to ensure product quality can be time-consuming and expensive.

Aspects of the present invention are directed to the use of small peptides as stimulants of immune cell proliferation. The small peptides can replace the corresponding recombinant cytokines in the manufacture of immune cells and cell therapy products.

IL-7Rαγc peptides that function as IL-7R agonists are disclosed in U.S. Application Publication No. 2021/0253670 A1, which is incorporated by reference in its entirety.

### SUMMARY

According to the present disclosure, culture media for expanding a target immune cell population comprise a first stimulant of proliferation for the target immune cell population, wherein the first stimulant comprises an IL-7Rαγc agonist peptide.

According to the present disclosure, methods of expanding a target immune cell population of an initial immune cell population comprise incubating an initial immune cell population in a culture medium provided by the present disclosure to provide an expanded target immune cell population.

According to the present disclosure, methods of manufacturing an immune cell population comprise activating a target immune cell population of a population of primary cells to provide an activated target immune cell population; and expanding the activated target immune cell population, wherein expanding comprises incubating the activated target immune cell population in the presence of an IL-7Rαγc agonist peptide to provide an expanded target immune cell population.

According to the present disclosure, an enriched population of immune cells is prepared using a culture medium according to the present disclosure.

According to the present disclosure, an enriched population of immune cells is prepared using a method of expanding an immune cell population according to the present disclosure.

According to the present disclosure, an enriched population of immune cells is prepared using a method of manufacturing an immune cell population according to the present disclosure.

According to the present disclosure, pharmaceutical compositions comprise an enriched population of immune cells according to the present disclosure.

According to the present disclosure, methods of treating a disease in a patient comprise administering to a patient in need of such treatment a therapeutically effective amount of a pharmaceutical composition according to the present disclosure.

According to the present disclosure, articles of manufacture comprise a culture medium according to the present disclosure.

According to the present disclosure, articles of manufacture comprise an enriched population of immune cells according to the present disclosure.

According to the present disclosure, articles of manufacture comprise a pharmaceutical composition according to the present disclosure.

According to the present disclosure, immobilized IL-7Rαγc peptide agonists comprise IL-7Rαγc peptide agonists bound to a substrate.

### BRIEF DESCRIPTION OF THE DRAWINGS

The drawings described herein are for illustration purposes only. The drawings are not intended to limit the scope of the present disclosure.

FIGS. 1A-1D show STAT5 phosphorylation in resting PBMCs by an IL-2Rβγc agonist peptide (SEQ ID NO: 98) and by an IL-7Rαγc agonist peptide (SEQ ID NO: 395-Ac).

FIGS. 2A-2D show STAT5 phosphorylation in activated PBMCs by an IL-2Rβγc agonist peptide (SEQ ID NO: 98) and by an IL-7Rαγc agonist peptide (SEQ ID NO: 395-Ac).

FIGS. 3A-3E show the cell count of CD8 memory cell subpopulations following incubation of resting PBMCs for four weeks with an IL-2Rβγc agonist peptide (SEQ ID NO: 99, with an IL-7Rαγc agonist peptide (SEQ ID NO: 395-Ac), or without an agonist peptide.

FIGS. 4A-4E show the cell count of CD8 memory cell subpopulations following incubation of activated PBMCs for four weeks with an IL-2Rβγc agonist peptide (SEQ ID NO: 99, with an IL-7Rαγc agonist peptide (SEQ ID NO: 395-Ac), or without an agonist peptide.

FIGS. 5A-5B show NK subpopulations in resting NK cells following incubation without or with an IL-2Rβγc agonist peptide (SEQ ID NO: 99 for twenty-one days.

FIGS. 6A-6B show NK subpopulations in resting NK cells following incubation without or with an IL-7Rαγc agonist peptide (SEQ ID NO: 395-Ac) for twenty-one days.

FIGS. 7A-7C show NK subpopulations following incubation of resting NK cells without a agonist peptide, with an IL-2Rβγc agonist peptide (SEQ ID NO: 99, or with an IL-7Rαγc agonist peptide (SEQ ID NO: 395-Ac).

FIGS. 8A-8C show NK subpopulations following incubation of activated NK cells without a agonist peptide, with an IL-2Rβγc agonist peptide (SEQ ID NO: 99, or with an IL-7Rαγc agonist peptide (SEQ ID NO: 395-Ac).

FIG. 9A shows activation of NK-92 cells following exposure to IL-2 or an IL-2Rβγc agonist peptide having SEQ ID NO: 359-Ac.

FIG. 9B shows proliferation of NK cells in human PBMCs following exposure to different concentrations of IL-2 or an IL-2Rβγc agonist peptide having SEQ ID NO: 359-Ac.

### DETAILED DESCRIPTION

A dash (“-”) that is not between two letters or symbols is used to indicate a point of attachment for a moiety or substituent. For example, —CONH₂ is attached to a compound through the carbon atom and —X¹—X²— denotes amino acids X¹ and X² covalently bound together through a single bond.

“Alkyl” refers to a saturated or unsaturated, branched, or straight-chain, monovalent hydrocarbon radical derived by the removal of one hydrogen atom from a single carbon atom of a parent alkane, alkene, or alkyne. Examples of alkyl groups include methyl; ethyls such as ethanyl, ethenyl, and ethynyl; propyls such as propan-1-yl, propan-2-yl, prop-1-en-1-yl, prop-1-en-2-yl, prop-2-en-1-yl (allyl), prop-1-yn-1-yl, prop-2-yn-1-yl, etc.; butyls such as butan-1-yl, butan-2-yl, 2-methyl-propan-1-yl, 2-methyl-propan-2-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, but-1-yn-1-yl, but-1-yn-3-yl, but-3-yn-1-yl, etc.; and the like. The term “alkyl” is specifically intended to include groups having any degree or level of saturation, i.e., groups having exclusively carbon-carbon single bonds, groups having one or more carbon-carbon double bonds, groups having one or more carbon-carbon triple bonds, and groups having combinations of carbon-carbon single, double, and triple bonds. Where a specific level of saturation is intended, the terms alkanyl, alkenyl, and alkynyl are used. In certain embodiments, an alkyl group is C₁₋₆ alkyl, C₁₋₅ alkyl, C₁₋₄ alkyl, C₁₋₃ alkyl, and in certain embodiments, ethyl or methyl.

“Cycloalkyl” refers to a saturated or partially unsaturated cyclic alkyl radical. A cycloalkyl can be, for example, C₃₋₆ cycloalkyl, C₃₋₅ cycloalkyl, C₅₋₆ cycloalkyl, cyclopropyl, cyclopentyl, and in certain embodiments, cyclohexyl. Cycloalkyl can be selected from cyclopropyl, cyclobutyl, cyclopentyl, and cyclohexyl.

“Cyclized” refers to a reaction in which one part of a peptide or polypeptide molecule becomes linked to another part of the peptide or polypeptide molecule to form a closed ring, such as by forming a disulfide bridge or other similar bond, such as a lactam bond. Peptide monomer compounds or monomer subunits of peptide dimer compounds can be cyclized via an intramolecular bond between two amino acid residues present in the peptide monomer or monomer subunit. A peptide such as an IL-7Rαγc agonist peptide can include cysteines that are bound together through disulfide bonds and thereby are cyclized IL-7Rαγc agonist peptides.

“Heterocycloalkyl” by itself or as part of another substituent refers to a saturated cyclic alkyl radical in which one or more carbon atoms (and certain associated hydrogen atoms) are independently replaced with the same or a different heteroatom; or to a parent aromatic ring system in which one or more carbon atoms (and certain associated hydrogen atoms) are independently replaced with the same or a different heteroatom such that the ring system violates the Hückel-rule. Examples of heteroatoms to replace the carbon atom(s) include N, P, O, S, and Si. Examples of heterocycloalkyl groups include groups derived from epoxides, azirines, thiiranes, imidazolidine, morpholine, piperazine, piperidine, pyrazolidine, pyrrolidine, quinuclidine, and the like. A heterocycloalkyl can be C₅ heterocycloalkyl and is selected from pyrrolidinyl, tetrahydrofuranyl, tetrahydrothiophenyl, imidazolidinyl, oxazolidinyl, thiazolidinyl, doxolanyl, and dithiolanyl. A heterocycloalkyl can be C₆ heterocycloalkyl and is selected from piperidinyl, tetrahydropyranyl, piperizinyl, oxazinyl, dithianyl, and dioxanyl. A heterocycloalkyl group can be C₃₋₆ heterocycloalkyl, C₃₋₅ heterocycloalkyl, C₅₋₆ heterocycloalkyl, and in certain embodiments, C₅ heterocycloalkyl or C₆ heterocycloalkyl. A heteroatomic group can be selected from —O—, —S—, —NH—, —N(—CH₃)—, —SO—, and —SO₂—, in certain embodiments, a heteroatomic group is selected from —O— and —NH—, and in certain embodiments, a heteroatomic group is —O— or —NH—.

“Binding affinity” refers to the strength of the binding interaction between a single biomolecule and its ligand/binding partner. Binding affinity is expressed as the IC₅₀. For example, the binding affinity of a compound such as an IL-7Rαγc agonist peptide refers to the IC₅₀ as determined using, for example, a method described in the examples.

“Direct binding” refers to the binding interaction between a single biomolecule and its binding partner, such as, for example, the interaction of an IL-7Rα ligand and the hu-IL-7Rα subunit or the interaction of an IL-7Rαγc agonist peptide and IL-7R. Direct binding can be determined using phage ELISA assays.

“Agonist” refers to a biologically active ligand that binds to its complementary biologically active receptor or receptor subunit(s) and activates the receptor to cause a biological response mediated by the receptor or to enhance a preexisting biological activity mediated by the receptor.

“Partial agonist” refers to a compound that provides a level of activation, that is, for example, less than 75% of maximum activation, less than 50%, less than 25%, less than 10%, or less than 1% of the maximum activation. A partial IL-7R agonist exhibits a level of activation that is less than the level of activation provided by IL-7.

“Antagonist” refers to a biologically active ligand or compound that binds to its complementary receptor or subunit(s) and blocks or reduces a biological response of the receptor. An IL-7R antagonist binds to IL-7R with an IC₅₀ of less than 100 μM and inhibits the functional activity of IL-7 as determined, for example, using a functional assay such as any of the functional assays disclosed in the examples.

Amino acid residues are abbreviated as follows: alanine is Ala or A; arginine is Arg or R; asparagine is Asn or N; aspartic acid is Asp or D; cysteine is Cys or C; glutamic acid is Glu or E; glutamine is Gln or Q; glycine is Gly or G; histidine is His or H; isoleucine is Ile or I; leucine is Leu or L; lysine is Lys or K; methionine is Met or M; phenylalanine is Phe or F; proline is Pro or P; serine is Ser or S; threonine is Thr or T; tryptophan is Trp or W; tyrosine is Tyr or Y; and valine is Val or V.

“Non-natural amino acids” include, for example, β-amino acids, homo-amino acids, proline and pyruvic acid derivatives, histidine derivatives with alkyl or heteroatom moieties bound to the imidazole ring, amino acids with pyridine-containing side chains, 3-substituted alanine derivatives, glycine derivatives, ring-substituted phenylalanine, and tyrosine derivatives, and N-methyl amino acids.

Amino acids having a large hydrophobic side chain include, for example, isoleucine (I), leucine (L), methionine (M), valine (V), phenylalanine (F), tyrosine (Y), and tryptophan (W).

Amino acids having a small hydrophobic side chain include, for example, alanine (A), glycine (G), proline (P), serine (S), and threonine (T).

Amino acids having a basic side chain include, for example, arginine (R), lysine (K), and histidine (H).

Amino acids having an acidic side chain include, for example, aspartate (D) and glutamate (E).

Amino acids having a polar/neutral side chain include, for example, histidine (H), asparagine (N), glutamine (Q), serine (S), threonine (T), and tyrosine (Y).

Amino acids having an aromatic side chain include, for example, phenylalanine (F), histidine (H), tryptophan (W), and tyrosine (Y).

Amino acids having a hydroxyl side chain include, for example, serine (S), threonine (T), and tyrosine (Y).

“Conservative amino acid substitution” means that amino acids within each of the following groups can be substituted with another amino acid within the group such as, for example, amino acids having a small hydrophobic side chain comprising alanine (A), glycine (G), proline (P), serine (S), and threonine (T); amino acids having a hydroxyl-containing side chain comprising serine (S), threonine (T), and tyrosine (Y); amino acids having an acidic side chain comprising aspartate (D) and glutamate (E); amino acids comprising a polar-neutral side chain comprising histidine (H), asparagine (N), glutamine (Q), serine (S), threonine (T), and tyrosine (Y); amino acids having a basic side chain comprising arginine (R), lysine (K), and histidine (H); amino acids having a large hydrophobic side chain comprising isoleucine (I), leucine (L), methionine (M), valine (V), phenylalanine (F), tyrosine (Y), and tryptophan (W); and amino acids having an aromatic side chain comprising phenylalanine (F), histidine (H), tryptophan (W), and tyrosine (Y).

“PEG,” “polyethylene glycol” and “poly(ethylene glycol)” refer to any nonpeptidic water-soluble poly(ethylene oxide). PEGs can comprise a structure —(OCH₂CH₂)ₙ— where n is, for example, an integer from 1 to 4,000. A PEG can also include moieties such as —CH₂CH₂—O(CH₂CH₂O)ₙ—CH₂CH₂— and/or —(OCH₂CH₂)ₙO— moieties, depending upon whether or not the terminal oxygens have been displaced, e.g., during a synthetic transformation. A PEG can be capped with a suitable end group. At least 50% of the repeating subunits of a PEG can have the structure —CH₂CH₂—. A PEG can have any suitable molecular weight, structure, and/or geometry such as branched, linear, forked, or multifunctional.

A “physiologically cleavable” or “hydrolyzable” or “degradable” bond is a bond that reacts with water (i.e., is hydrolyzed) under physiological conditions. The tendency of a bond to hydrolyze in water will depend not only on the general type of linkage connecting two central atoms but also on the substituents bound to these central atoms. Suitable hydrolytically unstable or weak linkages include but are not limited to carboxylate ester, phosphate ester, anhydrides, acetals, ketals, acyloxyalkyl ether, imines, orthoesters, peptides and oligonucleotides.

An “enzymatically degradable linkage” means a linkage that can be degraded or cleaved by one or more enzymes.

A “hydrolytically stable” linkage or bond refers to a chemical bond, such as a covalent bond, that is substantially stable in water such that the chemical bond does not undergo hydrolysis under physiological conditions to any appreciable extent over an extended period of time. Examples of hydrolytically stable linkages include, but are not limited to, the following: carbon-carbon bonds (e.g., in aliphatic chains), ethers, amides, urethanes, and the like. Generally, a hydrolytically stable linkage is one that exhibits a rate of hydrolysis of less than about 1% to 2% per day under physiological conditions.

“Covalently bonded” refers to a chemical bond that involves sharing of electrons to form electron pairs between atoms.

An “enzymatically degradable linkage” refers to a chemical linkage that can be degraded or cleaved by one or more enzymes.

A “hydrolytically stable” linkage or bond refers to a chemical bond, such as a covalent bond, that is substantially stable in water such that the chemical bond does not undergo hydrolysis under physiological conditions to any appreciable extent over an extended period of time. Examples of hydrolytically stable linkages include, but are not limited to, the following: carbon-carbon bonds (e.g., in aliphatic chains), ethers, amides, urethanes, and the like. Generally, a hydrolytically stable linkage is one that exhibits a rate of hydrolysis of less than about 1% to 2% per day under physiological conditions.

An “IL-7Rα ligand” refers to a peptide capable of binding to the IL-7Rα subunit of a mammalian IL-7 receptor, such as the hu-IL-7 receptor, with an IC₅₀ of less than 100 μM.

An “IL-2Rγc ligand”, an “IL-2Rγc ligand provided by the present disclosure”, an “Rγc ligand” refers to a peptide capable of binding to the Rγc subunit of a mammalian IL-7 receptor, such as the hu-IL-7 receptor, with an IC₅₀ of less than 100 μM.

The “hu-IL-7Rα subunit” refers to the human (Homo sapiens) interleukin-7 receptor subunit α precursor NCBI Reference Sequence NP_002176.2.

The “Rγc subunit” refers to the human (Homo sapiens) interleukin-7 receptor subunit γc precursor NCBI Reference Sequence NP_000197.1.

An “IL-7Rαγc agonist peptide” refers to a compound consisting of or comprising one or more IL-7Rα ligands and one or more Rγc ligands. The one or more IL-7Rα ligands and one or more Rγc ligands can be bound to an IL-7Rαγc linker. An IL-7Rαγc agonist peptide can comprise an IL-7Rαγc agonist peptide comprising two or more IL-7Rαγc agonist peptides, or an IL-7Rαγc agonist peptide can comprise a single ligand that simultaneously binds to both the hu-IL-7Rα subunit and the hu-IL-7Rγc subunit. An IL-7Rαγc agonist peptide is capable of binding to the Hu-IL-7Rα subunit and to the hu-IL-7Rγc subunit of hu-IL-7R with an IC₅₀ of less than 100 μM. An IL-7Rαγc agonist peptide is capable of binding to hu-IL-7R with an IC₅₀ of less than 100 μM.

An “IL-2Rβγc agonist peptide” refers to a compound consisting of or comprising one or more IL-2Rβ ligands and one or more IL-2Rγc ligands. The one or more IL-2Rβ ligands and one or more IL-2Rγc ligands can be bound to an IL-2Rβγc linker. An IL-2Rβγc agonist peptide can comprise an IL-2Rβγc agonist peptide comprising two or more IL-2Rβγc agonist peptides, or it can comprise a single ligand that simultaneously binds to both the IL-2Rβ subunit and the IL-2Rγc subunit. An IL-2Rβγc agonist peptide is capable of binding to the IL-2Rβ subunit and to the IL-2Rγc subunit of IL-2R with an IC₅₀ less than 100 μM.

An “IL-2Rβγc agonist peptide” refers to a compound capable of binding to both the hu-IL-2Rβ and to the hu-IL-2Rγc subunit and includes an IL-2Rβ ligand and an IL-2Rγc ligand. An IL-2Rβγc agonist peptide can bind to both the IL-2Rβ subunit and the IL-2Rγc subunit with an IC₅₀, for example, of less than 100 μM such as less than 10 μM, less than 1 μM, or less than 0.1 μM.

Bioisosteres are atoms or molecules that fit the broadest definition for isosteres. The concept of bioisosterism is based on the concept that single atoms, groups, moieties, or whole molecules, which have chemical and physical similarities, produce similar biological effects. A bioisostere of a parent compound can still be recognized and accepted by its appropriate target, but its functions will be altered as compared to the parent molecule. Parameters influenced by bioisosteric replacements include, for example, size, conformation, inductive and mesomeric effects, polarizability, capacity for electrostatic interactions, charge distribution, H-bond formation capacity, pKa (acidity), solubility, hydrophobicity, lipophilicity, hydrophilicity, polarity, potency, selectivity, reactivity, or chemical and metabolic stability, ADME (absorption, distribution, metabolism, and excretion). Although common in pharmaceuticals, carboxyl groups or carboxylic acid functional groups (—CO₂H) in a parent molecule may be replaced with a suitable surrogate or (bio)isostere to overcome chemical or biological shortcomings while retaining the desired attributes of the parent molecule bearing one or more carboxyl groups or carboxylic acid functional groups (—CO₂H). IL-7Rαγc agonist peptides include bioisosteres of the IL-7Rαγc agonist peptides provided by the present disclosure.

“Isostere” or “isostere replacement” refers to any amino acid or other analog moiety having physiochemical and/or structural properties similar to a specified amino acid. An “isostere” or “suitable isostere” of an amino acid is another amino acid of the same class, wherein amino acids belong to the following classes based on the propensity of the side chain with respect to a polar solvent such as water:hydrophobic (low propensity to be in contact with water), polar or charged (energetically favorable contact with water). Examples of charged amino acid residues include lysine (+), arginine (+), aspartate (−), and glutamate (−). Examples of polar amino acids include serine, threonine, asparagine, glutamine, histidine, and tyrosine. Illustrative hydrophobic amino acids include alanine, valine, leucine, isoleucine, proline, phenylalanine, tryptophan, cysteine, and methionine. The amino acid glycine does not have a side chain and is difficult to assign to one of the above classes. However, glycine is often found at the surface of proteins, often within loops, providing high flexibility to these regions, and an isostere may have a similar feature. Proline has the opposite effect, providing rigidity to the protein structure by imposing certain torsion angles on the segment of the polypeptide chain. An isostere can be a derivative of an amino acid, e.g., a derivative having one or more modified side chains as compared to the reference amino acid. IL-7Rαγc agonist peptides include isosteres of the IL-7Rαγc agonist peptides provided by the present disclosure.

“Patient” refers to a mammal, for example, a human.

“Peptide” refers to a polymer in which the monomers include amino acids joined together through amide bonds. A peptide can comprise, for example, less than 200 amino acids, less than 100 amino acids, less than 50 amino acids, less than 40 amino acids, less than 30 amino acids, or less than 20 amino acids. A peptide can comprise naturally occurring amino acids, non-naturally occurring amino acids, or a combination thereof.

In addition to peptides consisting only of naturally occurring amino acids, peptidomimetics or peptide analogs are also provided. A peptide mimetic can be functionally and/or structurally similar to another peptide. Peptide mimetics that are functionally and/or structurally similar to therapeutically useful peptides may be used to produce an equivalent or enhanced therapeutic or prophylactic effect.

Generally, peptidomimetics can be structurally similar to a paradigm peptide, for example, a peptide that has a biological or pharmacological activity, such as a naturally occurring receptor-binding peptide, but have one or more peptide linkages optionally replaced by a linkage such as —CH₂—NH—, —CH₂—S—, —CH₂—CH₂—, —CH═CH— (cis and trans), —COCH₂—, —CH(OH)CH₂—, and —CH₂SO—, by methods known in the art.

Substitution of one or more amino acids of a consensus sequence with a D-amino acid of the same type, such as D-lysine in place of L-lysine, may be used to generate more stable peptides. In addition, constrained peptides comprising a consensus sequence, or a substantially identical consensus sequence variation may be generated by methods known in the art; for example, by adding internal cysteine residues capable of forming intramolecular disulfide bridges which cyclize the peptide.

Synthetic or non-naturally occurring amino acids refer to amino acids that do not naturally occur in vivo but which, nevertheless, can be incorporated into the peptide ligands provided by the present disclosure. Suitable examples of synthetic amino acids include the D-α-amino acids of naturally occurring L-α-amino acid as well as non-naturally occurring D- and L-α-amino acids represented by the formula H₂NCHR⁵COOH where R is C₁₋₆ alkyl, C₃₋₈ cycloalkyl, C₃₋₈ heterocycloalkyl; an aromatic residue of from 6 to 10 carbon atoms optionally having from 1 to 3 substituents on the aromatic nucleus selected from hydroxyl, lower alkoxy, amino, and carboxyl; -alkylene-Y where alkylene is an alkylene group of from 1 to 7 carbon atoms and Y is selected from a hydroxyl, amino, cycloalkyl, and cycloalkenyl having from 3 to 7 carbon atoms; aryl of from 6 to 10 carbon atoms, such as from 1 to 3 substituents on the aromatic nucleus selected hydroxyl, lower alkoxy, amino and carboxyl; heterocyclic of from 3 to 7 carbon atoms and 1 to 2 heteroatoms selected from oxygen, sulfur, and nitrogen; —C(O)R² where R² is selected from hydrogen, hydroxy, lower alkyl, lower alkoxy, and —NR³R⁴ where each of R³ and R⁴ is independently selected from hydrogen and lower alkyl; —S(O)ₙR⁶ where n is 1 or 2 and R⁶ is C₁₋₆ alkyl, and with the proviso that R⁶ does not define a side chain of a naturally occurring amino acid.

Examples of other synthetic amino acids include amino acids in which the amino group is separated from the carboxyl group by more than one carbon atom, such as β-alanine and γ-aminobutyric acid.

Examples of suitable synthetic amino acids include the D-amino acids of naturally occurring L-amino acids, L-1-naphthyl-alanine, L-2-naphthylalanine, L-cyclohexylalanine, L-2-amino isobutyric acid, the sulfoxide and sulfone derivatives of methionine, such as HOOC—(H₂NCH)CH₂CH₂—S(O)ₙR⁶, where n and R⁶ are as defined above as well as the lower alkoxy derivative of methionine, such as HOOC—(H₂NCH)CH₂CH₂OR⁶ where R⁶ is as defined above.

“N-terminus” refers to the end of a peptide or polypeptide, such as an N-terminus of an IL-7Rαγc agonist peptide, an IL-7Rα ligand, or an Rγc ligand that bears an amino group in contrast to the carboxyl end bearing a carboxyl acid group.

“C-terminus” refers to the end of a peptide or polypeptide, such as a C-terminus of an IL-7Rαγc agonist peptide, an IL-7Rα ligand or an Rγc ligand that bears a carboxylic acid group in contrast to the amino terminus bearing an amino group. In certain synthetic peptides, the N-terminus does not bear an amino group and/or the C-terminus does not bear a carboxyl group. In such cases, the nomenclature refers to the direction of the peptide backbone.

“Pharmaceutically acceptable” refers to approved or approvable by a regulatory agency of the Federal or a state government or listed in the U.S. Pharmacopoeia or other generally recognized pharmacopeia for use in animals, and more particularly in humans.

“Pharmaceutically acceptable salt” refers to a salt of a compound, which possesses a desired pharmacological activity of the parent compound. Such salts include acid addition salts formed with inorganic acids and one or more protonate-able functional groups such as primary, secondary, or tertiary amines within the parent compound. Examples of inorganic acids include hydrochloric acid, hydrobromic acid, sulfuric acid, nitric acid, and phosphoric acid. A salt can be formed with organic acids such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, or muconic acid. A salt can be formed when one or more acidic protons present in the parent compound are replaced by a metal ion, e.g., an alkali metal ion, an alkaline earth ion, or an aluminum ion, or combinations thereof, or coordinates with an organic base such as ethanolamine, diethanolamine, triethanolamine, or N-methylglucamine. A pharmaceutically acceptable salt can be a hydrochloride salt. A pharmaceutically acceptable salt can be a sodium salt. A compound can have two or more ionizable groups, and a pharmaceutically acceptable salt can comprise one or more counterions, such as a bi-salt, for example, a dihydrochloride salt.

“Pharmaceutically acceptable salt” refers to hydrates and other solvates, as well as salts in crystalline or non-crystalline form. Where a particular pharmaceutically acceptable salt is disclosed, it is understood that the particular salt (e.g., a hydrochloride salt) is an example of a salt and that other salts may be formed using techniques known to one of skill in the art. Additionally, a pharmaceutically acceptable salt to the corresponding compound, free base and/or free acid, using techniques generally known in the art. See also: Stahl and Wermuth, C.G. (Editors), Handbook of Pharmaceutical Salts, Wiley-VCH, Weinheim, Germany, 2008.

“Pharmaceutically acceptable vehicle” refers to a pharmaceutically acceptable diluent, a pharmaceutically acceptable adjuvant, a pharmaceutically acceptable excipient, a pharmaceutically acceptable carrier, or a combination of any of the foregoing with which a compound provided by the present disclosure may be administered to a patient and which does not destroy the pharmacological activity thereof and which is non-toxic when administered in doses sufficient to provide a therapeutically effective amount of the compound.

“Pharmaceutical composition” refers to a composition comprising an IL-7Rαγc agonist peptide provided by the present disclosure and at least one pharmaceutically acceptable vehicle or excipient with which the IL-7Rαγc agonist peptide or a pharmaceutically acceptable salt thereof is administered to a patient.

A “physiologically cleavable” or “hydrolyzable” or “degradable” bond is a bond that reacts with water (i.e., is hydrolyzed) under physiological conditions. The tendency of a bond to hydrolyze in water will depend not only on the general type of linkage connecting two central atoms but also on the substituents bound to these central atoms. Suitable hydrolytically unstable or weak linkages include, for example, carboxylate ester, phosphate ester, anhydrides, acetals, ketals, acyloxyalkyl ether, imines, orthoesters, peptides, and oligonucleotides.

“Preventing” or “prevention” refers to a reduction in risk of acquiring a disease or disorder (i.e., causing at least one of the clinical symptoms of the disease not to develop in a patient that may be exposed to or predisposed to the disease but does not yet experience or display symptoms of the disease). In some embodiments, “preventing” or “prevention” refers to reducing symptoms of the disease by taking the compound in a preventative fashion.

“Therapeutically effective amount” refers to the amount of a compound that, when administered to a patient for treating a disease, or at least one of the clinical symptoms of a disease, is sufficient to treat the disease or symptom thereof. A “therapeutically effective amount” may vary depending, for example, on the compound, the disease and/or symptoms of the disease, severity of the disease and/or symptoms of the disease or disorder, the age, weight, and/or health of the patient to be treated, and the judgment of a prescribing physician. An appropriate amount in any given instance may be ascertained by those skilled in the art or capable of determination by routine experimentation.

“Therapeutically effective dose” refers to a dose that provides effective treatment of a disease or disorder in a patient. A therapeutically effective dose may vary from compound to compound, and from patient to patient, and may depend upon factors such as the condition of the patient and the route of delivery. A therapeutically effective dose may be determined in accordance with routine pharmacological procedures known to those skilled in the art.

“Treating” or “treatment” of a disease refers to arresting or ameliorating a disease or at least one of the clinical symptoms of a disease or disorder, reducing the risk of acquiring a disease or at least one of the clinical symptoms of a disease, reducing the development of a disease or at least one of the clinical symptoms of the disease or reducing the risk of developing a disease or at least one of the clinical symptoms of a disease. “Treating” or “treatment” also refers to inhibiting the disease, either physically, (e.g., stabilization of a discernible symptom), physiologically, (e.g., stabilization of a physical parameter), or both, and to inhibiting at least one physical parameter or manifestation that may or may not be discernible to the patient. In certain embodiments, “treating” or “treatment” refers to delaying the onset of the disease or at least one or more symptoms thereof in a patient who may be exposed to or predisposed to a disease or disorder even though that patient does not yet experience or display symptoms of the disease.

“Tregs” or “Treg cells” refer to regulatory T-cells. Regulatory T-cells are a class of T-cells that suppress the activity of other immune cells and are defined using flow cytometry by the cell marker phenotypes CD4+/CD25+/FOXP3+, CD4+CD25+CD127lo, or CD4+/CD25+/FOXP3+/CD127lo. Because FOXP3 is an intracellular protein and requires cell fixation and permeabilization for staining, the cell surface phenotype CD4+CD25+CD127lo− can be used for defining live Tregs. Tregs also include various Treg subclasses, such as tTregs (thymus-derived) and pTregs (peripherally derived, differentiated from naive T-cells in the periphery). Tregs play a critical role in the induction and maintenance of peripheral self-tolerance to antigens, including those expressed by tumors.

“CD4+ T cells” are a type of lymphocyte that functions to coordinate the immune response by stimulating other immune cells such as macrophages, B lymphocytes (B cells), and CD8+T lymphocytes (CD8+ T cells) to fight infection. CD4+ T cells recognize peptides presented on MHC Class II molecules, which are found on antigen-presenting cells.

As with CD4+ T cells, “CD8+(cytotoxic) T-cells” are generated in the thymus and express the T-cell receptor. Cytotoxic T-cells express a dimeric co-receptor, CD8, which typically comprises one CD8a and one CD80 chain. CD8+ Tcells recognize peptides presented by MHC Class 1 molecules found on most nucleated cells. The CD8 heterodimer binds to a conservative portion of MHC Class 1 during T-cell/antigen-presenting cell interactions. CD8+ T-cells (cytotoxic T lymphocytes, or CTLs) are important for immune defense against intracellular pathogens, including viruses and bacteria, and for tumor surveillance.

“NK (natural killer) cells” are lymphocytes of the innate immune system and are classified as group I innate lymphocytes (ILCs). NK cells respond to a wide variety of pathological challenges, including by killing virally infected cells and detecting and controlling early signs of cancer.

IL-7 mediates a variety of responses in lymphocytes and other immune cell types. Assays for such responses include stimulation of pSTAT5, cell proliferation or markers of proliferation such as Ki67, change in immune cell type ratios, and stimulation of the levels of effector proteins.

“Effector cells” refers to a population of lymphocytes that mediate the helper (CD4+ cells) and cytotoxic (CD8+ and NK cells) effects. Effector cells include effector T-cells such as CD4+ helper T-cells, CD8+ cytotoxic T-cells, NK cells, lymphokine-activated killer (LAK) cells, and macrophages/monocytes.

“Naïve T-cells” refer to a T-cell that has differentiated in bone marrow and undergone the positive and negative processes of central selection in the thymus. Naïve T-cells include naive forms of helper T cells, CD4+ T-cells) and naïve cytotoxic T-cells (CD8+ T-cells). Naïve T-cells are commonly characterized by the surface expression of L-selectin (CD62L) and C—C chemokine receptor type 7 (CCR7) and the expression of IL-7R (CD127) and the absence of the activation markers CD25, CD44, and CD69.

An “immune cell” refers to a cell that plays a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes. Immune cells can comprise T cells or NK cells.

T-cells can include, for example, CD4+ helper T-cells CD8+T-cytotoxic T cell; memory T cells such as (i) stem memory Tscm cells which express CCR7, CD45RA, CD62L (L-selectin), CD2, CD28, IL-7Rα, CD95, IL-2R, CXCR3, and LFA-1; (ii) central memory Tem cells that express CD62L, CCR7 and CD45RO+ and secrete IL-2, but not IFNγ or IL-4, and (iii) effector memory Tem cells that do not express L-selectin or CCR7 but express CD45RO and produce effector cytokines like IFNγ and IL-4; (iv) terminal differentiated effector memory Temra cells that re-express CD45RA but CCR7−; regulatory T-cells such as Tregs, suppressor T cells, or CD4+CD25+ regulatory T cells; natural killer T cells (NKT); and γδT cells. T cells found within tumors are referred to as tumor infiltrating lymphocytes or TILs. A naïve T cell refers to a mature T cell that remains immunologically undifferentiated. Following positive and negative selection in the thymus, T cells emerge as either CD4+ or CD8+ naive T cells. In the naive state, T cells express L-selectin (CD62L), IL-7 receptor-α (IL-7Rα), and CD132, but do not express CD25, CD44, CD69, or CD45RO. I “Immature” can also refer to a T cell which exhibits a phenotype characteristic of either a naive T cell or an immature T cell, such as a Tscm cell or a Tem cell. For example, an immature T cell can express one or more of L-selectin (CD62L), IL-7Rα, CD132, CCR7, CD45RA, CD45RO, CD27, CD28, CD95, CXCR3, and LFA-1. Naive or immature T cells can be contrasted with terminal differentiated effector T cells, such as Teff cells.

“Memory T-cells” are a subset of T lymphocytes including both CD4+ and CD8+. The primary function of memory cells is rapid augmented immune response after reactivation of those cells by reintroduction of a relevant antigen or pathogen into the body. Memory T cells refers to T cells that have previously encountered and responded to a cognate antigen (e.g., in vivo, in vitro, or ex vivo) or which have been stimulated in vitro or ex vivo with an antibody, such as, for example, with an anti-CD3 antibody. Immune cells having a memory-like phenotype upon secondary exposure, such memory T cells can mount a faster and stronger immune response than during the primary exposure. Memory T cells can comprise central memory T cells (Tem cells), effector memory T cells (Tem cells), tissue resident memory T cells (Trm cells), stem cell-like memory T cells (Tscm cells), or any combination thereof.

“Stem cell-like memory T cells,” “T memory stem cells,” or “Tscm cells” refer to memory T cells that express CD95, CD45RA, CCR7, and CD62L and are endowed with the stem cell-like ability to self-renew and the multipotent capacity to reconstitute the entire spectrum of memory and effector T cell subsets.

“Central memory T cells” or “Tem cells” refer to memory T cells that express CD45RO, CCR7, and CD62L. Central memory T cells are generally found within the lymph nodes and in the peripheral circulation.

“Effector memory T cells” or “Tem cells” refer to memory T cells that express CD45RO but lack expression of CCR7 and CD62L. Because effector memory T cells lack lymph node-homing receptors such as CCR7 and CD62L, these cells are typically found in the peripheral circulation and in non-lymphoid tissue.

“Terminally differentiated memory T cells” or “Temra cells” refer to memory T cells express CD45RA but lack expression of CCR7 and CD62L.

“Tissue resident memory T cells” or “TRM cells” refer to memory T cells that do not circulate and remain resident in peripheral tissue, such as skin, lung, and the gastrointestinal tract. Tissue resident memory T cells can also be effector memory T cells.

“Naive T cells” or “Tn cells” refer to T cells that express CD45RA, CCR7, and CD62L, but do not express CD95. The interaction between a Tn cell and an antigen presenting cell (APC) induces differentiation of the Tn cell towards an activated Teff cell and an immune response.

“Cytokine” refers to small, secreted proteins released by cells that have a specific effect on the interactions and communications between cells. Examples of cytokines include interleukins such as interleukin (IL)-1, IL-2, IL-4, IL-7, IL-9, IL-13, IL-15, IL-3, IL-5, IL-6, IL-11, IL-10, IL-20, IL-14, IL-16, IL-17, IL-21 and IL-23, interferons such as IFN; e.g., IFN-α, IFN-β, and IFN-γ), tumor necrosis factors (TNF), and transforming growth factors (TGF).

“Antigen binding moiety” refers to a polypeptide molecule that specifically binds to an antigenic determinant. An antigen binding moiety can direct, for example, the entity to which it is bonded, such as a cytokine or a second antigen binding moiety, to a target site, for example, to a specific type of tumor cell or tumor stroma bearing the antigenic determinant. Antigen binding moieties include antibodies and fragments thereof. Examples of antigen binding moieties include an antigen binding domain of an antibody comprising an antibody heavy chain variable region and an antibody light chain variable region. An antigen binding moiety can include antibody constant regions. Useful heavy chain constant regions can include any of the five isotypes: α, δ, ε, γ, or μ. Useful light chain constant regions can include any of the two isotypes K and A.

“Antibody” in the broadest sense encompasses various antibody structures including, for example, monoclonal antibodies, polyclonal antibodies, multi-specific antibodies such as bispecific antibodies, and antibody fragments that exhibit a desired antigen binding activity. The term “antibody” can be abbreviated as “ab” or “Ab” such as in the expression Fab or anti-phage Ab. Antibody refers to a polypeptide that includes canonical immunoglobulin sequence elements sufficient to confer specific binding to a particular target antigen. Intact antibodies as produced in nature are approximately 150 kD tetrameric agents comprising two identical heavy chain polypeptides (about 50 kD each) and two identical light chain polypeptides (about 25 kD each) that associate with each other into what is commonly referred to as a Y-shaped structure. Each heavy chain comprises at least four domains with each about 110 amino acids long, an amino-terminal variable (VH) domain located at the tips of the Y structure, followed by three constant domains: CHI, CH2, and the carboxy-terminal CH3 located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another switch.

“Full-length antibody,” “intact antibody,” and “whole antibody” refer to an antibody having a structure substantially similar to a native antibody structure or having heavy chains that contain both Fab and an Fc region.

“Antibody fragment” refers to a molecule other than an intact antibody that comprises a portion of an intact antibody that binds the antigen to which the intact antibody binds. Examples of antibody fragments include Fv, Fab, Fab′, Fab′-SH, F(ab′)2, diabodies, linear antibodies, single-chain antibody molecules such as scFv, and multi-specific antibodies formed from antibody fragments. Diabodies are antibody fragments with two antigen-binding sites that may be bivalent or bispecific. Antibody fragments can be made by various techniques, including but not limited to proteolytic digestion of an intact antibody as well as production by recombinant host cells such as E. coli or phage.

“Fab” or “Fab region” refers to a polypeptide that comprises the VH, CHI, VL, and CL immunoglobulin domains, generally on two different polypeptide chains such as VH-CH1 on one chain and VL-CL on the other. Fab may refer to this region in isolation or this region in the context of a bispecific antibody. In the context of a Fab, the Fab comprises an Fv region in addition to the CHI and CL domains.

“Fv” or “Fv fragment” or “Fv region” refers to a polypeptide that comprises the VL and VH domains of an antibody or “Fab”. Fv regions can be formatted as both Fabs (generally two different polypeptides that also include the constant regions) and scFvs, where the vi and vh domains are combined (generally with a linker as discussed) to form an scFv.

“Single chain Fv” or “scFv” refers to a variable heavy domain covalently bound to a variable light domain, generally using a scFv linker as discussed herein, to form a scFv or scFv domain. A scFv domain can be in either orientation with the VL domain at the N- or C-terminus of the polypeptide, and conversely for the VH domain.

“Effector function” refers to a biochemical event that results from the interaction of an antibody Fc region with an Fc receptor or ligand. Effector functions include, for example, antibody-dependent cellular toxicity (ADCC), antibody-dependent cellular phagocytosis (ADCP), and complement-dependent cytotoxicity (CDC).

“Fc” or “Fc region” or “Fe chain” refers to polypeptide comprising the constant region of an antibody, in some instances, excluding all or a portion of the first constant region immunoglobulin domain (e.g., CHI) or a portion thereof, and in some cases, further excluding all or a portion of the hinge. Thus, an Fc can refer to the last two constant region immunoglobulin domains (e.g., CH2 and CH3) of IgA, IgD, and IgG, the last three constant region immunoglobulin domains of IgE and IgM, and optionally, all or a portion of the flexible hinge N-terminal to these domains. For IgA and IgM, Fc may include the J chain. For IgG, the Fc chain comprises immunoglobulin domains CH2 and CH3 (Cy2 and Cy3), and optionally all or a portion of the hinge region between CHI (Cy1) and CH2 (Cy2). Although the boundaries of the Fc region may vary, the hu-IgG heavy chain Fc region is usually defined to include residues E216, C226, or A231 to its carboxyl-terminus, wherein the numbering is according to the EU index as in Kabat. An amino acid modification can be made to the Fc region, for example to alter binding to one or more FcyR or to the FcRn. In EU numbering for hu-IgG1, the CH2-CH3 domain comprises amino acids 231 to 447, and the hinge is 216 to 230. Thus, the definition of Fc chain includes both amino acids 231-447 (CH2-CH3) or 216-447 (hinge-CH2-CH3), or fragments thereof. An Fc fragment can contain fewer amino acids from either or both of the N- and C-termini that retains the ability to form a dimer with another Fc chain or Fc fragment as can be detected using standard methods, generally based on size (e.g., non-denaturing chromatography, size exclusion chromatography, etc.). Hu-IgG Fc chains are of particular use and can be the Fc chain from hu-IgG1, IgG2, or IgG4.

“Heavy constant region” refers to the CH1-hinge-CH2-CH3 portion of an antibody or fragments thereof, excluding the variable heavy domain; in EU numbering of hu-IgG1, such as amino acids 118-447. “Heavy chain constant region fragment” refers to a heavy chain constant region that contains fewer amino acids from either or both of the N- and C-termini that retains the ability to form a dimer with another heavy chain constant region.

“Immunoglobulin” refers to a protein having the structure of a naturally occurring antibody. For example, immunoglobulins of the IgG class are heterotetrameric glycoproteins of about 150,000 Da, composed of two light chains and two heavy chains that are bonded together through disulfide bonds. From N- to C-terminus, each heavy chain has a variable region (VH), also called a variable heavy domain or a heavy chain variable domain, followed by three constant domains (CHI, CH2, and CH3), also called a heavy chain constant region. Similarly, from N- to C-terminus, each light chain has a variable region (VL), also called a variable light domain or a light chain variable domain, followed by a constant light (CL) domain, also called a light chain constant region. The heavy chain of an immunoglobulin may be assigned to one of five classes, called α (IgA), δ (IgD), ε (IgE), γ (IgG), or μ(IgM), some of which may be further divided into subclasses, such as γ1 (IgG1), γ2 (IgG2), γ3 (IgG3), γ4(IgG4), α1 (IgA1) and α2 (IgA2). The light chain of an immunoglobulin may be assigned to one of two types, kappa (κ) or lambda (λ), based on the amino acid sequence of its constant domain. An immunoglobulin essentially consists of two Fab molecules and an Fc chain, linked via the immunoglobulin hinge region.

“Linker” refers to a moiety that binds one compound to another compound. Linkers can include IL-7Rαγc linkers and tandem IL-7Rαγc linkers. A linker can be a synthetic linker. A linker can be an amino acid linker. In general, linkers provided by the present disclosure facilitate the ability of an IL-7Rαγc agonist peptide to interact with IL-7R, to bind to IL-7R with high affinity, and/or to activate IL-7R. A linker can comprise a peptide or a non-peptide. Non-peptide linkers include those containing, for example, a triazole moiety derived from a Cu(I) catalyzed reaction of alkyne and azide functionalities. An IL-7Rαγc linker refers to a moiety that binds at least one IL-7R ligand such as an IL-7Rα ligand and/or an Rγc ligand to another IL-7R ligand. For example, a ligand linker can bind an IL-7Rα ligand to an Rγc ligand. A linker can bind to another IL-7R ligand which can be the same IL-7R ligand or a different IL-7R ligand. A linker can also bind to one or more additional moieties that provide a desired physiological function. A linker can be divalent or multivalent. A linker can be hydrolytically stable or may include a physiologically hydrolyzable or enzymatically degradable or cleavable linkage. A linker can bind IL-7R ligands to form dimers, trimers, or higher order multi-ligand peptides (heteromers) and compounds. A linker can also bind to one or more additional moieties that provide a desired physiological function. For example, a construct linker can bind an IL-2Rβγc peptide agonist or an IL-7Rαγc agonist to a construct partner such as an antibody or an antibody fragment. A linker can be divalent or multivalent.

A linker can be divalent or multivalent. A ligand linker can be hydrolytically stable or can include a physiologically hydrolyzable or enzymatically degradable linkage. A linker can bind IL-7Rα ligands and/or Rγc ligands to form dimers, trimers, or higher order multi-ligand peptides (heteromers) and compounds. A linker can be a peptidyl linker or a chemical linker.

A “flexible linker” refers to a peptidyl linker comprising flexible amino acids such as glycine and serine. A flexible linker can comprise, for example, from 1 to 100 amino acids such as from 1 to 50, from 1 to 40, from 1 to 30, from 1 to 20, from 1 to 10, or from 1 to 5 amino acids, where each amino acid is independently selected from glycine and serine. Examples of flexible linkers include (G)ₙ (SEQ ID NO: 2), (GS)ₙ (SEQ ID NO: 3), (GGS)ₙ (SEQ ID NO: 4), (GGGS)ₙ (SEQ ID NO: 5), or (GGGGS)ₙ (SEQ ID NO: 6) where n can be an integer from 1 to 20; (G)ₙ (SEQ ID NO: 7), (GS)ₙ (SEQ ID NO: 8), (GGS)ₙ (SEQ ID NO: 9), (GGGS)ₙ (SEQ ID NO: 10), or (GGGGS)ₙ (SEQ ID NO: 11) where n can be an integer from 1 to 10; or (G)ₙ (SEQ ID NO: 12), (GS)ₙ (SEQ ID NO: 13), (GGS)ₙ (SEQ ID NO: 14), (GGGS)ₙ (SEQ ID NO: 15), or (GGGGS)ₙ (SEQ ID NO: 16) where n can be an integer from 1 to 5. A flexible linker can have the amino acid sequence, for example, (GGGGS) (SEQ ID NO: 17), (GGGGS)₂ (SEQ ID NO: 18), (GGGGS)₃ (SEQ ID NO: 19), (GGGGS)₄ (SEQ ID NO: 20), (GG) (SEQ ID NO: 21), (GGG) (SEQ ID NO: 22), (GGGG) (SEQ ID NO: 23), (GGS) (SEQ ID NO: 24), (GGGS) (SEQ ID NO: 25), (GGGGSGG) (SEQ ID NO: 26), (GGS)₂ (SEQ ID NO: 27), (G)₅ (SEQ ID NO: 28), or (GS)₁₀ (SEQ ID NO: 29).

A “rigid linker” refers to a peptidyl linker that is proline rich and can include other amino acids such as alanine, lysine, and/or glutamic acid. A rigid linker can comprise, for example, from 1 to 100 amino acids such as from 1 to 50, from 1 to 40, from 1 to 30, from 1 to 20, from 1 to 10, or from 1 to 5 amino acids, where each amino acid is independently selected from proline, alanine, lysine, and glutamic acid. A rigid linker can comprise, for example, from 1 to 100 amino acids such as from 1 to 50, from 1 to 40, from 1 to 30, from 1 to 20, from 1 to 10, or from 1 to 5 amino acids, where each amino acid is independently selected from proline and alanine. A rigid linker can have the sequence (P)ₙ (SEQ ID NO: 30) or (PA)ₙ (SEQ ID NO: 31), where n is an integer from 1 to 20. A rigid linker can have the sequence (P)ₙ (SEQ ID NO: 32) or (PA)ₙ (SEQ ID NO: 33), where n is an integer from 1 to 10. A rigid linker can have the sequence (P)ₙ (SEQ ID NO: 34) or (PA)ₙ (SEQ ID NO: 35), where n is an integer from 1 to 5. A rigid linker can have, for example, the sequence (PA)₅ (SEQ ID NO: 36), (PA)₆ (SEQ ID NO: 40), (PA)₇ (SEQ ID NO: 37), (PA)₈ (SEQ ID NO: 41), (PA)₉ (SEQ ID NO: 42), or (PA)₁₀ (SEQ ID NO: 38).

“Percent (%) sequence similarity” is determined by comparing the number of amino acids that are the same in a subject peptide and a reference peptide. A peptide provided by the present disclosure can comprise, for example, greater than 60% sequence similarity, greater than 70%, greater than 80%, or greater than 90% sequence similarity to a reference peptide. For example, based on a reference peptide having SEQ ID NO: 352, peptides having SEQ ID NO: 353-358, have either 1, 2, 3, 4, or 5 amino acids in which an amino acid of the reference peptide has been substituted or replaced with the amino acid, alanine. Peptides having SEQ ID NO: 353-358 are characterized by a 95%, 90%, 85%, 80%, 75%, or 70% sequence similarity, respectively, to the amino acid sequence of the reference peptide having SEQ ID NO: 352.

SEQ ID NO: 352
Y P C W L A R V G E L C D L D S G D V H
SEQ ID NO: 353
A P C W L A R V G E L C D L D S G D V H
SEQ ID NO: 354
A P C A L A R V G E L C D L D S G D V H
SEQ ID NO: 355
A P C A L A A V G E L C D L D S G D V H
SEQ ID NO: 356
A P C A L A A V G A L C D L D S G D V H
SEQ ID NO: 357
A P C A L A A V G A L C D L A S G D V H
SEQ ID NO: 358
A P C A L A A V G A L C D L A A G D V H

A peptide can have an amino acid sequence in which, for example, from 1 to 10 or from 1 to 5 amino acids of a reference amino acid sequence is substituted with another amino acid.

For example, a peptide derived from a reference peptide can have from 1 to 10 amino acid substitutions, from 1 to 5 amino acid substitutions, from 1 to 4, from 1 to 3, or from 1 to 2 amino acid substitutions. For example, a peptide derived from a reference peptide can have 1 amino acid substitution, 2 amino acid substitutions, 3 amino acid substitutions, 4 amino acid substitutions, 5 amino acid substitutions, 6 amino acid substitutions, 7 amino acid substitutions, 8 amino acid substitutions, 9 amino acid substitutions, or 10 amino acid substitutions.

An amino acid substitution can be independent of the other amino acid substitutions.

Each amino acid substitution can independently be a conservative amino acid substitution or a non-conservative amino acid substitution.

A conservative amino acid substitution refers to one of the following amino acid substitutions: amino acids having a small hydrophobic side chain comprising alanine (A), glycine (G), proline (P), serine (S), or threonine (T); amino acids having a hydroxyl-containing side chain comprising serine (S), threonine (T), or tyrosine (Y); amino acids having an acidic side chain comprising aspartate (D) or glutamate (E); amino acids having a polar-neutral side chain comprising histidine (H), asparagine (N), glutamine (Q), serine (S), threonine (T), or tyrosine (Y); amino acids having a basic side chain comprising arginine (R), lysine (K), or histidine (H); amino acids having a large hydrophobic side chain comprising isoleucine (I), leucine (L), methionine (M), valine (V), phenylalanine (F), tyrosine (Y), or tryptophan (W); and amino acids having an aromatic side chain comprising phenylalanine (F), histidine (H), tryptophan (W), or tyrosine (Y).

For example, a reference peptide can have the amino acid sequence of SEQ ID NO: 346.

SEQ ID NO: 346
Y W C W M A Q V G E L C D L
SEQ ID NO: 347
Y H C W M A Q V G E L C D L
SEQ ID NO: 348
Y H C W M G Q V G E L C D L
SEQ ID NO: 349
Y H C W M G Q M G E L C D L
SEQ ID NO: 350
Y H C W M G Q M G E L C E L
SEQ ID NO: 351
Y H C W M G Q M G E L C E M

Peptides having SEQ ID NO: 347-351 represent peptides in which the reference peptide having SEQ ID NO: 346 has been substituted with from 1 to 5 conservative amino acid substitutions, respectively.

A peptide can comprise a truncated peptide. A truncated peptide refers to a peptide in which, for example, from 1 to 5 amino acids have independently been removed from the N-terminus, the C-terminus, or from both the N-terminus and the C-terminus of the corresponding reference peptide. A truncated peptide derived from a corresponding reference peptide can independently have from 1 to 5 amino acids, such as from 1 to 4 amino acids, from 1 to 3 amino acids, or from 1 to 2 amino acids independently removed from the N-terminus, the C-terminus, or from both the N-terminus and the C-terminus of the reference peptide. A truncated peptide derived from a corresponding reference peptide can independently have, for example, 1 amino acid, 2 amino acids, 3 amino acids, 4 amino acids, or 5 amino acids removed from the N-terminus, the C-terminus, or from both the N-terminus and the C-terminus of the reference peptide.

For example, a reference peptide can have the amino acid sequence of SEQ ID NO: 328. Examples of truncated peptides derived from the reference peptide having SEQ ID NO: 328 include truncated peptides having the amino acid sequence of SEQ ID NO: 329-336.

SEQ ID NO: 328
M G F Y P C W T A Q L G E L C D L S V D
SEQ ID NO: 329
G F Y P C W T A Q L G E L C D L S V D
SEQ ID NO: 330
F Y P C W T A Q L G E L C D L S V D
SEQ ID NO: 331
Y P C W T A Q L G E L C D L S V D
SEQ ID NO: 332
M G F Y P C W T A Q L G E L C D L S V
SEQ ID NO: 333
M G F Y P C W T A Q L G E L C D L S
SEQ ID NO: 334
M G F Y P C W T A Q L G E L C D L
SEQ ID NO: 335
G F Y P C W T A Q L G E L C D L S V
SEQ ID NO: 336
F Y P C W T A Q L G E L C D L

The truncated peptides of SEQ ID NO: 329-331 have amino acids removed from the N-terminus of the reference peptide; truncated peptides of SEQ ID NO: 332-334 have amino acids removed from the C-terminus of the reference peptide; and truncated peptides of SEQ ID NO: 335-336 have amino acids removed from both the N-terminus and from the C-terminus of the reference peptide.

As another example, a reference peptide can comprise an amino acid sequence of Formula (A):

—X⁵⁰⁰—X⁵⁰¹—C—X⁵⁰²—X⁵⁰³—X⁵⁰⁴—X⁵⁰⁵—X⁵⁰⁶—X⁵⁰⁷—X⁵⁰⁸—X⁵⁰⁹—C—X⁵¹⁰—X⁵¹¹— (A)

where each —X— independently represents an amino acid. Amino acid sequences of Formula (A1)-(A5) represent truncated peptides derived from the reference peptide comprising the amino acid sequence of Formula (A):

—X⁵⁰¹—C—X⁵⁰²—X⁵⁰³—X⁵⁰⁴—X⁵⁰⁵—X⁵⁰⁶—X⁵⁰⁷—X⁵⁰⁸—X⁵⁰⁹—C—X⁵¹⁰—X⁵¹¹— (A1)

—C—X⁵⁰²—X⁵⁰³—X⁵⁰⁴—X⁵⁰⁵—X⁵⁰⁶—X⁵⁰⁷—X⁵⁰⁸—X⁵⁰⁹—C—X⁵¹⁰—X⁵¹¹— (A2)

—C—X⁵⁰²—X⁵⁰³—X⁵⁰⁴—X⁵⁰⁵—X⁵⁰⁶—X⁵⁰⁷—X⁵⁰⁸—X⁵⁹—C— (A3)

—X⁵⁰²—X⁵⁰³—X⁵⁰⁴—X⁵⁰⁵—X⁵⁰⁶—X⁵⁰⁷—X⁵⁰⁸—X⁵⁰⁹—C—X⁵¹⁰— (A4)

—X⁵⁰²—X⁵⁰³—X⁵⁰⁴—X⁵⁰⁵—X⁵⁰⁶—X⁵⁰⁷—X⁵⁰⁸—X⁵⁰⁹— (A5)

A peptide provided by the present disclosure can comprise an amino acid sequence in which, for example, from 1 to 3 glycines are independently bonded to the N-terminus, to the C-terminus, or to both the N-terminus and to the C-terminus of a reference peptide.

For example, a reference peptide can have SEQ ID NO: 337. Peptides having SEQ ID NO: 338-340 include from 1 to 3 glycines bonded to the N-terminus of the reference peptide, respectively; peptides having SEQ ID NO: 341-343 include from 1 to 3 glycines bonded to the C-terminus of the reference peptide, respectively; and peptides having SEQ ID NO: 344-345 independently include 1 or 2 glycines (SEQ ID NO: 21) bonded to both the N-terminus and to the C-terminus of the reference peptide having SEQ ID NO 337

| | |
|---|---|
| SEQ ID NO: 337 | K Y C G F A Q L G E L C V L |
| SEQ ID NO: 338 | G K Y C G F A Q L G E L C V L |
| SEQ ID NO: 339 | G G K Y C G F A Q L G E L C V L |
| SEQ ID NO: 340 | G G G K Y C G F A Q L G E L C V L |
| SEQ ID NO: 341 | K Y C G F A Q L G E L C V L G |
| SEQ ID NO: 342 | K Y C G F A Q L G E L C V L G G |
| SEQ ID NO: 343 | K Y C G F A Q L G E L C V L G G G |
| SEQ ID NO: 344 | G K Y C G F A Q L G E L C V L G |
| SEQ ID NO: 345 | G G K Y C G F A Q L G E L C V L G |

An IL-7Rαγc agonist peptide can comprise a truncated peptide in which, for example, from 1 to 3 glycines are independently bonded to the N-terminus, to the C-terminus, or to both the N-terminus and to the C-terminus of a reference truncated peptide.

“IL-7R”, “IL-7Rα subunit”, and “IL-7Rγc subunit” refer to a mammalian “IL-7R”, “IL-7Rα subunit”, and “IL-7Rγc subunit” respectively, such as the hu-IL-7R, the hu-IL-7Rα subunit, and the hu-IL-7Rγc subunit, respectively.

An Il-2Rγc ligand is the same as an IL-7Rγc ligand and can be referred to as an Rγc ligand.

“Concentration of the target immune cell population” refers to the number of cells per milliliter of blood or culture medium, where the number of cells can be determined using flow cytometry.

“Target immune cell population” refers to a population or subpopulation of immune cells having a specific, desired phenotype. A target immune cell population can comprise a single subpopulation of immune cells. A target immune cell population can comprise two or more subpopulations of immune cells where each of the subpopulations of immune cells has a different phenotype.

“IL-2Rβγc agonist peptide” refers to a peptide that binds to the hu-IL-2R with an IC50 less than 100 μM, as determined using phage display ELISA assays. An IL-2Rβγc agonist peptide can exhibit an EC50 for STAT5 phosphorylation in hu-PBMCs and in TF-1-7α cells of less than 100 μM. An IL-2Rβγc agonist peptide can activate the STAT5 phosphorylation pathway, the AKT phosphorylation pathway, and the ERK1/2 phosphorylation pathway in CD4+ and CD8+ cells. An IL-2Rβγc can comprise an amino acid sequence of any one of SEQ ID NO: 100-117, 118-193, 53-245, and 43-46.

“IL-15Rβγc agonist peptide” refers to a peptide that binds to the hu-IL-15R with an IC₅₀ less than 100 μM, as determined using phage display ELISA assays. An IL-15Rβγc agonist peptide can exhibit an EC₅₀ for STAT5 phosphorylation in hu-PBMCs and in TF-1-7α cells of less than 100 μM. An IL-15Rβγc agonist peptide can activate the STAT5 phosphorylation pathway, the AKT phosphorylation pathway, and the ERK1/2 phosphorylation pathway in CD4+ and CD8+ cells.

“Anti-CD3 monoclonal antibody” refers to a monoclonal antibody that binds to the CD3 antigen, a human glycoprotein that is primarily expressed on T cells, certain NKT cells, and thymocytes during T cell differentiation. Binding of an anti-CD3 monoclonal antibody to CD3 stimulates T cell activation. Anti-CD3 monoclonal antibodies can be used to expand T cells in culture.

“Anti-CD28 monoclonal antibody” refers to a monoclonal antibody that binds to the CD80 and CD86 antigen. CD28 is expressed on most T cell lineages, NK cell subsets, and plasma cells. CD28 binding induces T cell activation, IL-2 synthesis, and prevents cell death. In vitro, CD28 binding to T cells provides a costimulatory signal required for T cell activation and proliferation.

An “enriched immune cell population provided by the present disclosure” refers to a population of immune cells, a subpopulation of immune cells, a combination of subpopulations of immune cells that are produced using a culture medium provided by the present disclosure, an immobilized IL-7Rαγc agonist peptide provided by the present disclosure, a method of enrichment provided by the present disclosure, a method of manufacturing provided by the present disclosure or a combination of any of the foregoing. An enriched immune cell population has a higher cell count per volume of a population or subpopulation of immune cells than the cell count per volume of the population or subpopulation of immune cells in the initial source and before expansion of a targeted immune cell population.

The expression “at least one” refers to “one or more.” For example, the expression “at least one” can refer to from 1 to 10, from 1 to 8, from 1 to 6, from 1 to 5, from 1 to 4, from 1 to 3, or from 1 to 2. For example, the expression “at least one” can refer to 1, 2, 3, 4, 5, 6, 7, 8, 9, or 10.

Reference is now made in detail to certain embodiments of compounds, compositions, and methods. The disclosed embodiments are not intended to be limiting of the claims. On the contrary, the claims are intended to cover all alternatives, modifications, and equivalents.

Interleukin-7 (IL-7) is required for development and maintenance of T-cell homeostasis and plays an important role in the establishment of the B-cell repertoire. Unlike most interleukins, IL-7 is primarily produced by non-hematopoietic stromal cells rather than leukocytes. Under normal conditions, free IL-7 levels are limiting, but accumulate during lymphopenia, leading to increased T cell proliferation and replenishment of T-cell populations. Under certain physiological conditions, recombinant human IL-7 administered to humans, non-human primates, and mice produces widespread T cell proliferation, increased T cell numbers, modulation of peripheral T cell subsets and increased T cell receptor repertoire diversity. These effects may be therapeutically useful in a variety of clinical settings.

IL-7 is a member of the common γ chain (γc, CD132) family of cytokines that includes interleukin-2 (IL-2), IL-4, IL-7, IL-9, IL-15, and IL-21. IL-7 signals via an active complex formed with its unique α-receptor, IL-7Rα (CD127), and the common γc receptor (Rγc). Receptor activation leads to signaling through an array of pathways, including JAK-STAT, P13K-AKT, and Src kinases.

The IL-7Rα receptor subunit exists in two states: a full-length membrane-bound form that, with Rγc, mediates IL-7R signal transduction; and soluble (alternatively-spliced, secreted, or shed) forms of the extracellular domain that may provide regulation of extracellular IL-7 levels and modulation of IL-7R signaling.

The cell surface signaling-competent form of IL-7Rα is expressed on most resting T-cells and is down regulated upon T-cell activation, while naïve memory T-cells continue to express IL-7Rα; and regulatory cells typically express very low levels of IL-7Rα. IL-7R signaling is necessary for long-term maintenance of T cell populations, in part by modulating apoptosis. Both CD4+ and CD8+ memory T-cells are dependent on IL-7 for long-term survival.

Emerging evidence suggests IL-7R agonists may be useful in immuno-oncology therapy. For example, IL-7 is effective in increasing cytotoxic CD8+T lymphocytes (CD8+ T-cell), and long-term tumor antigen specific CD8+ T-cell responses are enhanced by IL-7 treatment.

IL-7 exhibits inhibitory effects in tumors such as glioma, melanoma, lymphoma, leukemia, prostate cancer, and glioblastoma; and administration of IL-7 in murine tumor models has shown decreased cancer cell growth. IL-7 has been shown to enhance the antitumor effect of interferon-γ (IFNγ) in rat glioma tumors, and can induce the production of IL-1α, IL-1β, and TNF-α by monocytes, which can inhibit tumor growth.

IL-7 has been shown to have potential in the treatment of lymphopenias, septic shock, and infectious disease as well as immune deficiencies of aging (immuno-senescence), and enhancement of response to vaccination. IL-7 prevents or reverses T-cell exhaustion and induces rejuvenation and increased activity of transferred CAR-T cells. IL-7 is currently being studied to prevent or reverse lymphopenia associated with COVID-19. IL-7/IL-7R signaling has also been implicated in autoimmune, chronic inflammatory diseases, and cancer, and therefore therapeutic targeting of the IL-7/IL-7R pathway is expected to have clinical benefit.

The present disclosure is directed to IL-7Rαγc agonist peptides, culture media comprising the IL-7Rαγc agonist peptides, and to methods of expanding a selected immune cell population or subpopulation and methods of manufacturing a selected immune cell population or subpopulation using IL-7Rαγc agonist peptides.

An IL-7Rαγc agonist peptide refers to a peptide comprising an IL-7Rα ligand and an Rγc ligand, where the IL-7Rαγc agonist peptide has an EC₅₀ for STAT5 phosphorylation of TF-1-7α cells of less than 100 μM, less than 10 μM, less than 1 μM, less than 100 μM, less than 10 μM, or less than 1 μM.

An IL-7Rαγc agonist peptide can comprise an IL-7Rα ligand, an Rγc ligand, and an IL-7Rαγc linker coupling the IL-7Rα and Rγc ligands. An IL-7Rαγc agonist peptide can be an IL-7R agonist, or a partial IL-7R agonist.

An IL-7Rαγc agonist peptide can be capable of binding to a unique binding site on the IL-7Rα subunit and/or the IL-7Rγc subunit and bind to the unique binding site with an IC₅₀, for example, of less than 100 μM, less than 10 μM, less than 1 μM, less than 100 nM, less than 10 nM, or less than 1 nM.

An IL-7Rαγc agonist peptide can comprise at least one IL-7Rα ligand. Examples of suitable IL-7Rα ligands are disclosed in U.S. Pat. No. 11,254,729, which is incorporated by reference in its entirety.

An IL-7Rα ligand can bind to the hu-IL-7Rα subunit with an IC₅₀, for example, of less than 100 μM, less than 10 μM, less than 1 μM, less than 0.1 μM, or less than 0.01 μM.

An IL-7Rα ligand can bind to the hu-IL-7Rα subunit with an IC₅₀, for example, from 1 μM to 100 μM, from 10 μM to 10 μM, from 100 μM to 1 μM, from 0.001 μM to 1 μM, or from 0.01 μM to 1 μM.

An IL-7Rα ligand can bind to a mammalian IL-7Rα subunit with an IC₅₀, for example, of less than 100 μM, less than 10 μM, less than 1 μM, less than 0.1 μM, or less than 0.01 μM.

An IL-7Rα ligand can bind to a mammalian IL-7Rα subunit with an IC₅₀, for example, from 1 μM to 100 μM, from 10 μM to 10 μM, from 100 μM to 1 μM, from 0.001 μM to 1 μM, or from 0.01 μM to 1 μM.

An IL-7Rα ligand can comprise an amino acid sequence of Formula (1) (SEQ ID NO: 43), an amino acid sequence of Formula (1a) (SEQ ID NO: 44), an amino acid sequence of Formula (1b) (SEQ ID NO: 45), or an amino acid sequence of Formula (1c) (SEQ ID NO: 46):

—X²⁰¹—X²⁰²—X²⁰³—X²⁰⁴—X²⁰⁵—X²⁰⁶—X²⁰⁷—X²⁰⁸—X²⁰⁹—X²¹⁰—X²¹¹—X²¹²—X²¹³—X²¹⁴—X²¹⁵—X²¹⁶— (1)

—X²⁰²—X²⁰³—X²⁰⁴—X²⁰⁵—X²⁰⁶—X²⁰⁷—X²⁰⁸—X²⁰⁹—X²¹⁰—X²¹¹—X²¹²—X²¹³—X²¹⁴—X²¹⁵— (1a)

—X²⁰³—X²⁰⁴—X²⁰⁵—X²⁰⁶—X²⁰⁷—X²⁰⁸—X²⁰⁹—X²¹⁰—X²¹¹—X²¹²—X²¹³—X²¹⁴— (1b)

—X²⁰⁴—X²⁰⁵—X²⁰⁶—X²⁰⁷—X²⁰⁸—X²⁰⁹—X²¹⁰—X²¹¹—X²¹²—X²¹³— (1c)

wherein each of X²⁰¹ to X²¹⁶ independently comprises an amino acid.

In an IL-Ra ligand of any one of Formula (1)-(1c),
X²⁰¹ can be selected from an amino acid comprising a large hydrophobic side chain;
X²⁰² can be selected from an amino acid comprising a small hydrophobic side chain or cysteine;
X²⁰³ can be selected from an amino acid comprising a large hydrophobic side chain;
X²⁰⁴ can be selected from an amino acid comprising a basic side chain or cysteine;
X²⁰⁵ can be selected from an amino acid comprising a large hydrophobic side chain or an amino acid comprising small hydrophobic side chain;
X²⁰⁶ can be selected from an amino acid comprising a large hydrophobic side chain or an amino acid comprising an acidic side chain;
X²⁰⁷ can be selected from an amino acid comprising an acidic side chain;
X²⁰⁸ can be selected from an amino acid comprising an acidic side chain or an amino acid comprising a small hydrophobic side chain;
X²⁰⁹ can be selected from an amino acid comprising a small hydrophobic side chain;
X²¹⁰ can be selected from an amino acid comprising a large hydrophobic side chain or an amino acid comprising a small hydrophobic side chain;
X²¹¹ can be selected from an amino acid comprising a large hydrophobic side chain;
X²¹² can be selected from an amino acid comprising a polar/neutral side chain;
X²¹³ can be selected from cysteine;
X²¹⁴ can be selected from an amino acid comprising a small hydrophobic side chain or an amino acid comprising a large hydrophobic side chain;
X²¹⁵ can be selected from an amino acid comprising a large hydrophobic side chain; and
X²¹⁶ can be selected from an amino acid comprising a large hydrophobic side chain.

In IL-7Rα ligands of Formula (1), X²⁰¹ can be selected from H, I, Q, and V.

In IL-7Rα ligands of Formula (1), X²⁰¹ can be selected from I, Q, and V.

In IL-7Rα ligands of Formula (1), X²⁰¹ can be I.

In IL-7Rα ligands of Formula (1)-(1a), X²⁰² can be selected from C, P, and R.

In IL-7Rα ligands of Formula (1)-(1a), X²⁰² can be selected from C and P.

In IL-7Rα ligands of Formula (1)-(1b), X²⁰³ can be selected from I, K, L, S, V, and W.

In IL-7Rα ligands of Formula (1)-(1b), X²⁰³ can be W.

In IL-7Rα ligands of Formula (1)-(1c), X²⁰⁴ can be selected from C and H.

In IL-7Rα ligands of Formula (1)-(1c), X²⁰⁴ can be C.

In IL-7Rα ligands of Formula (1)-(1c), X²⁰⁵ can be selected from A, I, L, M, T, and W.

In IL-7Rα ligands of Formula (1)-(1c), X²⁰⁵ can be selected from T and W.

In IL-7Rα ligands of Formula (1)-(1c), X²⁰⁶ can be selected from D, L, and W.

In IL-7Rα ligands of Formula (1)-(1c), X²⁰⁶ can be selected from D and L.

In IL-7Rα ligands of Formula (1)-(1c), X²⁰⁷ can be selected from D, I, L, and Q.

In IL-7Rα ligands of Formula (1)-(1c), X²⁰⁷ can be selected from D and L.

In IL-7Rα ligands of Formula (1)-(1c), X²⁰⁷ can be D.

In IL-7Rα ligands of Formula (1)-(1c), X²⁰⁸ can be selected from D, E, and P.

In IL-7Rα ligands of Formula (1)-(1c), X²⁰⁸ can be selected from E and P.

In IL-7Rα ligands of Formula (1)-(1c), X²⁰⁸ can be P.

In IL-7Rα ligands of Formula (1)-(1c), X²⁰⁹ can be selected from G, S, and T.

In IL-7Rα ligands of Formula (1)-(1c), X²⁰⁹ can be selected from G and S.

In IL-7Rα ligands of Formula (1)-(1c), X²⁰⁹ can be G.

In IL-7Rα ligands of Formula (1)-(1c), X²¹⁰ can be selected from A, G, L, and S.

In IL-7Rα ligands of Formula (1)-(1c), X²¹⁰ can be selected from L and S.

In IL-7Rα ligands of Formula (1)-(1c), X²¹¹ can be selected from F, I, L, and M.

In IL-7Rα ligands of Formula (1)-(1c), X²¹¹ can be L.

In IL-7Rα ligands of Formula (1)-(1c), X²¹² can be selected from G, H, L, N, Q, and S.

In IL-7Rα ligands of Formula (1)-(1c), X²¹² can be selected from Q and S.

In IL-7Rα ligands of Formula (1)-(1c), X²¹² can be Q.

In IL-7Rα ligands of Formula (1)-(1c), X²¹³ can be C.

In IL-7Rα ligands of Formula (1)-(1b), X²¹⁴ can be selected from A, E, I, L, S, T, and V.

In IL-7Rα ligands of Formula (1)-(1b), X²¹⁴ can be selected from A and V.

In IL-7Rα ligands of Formula (1)-(1a), X²¹⁵ can be selected from F, R, W, and Y.

In IL-7Rα ligands of Formula (1)-(1a), X²¹⁵ can be W.

In IL-7Rα ligands of Formula (1), X²¹⁶ can be selected from E, L, Q, and W.

In IL-7Rα ligands of Formula (1), X²¹⁶ can be L.

In IL-7Rα ligands of Formula (1), the IL-7Rα ligand can be defined by any combination of X²⁰¹—X²¹⁶ as defined in the immediately preceding thirty-five (35) paragraphs.

In IL-7Rα ligands of Formula (1)-(1c),
X²⁰¹ can be selected from H, I, Q, and V;
X²⁰² can be selected from C, P, and R;
X²⁰³ can be selected from I, K, L, S, V, and W;
X²⁰⁴ can be selected from C and H;
X²⁰⁵ can be selected from A, I, L, M, T, and W;
X²⁰⁶ can be selected from D, L, and W;
X²⁰⁷ can be selected from D, I, L, and Q;
X²⁰⁸ can be selected from D, E, and P;
X²⁰⁹ can be selected from G, S, and T;
X²¹⁰ can be selected from A, G, L, and S;
X²¹ can be selected from F, I, L, and M;
X²¹² can be selected from G, H, L, N, Q, and S;
X²¹³ can be C;
X²¹⁴ can be selected from A, E, I, L, S, T, and V;
X²¹⁵ can be selected from F, R, W, and Y; and
X²¹⁶ can be selected from E, L, Q, and W.

In IL-7Rα ligands of Formula (1)-(1c),
X²⁰¹ can be selected from I, Q, and V;
X²⁰² can be selected from C and P;
X²⁰³ can be W;
X²⁰⁴ can be selected from C and H;
X²⁰⁵ can be selected from T and W;
X²⁰⁶ can be selected from D and L;
X²⁰⁷ can be selected from D and L;
X²⁰⁸ can be selected from E and P;
X²⁰⁹ can be selected from G and S;
X²¹⁰ can be selected from L and S;
X²¹¹ can be L;
X²¹² can be selected from Q and S;
X²¹³ can be C;
X²¹⁴ can be selected from A and V;
X²¹⁵ can be W; and
X²¹⁶ can be L.

In IL-7Rα ligands of Formula (1)-(1c),
X²⁰¹ can be I;
X²⁰² can be selected from C and P;
X²⁰³ can be W;
X²⁰⁴ can be selected from C and H;
X²⁰⁵ can be selected from T and W;
X²⁰⁶ can be selected from D and L;
X²⁰⁷ can be D;
X²⁰⁸ can be P;
X²⁰⁹ can be G;
X²¹⁰ can be selected from L and S;
X²¹¹ can be L;
X²¹² can be Q;
X²¹³ can be C;
X²¹⁴ can be selected from A and V;
X²¹⁵ can be W; and
X²¹⁶ can be L.

In IL-7Rα ligands of Formula (1)-(1c),
X²⁰¹ can be Q;
X²⁰² can be C;
X²⁰³ can be selected from I, L, K, and V;
X²⁰⁴ can be H;
X²⁰⁵ can be W;
X²⁰⁶ can be D;
X²⁰⁷ can be selected from I and L;
X²⁰⁸ can be E;
X²⁰⁹ can be selected from S and T;
X²¹⁰ can be L;
X²¹¹ can be L;
X²¹² can be selected from G, L, N, and S;
X²¹³ can be C;
X²¹⁴ can be selected from I, L, and V;
X²¹⁵ can be R; and
X²¹⁶ can be E.

In IL-7Rα ligands of Formula (1)-(1c),
X²⁰¹ can be selected from I and V;
X²⁰² can be P;
X²⁰³ can be W;
X²⁰⁴ can be C;
X²⁰⁵ can be T;
X²⁰⁶ can be L;
X²⁰⁷ can be D;
X²⁰⁸ can be P;
X²⁰⁹ can be G;
X²¹⁰ can be selected from L and S;
X²¹¹ can be L;
X²¹² can be Q;
X²¹³ can be C;
X²¹⁵ can be A;
X²¹⁵ can be W; and
X²¹⁶ can be L.

An IL-7Rα ligand can comprise an amino acid sequence or a truncated amino acid sequence selected from any one of SEQ ID NO: 100 to SEQ ID NO: 117:

SEQ ID NO: 100
H C L H W N I E T L M S C V Y G N F E E
SEQ ID NO: 101
H C K H W D L E S L L L C V
SEQ ID NO: 102
H L G V P W C T L D P G S I Q C A W L A K H
SEQ ID NO: 103
I R S C L W Q P G A L H C T W W A E E E P V
SEQ ID NO: 104
I P W C L L D P G G L Q C V W L
SEQ ID NO: 105
K A G S W F I P W C T L D P G S L Q C A F L
SEQ ID NO: 106
N P F R S V V P W C A L D P G S L Q C A W L
SEQ ID NO: 107
Q C I H W D I E T L L S C V
SEQ ID NO: 108
Q C I H W D L E S L L N C L R E L K E P
SEQ ID NO: 109
Q C V H W D L D T L F G C I R E Q L E L
SEQ ID NO: 110
R H F D D I I P W C T L D P G S L Q C A Y L
SEQ ID NO: 111
S A K V L K Q C L H W D L E S L L S C L
SEQ ID NO: 112
S L T V P W C T L D P G S M Q C A W L Q N R
SEQ ID NO: 113
V P W C M L D P G S M Q C A W L
SEQ ID NO: 114
V H R I P W C T L D P G G L Q C A W L R Q M
SEQ ID NO: 115
W V T I P W C I L D P G S L Q C E W Q T K V
SEQ ID NO: 116
G W G I P W C T LD P G S L Q C A W L G K H
SEQ ID NO: 117
Y R S G H G I P W C M L D P G G L Q C S W L

An IL-7Rα ligand can comprise a truncated amino acid sequence of any one of SEQ ID NO: 100-117 and 43-46.

An IL-7Rα ligand can comprise an amino acid sequence selected from any one of SEQ ID NO: 100-117 and 43-46, or a truncated amino acid sequence of any one SEQ ID NO: 100-117 and 43-46, wherein the amino acid sequence can independently comprise from 1 to 5 glycines (G) (SEQ ID NO: 12) on the N-terminus, on the C-terminus, or on both the N- and C-termini.

An IL-7Rα ligand can comprise an amino acid sequence selected from any one of SEQ ID NO: 100-117 and 43-46, or a truncated amino acid sequence of any one of SEQ ID NO: 100-117 and 43-46, wherein the amino acid sequence comprises one or more amino acid substitutions such as from 1 to 5 amino acid substitutions. Each amino acid substitution can independently comprise a conservative amino acid substitution or a non-conservative amino acid substitution.

An IL-7Rα ligand can comprise an amino acid sequence similarity greater than 60%, greater than 70%, greater than 75%, greater than 80%, greater than 85%, greater than 90%, or greater than 95% to the amino acid sequence of any one of SEQ ID NO: 100-117 and 43-46, or a truncated amino acid sequence of any one of SEQ ID NO: 100-117 and 43-46.

Certain of the IL-7Rα ligands of any one of SEQ ID NO: 43-46 can bind to the hu-IL-7Rα subunit with an IC₅₀ of less than 10 μM as determined using phage ELISA competition assays.

An IL-7Rα ligand of any one of SEQ ID NO: 100-117 binds to the hu-IL-7Rα subunit with an IC₅₀ of less than 10 μM as determined using phage ELISA competition assays.

Certain IL-7Rα ligands provided by the present disclosure can bind to a unique binding site on the IL-7Rα subunit that is different from the binding site on the IL-7Rα subunit to which IL-7 binds.

IL-7Rα ligands having SEQ ID NO: 455-459 do not bind competitively with IL-7 binding to the IL-7Rα subunit, indicating that the IL-7Rα ligand binding site for these compounds is distinct from that of IL-7.

SEQ ID NO: 455
Q C I H W D I E T L L S C V
SEQ ID NO: 456
V Y C A E I G E Y R V C R Q
SEQ ID NO: 457
Y M A C S S G L S L C R L S
SEQ ID NO: 458
Q C V H W D L D T L F G C I R E Q L E L
SEQ ID NO: 459
G G V P W C T L D P G S L Q C A W F

This group of IL-7Rα ligands bind to a unique binding site on the IL-7Rα subunit with an IC₅₀ of less than 10 μM.

A unique binding site on the IL-7Rα subunit can be characterized by at least the following properties: (1) a group of IL-7Rα ligands bind to each unique binding site on the IL-7Rα subunit with an IC₅₀ of less than 10 μM; (2) each of the IL-7Rα ligands within the group competitively bind to the unique binding site on the IL-7Rα subunit with each of the other IL-7Rα ligands within the group; (3) a peptide having the amino acid sequence of SEQ ID NO: 261 does not compete for binding to the unique binding site on the IL-7Rα subunit with the peptides within the group of IL-7Rα ligands; and (4) IL-7Rα ligands having SEQ ID NO: 455-459 do not bind competitively with IL-7 binding to the IL-7Rα subunit, indicating that this IL-7Rα ligand binding site is distinct from that of IL-7.

The group of IL-7Rα ligands that bind to the unique binding site comprises at least the IL-7Rα ligands having the amino acid sequence of any one of SEQ ID NO: 455-459.

The unique binding site of the IL-7Rα subunit for these IL-7Rα ligands can be characterized using competitive binding assays as described, for example, in Example 12.

An IL-7Rα ligand can comprise an amino acid sequence selected from any one of SEQ ID NO: 118 to SEQ ID NO: 184:

| | |
|---|---|
| SEQ ID NO: 118 | I P W C T L D P G G L Q C A W L R Q M |
| SEQ ID NO: 119 | I P W C T L D P G G L Q C A A L R Q M |
| SEQ ID NO: 120 | I P W C T L D P G G L Q C A F L R Q M |
| SEQ ID NO: 121 | I P W C T L D P G G L Q C A Y L R Q M |
| SEQ ID NO: 122 | I P W C T L D P G G L Q C A H L R Q M |
| SEQ ID NO: 123 | I P W C T L D P G G L Q C A W A R Q M |
| SEQ ID NO: 124 | I P W C T L D P G G L Q C A W I R Q M |
| SEQ ID NO: 125 | I P W C T L D P G G L Q C A W V R Q M |
| SEQ ID NO: 126 | I P W C T L D P G G L Q C A W L A Q M |
| SEQ ID NO: 127 | I P W C T L D P G G L Q C A W L K Q M |
| SEQ ID NO: 128 | I P W C T L D P G G L Q C A W L H Q M |
| SEQ ID NO: 129 | I P W C T L D P G G L Q C A W L R A M |
| SEQ ID NO: 130 | I P W C T L D P G G L Q C A W L R Q A |
| SEQ ID NO: 131 | I P W C T L D P G G L Q C A W L R A A |
| SEQ ID NO: 132 | I P W C T L D P G G L Q C A W L A A A |
| SEQ ID NO: 133 | I P W C T L D P G G L Q C A W L R Q |
| SEQ ID NO: 134 | I P W C T L D P G G L Q C A W L R |
| SEQ ID NO: 135 | I P W C T L D P G G L Q C A W L |
| SEQ ID NO: 136 | I P W C T L D P G G L Q C A W |
| SEQ ID NO: 137 | I P W C T L D P G G L Q C A W L R Q M G G |
| SEQ ID NO: 138 | I P W C T L D P G G L Q C A W L R Q G G |
| SEQ ID NO: 139 | I P W C T L D P G G L Q C A W L R G G |
| SEQ ID NO: 140 | I P W C T L D P G G L Q C A W L G G |
| SEQ ID NO: 141 | I P W C T L D P G G L Q C A W G G |
| SEQ ID NO: 142 | G G I P W C T L D P G G L Q C A W L R Q M |
| SEQ ID NO: 143 | G G I P W C T L D P G G L Q C A W L R Q |
| SEQ ID NO: 144 | G G I P W C T L D P G G L Q C A W L R |
| SEQ ID NO: 145 | G G I P W C T L D P G G L Q C A W L |
| SEQ ID NO: 146 | G G I P W C T L D P G G L Q C A W |
| SEQ ID NO: 147 | G G I P W C T L D P G G L Q C A W L R Q M G G |
| SEQ ID NO: 148 | G G I P W C T L D P G G L Q C A W L R Q G G |
| SEQ ID NO: 149 | G G I P W C T L D P G G L Q C A W L R G G |
| SEQ ID NO: 150 | G G I P W C T L D P G G L Q C A W L G G |
| SEQ ID NO: 151 | G G I P W C T L D P G G L Q C A W G G |
| SEQ ID NO: 152 | V H R I P W C T L D P G G L Q C A W L R Q M G G |
| SEQ ID NO: 153 | G G V H R I P W C T L D P G G L Q C A W L R Q M |
| SEQ ID NO: 154 | G G V H R I P W C T L D P G G L Q C A W L R Q M G G |
| SEQ ID NO: 155 | V H R I P W C T L D P G G L Q C A W L R Q |
| SEQ ID NO: 156 | V H R I P W C T L D P G G L Q C A W L R |
| SEQ ID NO: 157 | V H R I P W C T L D P G G L Q C A W L R M |
| SEQ ID NO: 158 | G G V H R I P W C T L D P G G L Q C A W L R Q |
| SEQ ID NO: 159 | G G V H R I P W C T L D P G G L Q C A W L R |
| SEQ ID NO: 160 | G G V H R I P W C T L D P G G L Q C A W L R M |
| SEQ ID NO: 161 | V H R I P W C T L D P G G L Q C A W A R Q M |
| SEQ ID NO: 162 | V H R I P W C T L D P G G L Q C A W A R Q M G G |
| SEQ ID NO: 163 | G G V H R I P W C T L D P G G L Q C A W A R Q M |
| SEQ ID NO: 164 | G G V H R I P W C T L D P G G L Q C A W A R Q M G G |
| SEQ ID NO: 165 | V H R I P W C T L D P G G L Q C A W V R Q M |
| SEQ ID NO: 166 | V H R I P W C T L D P G G L Q C A W V R Q M G G |
| SEQ ID NO: 167 | G G V H R I P W C T L D P G G L Q C A W V R Q M |
| SEQ ID NO: 168 | G G V H R I P W C T L D P G G L Q C A W V R Q M G G |
| SEQ ID NO: 169 | V H R I P W C T L D P G G L Q C A W I R Q M |
| SEQ ID NO: 170 | V H R I P W C T L D P G G L Q C A W I R Q M G G |
| SEQ ID NO: 171 | G G V H R I P W C T L D P G G L Q C A W I R Q M |
| SEQ ID NO: 172 | G G V H R I P W C T L D P G G L Q C A W I R Q M G G |
| SEQ ID NO: 173 | I E G R G G Q C I H W D I E T L L S C V |
| SEQ ID NO: 174 | I E G R G G V P W C T L D P G S L Q C A W F |
| SEQ ID NO: 175 | I E G R G G R Y E C A D L P G G L H C E F R |
| SEQ ID NO: 176 | R S C L W Q P G A L H C T W W A E E E P V |
| SEQ ID NO: 177 | G G I E G R G G Q C I H W D I E T L L S C V |
| SEQ ID NO: 178 | G G I E G R G G V P W C T L D P G S L Q C A W F |
| SEQ ID NO: 179 | G G I E G R G G R Y E C A D L P G G L H C E F R |
| SEQ ID NO: 180 | G G R S C L W Q P G A L H C T W W A E E E P V |
| SEQ ID NO: 181 | Q C V H W D L D T L F G C I R E Q L E L G G |
| SEQ ID NO: 182 | G G Q C V H W D L D T L F G C I R E Q L E L |
| SEQ ID NO: 183 | G G Q C V H W D L D T L F G C I R E Q L E L G G |
| SEQ ID NO: 184 | G G H L G V P W C T L D P G S I Q C A W L A K H G G |

A truncated IL-7Rα ligand can include a truncated IL-7Rα ligand based on SEQ ID NO: 114 and 152 such as an IL-7Rα ligand having any one of SEQ ID NO: 155, 156, 203, 185-187, 152, and 188-193:

| | |
|---|---|
| SEQ ID NO: 114 | V H R I P W C T L D P G G L Q C A W L R Q M |
| SEQ ID NO: 152 | V H R I P W C T L D P G G L Q C A W L R Q M G G |
| SEQ ID NO: 155 | V H R I P W C T L D P G G L Q C A W L R Q |
| SEQ ID NO: 156 | V H R I P W C T L D P G G L Q C A W L R |
| SEQ ID NO: 203 | V H R I P W C T L D P G G L Q C A W L |
| SEQ ID NO: 185 | V H R I P W C T L D P G G L Q C A W |
| SEQ ID NO: 186 | V H R I P W C T L D P G G L Q C A |
| SEQ ID NO: 187 | V H R I P W C T L D P G G L Q C |
| SEQ ID NO: 188 | H R I P W C T L D P G G L Q C A W L R Q M G G |
| SEQ ID NO: 189 | R I P W C T L D P G G L Q C A W L R Q M G G |
| SEQ ID NO: 190 | I P W C T L D P G G L Q C A W L R Q M G G |
| SEQ ID NO: 191 | P W C T L D P G G L Q C A W L R Q M G G |
| SEQ ID NO: 192 | W C T L D P G G L Q C A W L R Q M G G |
| SEQ ID NO: 193 | C T L D P G G L Q C A W L R Q M G G |

An IL-7Rα ligand provided by the present disclosure can comprise an amino acid sequence of Formula (2) (SEQ ID NO: 53), an amino acid sequence of Formula (2a) (SEQ ID NO: 54), an amino acid sequence of Formula (2b) (SEQ ID NO: 55), an amino acid sequence of Formula (2c) (SEQ ID NO: 56), an amino acid sequence of Formula (2d) (SEQ ID NO: 57), an amino acid sequence of Formula (2e) (SEQ ID NO: 58), an amino acid sequence of Formula (2f) (SEQ ID NO: 59), or an amino acid sequence of Formula (2g) (SEQ ID NO: 60):

—X¹⁹⁸—X¹⁹⁹—X²⁰⁰-I-P-W-C-T-L-D-P-G-X²¹⁰-L-Q-C-A-W-L-X²¹⁷—X²¹⁸—X²¹⁹— (2)

—X¹⁹⁹—X²⁰⁰-I-P-W-C-T-L-D-P-G-X²¹⁰-L-Q-C-A-W-L-X²¹⁷—X²¹⁸—X²¹⁹— (2a)

—X²⁰⁰-I-P-W-C-T-L-D-P-G-X²¹⁰-L-Q-C-A-W-L-X²¹⁷—X²¹⁸—X²¹⁹— (2b)

-I-P-W-C-T-L-D-P-G-X²¹⁰-L-Q-C-A-W-L-X²¹⁷—X²¹⁸—X²¹⁹— (2c)

—X¹⁹⁸—X¹⁹⁹—X²⁰⁰-I-P-W-C-T-L-D-P-G-X²¹⁰-L-Q-C-A-W-L-X²¹⁷—X²¹⁸— (2d)

—X¹⁹⁸—X¹⁹⁹—X²⁰⁰-I-P-W-C-T-L-D-P-G-X²¹⁰-L-Q-C-A-W-L-X²¹⁷— (2e)

—X¹⁹⁸—X¹⁹⁹—X²⁰⁰-I-P-W-C-T-L-D-P-G-X²¹⁰-L-Q-C-A-W-L- (2f)

—X¹⁹⁸—X¹⁹⁹—X²⁰⁰-I-P-W-C-T-L-D-P-X²¹⁰-L-Q-C-A-W-L- (2g).

wherein each of X¹⁹⁸—X²⁰⁰, X²¹⁰, and X²¹⁷⁻²¹⁹ is independently an amino acid.

In an IL-7Rα ligand of any one of Formula (2)-(2g),
X¹⁹⁸ can be selected from A, G, P, S, T, and V;
X¹⁹⁹ can be selected from F, H, W, and Y;
X²⁰⁰ can be selected from A, G, H, K, P, R, S, and T;
X²¹⁰ can be selected form A, G, P, S, and T;
X²¹⁷ can be selected from A, G, H, K, P, R, S, and T;
X²¹⁸ can be selected from an amino acid and a single bond; and
X²¹⁹ can be selected from an amino acid and a single bond.

In an IL-7Rα ligand of any one of Formula (2)-(2g),
X¹⁹⁸ can be selected from V and G;
X¹⁹⁹ can be selected from H and W;
X²⁰⁰ can be selected from R and G;
X²¹⁰ can be selected form G and S;
X²¹⁷ is selected from R and G;
X²¹⁸ can be selected from Q, G, K and a single bond; and
X²¹⁹ can be selected from G, H, M, and a single bond.

In an IL-7Rα ligand of any one of Formula (2)-(2g), X¹⁹⁸ can be V, X¹⁹⁹ can be H, and X²⁰⁰ can be R.

In an IL-7Rα ligand of any one of Formula (2)-(2g), X¹⁹⁸ can be G, X¹⁹⁹ can be W, and X²⁰⁰ can be G.

In an IL-7Rα ligand of any one of Formula (2)-(2g), X²¹⁰ can be G.

In an IL-7Rα ligand of any one of Formula (2)-(2g), X²¹⁰ can be S.

In an IL-7Rα ligand of any one of Formula (2)-(2g), X²¹⁷ can be R.

In an IL-7Rα ligand of any one of Formula (2)-(2g), X²¹⁷ can be R, X²¹⁸ can be Q, and X²¹⁹ can be M.

In an IL-7Rα ligand of any one of Formula (2)-(2g), X²¹⁷ can be G, X²¹⁸ can be K, and X²¹⁹ can be H.

In an IL-7Rα ligand of Formula (2)-(2g), the IL-7Rα ligand can be defined by any combination of variables as defined in the immediately preceding nine (9) paragraphs.

An IL-7Rα ligand can comprise an amino acid sequence or a truncated amino acid sequence selected from any one of SEQ ID NO: 133-135, 156, 157, and 197-245:

| | |
|---|---|
| SEQ ID NO: 135 | I P W C T L D P G G L Q C A W L |
| SEQ ID NO: 134 | I P W C T L D P G G L Q C A W L R |
| SEQ ID NO: 133 | I P W C T L D P G G L Q C A W L R Q |
| SEQ ID NO: 197 | I P W C T L D P G G L Q C A W L R G |
| SEQ ID NO: 198 | I P W C T L D P G G L Q C A W L R Q G |
| SEQ ID NO: 199 | I P W C T L D P G G L Q C A W L R G G |
| SEQ ID NO: 200 | I P W C T L D P G G L Q C A W L G |
| SEQ ID NO: 201 | I P W C T L D P G G L Q C A W L G K |
| SEQ ID NO: 202 | I P W C T L D P G G L Q C A W L G K H |
| SEQ ID NO: 203 | V H R I P W C T L D P G G L Q C A W L |
| SEQ ID NO: 156 | V H R I P W C T L D P G G L Q C A W L R |
| SEQ ID NO: 157 | V H R I P W C T L D P G G L Q C A W L R Q |
| SEQ ID NO: 204 | V H R I P W C T L D P G G L Q C A W L R G |
| SEQ ID NO: 205 | V H R I P W C T L D P G G L Q C A W L R Q G |
| SEQ ID NO: 206 | V H R I P W C T L D P G G L Q C A W L R G G |
| SEQ ID NO: 207 | V H R I P W C T L D P G G L Q C A W L G |
| SEQ ID NO: 208 | V H R I P W C T L D P G G L Q C A W L G K |
| SEQ ID NO: 209 | V H R I P W C T L D P G G L Q C A W L G K H |
| SEQ ID NO: 210 | G W G I P W C T L D P G G L Q C A W L |
| SEQ ID NO: 211 | G W G I P W C T L D P G G L Q C A W L R |
| SEQ ID NO: 212 | G W G I P W C T L D P G G L Q C A W L R Q |
| SEQ ID NO: 213 | G W G I P W C T L D P G G L Q C A W L R G |
| SEQ ID NO: 214 | G W G I P W C T L D P G G L Q C A W L R Q G |
| SEQ ID NO: 215 | G W G I P W C T L D P G G L Q C A W L R G G |
| SEQ ID NO: 216 | G W G I P W C T L D P G G L Q C A W L G |
| SEQ ID NO: 217 | G W G I P W C T L D P G G L Q C A W L G K |
| SEQ ID NO: 218 | G W G I P W C T L D P G G L Q C A W L G K H |
| SEQ ID NO: 219 | I P W C T L D P G S L Q C A W L |
| SEQ ID NO: 220 | I P W C T L D P G S L Q C A W L R |
| SEQ ID NO: 221 | I P W C T L D P G S L Q C A W L R Q |
| SEQ ID NO: 222 | I P W C T L D P G S L Q C A W L R G |
| SEQ ID NO: 223 | I P W C T L D P G S L Q C A W L R Q G |
| SEQ ID NO: 224 | I P W C T L D P G S L Q C A W L R G G |
| SEQ ID NO: 225 | I P W C T L D P G S L Q C A W L G |
| SEQ ID NO: 226 | I P W C T L D P G S L Q C A W L G K |
| SEQ ID NO: 227 | I P W C T L D P G S L Q C A W L G K H |
| SEQ ID NO: 228 | V H R I P W C T L D P G S L Q C A W L |
| SEQ ID NO: 229 | V H R I P W C T L D P G S L Q C A W L R |
| SEQ ID NO: 230 | V H R I P W C T L D P G S L Q C A W L R Q |
| SEQ ID NO: 231 | V H R I P W C T L D P G S L Q C A W L R G |
| SEQ ID NO: 232 | V H R I P W C T L D P G S L Q C A W L R Q G |
| SEQ ID NO: 233 | V H R I P W C T L D P G S L Q C A W L R G G |
| SEQ ID NO: 234 | V H R I P W C T L D P G S L Q C A W L G |
| SEQ ID NO: 235 | V H R I P W C T L D P G S L Q C A W L G K |
| SEQ ID NO: 236 | V H R I P W C T L D P G S L Q C A W L G K H |
| SEQ ID NO: 237 | G W G I P W C T L D P G S L Q C A W L |
| SEQ ID NO: 238 | G W G I P W C T L D P G S L Q C A W L R |
| SEQ ID NO: 239 | G W G I P W C T L D P G S L Q C A W L R Q |
| SEQ ID NO: 240 | G W G I P W C T L D P G S L Q C A W L R G |
| SEQ ID NO: 241 | G W G I P W C T L D P G S L Q C A W L R Q G |
| SEQ ID NO: 242 | G W G I P W C T L D P G S L Q C A W L R G G |
| SEQ ID NO: 243 | G W G I P W C T L D P G S L Q C A W L G |
| SEQ ID NO: 244 | G W G I P W C T L D P G S L Q C A W L G K |
| SEQ ID NO: 245 | G W G I P W C T L D P G S L Q C A W L G K H |

An IL-7Rα ligand can comprise a truncated amino acid sequence of any one of SEQ ID NO: 118-193, 197-245 and 53-60.

An IL-7Rα ligand can comprise an amino acid sequence selected from any one of SEQ ID NO: 118-245, or a truncated amino acid sequence of any one of SEQ ID NO: 118-193, 197-245 and 53-60, wherein the amino acid sequence can independently comprise from 1 to 5 glycines (G) (SEQ ID NO: 28) on the N-terminus, on the C-terminus, or on both the N- and C-termini.

An IL-7Rα ligand can comprise an amino acid sequence selected from any one of SEQ ID NO: 118-193, 197-245 and 53-60, or a truncated amino acid sequence of any one of SEQ ID NO: 118-193, 197-245 and 53-60, wherein the amino acid sequence comprises one or more amino acid substitutions such as from 1 to 5 amino acid substitutions. Each amino acid substitution can independently comprise a conservative amino acid substitution or a non-conservative amino acid substitution.

An IL-7Rα ligand can comprise an amino acid sequence similarity greater than 60%, greater than 70%, greater than 75%, greater than 80%, greater than 85%, greater than 90%, or greater than 95% to the amino acid sequence of any one of SEQ ID NO: 118-193, 197-245 and 53-60 or to a truncated amino acid sequence of any one of SEQ ID NO: 118-193, 197-245 and 53-60.

Certain of the IL-7Rα ligands of SEQ ID NO: 118-193, 197-245 and 53-60 bind to the hu-IL-7Rα subunit with an IC₅₀ of less than 10 μM as determined using phage ELISA competition assays.

An IL-7Rα ligand can comprise:
an amino acid sequence of any one of SEQ ID NO: 43-46, 100-117, 118-193, 53-60, and 197-245;
a truncated amino acid sequence of any one of SEQ ID NO: 43-46, 100-117, 118-193, 53-60, and 197-245;
an amino acid sequence of any one of SEQ ID NO: 43-46, 100-117, 118-193, 53-60, and 197-245 having from 1 to 5 amino acid substitutions;
an amino acid sequence of any one of SEQ ID NO: 43-46, 100-117, 118-193, 53-60, 197-245 having from 1 to 5 flanking glycines on the N-terminus, the C-terminus, or both the N-terminus and the C-terminus;
an amino acid sequence having greater than 60% sequence similarity to an amino acid sequence of any one of SEQ ID NO: 43-46, 100-117, 118-193, 53-60, and 197-245; or
a combination of any of the foregoing.

An-IL-7Rα ligand can comprise an amino acid sequence of any one of SEQ ID NO: 100-117.

An-IL-7Rα ligand can comprise an amino acid sequence of any one of SEQ ID NO: 114, 152, 155, 156, 203, 185-187, 152-172, and 188-193.

An-IL-7Rα ligand can comprise an amino acid sequence of any one of SEQ ID NO: 114, 152, 155-154, 368, 369, 374, 383, 394-397, 405, 420, and 443.

An-IL-7Rα ligand can comprise an amino acid sequence of any one of SEQ ID NO: 114 and 152-154.

An-IL-7Rα ligand can comprise an amino acid sequence of SEQ ID NO: 114.

An-IL-7Rα ligand can comprise an amino acid sequence of SEQ ID NO: 152.

Examples of suitable IL-7Rα ligands are disclosed, for example, in U.S. Pat. No. 11,254,729, which is incorporated by reference in its entirety.

An Rγc ligand can have an IC₅₀ for binding to the hu-IL-7Rγc subunit of less than 100 μM, less than 10 μM, less than 1 μM, less than 0.1 μM, or less than 0.01 μM, as determined using phage ELISA competition assays.

An Rγc ligand can comprise the amino acid sequence of Formula (3) (SEQ ID NO: 47), Formula (3a) (SEQ ID NO: 48), Formula (3b) (SEQ ID NO: 49), Formula (3c) (SEQ ID NO: 50), Formula (3d) (SEQ ID NO: 51), or Formula (3e) (SEQ ID NO: 52):

—X¹⁷¹—X¹⁷²—X¹⁷³—X¹⁷⁴—X¹⁷⁵—C—X¹⁷⁶—X¹⁷⁷—X¹⁷⁸—X¹⁷⁹—X¹⁸⁰—X¹⁸¹—X¹⁸²—X¹⁸³—C—X¹⁸⁴—X¹⁸⁵—X¹⁸⁶—X¹⁸⁷—X¹⁸⁸— (3)

—X¹⁷²—X¹⁷³—X¹⁷⁴—X¹⁷⁵—C—X¹⁷⁶—X¹⁷⁷—X¹⁷⁸—X¹⁷⁹—X¹⁸⁰—X¹⁸¹—X¹⁸²—X¹⁸³—C—X¹⁸⁴—X¹⁸⁵—X¹⁸⁶—X¹⁸⁷— (3a)

—X¹⁷³—X¹⁷⁴—X¹⁷⁵—C—X¹⁷⁶—X¹⁷⁷—X¹⁷⁸—X¹⁷⁹—X¹⁸⁰—X¹⁸¹—X¹⁸²—X¹⁸³—C—X¹⁸⁴—X¹⁸⁵—X¹⁸⁶— (3b)

—X¹⁷⁴—X¹⁷⁵—C—X¹⁷⁶—X¹⁷⁷—X¹⁷⁸—X¹⁷⁹—X¹⁸⁰—X¹⁸¹—X¹⁸²—X¹⁸³—C—X¹⁸⁴—X¹⁸⁵— (3c)

—X¹⁷⁵—C—X¹⁷⁶—X¹⁷⁷—X¹⁷⁸—X¹⁷⁹—X¹⁸⁰—X¹⁸¹—X¹⁸²—X¹⁸³—C—X¹⁸⁴— (3d)

—C—X¹⁷⁶—X¹⁷⁷—X¹⁷⁸—X¹⁷⁹—X¹⁸⁰—X¹⁸¹—X¹⁸²—X¹⁸³—C— (3e)

wherein each of X¹⁷¹—X¹⁸⁸ is independently selected from an amino acid.

In an Rγc ligand of any one of Formula (3)-(3e), X¹⁷¹ can be selected from an amino acid comprising a basic side chain; X¹⁷² can be selected from an amino acid comprising a hydroxyl-containing side chain; X¹⁷³ can be selected from an amino acid comprising an acidic side chain or a large hydrophobic side chain; X¹⁷⁴ can be selected from an amino acid comprising a large hydrophobic side chain; X¹⁷⁵ can be selected from an amino acid comprising an acidic side chain or a large hydrophobic side chain; X¹⁷⁶ can be selected from an amino acid comprising an acidic side chain or a polar/neutral side chain; X¹⁷⁷ can be selected from an amino acid comprising an acidic side chain; X¹⁷⁸ can be selected from an amino acid comprising a large hydrophobic side chain or an aromatic side chain; X¹⁷⁹ can be selected from an amino acid comprising an acidic side chain or a polar/neutral side chain; X¹⁸⁰ can be G; X¹⁸¹ can be V; X¹⁸² can be E; X¹⁸³ can be L; X¹⁸⁴ can be W; X¹⁸⁵ can be selected from an amino acid comprising a large hydrophobic side chain; X¹⁸⁶ can be E; X¹⁸⁷ can be selected from an amino acid; and X¹⁸⁸ can be selected from an amino acid comprising an acidic side chain.

In Rγc ligands of Formula (3), (3a), (3b), (3c), (3d), and/or (3e), X¹⁷¹ can be selected from H, K, and R; X¹⁷² can be selected from S, T, and Y; X¹⁷³ can be selected from D, E, F, I, L, M, V, W, and Y; X¹⁷⁴ can be selected from F, I, L, M, V, W, and Y; X¹⁷⁵ can be selected from D, E, F, I, L, M, V, W, and Y; X¹⁷⁶ can be selected from D, E, H, N, Q, S, T, and Y; X¹⁷⁷ can be selected from D and E; X¹⁷⁸ can be selected from F, H, I, L, M, V, W, and Y; X¹⁷⁹ can be selected from D, E, H, N, Q, S, T, and Y; X¹⁸⁰ can be G; X¹⁸¹ can be V; X¹⁸² can be E; X¹⁸³ can be L; X¹⁸⁴ can be W; X¹⁸⁵ can be selected from F, I, L, M, V, W, Y, H, N, Q, S, and T; X¹⁸⁶ can be E; X¹⁸⁷ can be selected from an amino acid; and X¹⁸⁸ can be selected from D and E.

In Rγc ligands of Formula (3), (3a), (3b), (3c), (3d), and/or (3e), X¹⁷¹ can be selected from D, E, G, H, K, M, N, P, Q, R, S, and T; X¹⁷² can be selected from A, D, E, G, I, K, L, P, Q, R, S, T, V, W, and Y; X¹⁷³ can be selected from A, D, E, F, G, I, Q, S, T, V, W, and Y; X¹⁷⁴ can be selected from A, I, E, I, L, M, N, Q, R, S, T, and V; X¹⁷⁵ can be selected from A, E, I, L, M, N, Q, R, S, T, and V; X¹⁷⁶ can be selected from D, E, H, L, Q, R, and V; X¹⁷⁷ can be selected from D, E, N, T, and V; X¹⁷⁸ can be selected from F, S, W, and Y; X¹⁷⁹ can be selected from A, D, E, G, H, K, N, Q, R, and Y; X¹⁸⁰ can be selected from G and R; X¹⁸¹ can be V; X¹⁸² can be selected from D, E, and Y; X¹⁸³ can be selected from F, I, and L; X¹⁸⁴ can be W; X¹⁸⁵ can be selected from C, H, I, L, P, Q, T, V, and Y; X¹⁸⁶ can be selected from A, D, E, G, M, R, S, T, and V; X¹⁸⁷ can be selected from A, D, E, F, G, I, M, N, P, Q, R, S, T, V, W, and Y; and X¹⁸⁸ can be selected from A, C, D, E, F, G, I, K, L, N, P, Q, R, S, and V.

In Rγc ligands of Formula (3), (3a), (3b), (3c), (3d), and/or (3e), X¹⁷¹ can be selected from H, K, and R; X¹⁷² can be selected from S, T, and Y; X¹⁷³ can be selected from D, E, F, I, and V; X¹⁷⁴ can be selected from I and V; X¹⁷⁵ can be selected from E, I, L, M, and V; X¹⁷⁶ can be selected from D, E, and Q; X¹⁷⁷ can be selected from D and E; X¹⁷⁸ can be selected from F and W; X¹⁷⁹ can be selected from D, E, N, and Q; X¹⁸⁰ can be G; X¹⁸¹ can be V; X¹⁸² can be selected from D and E; X¹⁸³ can be L; X¹⁸⁴ can be W; X¹⁸⁵ can be selected from I, L, Q, and V; X¹⁸⁶ can be selected from D and E; X¹⁸⁷ can be selected from A, D, E, F, G, I, M, N, P, Q, R, S, T, V, W, and Y; and X¹⁸⁸ can be selected from D, E, N, and Q.

In Rγc ligands of Formula (3), (3a), (3b), (3c), (3d), and/or (3e), X¹⁷¹ can be selected from K and R; X¹⁷² can be selected from S, T, and Y; X¹⁷³ can be selected from D, E, F, I, and V; X¹⁷⁴ can be V; X¹⁷⁵ can be selected from E, L, M, and V; X¹⁷⁶ can be Q; X¹⁷⁷ can be selected from D and E; X¹⁷⁸ can be W; X¹⁷⁹ can be selected from D, E, N, and Q; X¹⁸⁰ can be G; X¹⁸¹ can be V; X¹⁸² can be E; X¹⁸³ can be L; X¹⁸⁴ can be W; X¹⁸⁵ can be selected from I, L, Q, and V; X¹⁸⁶ can be selected from D and E; X¹⁸⁷ can be selected from A, D, E, F, G, I, M, N, P, Q, R, S, T, V, W, and Y; and X¹⁸⁸ can be selected from D, E, N, and Q.

In Rγc ligands of Formula (3), X¹⁷¹ can be selected from H, K, and R.

In Rγc ligands of Formula (3)-(3a), X¹⁷² can be selected from S, T, and Y.

In Rγc ligands of Formula (3)-(3b), X¹⁷³ can be selected from D, E, F, I, L, M, V, W, and Y.

In Rγc ligands of Formula (3)-(3b), X¹⁷³ can be selected from D and E.

In Rγc ligands of Formula (3)-(3b), X¹⁷³ can be selected from F, I, L, M, V, W, and Y.

In Rγc ligands of Formula (3)-(3c), X¹⁷⁴ can be selected from F, I, L, M, V, W, and Y.

In Rγc ligands of Formula (3)-(3c), X¹⁷⁴ can be V.

In Rγc ligands of Formula (3)-(3d), X¹⁷⁵ can be selected from D, E, F, I, L, M, V, W, and Y.

In Rγc ligands of Formula (3)-(3d), X¹⁷⁵ can be selected from D and E.

In Rγc ligands of Formula (3)-(3d), X¹⁷⁵ can be selected from F, I, L, M, V, W, and Y.

In Rγc ligands of Formula (3)-(3e), X¹⁷⁶ can be selected from D, E, H, N, Q, S, T, and Y.

In Rγc ligands of Formula (3)-(3e), X¹⁷⁶ can be selected from E and Q.

In Rγc ligands of Formula (3)-(3e), X¹⁷⁷ can be selected from D and E.

In Rγc ligands of Formula (3)-(3e), X¹⁷⁸ can be selected from F, H, I, L, M, V, W, and Y.

In Rγc ligands of Formula (3)-(3e), X¹⁷⁸ can be selected from F, H, W, and Y.

In Rγc ligands of Formula (3)-(3e), X¹⁷⁸ can be W.

In Rγc ligands of Formula (3)-(3e), X¹⁷⁹ can be selected from D, E, H, N, Q, S, T, and Y.

In Rγc ligands of Formula (3)-(3e), X¹⁷⁹ can be selected from D, E, and Q.

In Rγc ligands of Formula (3)-(3e), X¹⁸⁰ can be G.

In Rγc ligands of Formula (3)-(3e), X¹⁸¹ can be V.

In Rγc ligands of Formula (3)-(3e), X¹⁸² can be E.

In Rγc ligands of Formula (3)-(3e), X¹⁸³ can be L.

In Rγc ligands of Formula (3)-(3d), X¹⁸⁴ can be W.

In Rγc ligands of Formula (3)-(3c), X¹⁸⁵ can be selected from F, I, L, M, V, W, and Y.

In Rγc ligands of Formula (3)-(3c), X¹⁸⁵ can be L.

In Rγc ligands of Formula (3)-(3b), X¹⁸⁶ can be E.

In Rγc ligands of Formula (3)-(3a), X¹⁸⁷ can be selected from an amino acid.

In Rγc ligands of Formula (3), X¹⁸⁸ can be selected from D and E.

In Rγc ligands of Formula (3), (3a), (3b), (3c), (3d), and/or (3e), X¹⁷¹ can be selected from H, K, and R; X¹⁷² can be selected from S, T, and Y; X¹⁷³ can be selected from D, E, F, I, L, M, V, W, and Y; X¹⁷⁴ can be selected from F, I, L, M, V, W, and Y; X¹⁷⁵ can be selected from D, E, F, I, L, M, V, W, and Y; X¹⁷⁶ can be selected from D, E, H, N, Q, S, T, and Y; X¹⁷⁷ can be selected from D and E; X¹⁷⁸ can be selected from F, H, I, L, M, V, W, and Y; X¹⁷⁹ can be selected from D, E, H, N, Q, S, T, and Y; X¹⁸⁰ can be G; X¹⁸¹ can be V; X¹⁸² can be E; X¹⁸³ can be L; X¹⁸⁴ can be selected from W; X¹⁸⁵ can be selected from F, I, L, M, V, W, and Y; X¹⁸⁶ can be E; X¹⁸⁷ can be selected from an amino acid; and X¹⁸⁸ can be selected from D and E.

In Rγc ligands of Formula (3), (3a), (3b), (3c), (3d), and/or (3e), X¹⁷¹ can be selected from H, K, and R; X¹⁷² can be selected from S, T, and Y; X¹⁷³ can be selected from D and E; X¹⁷⁴ can be V; X¹⁷⁵ can be selected from D and E; X¹⁷⁶ can be selected from E and Q; X¹⁷⁷ can be selected from D and E; X¹⁷⁸ can be selected from F, H, W, and Y; X¹⁷⁹ can be selected from D, E, and Q; X¹⁸⁰ can be G; X¹⁸¹ can be V; X¹⁸² can be E; X¹⁸³ can be L; X¹⁸⁴ can be W; X¹⁸⁵ can be selected from F, I, L, M, V, W, Y, H, N, Q, S, and T; X¹⁸⁶ can be E; X¹⁸⁷ can be selected from an amino acid; and X¹⁸⁸ can be selected from D and E.

In Rγc ligands of Formula (3), (3a), (3b), (3c), (3d), and/or (3e), X¹⁷¹ can be selected from H, K, and R; X¹⁷² can be selected from S, T, and Y; X¹⁷³ can be selected from F, I, L, M, V, W, and Y; X¹⁷⁴ can be V; X¹⁷⁵ can be selected from F, I, L, M, V, W, and Y; X¹⁷⁶ can be selected from E and Q; X¹⁷⁷ can be selected from D and E; X¹⁷⁸ can be selected from F, H, W, and Y; X¹⁷⁹ can be selected from D, E, and Q; X¹⁸⁰ can be G; X¹⁸¹ can be V; X¹⁸² can be E; X¹⁸³ can be L; X¹⁸⁴ can be W; X¹⁸⁵ can be selected from F, I, L, M, V, W, Y, H, N, Q, S, and T; X¹⁸⁶ can be E; X¹⁸⁷ can be selected from an amino acid; and X¹⁸⁸ can be selected from D and E.

In Rγc ligands of Formula (3), (3a), (3b), (3c), (3d), and/or (3e), X¹⁷¹ can be selected from H, K, and R; X¹⁷² can be selected from S, T, and Y; X¹⁷³ can be selected from D, E, F, I, L, M, V, W, and Y; X¹⁷⁴ can be V; X¹⁷⁵ can be selected from D, E, F, I, L, M, V, W, and Y; X¹⁷⁶ can be selected from D, E, H, N, Q, S, T, and Y; X¹⁷⁶ can be selected from E and Q; X¹⁷⁷ can be selected from D and E; X¹⁷⁸ can be W; X¹⁷⁹ can be selected from D, E, and Q; X¹⁸⁰ can be G; X¹⁸¹ can be V; X¹⁸² can be E; X¹⁸³ can be L; X¹⁸⁴ can be W; X¹⁸⁵ can be selected from F, I, L, M, V, W, Y, H, N, Q, S, and T; X¹⁸⁶ can be E; X¹⁸⁷ can be selected from an amino acid; and X¹⁸⁸ can be selected from D and E.

An Rγc ligand can comprise an amino acid sequence selected from any one of SEQ ID NO: 246-324:

SEQ ID NO: 246
I E C D T S Y G V Y I C W Q
SEQ ID NO: 247
I E C E E W R G V E L C W Q
SEQ ID NO: 248
P E G R E V V V C R D W Y G V E L C W Q
SEQ ID NO: 249
I W G R T V V E C Q D W E G V E L C W Q
SEQ ID NO: 250
L A L R K E V V C Q E Y Y G V E L C W I
SEQ ID NO: 251
H E A R E V V V C Q D W Y G V E L C W Q
SEQ ID NO: 252
M V N R E V V V C E D W Y G V E L C W Q
SEQ ID NO: 253
T A N Q T V V E C Q V W G G V E L C W Q
SEQ ID NO: 254
V E C Q E W G G V E L C W C
SEQ ID NO: 255
D V E C V D W G G V E L C W H
SEQ ID NO: 256
I V C E E W R G V E L C W L
SEQ ID NO: 257
D F E R S Y V V C Q D W D G V E L C W I
SEQ ID NO: 258
A H S R Q E V V C E E W Y G V E L C W I
SEQ ID NO: 259
S A P E R W V E C E D W Q G V E L C W V
SEQ ID NO: 260
Y S R E L Y V Q C E D W E G V E L C W I
SEQ ID NO: 261
V V C Q D W E G V E L C W Q
SEQ ID NO: 262
D V V C Q N W E G V D L C W H
SEQ ID NO: 263
S A G R Q E V V C Q D W N G V E L C W I
SEQ ID NO: 264
G Q G R E V V V C H D W Y G V E L C W Q
SEQ ID NO: 265
D W R R S V V E C Q D W Y G V E L C W Q
SEQ ID NO: 266
D V V C Q N W D G V D L C W H
SEQ ID NO: 267
T L G R T V V E C Q D W G G V E L C W Q
SEQ ID NO: 268
R L L N S V V E C L D W E G V E L C W Q
SEQ ID NO: 269
I V C E D W R G V E L C W I
SEQ ID NO: 270
V V C Q E W E G V E L C W C
SEQ ID NO: 271
G D R P K E V V C E D W K G V E L C W I
SEQ ID NO: 272
E R P R S F I E C Q E W E G V E L C W L
SEQ ID NO: 273
E G S T T T I E C E E W A G V E L C W L
SEQ ID NO: 274
A N Q N T V V E C Q D W H G V E L C W Q
SEQ ID NO: 275
R S D D E V V V C Q E W E G V E L C W Q
SEQ ID NO: 276
I E C E E W A G V E L C W L
SEQ ID NO: 277
T W N M S E L E C Q D W N G V E I C W H
SEQ ID NO: 278
G N D D S Y I V C E E W K G V E L C W I
SEQ ID NO: 279
F A H H G V V E C Q E W Y G V E L C W Q
SEQ ID NO: 280
L N R S V W I E C E E Y E G V E L C W L
SEQ ID NO: 281
W S K K A E V V C E E W G G V E F C W I
SEQ ID NO: 282
R S N Q T V V E C Q D W E G V E L C W Q
SEQ ID NO: 283
V V C Q E W E G V E L C W Y A G E C M Q
SEQ ID NO: 284
I L C Q E F E G V E L C W L E E S L A E
SEQ ID NO: 285
K S Q V E C Q D W E G V E L C W V V S E
SEQ ID NO: 286
K I T V E C Q D W D G V E L C W P T W I
SEQ ID NO: 287
R P Q I E C Q E W Q G V E L C W T R E E
SEQ ID NO: 288
V S C Q E W D G V E L C W V D G D L A A
SEQ ID NO: 289
I M C Q E W D G V E L C W L E R D K A N
SEQ ID NO: 290
G L E I A C E D W Y G V E L C W L R R A
SEQ ID NO: 291
G Y G V L C Q E W Q G V E L C W P V Q R E A G V
SEQ ID NO: 292
P Y G V V C Q D W A G V E L C W V E N R
SEQ ID NO: 293
K L T V E C Q D W D G V E L C W V G V E
SEQ ID NO: 294
I N C Q T W N G V E L C W V D E G L Y Q
SEQ ID NO: 295
V V C Q E W E G V E L C W V E P P L L P
SEQ ID NO: 296
R V Q V E C E D W N G V E L C W P V R V
SEQ ID NO: 297
D R Q V V C E E W D G V E L C W I E E S
SEQ ID NO: 298
K T T V A C Q D W G G V E L C W V E R V
SEQ ID NO: 299
R P E V V C Q E W E G V E L C W I S P L
SEQ ID NO: 300
R L G V E C Q E W E G V D L C W I S A F
SEQ ID NO: 301
K P V V V C E E W Q G V E L C W L E I Q
SEQ ID NO: 302
V V C E V F Q G V E L C W C E N E E F T
SEQ ID NO: 303
T D E V S C Q E W E G V E L C W I E R Q
SEQ ID NO: 304
P V E V R C Q E W E G V E L C W V V G I
SEQ ID NO: 305
G P E V V C E E F N R V E L C W V E Y N
SEQ ID NO: 306
K Y I V E C Q E W G G V E L C W P E M V
SEQ ID NO: 307
V T C Q E Y E G V E L C W T V G C A Y S
SEQ ID NO: 308
V V C Q E W E G V E L C W Q T G P G A H A
SEQ ID NO: 309
I V C E E Y N G V E L C W V E T S V K P
SEQ ID NO: 310
E Q Q V V C Q E W N G V E L C W I E A G
SEQ ID NO: 311
Q L G V E C Q N W R G V E L C W V S E I
SEQ ID NO: 312
T A E V V C Q E W D G V E L C W I E V L
SEQ ID NO: 313
S P S I V C E E W A G V E L C W V D Y S
SEQ ID NO: 314
A V C Q D W Y G V E L C W C M Q D I L D
SEQ ID NO: 315
V E C E E W G G V E L C W L A D E V M W
SEQ ID NO: 316
H S T V I C Q D W D G V E L C W I E N D
SEQ ID NO: 317
K K I V V C Q D W G G V E L C W T E D D
SEQ ID NO: 318
S V E V V C E E W H G V E L C W P V F I
SEQ ID NO: 319
R W A V S C Q D W Q G I E L C W P E W D
SEQ ID NO: 320
R T G V E C Q D W H G V E L C W P V W E
SEQ ID NO: 321
G Y G V V C E D F R G V E L C W L E R K
SEQ ID NO: 322
R T E V E C E D W E G V E L C W L
SEQ ID NO: 323
I L C E E W Q G V E L C W L E G G G S
SEQ ID NO: 324
V G I E C E E W A G V E L C W L

An Rγc ligand provided by the present disclosure can comprise a truncated amino acid sequence of any one of SEQ ID NO: 246-324 and 47-52.

An Rγc ligand provided by the present disclosure can comprise an amino acid sequence selected from any one of SEQ ID NO: 246-324 and 47-52, or a truncated amino acid sequence of any one of SEQ ID NO: 246-324 and 47-52, wherein the amino acid sequence can independently comprise from 1 to 5 glycines (G) (SEQ ID NO: 12) on the N-terminus, on the C-terminus, or on both the N- and C-termini.

An Rγc ligand provided by the present disclosure can comprise an amino acid sequence selected from any one of SEQ ID NO: 246-324 and 47-52, or a truncated amino acid sequence of any one of SEQ ID NO: 246-324 and 47-52 wherein the amino acid sequence comprises one or more amino acid substitutions such as from 1 to 5 amino acid substitutions. Each amino acid substitution can independently be a conservative amino acid substitution or a non-conservative amino acid substitution.

The Rγc ligands of SEQ ID NO: 47-52 can bind to the hu-IL-7Rγc subunit with an IC₅₀ of less than 100 μM.

The Rγc ligands of SEQ ID NO: 246-324 bind to the hu-IL-7Rγc subunit with an IC₅₀ of less than 100 μM.

An Rγc ligand can comprise an amino acid sequence having an amino acid sequence similarity greater than 60%, greater than 70%, greater than 75%, greater than 80%, greater than 85%, greater than 90%, or greater than 95% to the amino acid sequence of any one of SEQ ID NO: 246-324 and 47-52.

An Rγc ligand can comprise an amino acid sequence selected from any one of SEQ ID NO: 261 and 325-327:

| | |
|---|---|
| SEQ ID NO: 261 | V V C Q D W E G V E L C W Q |
| SEQ ID NO: 325 | V V C Q D W E G V E L C W Q G G |
| SEQ ID NO: 326 | G G V V C Q D W E G V E L C W Q |

| | |
|---|---|
| SEQ ID NO: 327 | G G V V C Q D W E G V E L C W Q G G |

A Rγc ligand provided by the present disclosure can comprise a truncated amino acid sequence of any one of SEQ ID NO: 261 and 325-327.

A Rγc ligand provided by the present disclosure can comprise an amino acid sequence selected from any one of SEQ ID NO: 261 and 325-327, or a truncated amino acid sequence of any one of SEQ ID NO: 325-327, wherein the amino acid sequence can independently comprise from 1 to 5 glycines (G) (SEQ ID NO: 12) on the N-terminus, on the C-terminus, or on both the N- and C-termini.

An Rγc ligand provided by the present disclosure can comprise an amino acid sequence selected from any one of SEQ ID NO: 261 and 325-327, or a truncated amino acid sequence of any one of SEQ ID NO: 261 and 325-327, wherein the amino acid sequence comprises one or more amino acid substitutions such as from 1 to 5 amino acid substitutions. Each amino acid substitution can independently be selected from a conservative amino acid substitution and a non-conservative amino acid substitution.

An Rγc ligand of any one of SEQ ID NO: 261 and 325-327 binds to the human Rγc subunit with an IC₅₀ of less than 100 μM as determined using a phage ELISA competition assay.

An Rγc ligand can comprise an amino acid sequence having an amino acid sequence similarity greater than 60%, greater than 70%, greater than 75%, greater than 80%, greater than 85%, greater than 90%, or greater than 95%, to the amino acid sequence of any one of SEQ ID NO: 261 and 325-327.

Certain Rγc ligands provided by the present disclosure can bind to a specific binding site on the IL-7Rγc subunit that is different than the Rγc binding site to which IL-7 binds.

These Rγc ligands do not compete for binding to the specific Rγc binding site with IL-7, have no detectable binding to the IL-7Rα subunit, and bind to the IL-7Rγc subunit with an IC₅₀ of less than 10 μM.

The specific binding site on the IL-7Rγc subunit can be characterized by at least the following properties: (1) a group of Rγc ligands bind to the specific binding site on the IL-7Rγc subunit with an IC₅₀ of less than 10 μM; (2) Rγc ligands within the group competitively bind to the specific binding site on the IL-7Rγc subunit with each of the other Rγc ligands within the group; and (3) the Rγc ligands within the group do not compete for binding to the specific binding site with an Rγc ligand having the amino acid sequence of SEQ ID NO: 466.

An IL-7Rα ligand having the amino acid sequence of SEQ ID NO: 460 does not compete for binding to the binding site with the group of Rγc ligands.

The group of Rγc ligands comprises Rγc ligands having an amino acid sequence of SEQ ID NO: 461-465.

Rγc ligands within the group of Rγc ligands can bind to the IL-7Rγc subunit with an IC₅₀ of less than 100 μM and can bind to the IL-7Rα subunit with an IC₅₀ of greater than 100 μM.

The specific binding site of the IL-7Rγc subunit for these Rγc ligands can be characterized using competitive binding assays.

An Rγc ligand can comprise:
an amino acid sequence of any one of SEQ ID NO: 246-327 and 47-52;
a truncated amino acid sequence of any one of SEQ ID NO: 246-327 and 47-52;
an amino acid sequence of any one of SEQ ID NO: 246-327 and 47-52 having from 1 to 5 amino acid substitutions;
an amino acid sequence of any one of SEQ ID NO: 246-327 and 47-52 having from 1 to 5 flanking glycines on the N-terminus, the C-terminus, or both the N-terminus and the C-termini;
an amino acid sequence having greater than 60% sequence similarity to an amino acid sequence of any one of SEQ ID NO: 246-327 and 47-52; or
a combination of any of the foregoing.

An Rγc ligand can comprise an amino acid sequence of any one of SEQ ID NO: 246-324.

An Rγc ligand can comprise an amino acid sequence of any one of SEQ ID NO: 261 and 325-327.

An Rγc ligand can comprise an amino acid sequence of SEQ ID NO: 261.

Examples of suitable Rγc ligands are disclosed, for example, in U.S. Pat. No. 10,689,417, which is incorporated by reference in its entirety.

IL-7Rα ligands and/or Rγc ligands can comprise one or more flanking amino acids bound to the N-terminus and/or to the C-terminus of the ligand.

The flanking amino acids can separate the portion of the ligand that interacts with IL-7R from other portions of the ligand and/or ligand construct.

An IL-7Rα ligand and/or Rγc ligand can comprise flanking amino acids such as, for example, from 1 to 20 amino acids, from 1 to 10 amino acids, such as from 1 to 8 amino acids, from 2 to 6 amino acids, or from 2 to 4 amino acids bound to the N-terminus and/or the C-terminus of the IL-7Rα and/or Rγc ligand.

Flanking amino acids can comprise any suitable naturally occurring or non-naturally occurring amino acids.

For example, flanking amino acids can be selected from serine and flexible amino acids such as serine.

An IL-7Rα ligand and/or an Rγc ligand can comprise flanking amino acids such as, for example, terminal glycine groups on the N-terminus and/or the C-terminus of the respective ligand. For example, an IL-7Rα and an Rγc ligand can comprise flanking amino acids such as (G)ₙ glycine groups where n is from 1 to 10 (SEQ ID NO: 7), from 1 to 8, from 2 to 6, from 2 to 4, or from 2 to 3. For example, each of an IL-7Rα ligand and an Rγc ligand can independently comprise flanking amino acids such as 1, 2 or 3 terminal glycine groups. For example, a ligand having SEQ ID NO: 107, -Q-C-I-H-W-D-I-E-T-L-L-S-C-V-, can independently include flanking glycines such as -G-, -G-G- (SEQ ID NO: 21), or -G-G-G- or (SEQ ID NO: 22) on both the N-terminus and the C-terminus such that the resulting ligand can have the amino acid sequence, for example, -G-Q-C-I-H-W-D-I-E-T-L-L-S-C-V-G- (SEQ ID NO: 194), -G-G-Q-C-I-H-W-D-I-E-T-L-L-S-C-V-G-G- (SEQ ID NO: 195), or -G-G-G-Q-C-I-H-W-D-I-E-T-L-L-S-C-V-G- (SEQ ID NO: 196), respectively.

An IL-7Rαγc agonist peptide can comprise an IL-7Rα ligand and an Rγc ligand in which the IL-7Rα ligand is directly bound to the Rγc ligand.

An IL-7Rαγc agonist peptide can comprise an IL-7Rα ligand and an Rγc ligand bound to an IL-7Rαγc linker.

IL-7Rαγc agonist peptides that contain more than 2 cysteines can have a preferred pattern of Cys-Cys bonds (disulfide bridges) that exhibit the greatest activity such as, for example, Cys 1-2, and Cys 3-4, and other disulfide patterns may exhibit desired activity, and have useful properties.

Each of an IL-7Rα ligand and an Rγc ligand can independently be covalently bound to an IL-7Rαγc linker through the N-terminus or through the C-terminus of the IL-7Rαγc linker. For example, an IL-7Rα ligand can be bound to the IL-7Rαγc linker through the N-terminus and an Rγc ligand can be bound to an IL-7Rαγc linker through the N-terminus; an IL-7Rα ligand can be bound to an IL-7Rαγc linker through the N-terminus and an Rγc ligand can be bound to the IL-7Rαγc linker through the C-terminus; an IL-7Rα ligand can be bound to the IL-7Rαγc linker through the C-terminus and an Rγc ligand can be bound to the IL-7Rαγc linker through the N-terminus; or an IL-7Rα ligand can be bound to the IL-7Rαγc linker through the C-terminus and an Rγc ligand can be bound to the IL-7Rαγc linker through the C-terminus.

IL-7Rαγc agonist peptides having various C/N orientations of the IL-7Rα ligand and the Rγc ligand can be synthesized using click chemistry. The triazole linkage is a schematic representation of a synthetic IL-7Rαγc ligand linker, which can comprise various chemical moieties and can have various lengths and properties.

An IL-7Rαγc linker can be configured to facilitate binding of an IL-7Rαγc agonist peptide to the IL-7Rα subunit and to the Rγc subunit of IL-7R. For example, an IL-7Rαγc linker can be configured to facilitate activation of IL-7R by the IL-7Rαγc agonist peptide. For example, an IL-7Rαγc agonist peptide can be configured to induce IL-7R-mediated STAT5 phosphorylation in TF-1-7α cells.

An IL-7Rαγc linker can have a length, for example, from 2 Å to 100 Å, from 2 Å to 80 Å, from 2 Å to 60 Å, from 2 Å to 40 Å, from 2 Å to 20 Å, from 4 Å to 18 Å, from 6 Å to 16 Å, or from 8 Å to 14 Å. A ligand linker can have a length, for example, less than 100 Å, less than 80 Å, less than 60 Å, less than 40 Å, less than 20 Å, less than 15 Å, or less than 10 Å.

An IL-7Rαγc linker can comprise a backbone having, for example, from 2 to 50 bonds, from 2 to 45 bonds, from 2 to 40 bonds, from 2 to 35 bonds, from 2 to 30 bonds, from 2 to 25 bonds, from 2 to 20 bonds, from 4 to 18 bonds, from 6 to 16 bonds, or from 8 to 14 bonds. An IL-7Rαγc linker can comprise a backbone having, for example, less than 50 bonds, less than 40 bonds, less than 30 bonds, less than 20 bonds, or less than 10 bonds.

An IL-7Rαγc linker provided by the present disclosure can comprise a peptidyl IL-7Rαγc linker or a chemical IL-7Rαγc linker.

An IL-7Rαγc linker provided by the present disclosure can comprise a peptidyl IL-7Rαγc linker.

A peptidyl ligand linker can comprise, for example, from 2 to 100 amino acids, from 2 to 80 amino acids, from 2 to 60 amino acids, from 2 to 40 amino acids, from 2 to 20 amino acids, from 5 to 10 amino acids, or from 2 to 5 amino acids. A peptidyl ligand linker can comprise, for example, less than 100 amino acids, less than 80 amino acids, less than 40 amino acids, less than 20 amino acids, less than 15 amino acids, less than 10 amino acids, or less than 5 amino acids. Amino acids forming a peptidyl IL-7Rαγc linker can comprise naturally occurring amino acids and/or non-naturally occurring amino acids.

A peptidyl IL-7Rαγc linker can comprise, for example, flexible amino acids such as glycine. Flexible linkers can include small, non-polar amino acids such as glycine or polar amino acids. The small size of these amino acids provides flexibility and allows for mobility of the connecting functional domains. Incorporation of serine or threonine can maintain the stability of the linker in aqueous solutions by forming hydrogen bonds with water molecules, and thereby reduces unfavorable interactions between the linker and protein moieties. Amino acids such as lysine and glutamic acid can be included to improve solubility. The length of a peptidyl IL-7Rαγc linker can be selected to provide a suitable separation between the IL-7Rα and Rγc ligands to favor a desired interaction with IL-7R such as enhancing agonist activity. Examples of flexible linkers include (G)ₙ (SEQ ID NO: 2), (GS)ₙ (SEQ ID NO: 3), (GGS)ₙ (SEQ ID NO: 4), (GGGS)ₙ (SEQ ID NO: 5), (GGGGS)ₙ (SEQ ID NO: 6), and a combination of any of the foregoing, where n can independently be, for example, an integer from 1 to 20, such as from 1 to 15, from 1 to 10, from 1 to 5, from 5 to 10, or from 5 to 15.

A peptidyl IL-7Rαγc linker can be a rigid linker. Rigid linkers can be proline rich and can include other amino acids such as alanine, lysine, and/or glutamic acid. A rigid linker can have the sequence (PX)ₙ (SEQ ID NO: 39), where X can be, for example, alanine, lysine, or glutamic acid, and n can be an integer, for example, from 1 to 20, such as from 1 to 15, from 1 to 10, from 1 to 5, from 5 to 10, or from 5 to 15. Examples of rigid linkers include (PA)ₙ where n can be, for example, an integer from 1 to 20 (SEQ ID NO: 31). The value of n can be, for example, greater than 5, greater than 10, greater than 20, greater than 30, greater than 40 or greater than 50.

A peptidyl IL-7Rαγc linker can comprise, for example, (G)ₙ (SEQ ID NO: 2), (GS)ₙ (SEQ ID NO: 3), (GGS)ₙ (SEQ ID NO: 4), (GGGS)ₙ (SEQ ID NO: 5), (GGGGS)ₙ (SEQ ID NO: 6), or combinations of any of the foregoing, where n is independently an integer from 1 to 20. The value of n can be, for example, greater than 5, greater than 10, greater than 20, greater than 30, greater than 40 or greater than 50. For example, a peptidyl ligand linker can be -G-G- (SEQ ID NO: 21), -G-G-G- (SEQ ID NO: 22), -G-G-S- (SEQ ID NO: 24), -G-G-G-S- (SEQ ID NO: 25), -G-G-G-G-S-G-G- (SEQ ID NO: 26), or -G-G-S-G-G-S- (SEQ ID NO: 27). IL-7Rαγc agonist peptides comprising a peptidyl IL-7Rαγc linker can be synthesized using non-recombinant methods such as using the solid phase synthesis as described in Example 1 or can be synthesized using recombinant DNA technology.

An IL-7Rαγc linker can comprise a chemical IL-7Rαγc linker. A chemical IL-7Rαγc linker refers to a linker that is synthesized using chemical methods and can include amino acids or may not include amino acids. A chemical IL-7Rαγc linker can comprise a triazole moiety.

A synthetic chemical ligand linker can have the structure, for example, of Formula (L1)-(L17) as shown in Table 1.

TABLE 1
IL-7Rαγc chemical linkers.

| Formula | |
|---|---|
| No. | Chemical Structure |
| (L1) | 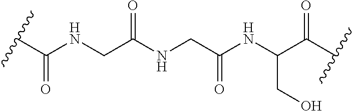 [SMILES: *C(=O)NCC(=O)NCC(=O)NC(CO)C(*)=O] |
| (L2) | 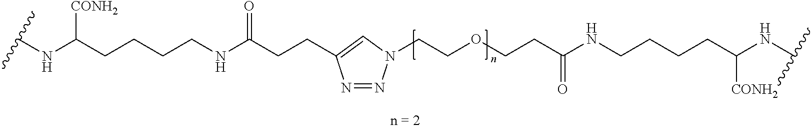 |
| (L3) | 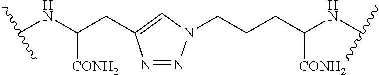 [SMILES: *NC(CCCn1cc(CC(N*)C(N)=O)nn1)C(N)=O] |
| (L4) | 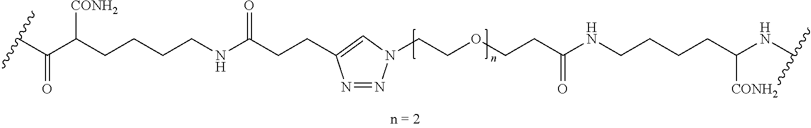 |
| (L5) | 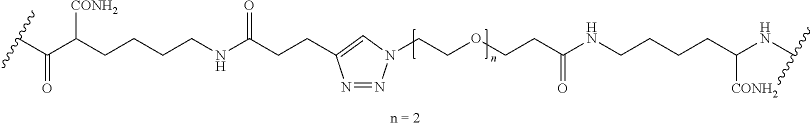 |
| (L6) | 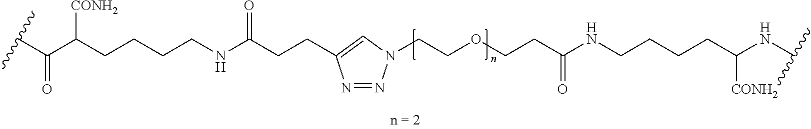 |
| (L7) | 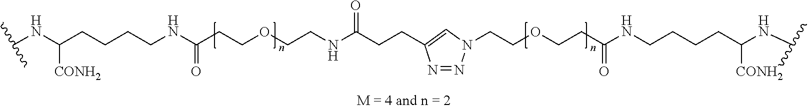 |
| (L8) | 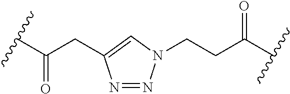 [SMILES: *C(=O)CCn1cc(CC(*)=O)nn1] |
| (L9) | 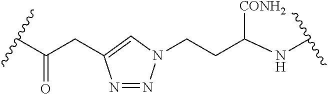 [SMILES: *NC(CCn1cc(CC(*)=O)nn1)C(N)=O] |
| (L10) | 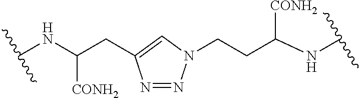 [SMILES: *NC(CCn1cc(CC(N*)C(N)=O)nn1)C(N)=O] |
| (L11) | 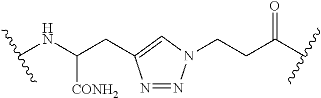 [SMILES: *NC(Cc1cn(CCC(*)=O)nn1)C(N)=O] |
| (L12) | 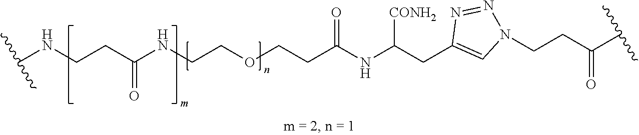 |
| (L13) | 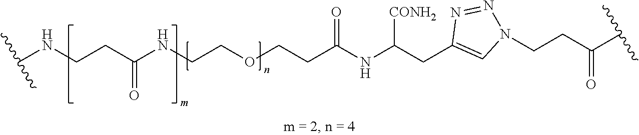 |
| (L14) | 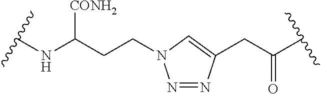 [SMILES: *NC(CCn1cc(CC(*)=O)nn1)C(N)=O] |
| (L15) | 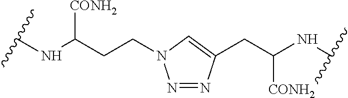 [SMILES: *NC(CCn1cc(CC(N*)C(N)=O)nn1)C(N)=O] |
| (L16) | 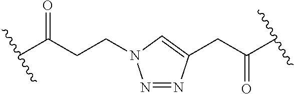 [SMILES: *C(=O)CCn1cc(CC(*)=O)nn1] |
| (L17) | 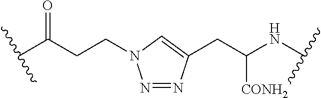 [SMILES: *NC(Cc1cn(CCC(*)=O)nn1)C(N)=O] |

In IL-7Rαγc linkers (L2), (L4)-(L7), (L12), and L13), each m and/or n can be independently an integer, for example, from 1 to 10.

A chemical IL-7Rαγc linker can be synthesized using click chemistry to provide IL-7Rαγc agonist peptides having various C/N orientations of the IL-7Rα and Rγc ligands. C/N orientation refers to the terminus of the IL-7Rα and Rγc which are bonded to the IL-7Rαγc linker. For example, for an IL-7Rαγc agonist peptide having a C/N orientation, the C-terminus of the IL-7Rα ligand is bonded to the IL-7Rαγc linker, and the N-terminus of the Rγc ligand is bonded to the IL-7Rαγc linker. As another example, for an IL-7Rαγc agonist peptide having an N/C orientation, the N-terminus of the IL-7Rα ligand is bonded to the IL-7Rαγc linker, and the C-terminus of the Rγc ligand is bound to the IL-7Rαγc linker.

IL-7Rα and Rγc ligands can be prepared using standard solid phase peptide synthesis and Fmoc-protected amino acids. A swollen resin can be treated with either an activated solution of Fmoc-propargyl glycine or 2-(Fmoc-NH)-azido-pentanoic acid to provide the corresponding Fmoc-protected resin. The alkyne-containing moiety and the azide-containing moiety can be configured to have, for example, a desired length, rigidity/flexibility, polarity, lipophilicity, and/or steric property. The protected resin can be subjected to repeated cycles of Fmoc-amino acid couplings with HATU activation and Fmoc removal to synthesize the respective IL-7Rα ligand or Rγc ligand. After Fmoc removal from the final amino acid of the IL-7Rα or Rγc ligand, and acylation of terminal amine groups, the ligands can be cleaved from the resin and purified.

The alkyne-containing moiety and azide-containing moiety bonded to an IL-7Rα ligand and a Rγc ligand can be reacted, for example, in the presence of CuSO₄ and a metal chelator to provide an IL-7Rαγc agonist peptide comprising a synthetic chemical IL-7Rαγc linker. The reacted alkyne-containing moiety and azide-containing moiety form the chemical linker. For example, referring to Tables 1-3, an alkyne-containing moiety of Formula (AL) in Table 2 can be reacted with an azide-containing moiety of Formula (AZ) in Table 3 to provide a synthetic IL-7Rαγc linker of Formula (L) in Table 1.

Using this click-chemistry method, IL-7Rαγc agonist peptides comprising IL-7Rα and Rγc ligands having differing N-terminal and C-terminal orientations and different ligand linker lengths can be synthesized.

Examples of alkyne-containing moieties are provided in Table 2 and examples of azide-containing moieties are provided in Table 3.

TABLE 2
Examples of alkyne-containing moieties.

| Formula | |
|---|---|
| No. | Chemical Structure |
| (AL1) | 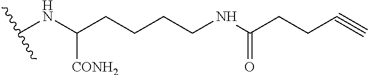 [SMILES: *NC(CCCCNC(=O)CCC#C)C(N)=O] |
| (AL2) | 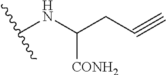 [SMILES: *NC(CC#C)C(N)=O] |
| (AL3) | 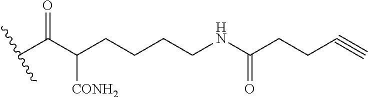 [SMILES: *C(=O)C(CCCCNC(=O)CCC#C)C(N)=O] |
| (AL4) | 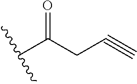 [SMILES: *C(=O)CC#C] |
| (AL5) | 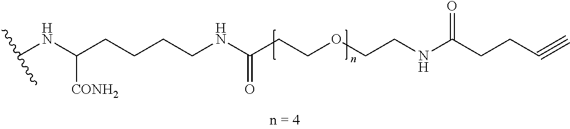 |
| (AL6) | 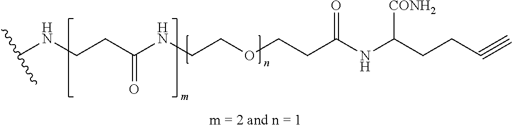 |
| (AL7) | 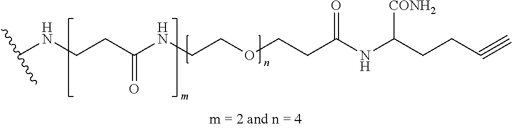 |
| (AL8) | 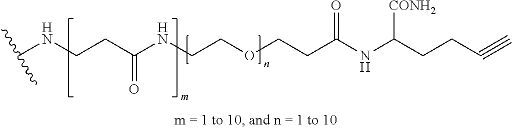 |
| (AL9) | 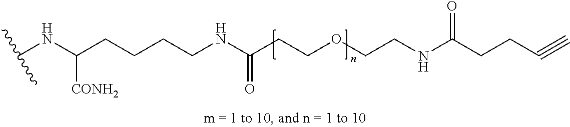 |

TABLE 3
Examples of azide-containing moieties.

| Formula | |
|---|---|
| No. | Chemical Structure |
| (AZ1) | 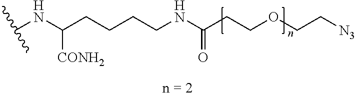 |
| (AZ2) | 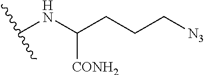 [SMILES: *NC(CCCN=[N+]=[N-])C(N)=O] |
| (AZ3) | 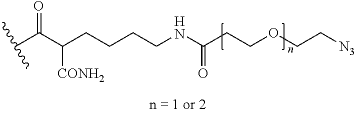 |
| (AZ4) | 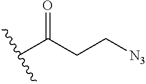 [SMILES: *C(=O)CCN=[N+]=[N-]] |
| (AZ5) | 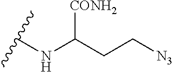 [SMILES: *NC(CCN=[N+]=[N-])C(N)=O] |

An IL-7Rαγc agonist peptide can comprise N- and/or C-terminal modifications to prevent or minimize degradation by aminopeptidases and carboxypeptidases. Examples of terminal groups include an acetyl group on the N-terminus and a carboxamide group on the C-terminus.

IL-7Rαγc agonist peptides provided by the present disclosure can comprise, for example, a moiety having the structure of Formula (4):

-AL-L-GL- (4)

where AL comprises an IL-7Rα ligand provided by the present disclosure, L comprises an IL-7Rαγc linker provided by the present disclosure, and GL comprises an Rγc ligand provided by the present disclosure.

A moiety of Formula (4) can be terminated in small chemical moieties and can have a molecular weight, for example, less than 12,000 Da, less than 11,000 Da, less than 10,000 Da, less than 9,000 Da, less than 8,000 Da, less than 7,000 Da, less than 6,000 Da, less than 5,000 Da, less than 4,000 Da, less than 3,000 Da, less than 2,000 Da, or less than 1,000 Da. An IL-7Rαγc agonist peptide can have a molecular weight, for example, from 1,000 Da to 12,000 Da, from 2,000 Da, to 11,000 Da, from 3,000 Da, to 10,000 Da, or from 4,000 Da to 9,000 Da.

In IL-7Rαγc agonist peptides of Formula (4), AL can comprise an IL-7Rα ligand having an amino acid sequence of any one of SEQ ID NO: 43-46, 55-60, 100-117, 118-193, and 197-245, a truncated amino acid sequence of any one of SEQ ID NO: 43-46, 55-60, 100-117, 118-193, and 197-245, or having an amino acid sequence having greater than 60%, greater than 70%, greater than 80%, greater than 85%, greater than 90%, or greater than 95% sequence similarity to any one of SEQ ID NO: 43-46, 55-60, 100-117, 118-193, and 197-245; GL can comprise an Rγc ligand having an amino acid sequence of any one of SEQ ID NO: 246-327 and 47-52, or having an amino acid sequence having greater than 60%, greater than 70%, greater than 80%, greater than 85%, greater than 90%, or greater than 95% sequence similarity to any one of SEQ ID NO: 246-327 and 47-52, and L can comprise a peptidyl IL-7Rαγc linker or a chemical IL-7Rαγc linker.

In IL-7Rαγc agonist peptides of Formula (4), either the N-terminus or the C-terminus of the IL-7Rα ligand can be bound to the IL-7Rαγc linker and either the N-terminus or the C-terminus of Rγc ligand can be bound to the IL-7Rαγc linker. For example, the C-terminus of the IL-7Rα ligand (BL) can be bound to the IL-7Rαγc linker (L) and the N-terminus of the Rγc ligand (GL) can be bound to the IL-7Rαγc linker (L).

In IL-7Rαγc agonist peptides of Formula (4) each of the IL-7Rα ligand and the Rγc ligand can comprise one or more flanking amino acids bound to the N-terminus and/or to the C-terminus of the ligand. For example, both the N-terminus and the C-terminus of the IL-7Rα ligand can comprise -(G)ₙ- and both the N-terminus and the C-terminus of the Rγc ligand can comprise (G)ₙ where n is an integer from 1 to 10 (SEQ ID NO: 7), such as from 1 to 8, from 2 to 6, or from 2 to 4. The flanking amino acids can be bound to the IL-7Rαγc linker.

IL-7Rαγc agonist peptides of Formula (4) can comprise an acetyl terminal group on the N-terminus and a carboxamide group on the C-terminus.

An IL-7Rαγc agonist peptide can comprise the structure of Formula (4a):

-(A)ₙ-AL-(A)ₙ-L-(A)ₙ-GL-(A)ₙ- (4a)

where,
each n can be independently an integer from 0 to 10;
AL can be an IL-7Rα ligand provided by the present disclosure;
GLₐ can be an Rγc ligand provided by the present disclosure;
each A can be an independently selected amino acid; and
L can be a peptidyl ligand linker comprising from 1 to 50 amino acids.

In IL-7Rαγc agonist peptides of Formula (4a),
each n can be independently an integer from 0 to 10;
   AL can be an IL-7Rα ligand comprising an amino acid sequence or truncated amino acid sequence selected from any one of SEQ ID NO: 43-46, 55-60, 100-117, 118-193, and 197-245, or comprising an amino acid sequence having greater than 60%, greater than 70%, greater than 80%, greater than 85%, or greater than 90% sequence similarity to any one of SEQ ID NO: 43-46, 55-60, 100-117, 118-193, and 197-245;
   GL can be an Rγc ligand comprising an amino acid sequence or a truncated amino acid sequence selected from any one of SEQ ID NO: 246-327 and 47-52, or comprising an amino acid sequence having greater than 60%, greater than 70%, greater than 80%, greater than 85%, or greater than 90% sequence similarity to any one of SEQ ID NO: 246-327 and 47-52;
   each A can be an independently selected amino acid; and
   L can be a peptidyl ligand linker comprising from 1 to 50 amino acids.

In IL-7Rαγc agonist peptides of Formula (4a), the C-terminus of the IL-7Rα ligand can be bound to the peptidyl ligand linker, and the N-terminus of the Rγc ligand can be bound to the peptidyl ligand linker.

In IL-7Rαγc agonist peptides of Formula (4a) each n can independently be selected from, for example, an integer from 0 to 8, from 0 to 6, from 0 to 4, or from 0 to 2. For example, n can be 0, 1, 2, or 3.

Each A can independently be selected from a naturally occurring or non-naturally occurring amino acid. Each A can independently be selected from a flexible amino acid such as glycine and serine.

Each A can be glycine.

Lₐ can comprise, for example, from 1 to 40 amino acids, from 1 to 30 amino acids, from 1 to 20 amino acids, from 1 to 10 amino acids, or from 1 to 5 amino acids. Lₐ can be selected from a peptidyl ligand linker. For example, Lₐ can be (PA)ₙ (SEQ ID NO: 1), (G)ₙ (SEQ ID NO: 7), (GS)ₙ (SEQ ID NO: 8), (GGS)ₙ (SEQ ID NO: 9), (GGGS)ₙ (SEQ ID NO: 10), (GGGGS)ₙ (SEQ ID NO: 11), or a combination of any of the foregoing, where p can independently be an integer from 1 to 10.

IL-7Rαγc agonist peptides provided by the present disclosure can comprise disulfide bonds. IL-7Rα ligands and Rγc ligands can comprise at least two cysteines. The at least two cysteines of an IL-7Rα ligand can be bound to another cysteine through a disulfide bond and each of the at least two cysteines of an Rγc ligand can be bound to a cysteine through a disulfide bond.

In an IL-7Rαγc agonist peptide, two cysteines of the IL-7Rα ligand can be bound together through a disulfide bond and/or two cysteines of the Rγc ligand can be bound together through a disulfide bond. In an IL-7Rαγc agonist peptide a cysteine of an IL-7Rα ligand can be bound to a cysteine of an Rγc ligand through a disulfide bond, or each of the two cysteines of an IL-7Rα ligand can be bound to a cysteine of an Rγc ligand. For example, in an IL-7Rαγc agonist peptide having the structure (5):

—X—C¹—X—C²—X-L-Y—C³—Y—C⁴—Y— (5)

where —X—C¹—X—C²—X-represents an amino acid sequence of an IL-7Rα ligand having two cysteines, C¹ and C², such as any one of SEQ ID NO: 43-46, 55-60, 100-117, 118-193, and 197-245, and where each X is independently one or more amino acids; —Y—C³—Y—C⁴—Y— represents an amino acid sequence of an Rγc ligand having two cysteines, C³ and C⁴, such as any one of SEQ ID NO: 246-327 and 47-52, and where each Y is independently one or more amino acids, and -L- is an IL-7Rαγc linker coupling the IL-7Rα ligand and the Rγc ligand.

In an IL-7Rαγc agonist peptide of Formula (5), C¹ can be bound to C² and C³ can be bound to C⁴ through disulfide bonds; C¹ can be bound to C³ and C² can be bound to C⁴ through disulfide bonds, or C¹ can be bound to C⁴ and C² can be bound to C³ through disulfide bonds.

IL-7Rαγc agonist peptides that contain more than 2 cysteines can have a preferred pattern of Cys-Cys bonds (disulfide bridges) that exhibit the greatest activity such as, for example, Cys 1-2, and Cys 3-4, and other disulfide patterns can exhibit desired activity and have useful properties.

An IL-7Rαγc agonist peptide can comprise an IL-7Rα ligand having SEQ ID NO: 114, 152-154, 368, 369, 374, 383, 394-397, 405, 420, and 442, a truncated amino acid sequence of SEQ ID NO: 114, 152-154, 368, 369, 374, 383, 394-397, 405, 420, and 442, or an amino acid sequence having greater than 60%, greater than 70%, greater than 75%, greater than 80%, greater than 85%, greater than 90%, or greater than 95% sequence similarity to SEQ ID NO: 100-245; and an Rγc ligand having SEQ ID NO: 261 or 325-327, a truncated amino acid sequence of SEQ ID NO: 261 or 325-327, or an amino acid sequence having greater than 60%, greater than 70%, greater than 75%, greater than 80%, greater than 85%, greater than 90%, or greater than 95% sequence similarity to SEQ ID NO: 261 or 325-327.

IL-7Rαγc agonist peptides can comprise an IL-7Rα ligand having SEQ ID NO: 114 or 152-154, a truncated amino acid sequence of SEQ ID NO: 114 or 152-154, or an amino acid sequence having greater than 60%, greater than 70%, greater than 75%, greater than 80%, greater than 85%, greater than 90%, or greater than 95% sequence similarity to SEQ ID NO: 114 or 152-154; and an Rγc ligand having SEQ ID NO: 261 or 325-327, a truncated amino acid sequence of SEQ ID NO: 261 or 325-327, or an amino acid sequence having greater than 60%, greater than 70%, greater than 75%, greater than 80%, greater than 85%, greater than 90%, or greater than 95% sequence similarity to SEQ ID NO: 261 or 325-327.

In IL-7Rαγc agonist peptides the C-terminus of the IL-7Rα ligand can be linked to the N-terminus of the Rγc ligand.

An IL-7Rαγc agonist peptide can comprise an IL-7Rα ligand having greater than 70%, greater than 80% or greater than 90% amino acid sequence similarity to SEQ ID NO: 116 or 118-159, and an Rγc ligand having greater than 70%, greater than 80% or greater than 90% amino acid sequence similarity to SEQ ID NO: 261 or 325-327.

An IL-7Rαγc agonist peptide can comprise an IL-7Rα ligand having greater than 70%, greater than 80% or greater than 90% amino acid sequence similarity to SEQ ID NO: 114 or 152-154, and an Rγc ligand having greater than 70%, greater than 80% or greater than 90% amino acid sequence similarity to SEQ ID NO: 261 or 325-327.

Each of the IL-7Rα ligand and the Rγc ligand can independently comprise one or more flanking amino acids such as one or more glycines. For example, each of the N-terminus and the C-terminus of the IL-7Rα ligand and the Rγc ligand can independently comprise glycines.

The N-terminus of the IL-7Rα ligand can be coupled to the C-terminus of the Rγc ligand through a flexible linker comprising, for example, from 1 to 50 amino acids, from 1 to 40, from 1 to 30, from 1 to 20, from 1 to 10, or from 1 to 5. A flexible linker can comprise, for example, greater than 5 amino acids, greater than 10, greater than 20, greater than 30, greater than 40, or greater than 50 amino acids. The linker can be, for example, (G)ₙ (SEQ ID NO: 12), (GS)ₙ (SEQ ID NO: 13), (GGS)ₙ (SEQ ID NO: 14), (GGGS)ₙ (SEQ ID NO: 15), (GGGGS)ₙ (SEQ ID NO: 16), or a combination of any of the foregoing, where n can independently be an integer from 1 to 5. For example, the linker can be GGGGS (SEQ ID NO: 17) or GGGGSGG (SEQ ID NO: 26).

An IL-7Rαγc agonist peptide can comprise the amino acid sequence of SEQ ID NO: 443 (-VHRIPWCTLDPGGLQCAWLRQMGGGGSGGVVCQDWEGVELCWQ-), or an amino acid sequence having greater than 60%, greater than 70%, greater than 75%, greater than 80%, greater than 85%, greater than 90%, or greater than 95% sequence similarity to SEQ ID NO: 443.

An IL-7Rαγc agonist peptide can comprise the amino acid sequence of SEQ ID NO: 368 (-VHRIPWCTLDPGGLQCAWLRQM-X³⁰⁰-GGVVCQDWEGVELCWQ-) or can comprise an amino acid sequence having greater than 60%, greater than 70%, greater than 80%, greater than 85%, greater than 90%, or greater than 95% amino acid sequence similarity to SEQ ID NO: 368, where X³⁰⁰ can include from 1 to 20 amino acids. For example, X³⁰⁰ can be selected from (G)ₙ (SEQ ID NO: 12), (GS)ₙ (SEQ ID NO: 13), (GGS) (SEQ ID NO: 14), (GGGS)ₙ (SEQ ID NO: 15), (GGGGS)ₙ (SEQ ID NO: 16) or a combination of any of the foregoing, where n can independently be an integer from 1 to 5. For example, X³⁰⁰ can be selected from (P)ₙ (SEQ ID NO: 30) or (PA)ₙ (SEQ ID NO: 31) where n is an integer from 1 to 20. X³⁰⁰ can comprise, for example, (PX)ₙ (SEQ ID NO: 39) where each X can independently be selected from alanine, lysine, or glutamic acid, and n can be an integer from 1 to 20. X³⁰⁰ can be (PA)ₙ where n can be, for example, an integer from 1 to 10 (SEQ ID NO: 33).

An IL-7Rαγc agonist peptide can comprise the amino acid sequence of SEQ ID NO: 369 (-VHRIPWCTLDPGGLQCAWLRQM-X³⁰¹-VVCQDWEGVELCWQ-) or an amino acid sequence having greater 60%, greater than 70%, greater than 80%, greater than 85%, greater than 90%, or greater than 95% amino acid sequence similarity to SEQ ID NO: 369, where X³⁰¹ includes from 1 to 20 amino acids. For example, X³⁰¹ can be selected from (G)ₙ (SEQ ID NO: 12), (GS)ₙ (SEQ ID NO: 13), (GGS)ₙ (SEQ ID NO: 14), (GGGS)ₙ (SEQ ID NO: 15), (GGGGS)ₙ (SEQ ID NO: 16), or a combination of any of the foregoing, where n can independently be an integer from 1 to 5.

In IL-7Rαγc agonist peptides of any one of SEQ ID NO: 443-454 and 368-369 or an amino acid sequence having greater than 60%, greater than 70%, greater than 75%, greater than 80%, greater than 85%, greater than 90%, or greater than 95% sequence similarity to any one of SEQ ID NO: 443-454 and 368-369, the cysteines of the IL-7Rα ligand can be bound together through a disulfide bond, and the cysteines of the Rγc ligand can be bound together through a disulfide bond. In certain IL-7Rαγc agonist peptides, the cysteines of the IL-7Rα ligand can be bound to the cysteines of the Rγc ligand.

An IL-7Rαγc agonist peptide provided by the present disclosure can comprise an amino acid sequence selected from any one of SEQ ID NO: 374-383:

| | |
|---|---|
| SEQ ID NO: 374 | VHRIPWCTLDPGGLQCAWLRQMGGGGSGGVVCQDWEGVELCWQPPA |
| SEQ ID NO: 375 | VHRIPWCTLDPGGLQCAWLRQGGGGSGGVVCQDWEGVELCWQPPA |
| SEQ ID NO: 376 | VHRIPWCTLDPGGLQCAWLRGGGGSGGVVCQDWEGVELCWQPPA |
| SEQ ID NO: 377 | GWGIPWCTLDPGSLQCAWLGKHGGGGSGGVVCQDWEGVELCWQPPA |
| SEQ ID NO: 378 | VHRIPWCTLDPGGLQCAWLRQGGGGGSGGVVCQDWEGVELCWQPPA |
| SEQ ID NO: 379 | VHRIPWCTLDPGGLQCAWLRGGGGGGSGGVVCQDWEGVELCWQPPA |
| SEQ ID NO: 380 | VHRIPWCTLDPGGLQCAWLRQGGGGSGGVVCQDWEGVELCWQGG |
| SEQ ID NO: 381 | VHRIPWCTLDPGGLQCAWLRGGGGSGGVVCQDWEGVELCWQGG |
| SEQ ID NO: 382 | GWGIPWCTLDPGSLQCAWLGKHGGGGSGGVVCQDWEGVELCWQGG |
| SEQ ID NO: 383 | VHRIPWCTLDPGGLQCAWLRQM(PA)₈GVVCQDWEGVELCWQGG |

An IL-7Rαγc agonist peptide can comprise an amino acid sequence selected from any one of SEQ ID NO: 370-373, 384-404, and 367:

| | |
|---|---|
| SEQ ID NO: 370 | IEGRGGQCIHWDIETLLSCVGGGGSGGVVCQDWEGVELCWQ |
| SEQ ID NO: 371 | IEGRGGVPWCTLDPGSLQCAWFGGGGSGGVVCQDWEGVELCWQ |
| SEQ ID NO: 372 | IEGRGGRYECADLPGGLHCEFRGGGGSGGVVCQDWEGVELCWQ |
| SEQ ID NO: 373 | RHFDDIIPWCTLDPGSLQCAYLGGGGSGGVVCQDWEGVELCWQ |
| SEQ ID NO: 384 | HLGVPWCTLDPGSIQCAWLAKHGGGGSGGVVCQDWEGVELCWQ |
| SEQ ID NO: 385 | VVCQDWEGVELCWQGGGGSGGRHFDDIIPWCTLDPGSLQCAYL |
| SEQ ID NO: 386 | VVCQDWEGVELCWQGGGGSGGHLGVPWCTLDPGSIQCAWLAKH |
| SEQ ID NO: 367 | VHRIPWCTLDPGGLQCAWLRQMGGGGSGGVVCQDWEGVELCWQ |
| SEQ ID NO: 387 | HCKHWDLESLLLCVGGGGSGGVVCQDWEGVELCWQ |
| SEQ ID NO: 388 | QCVHWDLDTLFGCIREQLELGGGGSGGVVCQDWEGVELCWQ |
| SEQ ID NO: 389 | VVCQDWEGVELCWQGGGGSGGQCVHWDLDTLFGCIREQLEL |
| SEQ ID NO: 390 | IRSCLWQPGALHCTWWAEEEPVGGGGSGGVVCQDWEGVELCWQ |
| SEQ ID NO: 391 | VVCQDWEGVELCWQGGGGSGGIRSCLWQPGALHCTWWAEEEPV |
| SEQ ID NO: 392 | IPWCLLDPGGLQCVWLGGGGSGGVVCQDWEGVELCWQ |
| SEQ ID NO: 393 | VVCQDWEGVELCWQGGGGSGGIPWCLLDPGGLQCVWL |
| SEQ ID NO: 394 | VHRIPWCTLDPGGLQCAWLRQMGGGGSGGVVCQDWEGVELCWQG |
| SEQ ID NO: 395 | VHRIPWCTLDPGGLQCAWLRQMGGGGSGGVVCQDWEGVELCWQGG |
| SEQ ID NO: 396 | GVHRIPWCTLDPGGLQCAWLRQMGGGGSGGVVCQDWEGVELCWQGG |
| SEQ ID NO: 397 | GGVHRIPWCTLDPGGLQCAWLRQMGGGGSGGVVCQDWEGVELCWQGG |
| SEQ ID NO: 398 | VHRIPWCTLDPGGLQCAWLRGGGGSGGVVCQDWEGVELCWQ |
| SEQ ID NO: 399 | WGIPWCTLDPGSLQCAWLGKHGGGGSGGVVCQDWEGVELCWQ |
| SEQ ID NO: 400 | VHRIPWCTLDPGGLQCAWLRQGGGGSGGVVCQDWEGVELCWQ |
| SEQ ID NO: 401 | VHRIPWCTLDPGGLQCAWLRMGGGGSGGVVCQDWEGVELCWQ |
| SEQ ID NO: 402 | VHRIPWCTLDPGGLQCAWIRQMGGGGSGGVVCQDWEGVELCWQ |
| SEQ ID NO: 403 | VHRIPWCTLDPGGLQCAWVRQMGGGGSGGVVCQDWEGVELCWQ |
| SEQ ID NO: 404 | VHRIPWCTLDPGGLQCAWARQMGGGGSGGVVCQDWEGVELCWQ |

An IL-7Rαγc agonist peptide can comprise an amino acid sequence selected from any one of SEQ ID NO: 405-419:

| | |
|---|---|
| SEQ ID NO: 405 | GVHRIPWCTLDPGGLQCAWLRQMGGGGSGGVVCQDWEGVELCWQG |
| SEQ ID NO: 406 | VHRIPWCTLDPGGLQCAWLRQGGGGSGGVVCQDEGVELCWQ |
| SEQ ID NO: 407 | VHRIPWCTLDPGGLQCAWLRGGGGSGGVVCQDWGVELCWQ |
| SEQ ID NO: 408 | VHRIPWCTLDPGGLQCAWLGKHGGGGSGGVVCQWEGVELCWQ |
| SEQ ID NO: 409 | VHRIPWCTLDPGGLQCAWLRMGGGGSGGVVCQDWGVELCWQ |
| SEQ ID NO: 410 | GWGIPWCTLDPGGLQCAWLRQMGGGGSGGVVCQDWEGVELCWQ |
| SEQ ID NO: 411 | GWGIPWCTLDPGGLQCAWLRQGGGGSGGVVCQDEGVELCWQ |
| SEQ ID NO: 412 | GWGIPWCTLDPGGLQCAWLRGGGGSGGVVCQDWGVELCWQ |
| SEQ ID NO: 413 | GWGIPWCTLDPGGLQCAWLGKHGGGGSGGVVCQWEGVELCWQ |
| SEQ ID NO: 414 | GWGIPWCTLDPGGLQCAWLRMGGGGSGGVVCQDWGVELCWQ |
| SEQ ID NO: 415 | IPWCTLDPGGLQCAWLRQMGGGGSGGVVCQDWEGVELCWQ |
| SEQ ID NO: 416 | IPWCTLDPGGLQCAWLRQGGGGSGGVVCQDEGVELCWQ |
| SEQ ID NO: 417 | IPWCTLDPGGLQCAWLRGGGGSGGVVCQDWGVELCWQ |
| SEQ ID NO: 418 | IPWCTLDPGGLQCAWLGKHGGGGSGGVVCQWEGVELCWQ |
| SEQ ID NO: 419 | IPWCTLDPGGLQCAWLRMGGGGSGGVVCQDWGVELCWQ |

An IL-7Rαγc agonist peptide provided by the present disclosure can comprise a truncated amino acid sequence of any one of SEQ ID NO: 370-419.

An IL-7Rαγc agonist peptide provided by the present disclosure can comprise an amino acid sequence selected from any one of SEQ ID NO: 370-419, or a truncated amino acid sequence of any one of SEQ ID NO: 370-419, wherein the amino acid sequence can independently comprise from 1 to 5 glycines (G) (SEQ ID NO: 28) on the N-terminus, on the C-terminus, or on both the N- and C-termini.

An IL-7Rαγc agonist peptide provided by the present disclosure can comprise an amino acid sequence selected from any one of SEQ ID NO: 370-419, or a truncated amino acid sequence of any one of SEQ ID NO: 370-419, wherein the amino acid sequence comprises one or more amino acid substitutions such as from 1 to 10 or from 1 to 5 amino acid substitutions. Each amino acid substitution can independently be a conservative amino acid substitution or a non-conservative amino acid substitution.

An IL-7Rαγc agonist peptide of SEQ ID NO: 370-419 can bind to the human Rγc subunit with an IC₅₀ of less than 100 μM.

An IL-7Rαγc agonist peptide can comprise an amino acid sequence having an amino acid sequence similarity greater than 60%, greater than 70%, greater than 75%, greater than 80%, greater than 85%, greater than 90%, or greater than 95% to the amino acid sequence of any one of SEQ ID NO: 370-419.

An IL-7Rαγc agonist peptide can comprise an amino acid sequence selected from any one of SEQ ID NO: 420-434:

| | |
|---|---|
| SEQ ID NO: 420 | VHRIPWCTLDPGGLQCAWLRQM-X⁴⁰⁰-VVCQDWEGVELCWQ |
| SEQ ID NO: 421 | VHRIPWCTLDPGGLQCAWLRQ-X⁴⁰⁰-VVCQDEGVELCWQ |
| SEQ ID NO: 422 | VHRIPWCTLDPGGLQCAWLR-X⁴⁰⁰-VVCQDWGVELCWQ |
| SEQ ID NO: 423 | VHRIPWCTLDPGGLQCAWLGKH-X⁴⁰⁰-VVCQWEGVELCWQ |
| SEQ ID NO: 424 | VHRIPWCTLDPGGLQCAWLRM-X⁴⁰⁰-VVCQDWGVELCWQ |
| SEQ ID NO: 425 | GWGIPWCTLDPGGLQCAWLRQM-X⁴⁰⁰-VVCQDWEGVELCWQ |
| SEQ ID NO: 426 | GWGIPWCTLDPGGLQCAWLRQ-X⁴⁰⁰-VVCQDEGVELCWQ |
| SEQ ID NO: 427 | GWGIPWCTLDPGGLQCAWLR-X⁴⁰⁰-VVCQDWGVELCWQ |
| SEQ ID NO: 428 | GWGIPWCTLDPGGLQCAWLGKH-X⁴⁰⁰-VVCQWEGVELCWQ |
| SEQ ID NO: 429 | GWGIPWCTLDPGGLQCAWLRM-X⁴⁰⁰-VVCQDWGVELCWQ |
| SEQ ID NO: 430 | IPWCTLDPGGLQCAWLRQM-X⁴⁰⁰-VVCQDWEGVELCWQ |
| SEQ ID NO: 431 | IPWCTLDPGGLQCAWLRQ-X⁴⁰⁰-VVCQDEGVELCWQ |
| SEQ ID NO: 432 | IPWCTLDPGGLQCAWLR-X⁴⁰⁰-VVCQDWGVELCWQ |
| SEQ ID NO: 433 | IPWCTLDPGGLQCAWLGKH-X⁴⁰⁰-VVCQWEGVELCWQ |
| SEQ ID NO: 434 | IPWCTLDPGGLQCAWLRM-X⁴⁰⁰-VVCQDWGVELCWQ |

In an IL-7Rαγc agonist peptide of any one of SEQ ID NO: 420-434, X⁴⁰⁰ can be selected from, for example, (G)ₙ (SEQ ID NO: 12), (GS)ₙ (SEQ ID NO: 13), (GGS)ₙ (SEQ ID NO: 14), (GGGS)ₙ (SEQ ID NO: 15), (GGGGS)ₙ (SEQ ID NO: 16) or a combination of any of the foregoing, where n can independently be an integer from 1 to 5.

In an IL-7Rαγc agonist peptide of any one of 420-434, X⁴⁰⁰ can be -GGGGSGG- (SEQ ID NO: 26).

In an IL-7Rαγc agonist peptide of any one of SEQ ID NO: 420-434, the ligand can comprise flanking glycines on each terminus such as two flanking glycines on one or both termini.

An IL-7Rαγc agonist peptide provided by the present disclosure can comprise a truncated amino acid sequence of any one of SEQ ID NO: 420-434.

An IL-7Rαγc agonist peptide provided by the present disclosure can comprise an amino acid sequence selected from any one of SEQ ID NO: 420-434, or a truncated amino acid sequence of any one of SEQ ID NO: 420-434, wherein the amino acid sequence can independently comprise from 1 to 5 glycines (G) (SEQ ID NO: 28) on the N-terminus, on the C-terminus, or on both the N- and C-termini.

An IL-7Rαγc agonist peptide provided by the present disclosure can comprise an amino acid sequence selected from any one of SEQ ID NO: 420-434, or a truncated amino acid sequence of any one of SEQ ID NO: 420-434, wherein the amino acid sequence comprises one or more amino acid substitutions such as from 1 to 5 amino acid substitutions. An amino acid substitution can be a conservative amino acid substitution.

An IL-7Rαγc agonist peptide of any one of SEQ ID NO: 420-434 can bind to the human Rγc subunit with an IC₅₀ of less than 100 μM.

An IL-7Rαγc agonist peptide can comprise an amino acid sequence having an amino acid similarity greater than 60%, greater than 70%, greater than 75%, greater than 80%, greater than 85%, greater than 90%, or greater than 95% to the amino acid sequence of any one of SEQ ID NO: 420-434.

An IL-7Rαγc agonist peptide can be selected from a peptide having the amino acid sequence of any one of an amino acid sequence of Formula (6) (SEQ ID NO: 435), an amino acid sequence of Formula (6a) (SEQ ID NO: 436), an amino acid sequence of Formula (6b) (SEQ ID NO: 437), an amino acid sequence of Formula (6c) (SEQ ID NO: 438), an amino acid sequence of Formula (6d) (SEQ ID NO: 439), an amino acid sequence of Formula (6e) (SEQ ID NO: 440), an amino acid sequence of Formula (6f) (SEQ ID NO: 441), an amino acid sequence of Formula (6g) (SEQ ID NO: 442), a truncated an amino acid sequence of any one Formula (6)-(6g), an amino acid sequence having greater than 80% sequence similarity to any one of Formula (6)-(6g), or a combination of any of the foregoing:

-X¹⁹⁸-X¹⁹⁹-X²⁰⁰-I-P-W-C-T-L-D-P-G-X²¹⁰-L-Q-C-A-W-
L-X²¹⁷-X²¹⁸-X²¹⁹-X⁴⁰⁰-VVCQDWEGVELCWQ-
(6)
-X¹⁹⁹-X^{200-I-P-W-C-T-L-D-P-G-X210}-L-Q-C-A-W-L-
X²¹⁷-X²¹⁸-X²¹⁹-X⁴⁰⁰-VVCQDWEGVELCWQ-
(6a)
-X^{200-I-P-W-C-T-L-D-P-G-X210}-L-Q-C-A-W-L-X²¹⁷-
X²¹⁸-X²¹⁹-X⁴⁰⁰-VVCQDWEGVELCWQ-
(6b)
-I-P-W-C-T-L-D-P-G-X²¹⁰-L-Q-C-A-W-L-X²¹⁷-X²¹⁸-
X²¹⁹-X⁴⁰⁰-VVCQDWEGVELCWQ-
(6c)
-X¹⁹⁸-X¹⁹⁹-X²⁰⁰-I-P-W-C-T-L-D-P-G-X²¹⁰-L-Q-C-A-W-
L-X²¹⁷-X²¹⁸-X⁴⁰⁰-VVCQDWEGVELCWQ-
(6d)
-X¹⁹⁸-X¹⁹⁹-X²⁰⁰-I-P-W-C-T-L-D-P-G-X²¹⁰-L-Q-C-A-W-
L-X²¹⁷-X⁴⁰-VVCQDWEGVELCWQ-
(6e)
-X¹⁹⁸-X¹⁹⁹-X²⁰⁰-I-P-W-C-T-L-D-P-G-X²¹⁰-L-Q-C-A-W-
L-X⁴⁰⁰-VVCQDWEGVELCWQ-
(6f)
-X¹⁹⁸-X¹⁹⁹-X²⁰⁰-I-P-W-C-T-L-D-P-X²¹⁰-L-Q-C-A-W-L-
X⁴⁰⁰⁻VVCQDWEGVELCWQ-
(6g)

wherein each of X¹⁹⁸—X²⁰⁰, X²¹⁰, X²¹⁷⁻²¹⁹, and X⁴⁰⁰ is independently an amino acid or can be absent.

An IL-7Rαγc agonist peptide of any one of Formula (6)-(6g),
X¹⁹⁸ can be selected from A, G, P, S, T, and V;
X¹⁹⁹ can be selected from F, H, W, and Y;
X²⁰⁰ can be selected from A, G, H, K, P, R, S, and T;
X²¹ can be selected from A, G, P, S, and T;
X²¹⁷ can be selected from A, G, H, K, P, R, S, and T;
X²¹⁸ can be a single bond or can be absent;
X²¹⁹ can be selected from an amino acid and absent; and
X⁴⁰⁰ can be selected from (G)ₙ (SEQ ID NO: 12), (GS)ₙ (SEQ ID NO: 13), (GGS)ₙ (SEQ ID NO: 14), (GGGS)ₙ (SEQ ID NO: 15), (GGGGS)ₙ (SEQ ID NO: 16), (GGGGSGG) (SEQ ID NO: 26), or a combination of any of the foregoing, where n is an integer from 1 to 5.

In an IL-7Rαγc agonist peptide of any one of Formula (6)-(6g),
X¹⁹⁸ can be selected from V and G;
X¹⁹⁹ can be selected from H and W;
X²⁰⁰ can be selected from R and G;
X²¹⁰ can be selected from G and S;
X²¹⁷ can be selected from R and G;
X²¹⁸ can be selected from Q, G, and K or can be absent; and
X²¹⁹ can be selected from G, H, and M, or can be absent.

In an IL-7Rαγc agonist peptide of any one of Formula (6)-(6g), X¹⁹⁸ can be V, X¹⁹⁹ can be H, and X²⁰⁰ can be R.

In an IL-7Rαγc agonist peptide of any one of Formula (6)-(6g), X¹⁹⁸ can be G, X¹⁹⁹ can be W, and X²⁰⁰ can be G.

In an IL-7Rαγc agonist peptide of any one of Formula (6)-(6g), X²¹⁰ can be G.

In an IL-7Rαγc agonist peptide of any one of Formula (6)-(6g), X²¹⁰ can be S.

In an IL-7Rαγc agonist peptide of any one of Formula (6)-(6g), X²¹⁷ can be R.

In an IL-7Rαγc agonist peptide of any one of Formula (6)-(6g), X²¹⁷ can be R, X²¹⁸ can be Q, and X²¹⁹ can be M.

In an IL-7Rαγc agonist peptide of any one of Formula (6)-(6g), X²¹⁷ can be G, X²¹⁸ can be K, and X²¹⁹ can be H.

In an IL-7Rαγc agonist peptide of any one of Formula (6)-(6g), the IL-7Rα ligand can be defined by any combination of variables as defined in the immediately preceding nine (9) paragraphs.

In an IL-7Rαγc agonist peptide of any one of 435-2139, X⁴⁰⁰ can be -GGGGSGG- (SEQ ID NO: 26).

In an IL-7Rαγc agonist peptide of any one of SEQ ID NO: 435-442, the ligand can comprise flanking glycines on each terminus such as two flanking glycines on each terminus.

An IL-7Rαγc agonist peptide provided by the present disclosure can comprise a truncated amino acid sequence of any one of SEQ ID NO: 435-442.

An IL-7Rαγc agonist peptide provided by the present disclosure can comprise an amino acid sequence selected from any one of SEQ ID NO: 435-442, or a truncated amino acid sequence of any one of SEQ ID NO: 380-400, wherein the amino acid sequence can independently comprise from 1 to 5 glycines (G) (SEQ ID NO: 28) on the N-terminus, on the C-terminus, or on both the N- and C-termini.

An IL-7Rαγc agonist peptide provided by the present disclosure can comprise an amino acid sequence selected from any one of SEQ ID NO: 435-442, or a truncated amino acid sequence of any one of SEQ ID NO: 380-405, wherein the amino acid sequence comprises one or more amino acid substitutions such as from 1 to 10 or from 1 to 5 amino acid substitutions. Each amino acid substitution can independently be a conservative amino acid substitution or a non-conservative amino acid substitution.

An IL-7Rαγc agonist peptide of SEQ ID NO: 435-442 can bind to the human Rγc subunit with an IC₅₀ of less than 100 μM.

An IL-7Rαγc agonist peptide can comprise an amino acid sequence having an amino acid similarity greater than 60%, greater than 70%, greater than 75%, greater than 80%, greater than 85%, greater than 90%, or greater than 95% to the amino acid sequence of any one of SEQ ID NO: 435-442.

An IL-7Rαγc agonist peptide provided by the present disclosure can bind to IL-7R such as hu-IL-7R with an IC₅₀ from 1 μM to 100 μM, from 10 μM to 10 μM, from 100 μM to 1 μM, from 0.001 μM to 1 μM, or from 0.01 μM to 1 μM.

An IL-7Rαγc agonist peptide provided by the present disclosure can bind to IL-7R such as hu-IL-7R with an IC₅₀ of less than 100 μM, less than 10 pm, less than 1 pm, less than 100 μM, less than 10 μM, or less than 1 μM.

An IL-7Rαγc agonist peptide provided by the present disclosure can bind to each of the IL-7Rα subunit and to the IL-7Rγc subunit, such as each of the hu-IL-7Rα subunit and the hu-IL-7Rγc subunit, with an IC₅₀ from 1 μM to 100 μM, from 10 μM to 10 μM, from 100 μM to 1 μM, from 0.001 μM to 1 μM, or from 0.01 μM to 1 μM.

An IL-7Rαγc agonist peptide provided by the present disclosure can bind to each of the IL-7Rα subunit and the IL-7Rγc subunit, such as each of the hu-IL-7Rα subunit and to the hu-IL-7Rγc subunit with an IC₅₀ of less than 100 pm, less than 10 pm, less than 1 pm, less than 100 μM, less than 10 μM, or less than 1 μM.

An IL-7Rαγc agonist peptide provided by the present disclosure can exhibit an EC₅₀ for STAT5 phosphorylation in TF-1-7α cells, for example, of less than 100 μM, less than 10 μM, less than 1 μM, less than 100 μM, less than 10 μM, or less than 1 μM.

An IL-7Rαγc agonist peptide provided by the present disclosure can exhibit an EC₅₀ for STAT5 phosphorylation in TF-1-7α cells, for example, from 1 μM to 100 μM, from 10 μM to 10 μM, from 100 μM to 1 μM, from 0.001 μM to 1 μM, or from 0.01 μM to 1 μM.

An IL-7Rαγc agonist peptide provided by the present disclosure can comprise:
an amino acid sequence of any one of SEQ ID NO: 443-454 and 368-454;
a truncated amino acid sequence of any one of SEQ ID NO: 443-454 and 368-454;
an amino acid sequence of any one of SEQ ID NO: 443-454 and 368-454 having from 1 to 5 amino acid substitutions;
an amino acid sequence of any one of SEQ ID NO: 443-454 and 368-454 having from 1 to 5 flanking glycines on the N-terminus, the C-terminus, or both the N-terminus and the C-terminus;
an amino acid sequence having greater than 60% sequence similarity to an amino acid sequence of any one of SEQ ID NO: 443-454 and 368-454; or
a combination of any of the foregoing.

An IL-7Rαγc agonist peptide can comprise an amino acid sequence of any one of SEQ ID NO: 367-369, 374-434, and 443.

An IL-7Rαγc agonist peptide can comprise an amino acid sequence of any one of SEQ ID NO: 394-404.

An IL-7Rαγc agonist peptide can comprise an amino acid sequence of any one of SEQ ID NO: 395.

An IL-7Rαγc ligand provided by the present disclosure can bind to each of the IL-7Rα subunit and the IL-7Rγc subunit, such as each of the hu-IL-7Rα subunit and to the hu-IL-7Rγc subunit with an IC₅₀ of less than 100 pm, less than 10 pm, less than 1 pm, less than 100 μM, less than 10 μM, or less than 1 μM.

Examples of suitable IL-7Rαγc agonist peptides are disclosed, for example, in U.S. Publication No. 2021/0253670 A1, which is incorporated by reference in its entirety.

An IL-7Rαγc agonist peptide can have the amino acid sequence of any one of SEQ ID NO: 2012-2023, or an amino acid sequence having greater than 60%, greater than 70%, greater than 80%, greater than 85%, greater than 90%, or greater than 95% amino acid sequence similarity to any one of SEQ ID NO: 443-454:

| | |
|---|---|
| SEQ ID NO: 443 | VHRIPWCTLDPGGLQCAWLRQMGGGGSGGVVCQDWEGVELCWQ |
| SEQ ID NO: 444 | VHRIPWCTLDPGGLQCAWLRQGGGGSGGVVCQDEGVELCWQ |
| SEQ ID NO: 445 | VHRIPWCTLDPGGLQCAWLRGGGGSGGVVCQDWGVELCWQ |
| SEQ ID NO: 446 | HRIPWCTLDPGGLQCAWLRQMGGGGSGGVVCQWEGVELCWQ |
| SEQ ID NO: 447 | HRIPWCTLDPGGLQCAWLRQGGGGSGGVVCQD |
| SEQ ID NO: 448 | HRIPWCTLDPGGLQCAWLRGGGGSGGVVCQDWGVELCWQ |
| SEQ ID NO: 449 | RIPWCTLDPGGLQCAWLRQMGGGGSGGVVCQWEGVELCWQ |
| SEQ ID NO: 450 | RIPWCTLDPGGLQCAWLRQGGGGSGGVVCQDEGVELCWQ |
| SEQ ID NO: 451 | RIPWCTLDPGGLQCAWLRGGGGSGGVVCQDWWEGVELCWQ |
| SEQ ID NO: 452 | IPWCTLDPGGLQCAWLRQMGGGGSGGVVCQWEGVELCWQ |
| SEQ ID NO: 453 | IPWCTLDPGGLQCAWLRQGGGGSGGVVCQDEGVELCWQ |
| SEQ ID NO: 454 | IPWCTLDPGGLQCAWLRGGGGSGGVVCQDWGVELCWQ |

An IL-7Rαγc agonist peptide can be full IL-7R agonists or partial IL-7R agonists.

An IL-7Rαγc agonist peptide can bind to IL-7Rα subunit and Rγc subunit and can activate the IL-7 receptor. An IL-7Rαγc agonist peptide can independently bind to the IL-7Rα subunit and to Rγc subunit with an IC₅₀, for example, of less than 100 μM, less than 10 μM, less than 1 μM, less than 100 nM, less than 10 nM, or less than 1 nM. An IL-7Rαγc agonist peptide can bind to the IL-7Rα subunit and/or to the Rγc either competitively or non-competitively with IL-7.

An IL-7Rαγc agonist peptide comprising an IL-7Rα ligand and an Rγc ligand can be configured to more potently activate cells expressing the IL-7Rα subunit and the Rγc subunit, thereby facilitating the ability to differentially activate IL-7R expressed on the surface of different cell types by controlling dose of the agonist. For example, when incubated with a heteromeric compound comprising an IL-7Rα ligand and Rγc ligand, primary human peripheral blood mononuclear cells (PBMC) expressing the IL-7R subunits phosphorylate signal transducer and activator of transcription 5 (STAT5).

An IL-7Rαγc agonist peptide can partially activate the IL-7 receptor. Partial activation refers to a level of activation, that is, for example, less than 75% of maximum activation, less than 50%, less than 25%, less than 10%, or less than 1% of the maximum activation. Maximum activation (Eₘₐₓ) is the amplitude of cellular signal (activation) achievable at high agonist concentration such as a high concentration of IL-7. Partial IL-7Rαγc agonist peptides can be effective in modulating the levels of response of IL-7R to activation of the IL-7Rα and Rγc subunits among different cell types expressing IL-7R. For example, different cell types are known to vary in expression levels of each of the IL-7R subunits, i.e., the IL-7Rα and Rγc subunits, and to exhibit different sensitivities to IL-7R agonists.

An IL-7Rαγc agonist peptide can comprise an IL-7Rα ligand and a modified Rγc ligand. Modified Rγc ligands can be selected or designed to bind and activate IL-7R, but with low or modest affinity and potency to IL-7R. Such IL-7R agonists can have greater differential sensitivity for IL-7R activation between cells that highly express IL-7Rα and cells having a low level of IL-7Rα expression. An IL-7Rαγc agonist peptide can comprise one or more IL-7Rα ligands and one or more Rγc ligands. The presence of multiple IL-7Rα ligands and multiple Rγc ligands can preferentially increase the potency of the IL-7Rαγc agonist peptide on cells that highly express IL-7Rα and/or Rγc compared to cells having low expression levels of IL-7Rα and/or Rγc.

IL-7Rαγc agonist peptides can be full agonists or partial agonists of IL-7R.

An IL-7Rαγc agonist peptide can bind to the IL-7Rα subunit and/or to the Rγc subunit of IL-7R and can activate IL-7R. An IL-7Rαγc agonist peptide can independently bind to the IL-7Rα subunit and/or to the Rγc subunit with an IC₅₀, for example, of less than 100 μM, less than 10 μM, less than 1 μM, less than 100 nM, less than 10 nM, or less than 1 nM.

An IL-7Rαγc agonist peptide can bind to the IL-7Rα subunit and/or to the Rγc subunit with an IC₅₀, for example, of less than 100 μM, less than 10 μM, less than 1 μM, less than 100 nM, less than 10 nM, or less than 1 nM. An IL-7Rαγc agonist peptide can bind to the IL-7Rα subunit and/or to the Rγc subunit either competitively or non-competitively with IL-7.

An IL-7Rαγc agonist peptide can be configured to more potently activate cells expressing the IL-7Rα subunit and the Rγc subunit, thereby facilitating the ability to differentially activate IL-7R expressed on the surface of different cell types by controlling a dose of an IL-7Rαγc agonist peptide. For example, when incubated with an IL-7Rαγc agonist peptide, primary human peripheral blood mononuclear cells (PBMC) expressing the IL-7Rαγc subunit phosphorylate signal transducer and activator of transcription 5 (STAT5).

The EC₅₀ for STAT5 phosphorylation in TF-1-7α or hu-PMBCs induced by an IL-7Rαγc agonist peptide can be, for example, less than 100 μM, less than 10 μM, less than 1 μM, less than 100 nM, less than 10 nM, or less than 1 nM.

The EC₅₀ for STAT5 phosphorylation in TF-1-7α cells induced by an IL-7Rαγc agonist peptide can be, for example, within a range from 1 μM to 100 pm, from 10 μM to 10 pm, or from 100 μM to 1 pm.

IL-7Rαγc agonist peptide provided by the present disclosure can activate the STAT5 phosphorylation pathway, the AKT phosphorylation pathway, and the ERK1/2 phosphorylation pathway in CD4+ and CD8+ cells.

An IL-7Rαγc agonist peptide can partially activate IL-7R. Partial activation refers to a level of activation, that is, for example, less than 75% of maximum activation, less than 50%, less than 25%, less than 10%, or less than 1% of the maximum activation. Maximum activation (Eₘₐₓ) of IL-7R refers to the amplitude of cellular signal (activation) achievable at high agonist concentration such as a high concentration of IL-7. Partial IL-7Rαγc agonist peptides can be effective in modulating the levels of response of IL-7R to activation of the IL-7Rα and Rγc subunits among different cell types expressing IL-7R. For example, different cell types are known to vary in expression levels of each of the IL-7R subunits, IL-7Rα and Rγc, and to exhibit different sensitivities to IL-7R agonists.

An IL-7Rαγc agonist peptide can comprise modified IL-7Rα ligands and/or Rγc ligands. Modified IL-7Rα and Rγc ligands can be selected or designed to bind and activate IL-7R, but with low or modest affinity and potency to IL-7R. Such IL-7Rαγc agonist peptides can have greater differential sensitivity for IL-7R activation between cells that highly express IL-7Rα and cells having a low level of IL-7Rα expression.

An IL-7Rαγc agonist peptide can act as a full agonist comparable to IL-7 with respect to STAT5 phosphorylation in TF-1 IL-7Rα (TF-1-7a) cells and in resting human PMBCs.

With respect to STAT5 phosphorylation in TF-1 IL-7Rα cells and in resting human PMBCs, an IL-7Rαγc agonist peptide can exhibit agonist activity at less than 1 nm (EC₅₀).

IL-7Rαγc agonist peptides can comprise two or more IL-7Rαγc agonist peptides. The two or more IL-7Rαγc agonist peptides can be linked together to form a linear or non-linear structure. For example, a IL-7Rαγc agonist peptide can have the structure of Formula (7a) or Formula (7b):

AGL-(-Lₜ₁-AGL-)ₙ₁-Lₜ₁-AGL (7a)

Lₜ₂{(-Lₜ₁-AGL-)ₙ₂-Lₜ₁-AGL}ₚ (7b)

where,
each AGL can independently be selected from an IL-7Rαγc agonist peptide;
Lₜ₁ can be a divalent linker;
Lₜ₂ can be a p-valent linker;
n1 can be an integer from 1 to 6;
n2 can be an integer from 0 to 6; and
p can be an integer from 3 to 8.

In IL-7Rαγc agonist peptides of Formula (7a) and (7b), each IL-7Rαγc agonist peptide can be the same.

In IL-7Rαγc agonist peptides of Formula (7a) and (7b), at least one IL-7Rαγc agonist peptide can be different than another IL-7Rαγc agonist peptide.

In IL-7Rαγc agonist peptides of Formula (7a) and (7b), each IL-7Rαγc agonist peptide can independently be bound to a tandem linker through the N-terminus or through the C-terminus of the respective IL-7Rαγc agonist peptide.

In IL-7Rαγc agonist peptides of Formula (7a) and (7b), each of the IL-7Rαγc agonist peptides can comprise one or more flanking amino acids.

A linker, Lₜ₁ and Lₜ₂, can be a peptidyl tandem linker and can have, for example, from 1 to 50 amino acids, from 2 to 40 amino acids, or from 5 to 30 amino acids.

A linker can comprise a chemical linker such as a triazole-containing linker provided by the present disclosure.

Each divalent linker Lₜa can be the same as each of the other divalent tandem linkers, or at least one of the divalent tandem linkers can be different than another tandem linker.

In a IL-7Rαγc agonist peptide of Formula (7a), n can be, for example, 1, 2, 3, 4, 5, or 6.

In a IL-7Rαγc agonist peptide of Formula (7b), each n can independently be selected from 0, 1, 2, 3, 4, 5, or 6.

In a IL-7Rαγc agonist peptide of Formula (7b), p can be, for example, 3, 4, 5, 6, 7, or 8.

A p-valent linker can comprise any suitable polyfunctional chemical moiety. For example, IL-7Rαγc agonist peptides of Formula (7a) and (7b) can have a molecular weight less than 10,000 Da, less than 6,000 Da, less than 2,000 Da, less than 1,000 Da, or less than 500 Da.

An IL-7Rαγc agonist peptide provided by the present disclosure can comprise one or more IL-7Rα ligands and one or more Rγc ligands. The one or more IL-7Rα ligands and one or more Rγc ligands can be bonded together to form a linear or non-linear structure. For example, an IL-7Rαγc agonist peptide can have the structure of Formula (8a) or Formula (8b):

L-(-Lₜ₁-L-)ₙ₁-Lₜ₁-L (8a)

Lₜ₂{(-Lₜ₁-L-)ₙ₂-Lₜ₁-L}ₚ (8b)

where,
each L can be independently selected from an IL-7Rα ligand and an Rγc ligand; and
Lₜ₁ can be a divalent linker;
Lₜ₂ can be a p-valent linker;
n1 can be an integer from 1 to 6;
n2 can be an integer from 0 to 6; and
p can be an integer from 3 to 8, and
wherein the IL-2Rβγc agonist peptide comprises at least one IL-7Rα ligand and at least one Rγc ligand.

In IL-7Rαγc agonist peptide of Formula (8a) and (8b), each IL-7Rα ligand and each Rγc ligand can be the same.

In IL-7Rαγc agonist peptides of Formula (8a) and (8b), at least one IL-7Rα ligand can be different than another IL-7Rα ligand and/or at least one Rγc ligand can be different than another Rγc ligand

In IL-7Rαγc agonist peptides of Formula (8a) and (8b), each IL-7Rα ligand and Rγc ligand can independently be bound to a linker through the N-terminus or through the C-terminus of the respective IL-7Rαγc agonist peptide.

In IL-7Rαγc agonist peptides of Formula (8a) and (8b), each IL-7Rα ligand and Rγc ligand can independently comprise one or more flanking amino acids.

A linker, Lₜ₁ and Lₜ₂, can be a peptidyl tandem linker and can, for example, from 1 to 50 amino acids, from 2 to 40 amino acids, or from 5 to 30 amino acids.

A linker can comprise a chemical linker such as a triazole-containing linker provided by the present disclosure.

Each divalent linker Lₜ₁ can be the same as each of the other divalent linkers, or at least one of the divalent linkers can be different than another divalent linker.

In an IL-7Rαγc agonist peptide of Formula (8a), n can be, for example, 1, 2, 3, 4, 5, or 6.

In an IL-7Rαγc agonist peptide of Formula (8b), each n can independently be selected from 0, 1, 2, 3, 4, 5, or 6.

In an IL-7Rαγc agonist peptide of Formula (8b), p can be, for example, 3, 4, 5, 6, 7, or 8.

A p-valent tandem linker can comprise any suitable polyfunctional chemical moiety. For example, tandem IL-7Rαγc agonist peptides of Formula (8a) and (8b) can have a molecular weight, for example, less than 10,000 Da, less than 6,000 Da, less than 2,000 Da, less than 1,000 Da, or less than 500 Da.

Aspects of the present invention include a culture medium comprising an IL-7Rαγc agonist peptide such as an IL-7Rαγc agonist peptide provided by the present disclosure.

A culture medium can comprise any suitable culture medium for expanding a target immune cell population of an initial population of immune cells.

A culture medium can comprise a base culture medium and an IL-7Rαγc agonist peptide.

A base culture medium can comprise any suitable culture medium for culturing immune cells such as T cells.

A base culture media refers to any suitable starting media that is supplemented with one or more stimulants of immune cell proliferation. For example, a base culture media can comprise a balanced salt solution such as PBS, DPBS, HBSS, EBSS, Dulbecco's Modified Eagle's Medium (DMEM), Click's medium, Minimal Essential Medium (MEM), Basal Medium Eagle (BME), F-10, F-12, RPMI 1640, Glasgow Minimal Essential Medium (GMEM), alpha Minimal Essential Medium (alpha MEM), Iscove's Modified Dulbecco's Medium (IMDM), M199, OPTMIZER™ Pro, OPTMIZER™ CTS™ T-Cell Expansion Basal Medium (ThermoFisher), OPTMIZER™, OPTMIZER™ Complete, IMMUNOCULT™ XF (STEMCELL™ Technologies), AIM V™, TEXMACS™ medium, PRIME-XV® T cell CDM, X-VIVO™ 15 (Lonza), TRANSACT™ TIL expansion medium, or any combination thereof. A base culture medium can be serum-free. A base culture media can comprise PRIME-XV® T cell CDM. A base culture medium can comprise OPTMIZER™. A base culture media can comprise OPTMIZER™ Pro. A base culture medium further comprises immune cell serum replacement (ICSR).

A culture media provided by the present disclosure can be used for culturing immune cells such as for example, expanding a target population of immune cells such as T cells.

A target immune cell population can comprise immune cells for which the growth of the target immune cells is stimulated relative to other immune cells in the culture medium upon incubation with an IL-7Rαγc agonist peptide.

For example, a target immune cell population can comprise a target T cell population.

For example, a target T cell population can comprise CD4+ T cells and CD8+ T cells.

A culture medium can comprise a concentration of an IL-7Rαγc agonist peptide, for example, greater than 1 nM, greater than 10 nM, greater than 100 nM, greater than 200 nM, greater than 500 nM, greater than 1,000 nM, or greater than 2,000 nM.

A culture medium can comprise a concentration of an IL-7Rαγc agonist peptide, for example, less than 5,000 nM, less than 2,000 nM, less than 1,000 nM, less than 500 nM, less than 200 nM, less than 100 nM, less than 50 nM, or less than 10 nM,

A culture medium can comprise a concentration of an IL-7Rαγc agonist peptide, for example, from 1 nM to 5,000 nM, from 10 nM to 1,000 nM, or from 50 nM to 500 nM.

A culture medium can comprise a concentration of an IL-7Rαγc agonist peptide sufficient to stimulate the proliferation of a target immune cell population, such as a population of CD4+ T cells and CD8+ T cells.

A culture medium can comprise a second stimulant of the proliferation of the target immune cell population in addition to an IL-7Rαγc agonist peptide.

For example, a second stimulant for the proliferation of the target immune cell population can comprise, for example, an IL-2Rβγc agonist peptide, an IL-15Rβγc agonist peptide, or a combination thereof.

An IL-2Rβγc agonist peptide refers to a peptide comprising an IL-2RO ligand and an Rγc ligand, wherein the IL-2Rβγc agonist peptide has an EC50 for STAT5 phosphorylation of TF-13 cells of less than 100 μM, less than 10 μM, less than 1 μM, less than 100 μM, less than 10 μM, or less than 1 μM.

An IL-2Rβγc agonist peptide can comprise an IL-2RO ligand bound to an Rγc ligand through a ligand linker.

An IL-2Rβ ligand can bind to the hu-IL-2Rβ subunit with an IC₅₀ of less than 100 μM, less than 10 μM, less than 1 μM, less than 0.1 μM, or less than 0.1 μM, where binding is determined using a phage ELISA assay.

Examples of suitable IL-2Rβ ligands are disclosed, for example, in U.S. Pat. No. 10,703,776 and in U.S. Application Publication No. 2021/0198336 A1, each of which is incorporated by reference in its entirety.

In an IL-2Rβγc agonist peptide, an Rγc ligand can be an Rγc ligand as disclosed herein and can be the same or different Rγc ligand as in the IL-7Rαγc agonist peptide.

An Rγc ligand can bind to the hu-IL-2Rγc subunit with an IC₅₀ of less than 100 μM, less than 10 μM, less than 1 μM, less than 0.1 μM, or less than 0.1 μM, where binding is determined using a phage ELISA assay.

Examples of suitable Rγc ligands are disclosed, for example, in U.S. Pat. No. 10,689,417 and in U.S. Application Publication No. 2021/0253670 A1, each of which is incorporated by reference in its entirety.

An IL-2Rβγc agonist peptide can comprise an amino acid sequence of any one of SEQ ID NO: 359-366, and 61-99.

| | |
|---|---|
| SEQ ID NO: 359 | -GGWYPCWMAQLGELCDLDGGGGSGGVVCQDWEGVELCWQGG- |
| SEQ ID NO: 360 | -WYPCWMAQLGELCDLDGGGGSGGVVCQDWEGVELCWQ- |
| SEQ ID NO: 361 | -WYPCWMAQLGELCDLDGG-X⁴⁰⁰-GGVVCQDWEGVELCWQ- |
| SEQ ID NO: 362 | -WYPCWMAQLGELCDLD-X⁴⁰⁰-VVCQDWEGVELCWQ- |
| SEQ ID NO: 363 | -GWYPCWMAQLGELCDLDGGGGSGGVVCQDWEGVELCWQG- |
| SEQ ID NO: 364 | -GGWYPCWMAQLGELCDLDGGGGSGGVVCQDWEGVELCWQG- |
| SEQ ID NO: 365 | GWYPCWMAQLGELCDLDGGGGSGGVVCQDWEGVELCWQGG- |
| SEQ ID NO: 366 | -(X⁴⁰¹)ₙ)-WYPCWMAQLGELCDLD-X⁴⁰⁰-VVCQDWEGVELCWQ-(X⁴⁰¹)ₙ- |
| SEQ ID NO: 61 | GGWYPCWIARVGELCDLEEGPVNRGGGGSGGVVCQDWEGVELCWQGG |
| SEQ ID NO: 62 | GGAVEFYPCWLARIGELCDLVEPGGGGSGGVVCQDWEGVELCWQGG |
| SEQ ID NO: 63 | GGWYPCWIARVGELCDMEGGGGSGGVVCQDWEGVELCWQGG |
| SEQ ID NO: 64 | GGEWFHDCFLAKVGDLCDLFLWGGGGSGGVVCQDWEGVELCWQGG |
| SEQ ID NO: 65 | GGRYVHDCFIAQVGDLCDLFLHGGGGSGGVVCQDWEGVELCWQGG |
| SEQ ID NO: 66 | GGRSLVDCFLVKVGDLCDFFNWGGGGSGGVVCQDWEGVELCWQGG |
| SEQ ID NO: 67 | GGWYPCWIARVGELCDLEGGGGSGGVVCQDWEGVELCWQGG |
| SEQ ID NO: 68 | GGWYPCWLAQVGELCDLDGGGGSGGVVCQDWEGVELCWQGG |
| SEQ ID NO: 69 | GGWYPCWIARVGELCDLEEGPVNRGGGGSGGGGSGGVVCQDWEGVELCWQGG |
| SEQ ID NO: 70 | GGWYPCWIARVGELCDLEEGPVNRGGGGSGGGGSGGGGSGGVVCQDWEGVEL |
| | CWQGG |
| SEQ ID NO: 71 | GGRYVHDCFIAQVGDLCDLFLHGGGGSGGGGSGGVVCQDWEGVELCWQGG |
| SEQ ID NO: 72 | GGRYVHDCFIAQVGDLCDLFLHGGGGSGGGGSGGGGSGGVVCQDWEGVELCW |
| | QGG |
| SEQ ID NO: 73 | GGRYVHDCFIAQVGDLCDLFLHGGGGSGGGGSGGGGSGGGGSGGVVCQDWEG |
| | VELCWQGG |
| SEQ ID NO: 74 | GGLVDCFKVKVGELCDLFGGGGSGGVVCQDWEGVELCWQGG |
| SEQ ID NO: 75 | GGRYVHDCFIAQVGDLCDLFLHGGGGSGGVVCQDWEGVELCWQGG |
| SEQ ID NO: 76 | GGWYSCWMAQLGELCDLDGGGGSGGVVCQDWEGVELCWQGG |
| SEQ ID NO: 77 | Ac-WYPCWMAQLGELCDLDGGGGSGGVVCQDWEGVELCWQ-OH |
| SEQ ID NO: 78 | Ac-WYPCWMAQLGELCDLDGGGGSGGVVCQDWEGVALCWQ-OH |
| SEQ ID NO: 79 | Ac-WYPCW(Abu)AQLGELCDLDGGGGSGGVVCQDWEGVELCWQ-OH |
| SEQ ID NO: 80 | Ac-WYPCW(Abu)AQLGELCDLDGGGGSGGVVCQDWEGVALCWQ-OH |
| SEQ ID NO: 81 | Ac-GGELLVDCFKVKVGELCDLFFGGGGGSGGVVCQDWEGVELCWQGG-OH |
| SEQ ID NO: 82 | Ac-GGRYVHDCFIAQVGDLCDLFLHGGGGSGGVVCQDWEGVELCWQGG-OH |
| SEQ ID NO: 83 | Ac-GGKWVHDCFLAKVGDVCDLFVVGGGGSGGVVCQDWEGVELCWQGG-OH |
| SEQ ID NO: 84 | Ac-GGRSLVDCFLVKVGDLCDFFNWGGGGSGGVVCQDWEGVELCWQGG-OH |
| SEQ ID NO: 85 | Ac-GGEWFHDCFLAKVGDLCDLFLWGGGGSGGVVCQDWEGVELCWQGG-OH |
| SEQ ID NO: 86 | Ac-GGGELLVDCFKVKVGELCDLFFGGGGGSGGVVCQDWEGVELCWQGG-OH |
| SEQ ID NO: 87 | Ac-GGGRYVHDCFIAQVGDLCDLFLHGGGGSGGVVCQDWEGVELCWQGG-OH |
| SEQ ID NO: 88 | Ac-GGGKWVHDCFLAKVGDVCDLFVVGGGGSGGVVCQDWEGVELCWQGG-OH |
| SEQ ID NO: 89 | Ac-GGGRSLVDCFLVKVGDLCDFFNWGGGGSGGVVCQDWEGVELCWQGG-OH |
| SEQ ID NO: 90 | Ac-GGGEWFHDCFLAKVGDLCDLFLWGGGGSGGVVCQDWEGVELCWQGG-OH |
| SEQ ID NO: 91 | -GGGELLVDCFKVKVGELCDLFFG-(PA)8-GVVCQDWEGVELCWQGG- |
| SEQ ID NO: 92 | -GGELLVDCFKVKVGELCDLFFG-(PA)8-GVVCQDWEGVELCWQGG- |
| SEQ ID NO: 93 | -ELLVDCFKVKVGELCDLFFG- (PA)8-GVVCQDWEGVELCWQ- |
| SEQ ID NO: 94 | -GGELLVDCFKVKVGELCDLFFG-X⁴⁰⁰-GVVCQDWEGVELCWQGG- |
| SEQ ID NO: 95 | -ELLVDCFKVKVGELCDLFFG-X⁴⁰⁰-GVVCQDWEGVELCWQ- |
| SEQ ID NO: 96 | -(X⁴⁰¹)ₙ)-ELLVDCFKVKVGELCDLFFG-X⁴⁰⁰-GVVCQDWEGVELCWQ-(X⁴⁰¹)ₙ)- |
| SEQ ID NO: 97 | -(X⁴⁰¹)ₙ)-ELLVDCFKVKVGELCDLFFG-(PA)8-GVVCQDWEGVELCWQ-(X⁴⁰¹)ₙ)- |
| SEQ ID NO: 98 | Ac-GGGELLVDCFKVKVGELCDLFFG-(PA)8-GVVCQDWEGVELCWQGG-OH |
| SEQ ID NO: 99 | Ac-GGELLVDCFKVKVGELCDLFFG-(PA)8-GVVCQDWEGVELCWQGG-OH |

In an IL-2Rβγc agonist peptide having an amino acid sequence of any one of SEQ ID NO: 361, 362, 366 and 94-96, X⁴⁰⁰ can include from 1 to 20 amino acids. For example, X⁴⁰⁰ can be selected from having an amino acid sequence of any one of SEQ ID NO: 2-29. For example, X⁴⁰⁰ can be GGS (SEQ ID NO: 24) or GGGGSGG (SEQ ID NO: 26). For example, X⁴⁰⁰ can be selected from a peptidyl linker having an amino acid sequence of any one of SEQ ID NO: 30-38.

In IL-2Rβγc agonist peptide having SEQ ID NO: 366, 96, and 97 each (X⁴⁰¹)ₙ can independently be selected from (G)ₙ where n is an integer from 1 to 10 (SEQ ID NO: 2).

An IL-2Rβγc agonist peptide can comprise:
an amino acid sequence of any one of SEQ ID NO: 359-366, 61-76, and 77-99;
a truncated amino acid sequence of any one of SEQ ID NO: 359-366, 61-76, and 77-99; an amino acid sequence of any one of SEQ ID NO: 359-366, 61-76, and 77-99 having from 1 to 5 amino acid substitutions;
an amino acid sequence of any one of SEQ ID NO: 359-366, 61-76, and 77-99 having from 1 to 5 flanking glycines on the N-terminus, the C-terminus, or both the N-terminus and the C-terminus;
an amino acid sequence having greater than 60%, greater than 70%, greater than 80%, or greater than 90% sequence similarity to an amino acid sequence of any one of SEQ ID NO: 359-366, 61-76, and 77-99; or
a combination of any of the foregoing.

An IL-2Rβγc agonist peptide can comprise an amino acid sequence of any one of SEQ ID NO: 91-99.

An IL-2Rβγc agonist peptide can comprise an amino acid sequence of any one of SEQ ID NO: 91 and 92.

An IL-2Rβγc agonist peptide can comprise an amino acid sequence of any one of SEQ ID NO: 98 and 99.

An IL-2Rβγc agonist peptide can bind to each of the IL-2Rβ subunit and to the IL-2Rγc subunit, such as each of the hu-IL-2Rβ subunit and to the hu-IL-2Rγc subunit with an IC₅₀ of less than 100 pm, less than 10 pm, less than 1 pm, less than 100 μM, less than 10 μM, or less than 1 μM.

Examples of suitable IL-2Rβγc agonist peptides are disclosed, for example, in U.S. Publication No. 2021/0253670 A1, which is incorporated by reference in its entirety.

A culture medium can comprise a concentration of an IL-2Rβγc agonist peptide, for example, of greater than 1 nM, greater than 10 nM, greater than 100 nM, greater than 200 nM, greater than 500 nM, greater than 1,000 nM, or greater than 2,000 nM.

A culture medium can comprise a concentration of an IL-2Rβγc agonist peptide, for example, of less than 5,000 nM, less than 2,000 nM, less than 1,000 nM, less than 500 nM, less than 200 nM, less than 100 nM, less than 50 nM, or less than 10 nM,

A culture medium can comprise a concentration of an IL-2Rβγc agonist peptide, for example, of from 1 nM to 5,000 nM, from 10 nM, to 1,000 nM, or from 50 nM to 500 nM.

A culture medium can comprise a concentration of an IL-2Rβγc agonist peptide sufficient to stimulate the proliferation of a target immune cell population, such as the same target immune cell population being proliferated by incubation with the IL-7Rαγc agonist peptide.

A culture medium provided by the present disclosure can comprise an antibody directed to the target immune cell population. The antibody can be a stimulant for the proliferation of the target immune cell population. The antibody can be a monoclonal antibody.

For example, for a culture medium comprising a target T cell population, the antibody can be directed to the target immune cell population, such as a target T cell population.

For example, when the target T cell population comprises CD4+ T cells and CD8+ T cells, the antibody can comprise an anti-CD3 antibody and anti-CD28 antibody.

A culture medium can comprise a concentration of an antibody, for example, of greater than 0.1 ng/mL, greater than 1 ng/mL, greater than 5 ng/mL, greater than 10 ng/mL, greater than 50 ng/mL, greater than 100 ng/mL, or greater than 500 ng/mL.

A culture medium can comprise a concentration of an antibody, for example, of less than 500 ng/mL, less than 100 ng/mL, less than 50 ng/mL, less than 10 ng/mL, less than 5 ng/mL, less than 1 ng/mL, or less than 0.5 ng/mL.

A culture medium can comprise a concentration of an antibody for example, of from 0.1 ng/mL to 500 ng/mL, from 1 ng/mL to 100 ng/mL, or from 5 ng/mL to 50 ng/mL.

A culture medium can comprise a concentration of an antibody sufficient to augment the proliferation of the target immune cell population.

Methods of expanding a target population of T cells provided by the present disclosure can comprise a desirable or intended phenotype of immune cells such as T cells.

For example, methods provided by the present disclosure can provide a population of T cells enriched in Tem cells and Tscm cells.

An immune cell growth medium comprising a first growth stimulant such as an IL-7Rαγc agonist peptide, a second growth stimulant such as an IL-2Rβγc agonist peptide, and optionally one or more antibodies can produce a subpopulation of T cells or a combination of subpopulations of T cells enriched in Tem and Tscm cells.

For example, a population of T cells enriched in Tem and/or Tscm cells can comprise greater than 60%, 65%, 70%, 75%, or 80% of combined Tem cells and Tscm cells, wherein percent is based on the total number of T cells in the sample.

A population of T cells (e.g., genetically modified T cells) enriched in Tem and Tscm cells can comprise from 60% to 90% of combined Tem cells and Tscm cells.

A population of T cells enriched in Tem and/or Tscm cells can comprise greater than 20%, 25%, 30%, 35%, 40%, 45%, 50%, 55%, or 60% Tscm cells, where percent is based on the total number of T cells.

Methods provided by the present disclosure comprise expanding a target immune cell population of an initial immune cell population by incubating the initial immune cell population in the presence of an IL-7Rαγc agonist peptide.

A target immune cell population can comprise immune cells for which cell growth is stimulated in the presence of an IL-7Rαγc agonist peptide, either alone or in combination with another growth stimulant for the target immune cell population.

For example, a target immune cell population can comprise a target T cell population.

A target immune cell population can comprise, for example, a target Tem cell population, a target Tscm cell population, or a target Tem cell population and a target Tscm cell population.

A target immune cell population can comprise, for example, a population of CD4+ cells, a population of CD8+ cells, or a population of CD4+ and CD8+ cells.

A target immune cell population can comprise a population of engineered immune cells.

A population of engineered immune cells can be engineered to express one or more chimeric antigen receptors.

An initial immune cell population can be derived from a sample obtained from a patient.

An initial immune cell population can be derived from a sample obtained from a donor.

An initial immune cell population can be derived from a suitable tissue.

An initial immune cell population can comprise allogenic immune cells.

An initial immune cell population can comprise autologous immune cells.

Incubating the initial immune cell population to expand the target immune cell population can comprise incubating the immune cells in a culture medium provided by the present disclosure.

Following incubation in the presence of an IL-7Rαγc agonist peptide, the concentration of the target immune cell population can be expanded, for example, by greater than 10-times, greater than 100-times, greater than 1,000-times, greater than 10,000-times, greater than 100,000, or greater than 1,000,000-times, compared to the concentration of the target immune cell population before incubation with the IL-7Rαγc agonist peptide.

The expansion of the target immune cell population can be determined, for example, at greater than 3 days, greater than 7 days, greater than 10 days, greater than 20 days, or greater than 30 days, after the beginning of incubation.

The expansion of the target immune cell population can be determined, for example, from 3 days to 50 days, from 3 days to 40 days, from 3 days to 30 days, or from 3 days to 20 days, after the beginning of incubation.

Incubating can comprise incubating the target immune cell population with a second growth stimulant for the target immune cell population. For example, a second growth stimulant can comprise an IL-2Rβγc agonist peptide, an IL-15Rβγc agonist peptide, or a combination thereof.

The target immune cell population can be expanded by incubating in a medium comprising both the IL-7Rαγc agonist peptide and a second growth stimulant such as an IL-2Rβγc agonist peptide.

The target immune cell population can be expanded by incubating in a medium comprising the IL-7Rαγc agonist peptide without a second growth stimulant and subsequently incubating in a medium comprising the second growth stimulant without the IL-7Rαγc agonist peptide. Thus, the target immune cell population can be expanded by incubating the target immune cell population in different growth media during the course of expansion, where at least one of the growth media comprises an IL-7Rαγc agonist peptide.

An immune cell subpopulation can be expanded by incubating an immune cell population in the presence of an IL-7Rαγc agonist peptide.

The immune cell population can be expanded by incubating an immune cell population in the presence of an IL-7Rαγc agonist peptide as the only stimulant of cell proliferation.

The immune cell population can be expanded by incubating an immune cell population in the presence of an IL-7Rαγc agonist peptide provided by the present disclosure in combination with a second stimulant of cell growth proliferation.

The second stimulant of cell growth proliferation can be, for example, an agent that stimulates a CD3 TCR complex; an anti-CD3 antibody or antigen-binding fragment thereof, an anti CD28 antibody or antigen-binding fragment thereof, an anti-CD2 antibody or antigen-binding fragment thereof; a protein kinase C activator, a growth factor such as a T cell growth factor, a peptide such as an IL-7Rαγc agonist peptide or an IL-15Rαγc agonist peptide, or a combination of any of the foregoing.

For example, a population of T cells can be incubated in the presence of an anti-CD3 antibody and an anti-CD28 antibody under conditions appropriate for stimulating the proliferation of the T cells.

An anti-CD3 antibody and an anti-CD28 antibody can be disposed or immobilized on a bead, plate, or other substrates.

A second stimulant of cell growth stimulation can be an IL-2Rβγc agonist peptide.

A suitable combination of second stimulants of cell growth proliferation can comprise, for example, an anti-CD3 antibody and an anti-CD28 antibody.

A suitable combination of second stimulants of cell growth proliferation can comprise, for example, an anti-CD3 antibody, an anti-CD28 antibody, and an IL-2Rβγc agonist peptide.

Immune cells can be incubated with an IL-7Rαγc agonist peptide provided by the present disclosure wherein the concentration of the IL-7Rαγc agonist peptide is greater than 1 nM, greater than 10 nM, greater than 100 nM, greater than 250 nM, greater than 500 nM, greater than 1,000 nM, or greater than 5,000 nM.

A concentration of an IL-7Rαγc agonist peptide can be, for example, greater than 0.1 μg/mL, greater than 0.5 μg/mL, greater than 1 μg/mL, greater than 5 μg/mL, or greater than 10 μg/mL. A concentration of an IL-7Rαγc agonist peptide in the cell growth medium can be, for example, from 0.01 μg/mL to 100 μg/mL, from 0.1 μg/mL to 10 μg/mL, or from 0.5 μg/mL to 5 μg/mL.

A concentration of an antibody such as an anti-CD3 antibody or an anti-CD28 antibody in the cell growth media can be, for example, greater than 0.1 ng/mL, greater than 0.5 ng/mL, greater than 1 ng/mL, greater than 5 ng/mL, greater than 10 ng/mL, greater than 50 ng/mL, greater than 100 ng/mL, or greater than 500 ng/mL.

A concentration of an antibody such as an anti-CD3 antibody or an anti-CD28 antibody in the cell growth media can be, for example, from 0.1 ng/mL to 1,000 ng/mL, from 1 ng/mL to 100 ng/mL, or from 5 ng/mL to 50 ng/mL.

During cell proliferation, fresh media containing the stimulants of cell proliferation can be provided, for example, every day, every two days, every three days, every four days, every five days, every six days, every week, every two weeks, every three weeks or every four weeks.

The immune cells, such as T cells can be activated and expanded, either prior to or after genetic modification of the immune cells. Immune cells can be activated and expanded prior to genetic modification of the immune cells. Immune cells can be activated and expanded after genetic modification of the immune cells, such as engineered immune cells

Immune cell expansion can be accomplished by incubating the targeted immune cells in any suitable culture medium.

An immune-cell expansion medium provided by the present disclosure can comprise an IL-2Rβγc agonist peptide provided by the present disclosure.

For example, a targeted immune cell population, such as a targeted T cell population, can be stimulated in vitro by contact with, for example, an anti-CD3 antibody and/or anti-CD28 antibody, or antigen-binding fragment thereof. For co-stimulation of an accessory molecule on the surface of the immune cells, a ligand that binds the accessory molecule can be used. For example, a population of immune cells can be contacted with an anti-CD3 antibody and an anti-CD28 antibody under conditions appropriate for stimulating the proliferation of the immune cells. The anti-CD3 antibody and an anti-CD28 antibody can be immobilized. For example, the antibody can be displayed on a bead, matrix, plate, or other substrates.

Methods provided by the present disclosure can comprise incubating a population of immune cells such as T cells with a second proliferation stimulant, such as anti-CD3 and anti-CD28 antibodies, and a cytokine such as IL-7 and/or IL-15. The second proliferation stimulant can be an IL-2Rβγc agonist peptide or an IL-15Rβγc agonist peptide. The antibodies can be immobilized on a substrate. For example, anti-CD3 and anti-CD28 antibodies can be attached to a bead such as a Dynabead® system, a CD3/CD28 activator/stimulator system for physiological activation of human immune cells such as human T cells. Immune cells can be activated and stimulated to proliferate with suitable antibodies and other cytokines, in addition to IL-7 and IL-15

In methods provided by the present disclosure, a targeted immune cell population, such as a targeted T cell population, can be expanded by activating a target immune cell population by incubating a population of primary cells with a suitable antibody and expanding the activated target immune cell population by incubating the activated target immune cell population in the presence of an IL-7Rαγc agonist peptide.

Methods provided by the present disclosure comprise culturing immune cells such as T cells using a cell growth medium comprising an IL-7Rαγc agonist peptide provided by the present disclosure. In addition to an IL-7Rαγc agonist peptide, the cells can be cultured with a second stimulant of immune cell proliferation such as an IL-2Rβγc peptide, an IL-15Rβγc peptide, or a combination thereof. The cells can be cultured for a duration, for example, of less than 1 week, for 1 week, for two weeks, for three weeks, for weeks, or for five weeks. Cells can be cultured, for example, for from 1 day to 7 days, from 1 day to 5 days, or from 1 day to 3 days. Cells can be cultured, for example, for 1 day, 2 days, 3 days, 4 days, 5 days, 6 days, or 7 days.

Cells can be cultured for a suitable duration to provide the desired enrichment of a target subpopulation of immune cells. For example, a suitable enrichment of a target subpopulation of immune cells such as T cells can increase the relative number of immune cells by greater than 10 times, greater than 100 times, greater than 200 times, greater than 400 times, greater than 600 times, greater than 800 times, or greater than 1,000 times.

Cells useful in the methods provided by the present disclosure can be derived from primary cells such as primary human cells. The primary cells can be derived from a biological sample such as, for example, tissue, fluid, and other samples taken directly from a subject or patient, as well as samples resulting from one or more processing steps, such as separation, centrifugation, genetic engineering such as transduction with a viral vector, washing, and/or incubation. The biological sample can be a sample obtained directly from a biological source or a biological sample that is processed. Biological samples include, for example, body fluids, such as blood, plasma, serum, cerebrospinal fluid, synovial fluid, urine and sweat, tissue and organ samples, including processed samples derived from any of the foregoing. Biological samples can be obtained, for example, from peripheral blood mononuclear cells, bone marrow, lymph node tissue, cord blood, thymus tissue, stem cell- or iPSC-derived immune cells, tissue from a site of infection, ascites, pleural effusion, spleen tissue, and tumors.

Cells suitable for culturing using the media and methods provided by the present disclosure include immune cells.

Cells can be derived from a healthy donor, from a patient diagnosed with a disease such as cancer, an autoimmune disease or a viral disease, or from a patient diagnosed with an infection. Cells can be part of a mixed population of cells that present different phenotypic characteristics.

Cells can be obtained from a subject or patient who will ultimately receive the expanded immune cells. Cells can be obtained from a donor, who is a different individual from the subject who will receive the expanded immune cells.

Immune cells can comprise T cells. T cells can be obtained from a number of sources, including peripheral blood mononuclear cells (PBMCs), bone marrow, lymph nodes tissue, cord blood, thymus tissue, stem cell- or iPSC-derived T cells, tissue from a site of infection, ascites, pleural effusion, spleen tissue, and tumors. T cells can be obtained from a volume of blood collected from a subject using any number of techniques known to the skilled person.

Cells can be obtained from the circulating blood of an individual by apheresis. An apheresis product can include lymphocytes, including T cells, monocytes, granulocytes, B cells, other nucleated white blood cells, red blood cells, and platelets. Cells collected by apheresis can be washed to remove the plasma fraction and placed in an appropriate buffer or media for subsequent processing.

PBMCs can be isolated by removing red blood cells and granulocytes using centrifugation through a density gradient.

PBMCs can be used directly for genetic modification with the immune cells, such as CARS or TCRs, using methods provided by the present disclosure. After isolating the PBMCs, T lymphocytes can be further isolated, and both cytotoxic and helper T lymphocytes can be sorted into naive, memory, and effector T cell subpopulations either before or after genetic modification and/or expansion.

A specific subpopulation of T cells, such as CCR7+, CD95+, CD122+, CD27+, CD69+, CD127+, CD3+, CD4+, CD8+, CD25+, CD62L+, CD45RA+, and CD45RO+ T cells can be further isolated by positive or negative selection techniques. A specific subpopulation of T cells can comprise CD4+ cells and CD8+ cells.

For example, enrichment of an immune cell population such as a T cell population by negative selection can be accomplished with a combination of antibodies directed to surface markers unique to the negatively selected cells. Flow cytometry and cell sorting can also be used to isolate cell populations of interest for use in the present disclosure.

In some embodiments, a population of T cells is enriched for CD4+ cells, CD8+ cells, or a mixture of both CD4+ and CD8+ cells.

T cells can be further sorted, for example, into naive, central memory, and effector cells by identifying cell surface antigens that are associated with each of these types of T cells. For example, CD4+ T cells can be sorted into naive, central memory, and effector cells by identifying cell populations with cell surface antigens.

Cell growth media and methods provided by the present disclosure can be useful for in vitro expansion of immune cells such as engineered immune cells such as CAR-T cells.

Engineered immune cells can be allogeneic or autologous immune cells.

An engineered immune cell can be a T cell such as an inflammatory a T-lymphocyte, a cytotoxic T-lymphocyte, a regulatory T-lymphocyte, a helper T-lymphocyte, a tumor-infiltrating lymphocyte (TIL), an NK cell, an NK-T-cell, a gamma delta T cell, a TCR-expressing cell, a dendritic cell, a killer dendritic cell, a mast cell, a B-cell, or a combination of any of the foregoing. An engineered immune cell can be derived, for example, from a CD4+T-lymphocyte and/or CD8+T-lymphocytes. An engineered immune cell can be a T cell.

An engineered immune cell can be derived, for example, from a stem cell. Stem cells can be adult stem cells, non-human embryonic stem cells, more particularly non-human stem cells, cord blood stem cells, progenitor cells, bone marrow stem cells, induced pluripotent stem cells, totipotent stem cells, hematopoietic stem cells, or combinations of any of the foregoing.

Cells can be obtained or prepared from peripheral blood. For example, cells can be obtained or prepared from peripheral blood mononuclear cells (PBMCs), from bone marrow, or from umbilical cord blood. Cells can be human cells. Cells can be transfected or transduced by a nucleic acid vector using methods such as electroporation, sonoporation, biolistics, lipid transfection, polymer transfection, nanoparticles, or polyplexes.

Engineered immune cells can comprise an antigen binding agent such as an antigen binding domain, an antibody, or an antibody fragment.

An engineered immune cell can comprise a population of CARs where each CAR comprises an extracellular antigen-binding domain. An engineered immune cell can comprise a population of CARs where each CAR comprises the same extracellular antigen-binding domains.

A chimeric antigen receptor (CAR) refers to a protein that specifically recognizes target antigens, such as target antigens on cancer cells. When bound to the target antigen, the CAR can activate an immune cell to attack and destroy the cell expressing the target antigen.

A CAR can incorporate costimulatory or signaling domains to increase potency.

A targeted immune cell population can be engineered to express an antigen binding moiety.

An antigen binding moiety can be directed to any specific antigen, such as, for example, an antigen expressed on the surface of a tumor cell or in a tumor cell environment, an antigen expressed on an immune cell, an antigen expressed on the surface of a cell expressing the IL-7Rα and Rγc subunits of IL-7R such as CD4+ T-cells, CD8+ T-cells, or NK cells.

Examples of suitable antigen targets expressed on tumor cells include fibroblast activation protein (FAP), the A1 domain of tenascin-C (TNC A1), the A2 domain of tenascin-C (TNC A2), the extra cellular domain B of fibronectin (EDB), carcinoembryonic antigen (CEA), and the melanoma-associated chondroitin sulfate proteoglycan (MCSP).

Other examples of suitable tumor antigens that can be used for targeting include MAGE, MART-1/Melan-A, gplOO, Dipeptidyl peptidase 4 (DPP-4), adenosine deaminase-binding protein (ADAbp), cyclophilin b, Colorectal associated antigen (CRC)-0017-A/GA733, Carcinoembryonic Antigen (CEA) and its immunogenic epitopes CAP-1 and CAP-2, etv6, amll, Prostate Specific Antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-AS, MAGE-A6, MAGE-A7, MAGE-AS, MAGE-A9, MAGE-AlO, MAGE-All, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-CS), GAGE-family of tumor antigens such as GAGE-I, GAGE-2, GAGE-3, GAGE-4, GAGE-5, GAGE-6, GAGE-7, GAGE-8, GAGE-9, BAGE, RAGE, LAGE-I, NAG, GnT-V, MUM-1, CDK4, tyrosinase, p53, MUC family, HER2/neu, p2Iras, RCAS1, α-fetoprotein, E-cadherin, a-catenin, -catenin and y-catenin, p120ctn, gplOO Pmel117, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, Imp-1, PIA, EBY-encoded nuclear antigen (EBNA)-1, brain glycogen phosphorylase, SSX-1, SSX-2 (HOM-MEL-40), SSX-1, SSX-4, SSX-5, SCP-1 and CT-7, and cerbB-2.

Examples of viral antigens include influenza virus hemagglutinin, Epstein-Barr virus LMP-1, hepatitis C virus E2 glycoprotein, HIV gp160, and HIV gp120.

Examples of ECM antigens include syndecan, heparanase, integrins, osteopontin, cadherins, laminin, laminin type EGF, lectin, fibronectin, notch, tenascin, and matrixin.

Targeted IL-7Rαγc ligand fusion proteins can be configured to bind, for example, to a cell surface antigen selected from FAP, Her2, EGFR, IGF-1R, CD2 (T-cell surface antigen), CD3 (heteromultimer associated with the TCR), CD22 (B-cell receptor), CD23 (low affinity IgE receptor), CD30 (cytokine receptor), CD33 (myeloid cell surface antigen), CD40 (tumor necrosis factor receptor), IL-6R (IL6 receptor), CD20, MCSP, and PDGFR (platelet-derived growth factor receptor).

A targeted IL-7Rαγc ligand construct can comprise an antigen binding moiety capable of binding to an antigen or to a receptor expressed on the surface of a cell that also expresses IL-7R. Examples of cells expressing IL-7R include, for example, naïve T-cells, memory T-cells, and activated T-cells in CD8+ T-cells, CD4+ T-cells.

A targeted IL-7Rαγc ligand construct can comprise an antigen binding moiety capable of binding to an antigen or a receptor expressed by a cell that expresses the IL-7Rα and Rγc subunits of IL-7R. Examples of cells expressing the IL-7Rα and Rγc subunits of IL-7R include, for example, naïve T-cells, memory T-cells, and activated T-cells in CD4+ T-cells, and CD8+ T-cells.

Examples of antigens expressed on the surface of naïve CD4+ T-cells include CD4, CD45RA, CCR7, CD27, and CD28.

Examples of antigens expressed on the surface of naïve CD8+ T-cells include CD8, CD45RA, CCR7, CD27, and CD28.

Examples of antigens expressed on the surface of CD4+T-cells include Th1 cell markers such as CD4, CXCR3, CCR5, and IL12Rβ2; Th2 cell markers such as CD4, CCR4, and IL4Rα; Th9 cell markers such as CD4, CCR3, and CCR5; Th17 cell markers such as CD4, CCR6, CCR4, IL21R, and IL23R; Th22 cell markers such as CD4, CCR10, CCR4, and CCR6; Treg cell markers such as CD4, CD127, CD25, and CTLA-4; and Tfh cell markers such as CD4, CXCR5, CD40L, PD-1, and ICOS.

Examples of antigens expressed on the surface of cytotoxic CD8+ T cells include CD8.

Examples of memory T-cell antigens include CCR5, CCR7, CD11a, CD27, CD28, CD45RA, CD45RO, CD57, CD95, and CD62L.

Examples of naive T-cell antigens include CD45RA, CCR7, CD62L, CD27, CD28, CD127, and CD132.

A targeted IL-7Rαγc ligand construct can comprise an antigen binding moiety capable of binding to an antigen or receptor expressed on the surface of cells, having a role in regulating the immune response.

Examples of antigens expressed by cells associated with regulating the immune response include PD-1, CTLA-4, CD20, and CD30.

A targeted IL-7Rαγc ligand construct can comprise an antigen binding moiety capable of binding to an antigen or receptor expressed on the surface of Treg cells such as CD25. For example, a Treg cell-targeted construct can comprise an IL-7Rαγc ligand/daclizumab antibody fusion.

An immune cell such as a T cell can be genetically modified prior to expansion using methods provided by the present disclosure.

The process for manufacturing allogeneic CAR T therapy involves first harvesting healthy, selected, screened, and tested T cells from healthy donors.

Immune cells such as T cells are engineered to express CARs, which recognize certain cell surface proteins expressed in hematologic or solid tumors. Allogeneic T cells can be gene edited to reduce the risk of graft versus host disease (GvHD) and to prevent allogeneic rejection. For example, a T cell receptor gene such as TCRa or TCR˜ can be knocked out to avoid GvHD. The CD52 gene can be knocked out to render the CAR T product resistant to anti-CD52 antibody treatment. Anti-CD52 antibody treatment can be used to suppress the host immune system and to allow the CAR T to stay engrafted to achieve full therapeutic impact. After gene editing, the engineered T cells then undergo a purification step and then cryopreserved in vials for delivery to patients.

Autologous chimeric antigen receptor (CAR) T cell therapy involves collecting T cells from a patient and genetically engineering the T cells to express CARs that recognize the target expressed on the cell surface of one or more specific cancer cells of the patient. The engineered T cells can be cryopreserved and subsequently administered to the patient.

Methods of manufacturing an immune cell population comprise activating a target immune cell population from primary cells to provide an activated target immune cell population; and expanding the activated target immune cell population, wherein expanding comprises incubating the activated target immune cell population in the presence of an IL-7Rαγc agonist peptide to provide an expanded target immune cell population.

The target immune cell population can comprise, for example, T cells and/or NK cells.

The population of primary cells can comprise any suitable populations in primary cells that comprise immune cells.

A population of primary cells can be obtained from any suitable source such as disclosed herein.

A population of primary cells can comprise human primary cells.

A population of human primary cells can comprise mononuclear cells.

A mononuclear cell refers to a cell found in blood that has a single, round nucleus such as a lymphocyte or a monocyte. Mononuclear cells can be obtained from body fluids such as blood and including, for example, peripheral blood, bone marrow, and cord blood by known methods such as centrifugation, magnetic beads, and flow cytometry. Mononuclear cells may be those derived from stem cells such as induced pluripotent stem cells, embryonic stem cells, and somatic stem cells. A mononuclear cell can be a peripheral blood mononuclear cell (PBMC).

Mononuclear cells can comprise peripheral blood mononuclear cells (PBMCs) obtained from a patient or comprise peripheral blood mononuclear cells (PBMCs) obtained from a donor.

A biological sample from which immune cells are derived or isolated can be blood or a blood-derived sample or can be derived from an apheresis or leukapheresis product. Suitable examples from which immune cells can be derived include whole blood, peripheral blood mononuclear cells (PBMCs), leukocytes, bone marrow, thymus, tissue biopsy, tumor, leukemia, lymphoma, lymph node, gut-associated lymphoid tissue, mucosa-associated lymphoid tissue, spleen, other lymphoid tissues, liver, lung, stomach, intestine, colon, kidney, pancreas, breast, bone, prostate, cervix, testes, ovaries, tonsil, or other organs.

Cell samples can also be obtained in the context of cell therapy, such as adoptive cell therapy, from autologous and allogeneic sources.

Immune cells for use in partial reprogramming methods can be derived from cell lines such as T cell lines. Immune cells can be obtained from a xenogeneic source such as, for example, from mouse, rat, non-human primate, and pig.

In partial reprogramming methods, T cells can be isolated from a source and partially reprogrammed to ameliorate aging and improve the function of the T cells. Examples of suitable source cells include peripheral blood mononuclear cells (PBMCs). T cells for use in the methods herein may include cultured T cells, e.g., primary T cells or T cells from a suitable cultured T cell line, or T cells obtained from a mammal. If obtained from a mammal, the source cells can be obtained from numerous sources, including blood, bone marrow, lymph node, tumor, thymus, spleen, or other tissues or fluids.

Source cells can also be enriched or purified. The T cells can be any type of T cells and can be of any developmental stage, including for example, CD4+CD8+ double-positive T cells, CD4+ helper T cells, e.g., Th1 and Th2 cells, CD8+ T cells such as cytotoxic T cells, peripheral blood mononuclear cells (PBMCs), peripheral blood leukocytes (PBLs), tumor-infiltrating cells (TILs), memory T cells, and naive T cells.

T cells can be isolated from tumors, such as tumor-infiltrating lymphocytes such as T cells for use in a partial reprogramming method.

A tumor-infiltrating lymphocytes or TILs refers to a population of cells originally obtained as cells that have left the bloodstream of a subject and migrated into a tumor. TILs include, for example, CD8+ cytotoxic T cells (lymphocytes), Th1 and Th17 CD4+ T cells, natural killer cells, dendritic cells, and tumor-associated macrophages. TILs include both primary and secondary TILs. “Primary TILs” refer to TILs obtained from patient tissue samples. TILs can be categorized as expressing a biomarker such as CD4, CD8, TCRa, CD27, CD28, CD56, CCR7, CD45RA, CD45RO, CD95, PD-1, CD25, or a combination of any of the foregoing.

A source cell can have a naive T cell (Tn) phenotype, a central memory T cell (Tcm) phenotype, an effector memory T cell (Tem) phenotype, or a stem-like T cell (Tscm).

The phenotypes of Tn, Tem and Tem cells are known in the art and are described elsewhere herein. For example, CCR7 and CD62L are expressed by Tn and Tem cells but are not expressed by Tem cells. The transcription factors LEF1, FOXP1 and KLF7 are expressed by Tn and Tem cells but are not expressed by Tem cells. CD45RO and KLRG1 are not expressed by Tn cells but are expressed by Tem cells. Alternatively, or additionally, Tn and Tem cells may be characterized by longer telomeres as compared to those of Tem cells.

A specific subpopulation of T cells such as, for example, CD3+, CD45+, CD137+, CD25+, CD28+, CD4+, CD8+, CD45RA+, GITR+, and/or CD45RO+ T cells, can be isolated by positive or negative selection techniques such as using fluorescence-based or magnetic-based cell sorting. For example, T cells can be isolated by incubation with any of a variety of commercially available antibody-conjugated beads, such as Dynabeads®, CELLection™, DETACHaBEAD™ (Thermo Fisher), or MACS® cell separation products (Miltenyi Biotec), for a duration sufficient for positive selection of the desired T cells or negative selection for removal of unwanted cells.

In methods provided by the present disclosure, a target immune cell population can be any suitable immune cell population for which proliferation is stimulated by incubation with an IL-7Rαγc agonist peptide.

For example, a target immune cell population can comprise CD4+ T cells and CD8+ T cells.

In methods of the present disclosure, activating the target immune cell population comprises incubating the target immune cell population in the presence of an IL-7Rαγc agonist peptide.

The IL-7Rαγc agonist peptide can be immobilized on a substrate such as, for example, a bead, matrix, or plate.

Activating a target immune cell population can comprise incubating a population of primary cells comprising the target immune cell population in the presence of an antibody to the target immune cell population.

For example, when the target immune cell population comprises CD4+ T cells and CD8+ T cells, the CD4+ T cells and CD8+ T cells can be incubated in the presence of an anti-CD3 monoclonal antibody and an anti-CD28 monoclonal antibody.

The antibody can be immobilized on a substrate, including any of the substrates disclosed herein.

Expansion of the activated target immune cell population can comprise incubating the activated target immune cell population in the presence of a second growth stimulant for the proliferation of the activated target immune cell population.

The second growth stimulant can be, for example, a suitable agonist peptide such an IL-2Rβγc agonist peptide, an IL-15Rβγc agonist peptide, a cytokine such as a natural cytokine or an engineered cytokine, an antibody, or a combination of any of the foregoing.

The second stimulant can be an agonist peptide. For example, the second stimulant can be an IL-2Rβγc agonist peptide, an IL-15Rβγc agonist peptide, or a combination thereof.

Expanding an activated target immune cell population can comprise incubating the activated target immune cell population in the presence of an IL-7Rαγc agonist peptide together with a second growth stimulant such as an IL-2Rβγc agonist peptide and/or an IL-15Rβγc agonist peptide.

Expanding an activated target immune cell population can comprise incubating the activated target immune cell population in the presence of an IL-7Rαγc agonist peptide in the absence of a second growth stimulant such as an IL-2Rβγc agonist peptide and/or an IL-15Rβγc agonist peptide.

Expanding an activated target immune cell population can comprise incubating the activated target immune cell population in the presence of an IL-7Rαγc agonist peptide. Expanding an activated target immune cell population can comprise incubating the activated target immune cell population in the presence of an IL-7Rαγc agonist peptide for a period of time and then incubating the activated target immune cell population in the presence of a second growth stimulant in the absence of an IL-7Rαγc agonist peptide for a period of time.

Activating a target immune cell population with an IL-7Rαγc agonist peptide can comprise activating IL-7R. Activating a target immune cell population with an IL-2Rβγc agonist peptide can comprise activating IL-2R. Activating a target immune cell population with an IL-15Rβγc agonist peptide can comprise activating IL-15R.

Methods of manufacturing provided by the present disclosure can comprise, for example, before activating the target immune cell population, enriching the population of primary cells with the target immune cell population.

Enriching the target immune cell population can comprise, for example, increasing the number of the target immune cell population by 10 times, 10² times, 10³ times, 10⁴ times, 10⁵ times, or 10⁶ times compared to the initial target immune cell population in the sample, where the number of target immune cells is based on the number of cells per volume.

For example, for in vitro sorting of a population of immune cells, where a subset of the population of immune cells comprises engineered immune cells expressing an antigen-specific CAR epitopes specific for monoclonal antibodies can be used. The method comprises contacting the population of immune cells with a monoclonal antibody specific for the epitopes and selecting the immune cells that bind to the monoclonal antibody to obtain a population of cells enriched in engineered immune cells expressing an antigen-specific CAR.

A monoclonal antibody specific for an epitope can be conjugated to a fluorophore. In this method, the step of selecting the cells that bind to the monoclonal antibody can be done by Fluorescence Activated Cell Sorting (FACS).

A monoclonal antibody specific for an epitope can be conjugated to a magnetic particle. In this method, the step of selecting the cells that bind to the monoclonal antibody can be done by Magnetic Activated Cell Sorting (MACS).

Methods of manufacturing provided by the present disclosure can comprise, for example, after activating the target immune cell population, engineering the activated target immune cell population to express a cell surface receptor.

Engineering can comprise, for example, transducing the activated target immune cell population with a viral vector to provide a transduced activated target immune cell population.

A viral vector can comprise, for example, a viral vector expressing a T cell receptor (TCR) or a chimeric antigen receptor (CAR).

A TCR or CAR can recognize a specific antigenic moiety on a surface of a target cell.

An antigenic moiety can be, for example, an MHC class I dependent antigenic moiety, an MHC class II-dependent antigenic moiety, or a combination thereof.

Methods of manufacturing provided by the present disclosure can comprise, for example, before activating the target immune cell population, enriching the population of primary cells with the target immune cell population.

For example, the target immune cell population can be enriched, for example, by greater than 10 times, greater than 10² times, or greater than 10³ times, greater than 10⁴ times, greater than 10⁵ times, or greater than 10⁶ times, or greater than 10⁷ times. Enrichment refers to the increased concentration of the target immune cell population in the enriched media compared to the concentration of the target immune cell population before incubation according to methods provided by the present disclosure. For example, for an initial concentration of the target immune cell population of 10³ cells/mL, and an enriched concentration of the target immune cell population of 10⁶ cells/mL, the concentration of the target immune cell population is increased or enriched by 10³ times.

In methods of manufacturing where the target immune cell population comprises CD4+ T cells and CD8+ T cells, the population of primary cells can be enriched for CD4+ T cells and CD8+ T cells.

In methods of manufacturing provided by the present disclosure, enriching can comprise removing or increasing the concentration of a specific cell population of the population of primary cells.

For example, enriching can comprise removing CD57+ T cells from the population of primary cells to provide a depleted population of CD57+ cells.

The production and manufacture of engineered immune cells can comprise harvesting immune cells from a patient followed by in vitro expansion of a target population of immune cells using methods provided by the present disclosure to provide a desired enrichment of the target population of immune cells having the desired cell phenotype or combination of phenotypes.

For example, T cell expansion can result in a desired combination of T cells that includes subsets of naive T cells (Tn), memory T cells including stem-cell like memory (Tscm), central memory (Tcm), and effector memory (Tem) T cells), and effector (Teff) T cells. The varying amounts of different T cell subsets can affect the therapeutic profile and efficacy of the resulting engineered T cells. In particular, T cell expansion methods that result in enriched amounts of T cells in early differentiation stages can be therapeutically useful.

Cell growth media provided by the present disclosure can be used for T-cell expansion in which the proportion of Tscm and/or Tem is enriched. Tscm cells are the least-differentiated type of memory T cells and, for adoptive T-cell therapy, can be useful in promoting prolonged in vivo T-cell proliferation following administration of the engineered cells to a patient. Such cell culture media and immune cells prepared using culture media provided by the present disclosure can result in more potent adoptive cell transfer therapies including, for example, CAR-T therapies.

Cell growth media comprising combinations of different stimulants of cell proliferation in combination with an extracellular modulator of cell metabolism can result in proliferation/expansion of a desired immune cell population having a specific cell phenotype or a combination of cells having different phenotypes.

A population of immune cells provided by the present disclosure can comprise a population of immune cells that are enriched in a target immune cell population compared to that of the initial population of primary cells.

For example, an enriched population of immune cells can have a concentration of a target population of immune cells that is greater than 10 times, greater than 10² times, greater than 10³ times, or greater than 10⁴ times that of the concentration of the initial population of primary cells.

An enriched population of immune cells can comprise, for example, an enriched population of T cells.

For example, an enriched population of immune cells provided by the present disclosure can comprise an enriched population of CD4+ T cells and CD8+ T cells.

An enriched population of immune cells provided by the present disclosure can be prepared using a culture medium provided by the present disclosure.

An enriched population of immune cells provided by the present disclosure can be prepared using a method of expanding a target immune cell population provided by the present disclosure.

An enriched population of immune cells provided by the present disclosure can be prepared using a method of cell manufacturing provided by the present disclosure.

An enriched population of T cells can be harvested from the culture medium, washed, concentrated, and combined with pharmaceutically acceptable excipients to provide a pharmaceutical composition suitable for administration to a patient. A pharmaceutical composition can be formulated for infusion. Examples of suitable infusion media include, for example, an isotonic medium formulation, such as normal saline, Normosol™ R (Abbott), or Plasma-Lyte™ A (Baxter), but also 5% dextrose in water or Ringer's lactate can be utilized. The infusion medium can be supplemented with human serum albumin.

A therapeutically effective number of T cells can be, for example, greater than 10⁴ cells, greater than 10⁶ cells, greater than 10⁸ cells, greater than 10¹⁰ cells, or greater than 10¹² cells.

A therapeutically effective concentration of T cells for administering to a patient can be, for example, greater than 10⁴ cells/mL, greater than 10⁶ cells/mL, or greater than 10⁸ cells/mL.

A therapeutically effective number of cells can range, for example, from 10⁵ to 10¹² cells per kg patient weight.

CAR treatments can be administered multiple times at dosages within these ranges. The cells can be autologous, allogeneic, or heterologous to the patient undergoing therapy.

A pharmaceutical composition provided by the present disclosure can comprise, for example, an enriched population of immune cells provided by the present disclosure, such as an enriched population of T cells such as an enriched population of CD4+ T cells and CD8+ T cells.

A pharmaceutical composition provided by the present disclosure can comprise a therapeutically effective concentration of an enriched population of immune cells provided by the present disclosure for treating a disease in a patient.

A pharmaceutical composition provided by the present disclosure can comprise an enriched population of immune cells provided by the present disclosure in combination with one or more pharmaceutically acceptable excipients.

A pharmaceutical composition provided by the present disclosure can be an intravenous formulation.

Aspects of the present invention include immobilized IL-7Rαγc agonist peptides.

An immobilized IL-7Rαγc agonist peptide comprises an IL-7Rαγc agonist peptide such as an IL-7Rαγc agonist peptide provided by the present disclosure bound to a substrate.

A substrate can comprise any suitable substrate that can be added to a culture medium. The substrate prevents or minimizes the IL-7Rαγc agonist peptides from being internalized into cells.

Examples of suitable substrates include beads, matrices, and plates.

An IL-7Rαγc agonist peptide such as an IL-7Rαγc agonist provided by the present disclosure can be bound to a substrate through a substrate linker.

A substrate linker can comprise, for example, a suitable peptidyl linker and/or a synthetic linker as disclosed herein.

Kits provided by the present disclosure can comprise an IL-7Rαγc agonist peptide provided by the present disclosure. An IL-7Rαγc agonist peptide can be provided, for example, as a lyophilizate or as a stable solution. A kit can comprise a culture medium containing an IL-7Rαγc agonist peptide provided by the present disclosure. An IL-7Rαγc agonist peptide can be provided as free peptide or can be bound to a substrate such as bead or matrix.

Kits can further comprise, for example, a second stimulant of immune cell proliferation such as an IL-2Rβγc agonist peptide, an IL-15βγc agonist peptide, or a combination thereof.

A kit can comprise a monoclonal antibody for activating a target immune cell population. For example, an antibody can comprise an anti-CD3 monoclonal antibody, and anti-CD28 monoclonal antibody, or a combination thereof.

An enriched immune cell population provided by the present disclosure can be incorporated into pharmaceutical compositions to be administered to a patient by any appropriate route of administration, including intradermal, intramuscular, intraperitoneal, intravenous, subcutaneous, intranasal, epidural, oral, peroral, sublingual, intracerebral, intravaginal, transdermal, rectal, inhalation, or topical. A pharmaceutical composition provided by the present disclosure can be an injectable formulation.

Pharmaceutical compositions provided by the present disclosure can be injectable intravenous formulations. Pharmaceutical compositions provided by the present disclosure can be oral formulations.

Oral formulations may be oral dosage forms. A pharmaceutical composition may be formulated for intravenous administration or subcutaneous administration.

Pharmaceutical compositions provided by the present disclosure may comprise a therapeutically effective amount of an enriched immune cell population together with a suitable amount of one or more pharmaceutically acceptable vehicles to provide a composition for proper administration to a patient.

Suitable pharmaceutical vehicles and methods of preparing pharmaceutical compositions are described in the art.

Accordingly, it is within the capability of those of skill in the art to assay and use IL-7Rαγc agonist peptides and/or pharmaceutical compositions thereof for therapy.

Enriched immune cell populations, and/or pharmaceutical composition thereof can generally be used in an amount effective to achieve the intended purpose. For use to treat a disease such as cancer, an autoimmune disease, or an inflammatory disease, an enriched immune cell population and/or pharmaceutical composition thereof, may be administered or applied in a therapeutically effective amount.

The amount of an enriched immune cell population and/or pharmaceutical composition of any of the foregoing that will be effective in the treatment of a particular disorder or condition disclosed herein will depend in part on the nature of the disorder or condition, and can be determined by standard clinical techniques known in the art. In addition, in vitro or in vivo assays may optionally be employed to help identify optimal dosage ranges. The amount of an enriched immune cell population and/or pharmaceutical composition of any of the foregoing administered will depend on, among other factors, the patient being treated, the weight of the patient, the severity of the affliction, the manner of administration and the judgment of the prescribing physician.

An enriched immune cell population can be assayed in vitro and in vivo, for the desired therapeutic activity, prior to use in humans. For example, in vitro assays may be used to determine whether the administration of a specific compound or a combination of compounds is preferred. The compounds can also be demonstrated to be effective and safe using animal model systems.

In certain embodiments, a therapeutically effective dose of an enriched immune cell population and/or pharmaceutical composition of any of the foregoing will provide therapeutic benefit without causing substantial toxicity. The toxicity of an enriched immune cell population and/or pharmaceutical compositions of any of the foregoing may be determined using standard pharmaceutical procedures and may be readily ascertained by the skilled artisan. The dose ratio between toxic and therapeutic effect is the therapeutic index. An enriched immune cell population and/or pharmaceutical composition of any of the foregoing exhibits a particularly high therapeutic index in treating disease and disorders. A dose of an enriched immune cell population and/or pharmaceutical composition of any of the foregoing will be within a range of circulating concentrations that include an effective dose with minimal toxicity.

An enriched immune cell population provided by the present disclosure can be included in a kit that may be used to administer the compound to a patient for therapeutic purposes. A kit may include a pharmaceutical composition comprising an enriched immune cell population provided by the present disclosure suitable for administration to a patient and instructions for administering the pharmaceutical composition to the patient. The kit can be a kit for treating cancer, for treating an autoimmune disease, or for treating an inflammatory disease. A kit for use in treating cancer in a patient can comprise an enriched immune cell population provided by the present disclosure, a pharmaceutically acceptable vehicle for administering the compound, and instructions for administering the compound to a patient.

The pharmaceutical compositions can be included in a container, pack, or dispenser together with instructions for administration.

Instructions supplied with a kit may be printed and/or supplied, for example, as an electronic-readable medium, a video cassette, an audiotape, a flash memory device, or may be published on an internet website or distributed to a patient and/or health care provider as electronic communication.

A method of treating a disease in a patient provided by the present disclosure can comprise administering to a patient in need of such treatment a therapeutically effective amount of a pharmaceutical composition provided by the present disclosure.

Enriched immune cell populations provided by the present disclosure can be used, for example, to treat diseases such as cancer, an autoimmune disease, or an infectious disease, including a viral disease such as COVID-19.

An enriched immune cell population provided by the present disclosure and pharmaceutical compositions of any of the foregoing may be administered to a patient to treat an organ transplant.

An enriched immune cell population provided by the present disclosure and pharmaceutical compositions of any of the foregoing may be administered to a patient together with another compound for treating an autoimmune disease in the subject. The at least one other therapeutic agent may be an enriched immune cell population provided by the present disclosure. An enriched immune cell population and the at least one other therapeutic agent may act additively or synergistically. The at least one additional therapeutic agent may be included in the same pharmaceutical composition or vehicle comprising the enriched immune cell population or may be in a separate pharmaceutical composition or vehicle. Accordingly, methods provided by the present disclosure further include, in addition to administering an enriched immune cell population, administering one or more therapeutic agents effective for treating an autoimmune disease or a different disease, disorder, or condition than an autoimmune disease. Methods provided by the present disclosure include administration of an enriched immune cell population and one or more other therapeutic agents, provided that the combined administration does not inhibit the therapeutic efficacy of an enriched immune cell population and/or does not produce adverse combination effects.

Enriched immune cell populations provided by the present disclosure may be used for treating cancer in a patient. The cancer can be, for example, a solid tumor or a metastasis.

Enriched immune cell populations provided by the present disclosure or pharmaceutical compositions thereof can be used to treat, for example, one or more of the following cancers: acute lymphoblastic leukemia, acute myeloid leukemia, adrenocortical carcinoma, appendix cancer, astrocytoma, atypical teratoid/rhabdoid tumor, basal cell carcinoma (nonmelanoma), B-cell lymphoma, bladder cancer, bone cancer, brain and spinal cord tumors, brain stem cancer, brain tumor, breast cancer, bronchial tumors, Burkitt lymphoma, carcinoid tumor, carcinoma of head and neck, central nervous system embryonal tumors, cerebellar astrocytoma, cerebral astrocytoma/malignant glioma, cervical cancer, chordoma, chronic lymphocytic leukemia, chronic myelogenous leukemia, colorectal cancer, craniopharyngioma, cutaneous T-cell lymphoma, desmoplastic small round cell tumor, ductal carcinoma, dye cancer, endocrine pancreas tumors (islet cell tumors), endometrial cancer, ependymoblastoma, esophageal cancer, esthesioneuroblastoma, Ewing family of tumors, extracranial germ cell tumor, extrahepatic bile duct cancer, gallbladder cancer, gastric cancer, gastrointestinal carcinoid tumor, gastrointestinal stromal tumor, gestational trophoblastic tumor, glioblastoma, glioma, hairy cell leukemia, head and neck cancer, heart cancer, hematopoetic tumors of the lymphoid lineage, hepatocellular cancer, Hodgkin lymphoma, hypopharyngeal cancer, hypothalamic and visual pathway glioma, IDs-related lymphoma, intraocular melanoma, islet cell tumors, Kaposi sarcoma, kidney cancer, Langerhans cell histiocytosis, laryngeal cancer, leukemia, lip and oral cavity cancer, male breast cancer, malignant fibrous histiocytoma, malignant germ cell tumors, malignant mesothelioma, medulloblastoma, melanoma, Merkel cell carcinoma, mesothelioma, mouth cancer, multiple endocrine neoplasia syndrome, multiple myeloma, mycosis fungoides, myelodysplastic, myeloproliferative neoplasms, nasal cavity and paranasal sinus cancer, nasopharyngeal cancer, neuroblastoma, non-Hodgkin lymphoma, non-small cell lung cancer, oral cancer, oropharyngeal cancer, osteosarcoma, ovarian cancer, ovarian epithelial cancer, ovarian germ cell tumor, ovarian low malignant potential tumor, pancreatic cancer, pancreatic neuroendocrine tumors (islet cell tumors), papillomatosis, paraganglioma, paranasal sinus and nasal cavity cancer, parathyroid cancer, penile cancer, pharyngeal cancer, pheochromocytoma, pineal parenchymal tumors, pineoblastoma and supratentorial primitive neuroectodermal tumors, pituitary tumor, plasma cell neoplasm/multiple myeloma, pleuropulmonary blastoma, pregnancy and breast cancer, primary central nervous system lymphoma, primary liver cancer, primary metastatic squamous neck cancer with occult, prostate cancer, rectal cancer, renal cell cancer, renal pelvis and ureter, respiratory tract carcinoma, retinoblastoma, rhabdomyosarcoma, salivary gland cancer, sarcoma, Sézary syndrome, skin cancer, small intestine cancer, soft tissue sarcoma, squamous cell carcinoma (nonmelanoma), stomach cancer, supratentorial primitive neuroectodermal tumors, T-cell lymphoma, testicular cancer, throat cancer, thymoma and thymic carcinoma, thyroid cancer, transitional cell cancer, urethral cancer, uterine sarcoma, vaginal cancer, visual pathway and hypothalamic glioma, vulvar cancer, Waldenström macroglobulinemia, Wilms tumor, and systemic and central metastases of any of the foregoing.

A cancer can be a solid tumor, a metastatic cancer, and/or a blood malignancy such as lymphoma or leukemia.

Enriched immune cell populations provided by the present disclosure or pharmaceutical compositions thereof can be used to treat solid tumors.

Enriched immune cell populations provided by the present disclosure or pharmaceutical compositions thereof can be used to treat tumor metastases.

Enriched immune cell populations provided by the present disclosure or pharmaceutical compositions thereof can be used to treat circulating tumor cells.

Enriched immune cell populations provided by the present disclosure or pharmaceutical compositions thereof can be used to treat, for example, a cancer selected from primary adult and childhood brain and CNS cancers including glioblastoma (GBM) and astrocytoma, skin cancers including melanoma, lung cancers including small cell lung cancers, non-small cell lung cancers (NSCLC), and large cell lung cancers, breast cancers including triple-negative breast cancer (TNBC), blood cancers including myelodysplastic syndrome (MDS), multiple myeloma (MM), and acute myeloid leukemia (AML), prostate cancer including castrate-resistant prostate cancer (CRPC), liver cancers including hepatocellular carcinoma (HCC), esophageal and gastric cancers, and any systemic and central metastases of any of the foregoing.

The amount of an enriched immune cell population provided by the present disclosure, or pharmaceutical composition thereof that will be effective in the treatment of a cancer can depend, at least in part, on the nature of the disease and may be determined by standard clinical techniques known in the art. In addition, in vitro or in vivo assays may be employed to help identify optimal dosing ranges. Dosing regimens and dosing intervals may also be determined by methods known to those skilled in the art. The amount of an enriched immune cell population provided by the present disclosure administered may depend on, among other factors, the patient being treated, the weight of the patient, the severity of the disease, the route of administration, and the judgment of the prescribing physician.

For systemic administration, a therapeutically effective dose may be estimated initially from in vitro assays. Initial doses may also be estimated from in vivo data, e.g., animal models, using techniques that are known in the art. Such information may be used to more accurately determine useful doses in humans. One having ordinary skill in the art may optimize administration to humans based on animal data.

A dose of an enriched immune cell population provided by the present disclosure and appropriate dosing intervals may be selected to maintain a sustained therapeutically effective concentration of the enriched immune cell population provided by the present disclosure in the blood of a patient, and in certain embodiments, without exceeding a minimum adverse concentration.

A pharmaceutical composition comprising an enriched immune cell population provided by the present disclosure may be administered, for example, once per week, every 2 weeks, every 3 weeks, every 4 weeks, every 5 weeks, or every 6 weeks. Dosing may be provided alone or in combination with other drugs and may continue as long as required for effective treatment of the disease. Dosing may also be undertaken using continuous or semi-continuous administration over a period of time. Dosing includes administering a pharmaceutical composition to a mammal, such as a human, in a fed or fasted state.

A pharmaceutical composition may be administered in a single dosage form or in multiple dosage forms or as a continuous or an accumulated dose over a period of time. When multiple dosage forms are used, the amount of an enriched immune cell population provided by the present disclosure contained within each of the multiple dosage forms may be the same or different.

Pharmaceutical compositions comprising an enriched immune cell population may be administered to treat a disease in a patient so as to provide a therapeutically effective concentration of the enriched immune cell population in the blood of a patient for an extended period of time such as, for example, for at least 1 day, for at least 1 week, at least 2 weeks, at least 4 weeks, at least 5 weeks, or at least 6 weeks.

The amount of an enriched immune cell population administered may vary during a treatment regimen.

Pharmaceutical compositions provided by the present disclosure may further comprise one or more pharmaceutically active compounds in addition to an enriched immune cell population provided by the present disclosure. Such compounds may be provided, for example, to treat the cancer being treated with the enriched immune cell population or to treat a disease, disorder, or condition other than the cancer being treated with the enriched immune cell population, to treat a side-effect caused by administering the enriched immune cell population, to augment the efficacy of the enriched immune cell population, and/or to modulate the activity of the enriched immune cell population.

An enriched immune cell population provided by the present disclosure may be used in combination with at least one other therapeutic agent. An enriched immune cell population may be administered to a patient together with another compound for treating cancer in the patient. The at least one other therapeutic agent can be a second, different enriched immune cell population. An enriched immune cell population and the at least one other therapeutic agent may act additively or, and in certain embodiments, synergistically with another enriched immune cell population. The at least one additional therapeutic agent may be included in the same pharmaceutical composition or vehicle comprising the enriched immune cell population or may be in a separate pharmaceutical composition or vehicle. Accordingly, methods provided by the present disclosure further include, in addition to administering an enriched immune cell population, administering one or more therapeutic agents effective for treating cancer or a different disease, disorder or condition than cancer. Methods provided by the present disclosure include administration of an enriched immune cell population and one or more other therapeutic agents provided that the combined administration does not inhibit the therapeutic efficacy of the enriched immune cell population and/or does not produce adverse combination effects.

A pharmaceutical composition comprising an enriched immune cell population may be administered concurrently with the administration of another therapeutic agent, which may be part of the same pharmaceutical composition, or in a different pharmaceutical composition, as that comprising an enriched immune cell population. An enriched immune cell population may be administered prior or subsequent to administration of another therapeutic agent. In certain combination therapies, the combination therapy may comprise alternating between administering an enriched immune cell population and a composition comprising another therapeutic agent, e.g., to minimize adverse drug effects associated with a particular drug. When an enriched immune cell population is administered concurrently with another therapeutic agent that potentially may produce an adverse drug effect including, for example, toxicity, the other therapeutic agent may be administered at a dose that falls below the threshold at which the adverse drug reaction is elicited.

A pharmaceutical composition comprising an enriched immune cell population provided by the present disclosure may be administered with one or more substances, for example, to enhance, modulate and/or control release, bioavailability, therapeutic efficacy, therapeutic potency, and/or stability, of the enriched immune cell population. For example, a pharmaceutical composition comprising an enriched immune cell population can be co-administered with an active agent having pharmacological effects that enhance the therapeutic efficacy of the enriched immune cell population.

An enriched immune cell population provided by the present disclosure, or a pharmaceutical composition thereof, may be administered in conjunction with an agent known or believed to be effective in treating a disease such as cancer, an autoimmune disease, or an inflammatory disease in a patient, such as the same disease being treated with the enriched immune cell population.

An enriched immune cell population, or a pharmaceutical composition thereof may be administered in conjunction with an agent known or believed to interfere with cell proliferation.

An enriched immune cell population, or a pharmaceutical composition thereof, may be administered in conjunction with an agent known or believed to interfere with cellular metabolism, to be an anti-metabolite, to interfere with RNA transcription, to interfere with RNA translation, to interfere with cellular protein synthesis, to interfere with synthesis of precursors for DNA synthesis and replication, to interfere with purine synthesis, to interfere with nucleoside synthesis, to interact with mTOR, to be an mTOR inhibitor, or to interfere with cell cycle checkpoints.

An enriched immune cell population or a pharmaceutical composition thereof may be administered in conjunction with a checkpoint inhibitor including a CTLA-4 inhibitor such as ipilimumab, a PD-1 inhibitor such as pembrolizumab and nivolumab, and/or a PD-LI inhibitor such as atezolizumab, avelumab, and durvalumab. An enriched immune cell population or a pharmaceutical composition thereof may be administered in conjunction with an immunomodulator such as CD137/4-1BB, CD27, GITR, and/or CD40.

An enriched immune cell population or a pharmaceutical composition thereof may be administered in conjunction with an agent known or believed to be cytotoxic, to cause DNA damage, to cause cell cycle arrest, or to cause mitotic catastrophe.

An enriched immune cell population or a pharmaceutical composition thereof may be administered in conjunction with an agent known or believed to modulate glutathione concentration, to modulate glutathione concentration within cells, to decrease glutathione concentration within cells, to reduce glutathione uptake into cells, to reduce glutathione synthesis, or to reduce glutathione synthesis within cells.

An enriched immune cell population or a pharmaceutical composition thereof may be administered in conjunction with an agent known or believed to interfere with neovascularization, to reduce neovascularization, or to promote neovascularization.

An enriched immune cell population or a pharmaceutical composition thereof may be administered in conjunction with an agent known or believed to interfere with hormone homeostasis, to interfere with hormone synthesis, to interfere with hormone receptor binding, or to interfere with hormone signal transduction.

An enriched immune cell population or a pharmaceutical composition thereof may be administered in conjunction with an agent known or believed to interfere with growth factor homeostasis, to interfere with growth factor receptor expression, to interfere with growth factor binding to growth factor receptors, to interfere with growth factor receptor signal transduction, to interfere with the Hedgehog (Hh) signaling, to inhibit the Hedgehog pathway signaling, to inhibit ALK (anaplastic lymphoma kinase) pathway signaling, or to inhibit the non-homologous end joining (NHEJ) pathway.

An enriched immune cell population or a pharmaceutical composition thereof may be administered in conjunction with one or more agents known or believed to be a VEGFR (vascular endothelial growth factor receptor) inhibitor, a RTK (receptor tyrosine kinase) inhibitor, a sodium channel current blocker, a FAK (focal adhesion kinase) inhibitor, a GLI (glioma-associated oncogene) inhibitor, a GLI1 inhibitor, a GLI2 inhibitor, a GLI3 inhibitor, a MAPK (mitogen-activated protein kinase) inhibitor, a MAPK/ERK pathway (also known as Ras-Raf-MEK-ERK pathways) inhibitor, a MEK1 inhibitor, a MEK2 inhibitor, a MEK5 inhibitor, a MEK5/ERK5 inhibitor, aRTA (renal tubular acidosis) inhibitor, a ALK (anaplastic lymphoma kinase) inhibitor, Aa LK kinase inhibitor, a nuclear translocation inhibitor, a PORCN (porcupine) inhibitor, a 5-ARI (5α-reductase inhibitor), topoisomerase inhibitor, a Ras (rat sarcoma) inhibitor, a K-ras inhibitor, a CERK (ceramide kinase) inhibitor, a PKB (protein kinase B, also known as AKT) inhibitor, a AKT1 inhibitor, EZH2 (enhancer of zeste homolog 2) inhibitor, a BET (bromodomain and extraterminal domain motif) inhibitor, a SYK (spleen tyrosine kinase) inhibitor, JAK (janus kinase) inhibitors, a SYK/JAK inhibitor, a IDO (indoleamine-pyrrole 2,3-dioxygenase) inhibitor, a IDO1 inhibitor, a RXR (retinoic X receptors) activating agent, a selective RXR activating agent, a p-glycoprotein inhibitor, a ERK inhibitor, a PI3K (phosphatidylinositol-4,5-bisphosphate 3-kinase) inhibitor, a BRD (bromodomain-containing protein) inhibitor, a BRD2 inhibitor, a BRD3 inhibitor, a BRD4 inhibitor, a BRDT (bromodomain testis-specific protein) inhibitor, a reverse transcriptase inhibitor, a NRT (nucleoside analog reverse-transcriptase) inhibitor, a PIM (proviral integrations of moloney virus) inhibitor, a EGFR (epidermal growth factor receptor) inhibitor, a photosensitizer, a radiosensitizer, a ROS (proto-oncogene, receptor tyrosine kinase) inhibitor, a ROS1 (ROS proto-oncogene 1) inhibitor, a CK (casein kinase) inhibitor, a CK2 inhibitor, a Bcr-Abl (breakpoint cluster region—Abelson proto-oncogene) tyrosine-kinase inhibitor such as dasatinib, a microtubule stabilizing agent, a microtubule depolymerization/disassembly inhibitor, a DNA intercalator, an androgen receptor antagonist, a chemoprotective agents, a HDAC (histone deacetylase) inhibitor, a DPP (dipeptidyl peptidase) inhibitor, a DPP-4 inhibitor, BTK (Bruton's tyrosine kinase) inhibitor, a kinase inhibitor such as imatinib, a tyrosine kinase inhibitor such as nilotinib, a ARP (poly (ADP-ribose) polymerase) inhibitor, a CDK (cyclin-dependent kinase) inhibitor, a CDK4 inhibitor, a CDK6 inhibitor, a CDK4/6 inhibitor, a HIF1α (hypoxia-inducible factor 1-α) inhibitor, a DNA ligase inhibitor, a DNA ligase IV inhibitor, a NHEJ (non-homologous end joining) inhibitor, a DNA ligase IV, a NHEJ inhibitor and a RAF inhibitor, a TKI and a RAF inhibitor, a TKI and RAF inhibitor such as sorafenib, a PDT (photodynamic therapy) sensitizer, an ATR (ataxia telangiectasia- and Rad3-related protein kinase) inhibitor, or a combination of any of the foregoing.

An enriched immune cell population or a pharmaceutical composition thereof may be administered in conjunction with one or more chemotherapeutic agents, such as, for example, a VEGFR inhibitor such as fruquintinib, motesanib/AMG-706, vatalanib; a RTK inhibitor such as ponatinib; a sodium channel blocker such as GS967; a FAK inhibitor such as TAE226; a GLI1 and GLI2 inhibitor such as GANT61, a MEK inhibitor such as binimetinib; a RTA inhibitor such as linifanib; an ALK inhibitor such as brigstinib; bromopyruvic acid; a DNA alkylating agent such as thiotepa; nuclear translocations factors such as JSH-23; a PORCn inhibitor such as Wnt-C59; a 5α-reductase inhibitor such as dutasteride; a topoisomerase inhibitor such as carubicin; a RAS inhibitor such as Kobe0065; a CerK inhibitor such as NVP-231; an AKT inhibitor such as uprosertib; a EZH2 inhibitor such as GSK-503; a BET bromodomain inhibitor such as OTX015; a MEK5/ERK5 inhibitor such as BIX02189; a Syl/JAK inhibitor such as cerdulatinib; an IDO1 inhibitor such as NLG919; a retinoic X receptor activating agent such as bexsrotene; a PGP inhibitor such as acotiamide or actotiamide HCl; an Erk inhibitor such SCH772984; a PI3K inhibitor such as gedatolisib; a JAK inhibitor such as ruxolitinib; an AKT inhibitor such as afuresertib or afuresertib HCl; an ALK1 inhibitor such as ceritinib; an HDAC inhibitor such as abexinostat; a DPP inhibitor such as oamarigliptin; an EGFR inhibitor such as gefittinib; an EZH2 inhibitor such as GSK126; a BTK inhibitor such as ibrutinib; a kinase inhibitor such as imatinin HCl; an IDO inhibitor such as INCB024360; a DNA crosslinker such as mitomycin C; a tyrosine kinase inhibitor such as nilotinib, a PARP inhibitor such as olaparib; a tubulin stabilization promoter such as paclitaxel; a CDK4/6 inhibitor such as palbociclib; a RTK inhibitor such as sunitinib; a PDT sensitizer such as tslsporfin; a p-glycoprotein inhibitor such as tariquidar; an ATR inhibitor such as VE-822; an HDAC inhibitor such as PCI-24781; a DPP inhibitor such as omarigliptin; an EGFR inhibitor such as gefinib; an EZH2 inhibitor such as GSK126; a BTK inhibitor such as irbrutinib; an IDO inhibitor such as INCB024360; or a combination of any of the foregoing.

For example, an enriched immune cell population or a pharmaceutical composition thereof may be administered in conjunction with another chemotherapeutic agent, such as, for example, N-acetyl cysteine (NAC), adriamycin, alemtuzumab, amifostine, arsenic trioxide, ascorbic acid, bendamustine, bevacizumab, bortezomib, busulfan, buthionine sulfoxime, carfilzomib, carmustine, clofarabine, cyclophosphamide, cyclosporine, cytarabine, dasatinib, datinomycin, defibrotide, dexamethasone, docetaxel, doxorubicin, etoposide, filgrastim, floxuridine, fludarabine, gemcitabine, interferon alpha, ipilimumab, lenalidomide, leucovorin, melphalan, mycofenolate mofetil, paclitaxel, palifermin, panobinostat, pegfilrastim, prednisolone, prednisone, revlimid, rituximab, sirolimus, sodium 2-mercaptoethane sulfonate (MESNA), sodium thiosulfate, tacrolimus, temozolomide, thalidomide, thioguanine, thiotepa, topotecan, velcade, or a combination of any of the foregoing.

An enriched immune cell population or a pharmaceutical compositions thereof can be used in combination therapy with other chemotherapeutic agents including one or more antimetabolites such as folic acid analogs; pyrimidine analogs such as fluorouracil, floxuridine, and cytosine arabinoside; purine analogs such as mercaptopurine, thiogunaine, and pentostatin; natural products such as vinblastine, vincristine, etoposide, tertiposide, dactinomycin, daunorubicin, doxurubicin, bleomycin, mithamycin, mitomycin C, L-asparaginase, and interferon alpha; platinum coordination complexes such as cis-platinum, and carboplatin; mitoxantrone; hydroxyurea; procarbazine; hormones and antagonists such as prednisone, hydroxyprogesterone caproate, medroxyprogesterone acetate, megestrol acetate, diethylstilbestrol, ethinyl estradiol, tamoxifen, testosterone propionate, fluoxymesterone, flutamide, and leuprolide, anti-angiogenesis agents or inhibitors such as angiostatin, retinoic acids, paclitaxel, estradiol derivatives, and thiazolopyrimidine derivatives; apoptosis prevention agents; triptolide; colchicine; luliconazole; and radiation therapy.

An enriched immune cell population or a pharmaceutical composition thereof may be co-administered with a compound that inhibits DNA repair such as, for example, O6-benzylguanine (O6-BG).

An enriched immune cell population or a pharmaceutical composition thereof may be administered in conjunction with one or more chemotherapeutic agents, such as, for example, abarelix, abiraterone, abiraterone acetate, n-acetyl cysteine, aclarubicin hydrochloride, adriamycin, adenine, afatinib, afatinib dimaleate, alemtuzumab, alendronate sodium, alitretinoin, allopurinol sodium, altretamine, amifostine, aminoglutethimide, aminolevulinic acid, amrubicin, amsacrine, anastrozole, angiostatin, apremilast, aprepitant, arsenic trioxide, ascorbic acid, 1-asparaginase, azacitidine, azathioprine sodium, bazedoxifene (serm), belinostat, bendamustine hcl, O6-benzylguanine, bevacizumab, bexarotene, bicalutamide, biricodar, bleomycin sulfate, bortezomib, bosutinib, brivudine, buserelin, busulfan, buthionine sulfoxime, cabazitaxel, cabozantinib, capecitabine, carboplatin, carboquone, carfilzomib, carmofur, carmustine, ceritinib, chlorambucil, cisplatin, cladribine, clodronate disodium, clofarabine, crizotinib, cyclophosphamide, cyclosporine, cytarabine, cytosine arabinoside, dabrafenib, dacarbazine, dactinomycin, dasatinib, datinomycin, daunorubicin, decitabine, defribrotide, degarelix acetate, dexamethasone, dexrazoxane hydrochloride, diaziquone, diethyl stilbestrol, docetaxel, doxifluridine, doxorubicin hydrochloride, doxorubicin free base, dromostanolone propionate, dutasteride, eltrombopag, enzalutamide, epirubicin hydrochloride, eribulin mesylate, erlotinib hydrochloride, estramustine phosphate sodium, ethinyl estradiol, etoposide phosphate, etoposide, everolimus, exemestane, fentanyl, filgrastim, fingolimod, floxuridine, fludarabine phosphate, fluorouracil, fluoxymesterone, flutamide, formestane, formylmelphalan, fosaprepitant, fotemustine, fulvestrant, gefitinib, gemcitabine hydrochloride, gemcitabine free base, glutathione, glyciphosphoramide, glyfosfin, goserelin acetate, granisetron hydrochloride, heptaplatin, hexyl 5-aminolevulinate, histrelin acetate, hydroxyprogesterone caproate, hydroxyurea, ibandronate sodium, ibrutinib, icotinib, idarubicin HCl, idelalisib, idoxuridine, ifosfamide, interferon alpha, imatinib mesylate, imiquimod, ingenol mebutate, ipilimumab, irinotecan hydrochloride, ixabepilone, lanreotide acetate, lapatinib free base, lapatinib ditosylate, lasofoxifene, lenalidomide, letrozole, leucovorin calcium, leuprolide acetate, levamisole hydrochloride, levoleucovorin calcium, iobenguane, lobaplatin, lomustine, maropitant, masoprocol, mechlorethamine hydrochloride, megestrol acetate, medroxyprogesterone acetate, melphalan hydrochloride, mercaptopurine, mercaptoethane sulfonate sodium, methotrexate, methoxsalen, methyl aminolevulinate, methylene blue, methylisoindigotin, mifamurtide, miltefosine, miriplatin, mithamycin, mitobronitol, mitomycin C, mitotane, mitoxantrone hydrochloride, mycophenolate mofetil, nabiximols, nafarelin, nandrolone, nedaplatin, nelarabine, netupitant, nilotinib, nilutamide, nimustine, nintedanib, nocodazole, octreotide, olaparib, omacetaxine mepesuccinate, ondansetron hydrochloride, oxaliplatin, paclitaxel, palbociclib, palifermin, palonosetron hydrochloride, pamidronate disodium, panobinostat, pasireotide, pazopanib hydrochloride, pegfilrastim, pemetrexed disodium, pentostatin, peplomycin, pipobroman, pirarubicin, plerixafor, plicamycin, pomalidomide, ponatinib, porfimer sodium, porfiromycin, pralatrexate, prednimustine, prednisolone, prednisone, procarbazine hydrochloride, quinagolide hydrochloride, raloxifene, raltitrexed, radotinib, ranimustine, retinoic acids, revlimide, rituxinab, romidepsin, ruxolitinib, ruxolitinib phosphate, semustine, sirolimus, sodium thiosulfate, sorafenib free base, sorafenib tosylate, streptozocin, sufentanil, sunitinib, tacrolimus, talaporfin sodium, tamibarotene, tamoxifen citrate, tapentadol, temoporfin, temozolomide, temsirolimus, teniposide, teriflunomide, tertiposide, testolactone, testosterone propionate, thalidomide, thioguanine, thiotepa, thymalfasin, toceranib phosphate, topotecan hydrochloride, toremifene citrate, trabectedin, trametinib, tretinoin, trilostane, triptorelin, tropisetron, uramustine, valrubicin, vandetanib, vedotin, vemurafenib, verteporfin, vinblastine, vincristine sulfate, vincristine free base, vindesine, vinorelbine tartrate, vorinostat, and zoledronic acid.

An enriched immune cell population or a pharmaceutical composition thereof may be administered in conjunction with one or more chemotherapeutic agents such as, for example, abemaciclib, abiraterone acetate, ABVD, ABVE, ABVE-PC, AC, acalabrutinib, AC-T, ADE, ado-trastuzumab emtansine, afatinib dimaleate, aldesleukin, alectinib, alemtuzumab, alpelisib, amifostine, aminolevulinic acid hydrochloride, anastrozole, apalutamide, aprepitant, arsenic trioxide, asparaginase Erwinia chrysanthemi, atezolizumab, avelumab, axicabtagene ciloleucel, axitinib, azacitidine, BEACOPP, belinostat, bendamustine hydrochloride, BEP, bevacizumab, bexarotene, bicalutamide, binimetinib, bleomycin sulfate, blinatumomab, bortezomib, bosutinib, brentuximab vedotin, brigatinib, BuMel, busulfan, cabazitaxel, cabozantinib-s-malate, CAF, calaspargase pegol-mknl, capecitabine, caplacizumab-yhdp, CAPOX, carboplatin, carboplatin-taxol, carfilzomib, carmustine, carmustine implant, CEM, cemiplimab-rwlc, ceritinib, cetuximab, CEV, chlorambucil, chlorambucil-prednisone, CHOP, cisplatin, cladribine, clofarabine, CMF, cobimetinib, copanlisib hydrochloride, COPDAC, COPP, COPP-ABV, crizotinib, CVP, cyclophosphamide, cytarabine, cytarabine liposome, dabrafenib mesylate, dacarbazine, dacomitinib, dactinomycin, daratumumab, darbepoetin a, dasatinib, daunorubicin hydrochloride, daunorubicin hydrochloride and cytarabine liposome, decitabine, defibrotide sodium, degarelix, denileukin diftitox, denosumab, dexamethasone, dexrazoxane hydrochloride, dinutuximab, docetaxel, doxorubicin hydrochloride, doxorubicin hydrochloride liposome, durvalumab, duvelisib, elotuzumab, eltrombopag olamine, emapalumab-lzsg, enasidenib mesylate, encorafenib, enzalutamide, epirubicin hydrochloride, EPOCH, epoetin alfa, erdafitinib, eribulin mesylate, erlotinib hydrochloride, etoposide, etoposide phosphate, everolimus, exemestane, fee, filgrastim, fludarabine phosphate, fluorouracil injection, fluorouracil—topical, flutamide, folfiri, folfiri-bevacizumab, folfiri-cetuximab, folfirinox, folfox, fostamatinib disodium, FU-LV, fulvestrant, gefitinib, gemcitabine hydrochloride, gemcitabine-cisplatin, gemcitabine-oxaliplatin, gemtuzumab ozogamicin, gilteritinib fumarate, glasdegib maleate, glucarpidase, goserelin acetate, granisetron, HPV bivalent vaccine, HPV bivalent vaccine, recombinant HPV nonavalent vaccine, HPV nonavalent vaccine, recombinant, HPV quadrivalent vaccine, HPV uadrivalent vaccine recombinant, hydroxyurea, hyper-CVAD, ibritumomab tiuxetan, ibrutinib, ICE, idarubicin hydrochloride, idelalisib, ifosfamide, imatinib mesylate, imiquimod, inotuzumab ozogamicin, interferon α-2b recombinant, iobenguane I¹³¹, ipilimumab, irinotecan hydrochloride, irinotecan hydrochloride liposome, ivosidenib, ixabepilone, ixazomib citrate, JEB, lanreotide acetate, lapatinib ditosylate, larotrectinib sulfate, lenalidomide, lenvatinib mesylate, letrozole, leucovorin calcium, leuprolide acetate, lomustine, lorlatinib, lutetium Lu 177-dotatate, mechlorethamine hydrochloride, megestrol acetate, melphalan, melphalan hydrochloride, mercaptopurine, mesna, methotrexate, methylnaltrexone bromide, midostaurin, mitomycin c, mitoxantrone hydrochloride, mogamulizumab-kpkc, moxetumomab pasudotox-tdfk, MVAC, necitumumab, nelarabine, neratinib maleate, netupitant and palonosetron hydrochloride, nilotinib, nilutamide, niraparib tosylate monohydrate, nivolumab, obinutuzumab, OEPA, ofatumumab, OFF, olaparib, olaratumab, omacetaxine mepesuccinate, ondansetron hydrochloride, OPPA, osimertinib mesylate, oxaliplatin, paclitaxel, paclitaxel albumin-stabilized nanoparticle formulation, PAD, palbociclib, palifermin, palonosetron hydrochloride, palonosetron hydrochloride and netupitant, pamidronate disodium, panitumumab, panobinostat, pazopanib hydrochloride, PCV, PEB, pegaspargase, pegfilgrastim, peginterferon α-2b, pembrolizumab, pemetrexed disodium, pertuzumab, plerixafor, polatuzumab vedotin-piiq, pomalidomide, ponatinib hydrochloride, pralatrexate, prednisone, procarbazine hydrochloride, propranolol hydrochloride, radium 223 dichloride, raloxifene hydrochloride, ramucirumab, rasburicase, ravulizumab-cwvz, R-CHOP, R-CVP, recombinant HPV bivalent vaccine, recombinant HPV nonavalent vaccine, recombinant HPV quadrivalent vaccine, recombinant interferon α-2b, regorafenib, R-EPOCH, ribociclib, R-ICE, rituximab, rituximab and hyaluronidase human, rolapitant hydrochloride, romidepsin, romiplostim, rucaparib camsylate, ruxolitinib phosphate, siltuximab, sipuleucel-t, sonidegib, sorafenib tosylate, STANFORD V, sunitinib malate, TAC, tagraxofusp-erzs, talazoparib tosylate, talc, talimogene laherparepvec, tamoxifen citrate, temozolomide, temsirolimus, thalidomide, thioguanine, thiotepa, tisagenlecleucel, tocilizumab, topotecan hydrochloride, toremifene, TPF, trabectedin, trametinib, trastuzumab, trastuzumab and hyaluronidase-oysk, trifluridine and tipiracil hydrochloride, uridine triacetate, VAC, Valrubicin, VAMP, vandetanib, VeIP, vemurafenib, venetoclax, vinblastine sulfate, vincristine sulfate liposome, vinorelbine tartrate, vip, vismodegib, vorinostat, XELIRI, XELOX, Ziv-aflibercept, zoledronic acid, and combinations of any of the foregoing.

The efficacy of an enriched immune cell population or a pharmaceutical composition thereof for treating cancer may be assessed using in vitro and animal studies and in clinical trials.

The suitability of an enriched immune cell population or a pharmaceutical composition thereof in treating cancer may be determined by methods described in the art.

An enriched immune cell population or a pharmaceutical composition thereof may be administered to a patient in need of such treatment to treat an autoimmune disease.

Examples of autoimmune diseases include Addison's disease, agammaglobulinemia, alopecia areata, amyloidosis, ankylosing spondylitis, anti-GBM/anti-TBN nephritis, antiphospholipid syndrome, autoimmune angioedema, autoimmune dysautonomia, autoimmune encephalomyelitis, autoimmune hepatitis, autoimmune inner ear disease, autoimmune myocarditis, autoimmune pancreatitis, autoimmune retinopathy, autoimmune urticaria, axonal and neuronal neuropathy, Balo's disease, Bechet's disease, benign mucosal pemphigoid, bullous pemphigoid, Castleman disease, celiac disease, Chagas disease, chronic inflammatory demyelinating polyneuropathy, chronic recurrent multifocal osteomyelitis, Churg-Strauss, cicatricial pemphigoid, Cogan's syndrome, cold agglutinin disease, congenital heart block, Coxsackie myocarditis, CREST syndrome, Crohn's disease, dermatitis herpetiformis, dermatomyositis, Devic's disease, discoid lupus, Dressler's syndrome, endometriosis, eosinophilic esophagitis, eosinophilic fasciitis, erythema nodosum, essential mixed cryoglobulinemia, Evans syndrome, fibromyalgia, fibrosing alveolitis, giant cell arteritis, giant cell myocarditis, glomerulonephritis, Goodpasture's syndrome, granulomatosis with polyangiitis, Graves' disease, Guillain-Barre syndrome, Hashimoto's thyroiditis, hemolytic anemia, Henoch-Schonlein purpura, herpes gestationis or pemphigoid gestationis, hypogammaglobulinemia, IgA nephropathy, IgG4-related sclerosing disease, immune thrombocytopenic purpura, inclusion body myositis, interstitial cystitis, juvenile arthritis, juvenile diabetes, juvenile myositis, Kawasaki disease, Lambert-Eaton syndrome, leukocytoclastic vasculitis, lichen planus, lichen sclerosis, ligneous conjunctivitis, linear IgA disease, lupus, Lyme disease chronic, Meniere's diseases, microscopic polyangiitis, mixed connective tissue disease, Mooren's ulcer, Mucha-Habermann disease, multiple sclerosis, myasthenia gravis, myositis, narcolepsy, neuromyelitis, optica, neutropenia, ocular cicatricial pemphigoid, optic neuritis, palindromic rheumatism, PANDAS, paraneoplastic cerebellar degeneration, paroxysmal nocturnal hemoglobinuria, Parry Romberg syndrome, pars planitis, Parsonage-Turner syndrome, pemphigus, peripheral neuropathy, perivenous encephalomyelitis, pernicious anemia, POEMS syndrome, polyarteritis nodosa, polyglandular syndromes, polymyalgia rheumatica, polymyositis, postmyocardial infarction syndrome, postpericardiotomy syndrome, primary biliary cirrhosis, primary sclerosing cholangitis, progesterone dermatitis, psoriasis, psoriatic arthritis, pure red cell aplasia, pyoderma gangrenosum, Raynaud's phenomenon, reactive arthritis, reflex sympathetic dystrophy, relapsing polychondritis, restless legs syndrome, retroperitoneal fibrosis, rheumatic fever, rheumatoid arthritis, sarcoidosis, Schmidt syndrome, scleritis, scleroderma, Sjogren's syndrome, sperm and testicular autoimmunity, stiff person syndrome, subacute bacterial endocarditis, Susac's syndrome, sympathetic ophthalmia, Takayasu's arteritis, temporal arteritis, thrombocytopenic purpura, Tolosa-Hunt syndrome, transverse myelitis, type 1 diabetes, ulcerative colitis, undifferentiated connective tissue disease, uveitis, vasculitis, vitiligo, and Wegener's granulomatosis.

An enriched immune cell population or a pharmaceutical composition thereof can be used to treat autoimmune disorders such as, for example, lupus, graft-versus-host disease, hepatitis C-induced vasculitis, Type I diabetes, multiple sclerosis, spontaneous loss of pregnancy, atopic diseases, and inflammatory bowel diseases.

An enriched immune cell population can be administered with one or more additional therapeutic agents for treating an autoimmune disease. An IL-7Rαγc binding compound or a pharmaceutical composition thereof may be administered in conjunction with one or more immunosuppressants including, for example, corticosteroids such as prednisone, budesonide, and prednisolone; Janus kinase inhibitors such as tofacitinib; calcineurin inhibitors such as cyclosporine and tacrolimus; mTOR inhibitors such as sirolimus and everolimus; IMDH inhibitors such as azathioprine, leflunomide, and mycophenolate; biologics such as abatacept adalimumab, anakinra, certolizumab, etanercept, golimumab, infliximab, ixekizumab, natalizumab, rituximab, secukinumab, tocilizumab, ustekinumab, and vedolizumab; and monoclonal antibodies such as basiliximab and daclizumab.

An enriched immune cell population or a pharmaceutical composition thereof may be administered to a patient to treat a disease associated with the activation, proliferation, metabolism, and/or differentiation of T-cells.

An enriched immune cell population or a pharmaceutical composition thereof may be administered to a patient to treat an organ transplant.

An enriched immune cell population or a pharmaceutical composition thereof may be administered in conjunction with an agent known or believed to interfere with proliferation, to interfere with mitosis, to interfere with DNA replication, or to interfere with DNA repair.

An enriched immune cell population or a pharmaceutical composition thereof may be administered to a patient to treat an immune deficiency disease.

Examples of primary immune deficiency disease include autoimmune lymphoproliferative syndrome, autoimmune polyglandular syndrome type 1, BENTA disease, caspase eight deficiency state, CARD9 deficiency, chronic granulomatous disease, common variable immunodeficiency, congenital neutropenia syndromes, CTLA4 deficiency, DOCK8 deficiency, GATA2 deficiency, glycosylation disorders, hyper-immunoglobulin E syndromes, hyper-immunoglobulin M syndromes, interferon γ, interleukin 12 and interleukin 23 deficiency, leukocyte adhesion deficiency, LRBA deficiency, PI2 kinase disease, PLCG2-associated antibody deficiency and immune dysregulation, severe combined immunodeficiency, STAT3 dominant-negative disease, STAT3 gain-of-function disease, warts, hypogammaglobulinemia, infections, and myelokathexis syndrome, Wiskott-Aldrich syndrome, X-linked agammaglobulinemia, X-linked lymphoproliferative disease, and XMEN disease.

Secondary immune deficiency disease occurs when the immune system is compromised due to an environmental factor such as infection, chemotherapy, severe burns, or malnutrition. Example of secondary immune deficiency diseases include newborn immunodeficiencies such as immature lymphoid organs, absent memory immunity, low maternal IgG levels, decreased neutrophil storage pool, decreased neutrophil function, and decreased natural killer cell activity; advanced age related immunodeficiencies such as decreased antigen-specific cellular immunity, T-cell oligoconality, and restricted B-cell repertoire; malnutrition related immunodeficiencies such as decreased cellular immune response and weekend mucosal barriers; diabetes mellitus related immunodeficiencies such as decreased mitogen-induced lymphoproliferation, defective phagocytosis, and decreased chemotaxis; chronic uremia related immunodeficiencies such as decreased cellular immune response, decreased generation of memory antibody responses, and decreased chemotaxis; genetic syndromes such as defective phagocytosis, defective chemotaxis, and variable defects of antigen-specific immune responses; and anti-inflammatory, immunomodulatory, and immuno-suppressive drug therapy related immune deficiencies such as lymphopenia, decreased cellular immune response and anergy, decreased proinflammatory cytokines, decreased phagocytosis, decreased chemotaxis, neutropenia, and weakened mucosal barriers; environmental conditions such as increased lymphocyte apoptosis, increased secretion of tolerogenic cytokines, cytopenia, decreased cellular immunity and anergy, and stress-induced nonspecific immune activation; and infectious diseases such as T-cell lymphopenia, decreased cellular immune response and anergy, and defective antigen-specific antibody responses.

An enriched immune cell population or a pharmaceutical composition thereof may be administered to a patient to increase the immune response in immuno-compromised patients.

An enriched immune cell population or a pharmaceutical composition thereof may be administered to a patient to increase the immune response in elderly patients.

An enriched immune cell population or a pharmaceutical composition thereof may be administered to a patient to treat an infectious disease such as a viral disease, where a viral disease refers to a disease caused by a virus.

A viral disease can be caused, for example, by a virus selected from herpes viruses, pox viruses, hepadnaviruses, papilloma viruses, adenoviruses, coronaviruses, orthomyxoviruses, paramyxoviruses, flaviviruses, and caliciviruses. For example, a viral disease can be caused by a virus selected from respiratory syncytial virus (RSV), influenza virus, herpes simplex virus, Epstein-Barr virus, varicella virus, cytomegalovirus, hepatitis A virus, hepatitis B virus, hepatitis C virus, human immunodeficiency virus (HIV), human T-lymphotropic virus, calicivirus, adenovirus, Arena virus, and COVID-19.

Examples of viral diseases include influenza, pneumonia, herpes, hepatitis, hepatitis A, hepatitis B, hepatitis C, chronic fatigue syndrome, sudden acute respiratory syndrome (SARS), gastroenteritis, enteritis, carditis, encephalitis, bronchiolitis, respiratory papillomatosis, meningitis, HIV/AIDS, and mononucleosis, and severe acute respiratory syndrome coronavirus 2 (SARS-CoV-2).

Examples of infectious diseases include Acinetobacter infections, actinomycosis, African sleeping sickness (African trypanosomiasis), AIDS (acquired immunodeficiency syndrome), amoebiasis, anaplasmosis, angiostrongyliasis, anisakiasis, anthrax, Arcanobacterium haemolyticum infection, Argentine hemorrhagic fever, ascariasis, aspergillosis, astrovirus infection, babesiosis, Bacillus cereus infection, bacterial meningitis, bacterial pneumonia, bacterial vaginosis, Bacteroides infection, balantidiasis, bartonellosis, Baylisascaris infection, Bejel, syphilis, yaws, BK virus infection, black piedra, blastocystosis, blastomycosis, Bolivian hemorrhagic fever, botulism (and Infant botulism), Brazilian hemorrhagic fever, brucellosis, bubonic plague, Burkholderia infection, buruli ulcer, calicivirus infection (Norovirus and Sapovirus), campylobacteriosis, candidiasis (Moniliasis; Thrush), capillariasis, carrion's disease, cat-scratch disease, cellulitis, Chagas disease (American trypanosomiasis), chancroid, chickenpox, chikungunya, chlamydia, Chlamydophila pneumoniae infection (Taiwan acute respiratory agent or TWAR), cholera, chromoblastomycosis, Chytridiomycosis, clonorchiasis, Clostridium difficile colitis, coccidioidomycosis, Colorado tick fever (CTF), common cold (acute viral rhinopharyngitis; Acute coryza, Coronavirus disease 2019 (COVID-19), Creutzfeldt-Jakob disease (CJD), Crimean-Congo hemorrhagic fever (CCHF), cryptococcosis, cryptosporidiosis, cutaneous larva migrans (CLM), cyclosporiasis, cysticercosis, cytomegalovirus infection, Dengue fever, desmodesmus infection, dientamoebiasis, diphtheria, diphyllobothriasis, dracunculiasis, Ebola hemorrhagic fever, echinococcosis, Ehrlichiosis, enterobiasis (pinworm infection), Enterococcus infection, enterovirus infection, epidemic typhus, Epstein-Barr virus infectious mononucleosis (Mono), erythema infectiosum (Fifth disease), fxanthem subitum (Sixth disease), fasciolosis, fasciolopsiasis, fatal familial insomnia (FFI), filariasis, food poisoning by Clostridium perfringens, free-living amebic infection, Fusobacterium infection, gas gangrene (Clostridial myonecrosis), geotrichosis, Gerstmann-Straussler-Scheinker syndrome (GSS), giardiasis, glanders, gnathostomiasis, gonorrhea, granuloma inguinale (Donovanosis), Group A streptococcal infection, Group B streptococcal infection, Haemophilus influenzae infection, hand, foot and mouth disease (HFMD), Hantavirus Pulmonary Syndrome (HPS), Heartland virus disease, Helicobacter pylori infection, hemolytic-uremic syndrome (HUS), hemorrhagic fever with renal syndrome (HFRS), Hendra virus infection, Hepatitis A, Hepatitis B, Hepatitis C, Hepatitis D, Hepatitis E, Herpes simplex, histoplasmosis, hookworm infection, human bocavirus infection, human ewingii ehrlichiosis, human granulocytic anaplasmosis (HGA), human metapneumovirus infection, human monocytic ehrlichiosis, human papillomavirus (HPV) infection, human parainfluenza virus infection, hymenolepiasis, influenza (flu), isosporiasis, Kawasaki disease, keratitis, Kingella kingae infection, Kuru, Lassa fever, Legionellosis (Legionnaires' disease), leishmaniasis, leprosy, leptospirosis, listeriosis, Lyme disease (Lyme borreliosis), lymphatic filariasis (elephantiasis), lymphocytic choriomeningitis, malaria, Marburg hemorrhagic fever (MHF), measles, melioidosis (Whitmore's disease), meningitis, meningococcal disease, metagonimiasis, microsporidiosis, Middle East respiratory syndrome (MERS), molluscum contagiosum (MC), monkeypox, mumps, murine typhus (Endemic typhus), mycetoma, Mycoplasma genitalium infection, mycoplasma pneumonia, myiasis, neonatal conjunctivitis (Ophthalmia neonatorum), Nipah virus infection, nocardiosis, Norovirus (children and babies), onchocerciasis (River blindness), opisthorchiasis, paracoccidioidomycosis (South American blastomycosis), paragonimiasis, pasteurellosis, pediculosis capitis (Head lice), pediculosis corporis (Body lice), pediculosis pubis (pubic lice, crab lice), pelvic inflammatory disease (PID), pertussis (whooping cough), plague, pneumococcal infection, pneumocystis pneumonia (PCP), pneumonia, poliomyelitis, Pontiac fever, Prevotella infection, primary amoebic meningoencephalitis (PAM), progressive multifocal leukoencephalopathy, psittacosis, Q fever, rabies, relapsing fever, respiratory syncytial virus infection, rhinosporidiosis, rhinovirus infection, rickettsial infection, rickettsialpox, Rift Valley fever (RVF), Rocky Mountain spotted fever (RMSF), rotavirus infection, rubella, salmonellosis, SARS (severe acute respiratory syndrome), scabies, scarlet fever, schistosomiasis, sepsis, shigellosis (bacillary dysentery), shingles (Herpes zoster), smallpox (variola), sporotrichosis, staphylococcal food poisoning, staphylococcal infection, strongyloidiasis, subacute sclerosing panencephalitis, taeniasis, tetanus (lockjaw), tinea barbae (barber's itch), tinea capitis (ringworm of the scalp), tinea corporis (ringworm of the body), tinea cruris (Jock itch), tinea manum (ringworm of the hand), tinea nigra, tinea pedis (athlete's foot), tinea unguium (onychomycosis), tinea versicolor (Pityriasis versicolor), toxocariasis (ocular larva migrans (OLM), toxocariasis (visceral larva migrans (VLM), toxoplasmosis, trachoma, trichinosis, trichomoniasis, trichuriasis (whipworm infection), tuberculosis, tularemia, typhoid fever, typhus fever, Ureaplasma urealyticum infection, valley fever, Venezuelan equine encephalitis, Venezuelan hemorrhagic fever, vibrio parahaemolyticus enteritis, vibrio vulnificus infection, viral pneumonia, West Nile fever, white piedra (tinea blanca), yellow fever, Yersinia pseudotuberculosis infection, yersiniosis, zeaspora, Zika fever, and zygomycosis.

An enriched immune cell population provided by the present disclosure can be used, either alone or in combination, to treat diseases including acute myeloid leukemia, B-cell lymphoma, chronic myelogenous leukemia, depression, gingival recession, hepatitis C, HIV infections, human papillomavirus, idiopathic CD4 lymphopenia, immunodeficiency secondary to organ transplantation, lipodystrophy, Kaposi sarcoma lymphoma, lymphopenia, mantle cell lymphoma, multiple sclerosis, myelodysplastic syndrome, non-Hodgkin lymphoma, recurrent adult diffuse large cell lymphoma, recurrent follicular lymphoma, rheumatoid arthritis, sepsis, and Type 2 diabetes.

An enriched immune cell population provided by the present disclosure can be used to treat cancers such as metastatic breast cancer, breast cancer, colon cancer, bladder cancer, metastatic prostate cancer, stage IV prostate cancer, castration-resistant prostate carcinoma, neuroblastoma, melanoma, kidney cancer, myeloproliferative neoplasm, sarcoma, and neurodermal tumors.

An enriched immune cell population provided by the present disclosure can be used in combination with temozolomide to treat glioblastoma, with atezolizumab to treat skin cancers such as MCC, C5CC and melanoma, with pembrolizumab to treat triple negative breast cancer, and in combination with CAR-T therapy to treat pediatric acute lymphoblastic leukemia.

Pharmaceutical compositions comprising an enriched immune cell population can be administered concurrently with the administration of another therapeutic agent, which may be part of the same pharmaceutical composition as, or in a different pharmaceutical composition than that comprising an enriched immune cell population. An enriched immune cell population may be administered prior or subsequent to administration of another therapeutic agent. In combination therapy, the combination therapy may comprise alternating between administering an enriched immune cell population and a composition comprising another therapeutic agent, e.g., to minimize adverse drug effects associated with a particular drug. When an enriched immune cell population is administered concurrently with another therapeutic agent that potentially may produce an adverse drug effect including, for example, toxicity, the other therapeutic agent may be administered at a dose that falls below the threshold at which the adverse drug reaction is elicited.

Pharmaceutical compositions comprising an enriched immune cell population may be administered with one or more substances to enhance, modulate and/or control release, bioavailability, therapeutic efficacy, therapeutic potency, stability, and the like of a compound of an enriched immune cell population. For example, to enhance the therapeutic efficacy of an enriched immune cell population, metabolite thereof, or a pharmaceutical composition of any of the foregoing may be co-administered with one or more active agents to increase the absorption or diffusion of the enriched immune cell population from the gastrointestinal tract to the systemic circulation, or to inhibit degradation of the enriched immune cell population in the blood of a subject. A pharmaceutical composition comprising an enriched immune cell population may be co-administered with an active agent having pharmacological effects that enhance the therapeutic efficacy of the enriched immune cell population.

An enriched immune cell population, or a pharmaceutical composition comprising any of the foregoing may be administered in conjunction with an agent known or believed to be effective in treating an inflammatory disease or an autoimmune disease in a patient.

An enriched immune cell population, or a pharmaceutical composition comprising any of the foregoing may be administered in conjunction with an agent known or believed to interfere with proliferation. An enriched immune cell population, or a pharmaceutical composition comprising any of the foregoing may be administered in conjunction with an agent known or believed to interfere with mitosis. An enriched immune cell population, or a pharmaceutical composition comprising any of the foregoing may be administered in conjunction with an agent known or believed to interfere with DNA replication. An enriched immune cell population, or a pharmaceutical composition comprising an enriched immune cell population may be administered in conjunction with an agent known or believed to interfere with DNA repair.

An enriched immune cell population or a pharmaceutical composition thereof may be administered to a patient together with another compound for treating an inflammatory disease or an autoimmune disease in the patient. The at least one other therapeutic agent may be a different enriched immune cell population provided by the present disclosure. An enriched immune cell population and the at least one other therapeutic agent may act additively or synergistically. The at least one additional therapeutic agent may be included in the same pharmaceutical composition or vehicle comprising the enriched immune cell population or may be in a separate pharmaceutical composition or vehicle.

Accordingly, methods provided by the present disclosure further include, in addition to administering an enriched immune cell population, administering one or more therapeutic agents effective for treating an inflammatory disease or an autoimmune disease or a different disease, disorder or condition than an inflammatory disease or an autoimmune disease. Methods provided by the present disclosure include administering an enriched immune cell population and one or more other therapeutic agents provided that the combined administration does not inhibit the therapeutic efficacy of the enriched immune cell population and/or does not produce adverse combination effects.

Enriched immune cell populations provided by the present disclosure can be administered either alone or as a pharmaceutical composition in combination with diluents and/or with other therapeutic agents, including cytokines such as IL-2, IL-7, and IL-15, immune cell populations, and/or compounds comprising IL-7Rαγc agonist peptides, IL-2Rβγc agonist peptides, or IL-15Rβγc agonist peptides.

Methods for treatment provided by the present disclosure comprise administering to a patient in need thereof an effective amount of at least one CAR, or immune cell comprising a CAR, wherein the CAR or immune cell is produced using methods or providing an enriched immune cell population provided by the present disclosure.

In certain methods of treatment provided by the present disclosure an immune cell-mediated immune response can be induced in a patient by administering an effective amount of an engineered immune cell population to the patient. The immune cell-mediated immune response can be directed against a target cell or population of cells. An engineered immune cell can comprise a chimeric antigen receptor (CAR). A target cell can be a tumor cell. A method for treating or preventing a malignancy can comprise administering to a patient an effective amount of at least one isolated antigen binding domain. A method for treating or preventing a malignancy can comprise administering to a patient an effective amount an enriched immune cell population, wherein the enriched immune cell population comprises at least one chimeric antigen receptor, a T cell receptor, and/or isolated antigen binding domain. CAR-containing immune cells can be used to treat malignancies involving the aberrant expression of biomarkers.

CAR-containing immune cells can be used to treat, for example, small cell lung cancer, melanoma, low grade gliomas, glioblastoma, medullary thyroid cancer, carcinoids, dispersed neuroendocrine tumors in the pancreas, bladder and prostate, testicular cancer, and lung adenocarcinomas with neuroendocrine features.

Methods of treatment provided by the present disclosure include methods for reducing the size of a tumor in a patient comprising administering to a patient an enriched engineered immune cell to the patient, wherein the enriched engineered immune cell comprises a chimeric antigen receptor comprising an antigen binding domain and binds to an antigen on the tumor.

An engineered immune cell can be delivered to a tumor bed.

An engineered immune cell provided by the present disclosure can be an autologous immune cell such as an autologous T cell.

An engineered immune cell provided by the present disclosure can be an allogeneic immune cell such as an allogeneic T cell.

An engineered immune cell provided by the present disclosure can be a heterologous immune cell such as a heterologous T cell.

An engineered immune cell provided by the present disclosure can be transfected or transduced in vivo.

An engineered immune cell can be transfected or transduced ex vivo.

### ASPECTS

The invention is further defined by one or more of the following aspects.

Aspect 1. A culture medium for expanding a target immune cell population comprising a first stimulant of proliferation for the target immune cell population proliferation, wherein the first stimulant comprises an IL-7Rαγc agonist peptide.

Aspect 2. The culture medium of aspect 2, wherein the IL-7Rαγc agonist peptide comprises:
(a) an IL-7Rα ligand, wherein the IL-7Rα ligand comprises:
   an amino acid sequence of any one of SEQ ID NO: 43-46, 100-117, 118-193, 197-245 and 53-60;
   a truncated amino acid sequence of any one of SEQ ID NO: 43-46, 100-117, 118-193, 197-245 and 53-60;
   an amino acid sequence of any one of SEQ ID NO: 43-46, 100-117, 118-193, 197-245 and 53-606 having from 1 to 5 amino acid substitutions;
   an amino acid sequence of any one of SEQ ID NO: 43-46, 100-117, 118-193, 197-245 and 53-60 having from 1 to 5 flanking glycines on the N-terminus, the C-terminus, or both the N-terminus and the C-terminus;
   an amino acid sequence having greater than 60% sequence similarity to an amino acid sequence of any one of SEQ ID NO: 43-46, 100-117, 118-193, 197-245 and 53-60; or
   a combination of any of the foregoing; and
(b) an Rγc ligand, wherein the Rγc ligand comprises:
   an amino acid sequence of any one of SEQ ID NO: 246-327 and 47-52;
   a truncated amino acid sequence of any one of SEQ ID NO: 246-327 and 47-52;
   an amino acid sequence of any one of SEQ ID NO: 246-327 and 47-52 having from 1 to 5 amino acid substitutions;
   an amino acid sequence of any one of SEQ ID NO: 246-327 and 47-52 having from 1 to 5 flanking glycines on the N-terminus, the C-terminus, or both the N-terminus and the C-terminus;
   an amino acid sequence having greater than 60% sequence similarity to an amino acid sequence of any one of SEQ ID NO: 246-327 and 47-52; or
   a combination of any of the foregoing.

Aspect 3. The culture medium of aspect 2, wherein the IL-7Rαγc agonist peptide comprises:
an IL-7Rα ligand comprising an amino acid sequence of any one of SEQ ID NO: 100-117, 118-193, 197-245 and 53-60; and
an Rγc ligand comprising an amino acid sequence of any one of SEQ ID NO: 246-327 and 47-52.

Aspect 4. The culture medium of aspect 3, wherein the IL-7Rα ligand comprises an amino acid sequence of any one of SEQ ID NO: 114 and 152.

Aspect 5. The culture medium of any one of aspects 2 to 4, wherein the Rγc ligand comprises an amino acid sequence of any one of SEQ ID NO: 261 and 325-327.

Aspect 6. The culture medium of any one of aspects 2 to 5, wherein the IL-7Rα ligand is bound to the Rγc ligand through a ligand linker.

Aspect 7. The culture medium of aspect 6, wherein the ligand linker comprises a peptidyl ligand linker.

Aspect 8. The culture medium of aspect 1, wherein the IL-7Rαγc agonist peptide comprises:
an amino acid sequence of any one of SEQ ID NO: 443-454 and 368-442;
   a truncated amino acid sequence of any one of SEQ ID NO: 443-454 and 368-442;
   an amino acid sequence of any one of SEQ ID NO: 443-454 and 368-442 having from 1 to 5 amino acid substitutions;
   an amino acid sequence of any one of SEQ ID NO: 443-454 and 368-442 having from 1 to 5 flanking glycines on the N-terminus, the C-terminus, or both the N-terminus and the C-terminus;
   an amino acid sequence having greater than 60% sequence similarity to an amino acid sequence of any one of SEQ ID NO: 443-454 and 368-442; or
   a combination of any of the foregoing.

Aspect 9. The culture medium of aspect 1, wherein the IL-7Rαγc agonist peptide comprises an amino acid sequence of any one of SEQ ID NO: 394-404.

Aspect 10. The culture medium of any one of aspects 1 to 9, wherein the IL-7Rαγc agonist peptide comprises an immobilized IL-7Rαγc agonist peptide.

Aspect 11. The culture medium any one of aspects 1 to 10, wherein the culture medium comprises a second stimulant of proliferation for the target immune cell population.

Aspect 12. The culture medium of aspect 11, wherein the second stimulant comprises an IL-2Rβγc agonist peptide.

Aspect 13. The culture medium of aspect 12, wherein the IL-2Rβγc agonist peptide comprises an amino acid sequence of any one of SEQ ID NO: 359-366, 61-76, and 77-99.

Aspect 14. The culture medium of aspect 13, wherein the IL-2Rβγc agonist peptide comprises an amino acid sequence of any one of SEQ ID NO: 91-99 and 359.

Aspect 15. The culture medium of aspect 11, wherein the second stimulant comprises an IL-15Rβγc agonist peptide.

Aspect 16. The culture medium of any one of aspects 1 to 15, wherein the culture medium comprises an antibody directed to the target immune cell population.

Aspect 17. The culture medium of any one of aspects 1 to 16, wherein the target immune cell population comprises a target T cell population.

Aspect 18. The culture medium of aspect 17, wherein the target T cell population comprises CD4+ T cells and CD8+ T cells.

Aspect 19. The culture medium of any one of aspects 17 to 18, wherein the culture medium comprises an antibody directed to the target T cell population.

Aspect 20. The culture medium of aspect 19, wherein the antibody comprises an anti-CD3 antibody, an anti-CD28 antibody, or a combination thereof.

Aspect 21. A method of expanding a target immune cell population of an initial immune cell population, comprising incubating an initial immune cell population in the culture medium of any one of aspects 1 to 20 to provide an expanded target immune cell population.

Aspect 22. The method of aspect 21, wherein the target immune cell population comprises a target T cell population

Aspect 23. The method of aspect 22, wherein the expanded target T cell population comprises Tem cells, Tscm cells, or both Tem cells and Tscm cells.

Aspect 24. The method of aspect 22, wherein the expanded target T cell population comprises CD4+ T cells and CD8+ T cells.

Aspect 25. The method of any one of aspects 21 to 24, wherein the number of cells per milliliter of blood of the target immune cell population expands by greater than 10 times, wherein percent is based on the number of cells per milliliter of blood of the target immune cell population prior to expansion.

Aspect 26. The method of any one of aspects 21 to 25, wherein the expanded immune cell population is greater than 100-times enriched in the expanded target immune cells as measured over a period from 10 days to 30 days compared to the initial population of target immune cells.

Aspect 27. The method of any one of aspects 21 to 26, wherein the target immune cell population comprises a population of engineered immune cells.

Aspect 28. The method of aspect 27, wherein the population of engineered immune cells express one or more chimeric antigen receptors.

Aspect 29. The method of any one of aspects 21 to 28, wherein the initial immune cell population comprises allogeneic immune cells.

Aspect 30. The method of any one of aspects 21 to 29, wherein the initial immune cell population comprises autologous immune cells.

Aspect 31. The method of any one of aspects 21 to 30, wherein incubating comprises incubating in the presence of an IL-2Rβγc agonist peptide.

Aspect 32. The method of aspect 31, wherein incubating in the presence of an IL-2Rβγc agonist peptide comprises incubating together with the IL-7Rαγc agonist peptide.

Aspect 33. The method of aspect 31, wherein incubating in the presence of an IL-2Rβγc agonist peptide comprises incubating in a culture medium that does not comprise an IL-7Rαγc agonist peptide.

Aspect 34. A method of manufacturing an immune cell population, comprising
activating a target immune cell population of a population of primary cells to provide an activated target immune cell population; and
expanding the activated target immune cell population, wherein expanding comprises incubating the activated target immune cell population in the presence of an IL-7Rαγc agonist peptide to provide an expanded target immune cell population.

Aspect 35. The method of aspect 34, wherein the primary cells comprise human primary cells.

Aspect 36. The method of aspect 35, wherein the human primary cells comprise mononuclear cells.

Aspect 37. The method of aspect 36, wherein the mononuclear cells comprise peripheral blood mononuclear cells (PBMCs) from a patient.

Aspect 38. The method of any one of aspects 36 to 37, wherein the mononuclear cells comprise peripheral blood mononuclear cells (PBMCs) from a donor.

Aspect 39. The method of any one of aspects 34 to 38, comprising, before activating the target immune cell population, enriching the population of primary cells with the target immune cell population.

Aspect 40. The method of any one of aspects 34 to 39, wherein, after activating the target immune cell population, engineering the activated target immune cell population to express a cell surface receptor.

Aspect 41. The method of aspect 40, wherein engineering comprises transducing the activated target immune cell population with a viral vector to provide a transduced activated target immune cell population.

Aspect 42. The method of aspect 41, wherein the viral vector comprises a viral vector expressing a T cell receptor (TCR) or a chimeric antigen receptor (CAR).

Aspect 43. The method of aspect 42, wherein the TCR or CAR recognizes a specific antigenic moiety on a surface of a target cell.

Aspect 44. The method of aspect 43, wherein the antigenic moiety is MHC class I and/or MHC class II dependent.

Aspect 45. The method of any one of aspects 34 to 44, wherein activating comprises incubating the target immune cell population in the presence of an antibody to the target immune cell population.

Aspect 46. The method of aspect 45, wherein the antibody is immobilized on a substrate.

Aspect 47. The method of any one of aspects 34 to 46, wherein the IL-7Rαγc agonist peptide is immobilized on a substrate.

Aspect 48. The method of any one of aspects 34 to 47, wherein expanding comprises incubating the activated target immune cell population in the presence of an IL-2Rβγc agonist peptide.

Aspect 49. The method of aspect 48, wherein incubating the activated target immune cell population in the presence of an IL-2Rβγc agonist peptide comprises incubating the activated target immune cell population together with the IL-7Rαγc agonist peptide.

Aspect 50. The method of aspect 48, wherein incubating the activated target immune cell population in the presence of an IL-2Rβγc agonist peptide comprises incubating the activated target immune cell population in a culture medium that does not comprise an IL-7Rαγc agonist peptide.

Aspect 51. The method of any one of aspects 34 to 50, wherein the target immune cell population comprises CD4+ T cells and CD8+ T cells.

Aspect 52. The method of aspect 51, comprising, before activating the CD4+ T cells and CD8+ T cells, enriching the population of primary cells for CD4+ T cells and CD8+ T cells.

Aspect 53. The method of aspect 52, wherein enriching comprises removing CD57+ T cells from the population of primary cells to provide a depleted population of CD57+ cells.

Aspect 54. The method of aspect 51, wherein activating comprises incubating the CD4+ T cells and CD8+ T cells in the presence of anti-CD3 monoclonal antibody and an anti-CD28 monoclonal antibody.

Aspect 55. The method of any one of aspects 34 to 54, comprising, before activating, enriching the population of primary cells with the target immune cell population.

Aspect 56. An enriched population of immune cells prepared using the culture medium of any one of aspects 1 to 20.

Aspect 57. An enriched population of immune cells prepared using the method of any one of aspects 21 to 33.

Aspect 58. An enriched population of immune cells prepared using the method of any one of aspects 34 to 55.

Aspect 59. The enriched population of immune cells of any one of aspects 56 to 58, wherein the enriched population of immune cells comprises T cells.

Aspect 60. The enriched population of immune cells of aspect 59, wherein the enriched population of T cells comprises CD4+ T cells and CD8+ T cells.

Aspect 61. A pharmaceutical composition comprising the enriched population of immune cells of any one of aspects 56 to 60.

Aspect 62. A method of treating a disease in a patient comprising administering to a patient in need of such treatment a therapeutically effective amount of the pharmaceutical composition of aspect 61.

Aspect 63. The method of aspect 62, wherein the disease is cancer, a viral disease, or an autoimmune disease.

Aspect 64. The method of aspect 62, wherein the cancer is selected from primary adult and childhood brain and CNS cancers including glioblastoma (GBM) and astrocytoma, skin cancers including melanoma, lung cancers including small cell lung cancers, non-small cell lung cancers (NSCLC), and large cell lung cancers, breast cancers including triple-negative breast cancer (TNBC), blood cancers including myelodysplastic syndrome (MDS), multiple myeloma (MM), and acute myeloid leukemia (AML), prostate cancer including castrate-resistant prostate cancer (CRPC), liver cancers including hepatocellular carcinoma (HCC), esophageal and gastric cancers, and any systemic and central metastases of any of the foregoing.

Aspect 65. The method of aspect 62, wherein the viral disease is selected from influenza, pneumonia, herpes, hepatitis, hepatitis A, hepatitis B, hepatitis C, chronic fatigue syndrome, sudden acute respiratory syndrome (SARS), gastroenteritis, enteritis, carditis, encephalitis, bronchiolitis, respiratory papillomatosis, meningitis, HIV/AIDS, and mononucleosis, and severe acute respiratory syndrome coronavirus 2 (SARS-CoV-2).

Aspect 66. The method of aspect 62, wherein the autoimmune disease is selected from lupus, graft-versus-host disease, hepatitis C-induced vasculitis, Type I diabetes, multiple sclerosis, spontaneous loss of pregnancy, atopic diseases, and inflammatory bowel diseases.

Aspect 67. An article of manufacture comprising the culture medium of any one of aspects 1 to 20.

Aspect 68. An article of manufacture comprising the enriched population of immune cells of any one of aspects 56 to 60.

Aspect 69. An article of manufacture comprising the pharmaceutical composition of aspect 61.

Aspect 70. An immobilized IL-7Rαγc agonist peptide, comprising an IL-7Rαγc agonist peptide bound to a substrate.

Aspect 71. The immobilized IL-7Rαγc agonist peptide of aspect 70, wherein the IL-7Rαγc agonist peptide comprises:
(a) an IL-7Rα ligand, wherein the IL-7Rα ligand comprises:
   an amino acid sequence of any one of SEQ ID NO: 43-46, 100-117, 118-193, 197-245 and 53-60;
   a truncated amino acid sequence of any one of SEQ ID NO: 43-46, 100-117, 118-193, 197-245 and 53-60;
   an amino acid sequence of any one of SEQ ID NO: 43-46, 100-117, 118-193, 197-245 and 53-60 having from 1 to 5 amino acid substitutions;
   an amino acid sequence of any one of SEQ ID NO: 43-46, 100-117, 118-193, 197-245 and 53-60 having from 1 to 5 flanking glycines on the N-terminus, the C-terminus, or both the N-terminus and the C-terminus;
   an amino acid sequence having greater than 60% sequence similarity to an amino acid sequence of any one of SEQ ID NO: 43-46, 100-117, 118-193, 197-245 and 53-60; or
   a combination of any of the foregoing; and
(b) an Rγc ligand, wherein the Rγc ligand comprises:
   an amino acid sequence of any one of SEQ ID NO: 246-327 and 47-52;
   a truncated amino acid sequence of any one of SEQ ID NO: 246-327 and 47-52;
   an amino acid sequence of any one of SEQ ID NO: 246-327 and 47-52 having from 1 to 5 amino acid substitutions;
   an amino acid sequence of any one of SEQ ID NO: 246-327 and 47-52 having from 1 to 5 flanking glycines on the N-terminus, the C-terminus, or both the N-terminus and the C-terminus;
   an amino acid sequence having greater than 60% sequence similarity to an amino acid sequence of any one of SEQ ID NO: 246-327 and 47-52; or
   a combination of any of the foregoing.

Aspect 72. The immobilized IL-7Rαγc agonist peptide of aspect 70, wherein the IL-7Rαγc agonist peptide comprises:
an IL-7Rα ligand comprising an amino acid sequence of any one of SEQ ID NO: 100-117, 118-193, 197-245 and 53-60; and an Rγc ligand comprising an amino acid sequence of any one of SEQ ID NO: 246-327 and 47-52.

Aspect 73. The immobilized IL-7Rαγc agonist peptide of any one of aspects 71 to 72, wherein the IL-7Rα ligand comprises an amino acid sequence of any one of SEQ ID NO: 114, 152-154, 368, 369, 374, 383, 394-397. 405, 420, and 443.

Aspect 74. The immobilized IL-7Rαγc agonist peptide of any one of aspects 72 to 72 wherein the Rγc ligand comprises an amino acid sequence of any one of SEQ ID NO: 261.

Aspect 75. The immobilized IL-7Rαγc agonist peptide of any one of aspects 71 to 73, wherein the IL-7Rα ligand is bound to the Rγc ligand through a ligand linker.

Aspect 76. The immobilized IL-7Rαγc agonist peptide of aspect 75, wherein the ligand linker comprises a peptidyl ligand linker.

Aspect 77. The immobilized IL-7Rαγc agonist peptide of aspect 71, wherein the IL-7Rαγc agonist peptide comprises:
an amino acid sequence of any one of SEQ ID NO: 443-454 and 368-442;
   a truncated amino acid sequence of any one of SEQ ID NO: 443-454 and 368-442;
   an amino acid sequence of any one of SEQ ID NO: 443-454 and 368-442 having from 1 to 5 amino acid substitutions;
   an amino acid sequence of any one of SEQ ID NO: 443-454 and 368-442 having from 1 to 5 flanking glycines on the N-terminus, the C-terminus, or both the N-terminus and the C-terminus;
   an amino acid sequence having greater than 60% sequence similarity to an amino acid sequence of any one of SEQ ID NO: 443-454 and 368-442; or
   a combination of any of the foregoing.

Aspect 78. The immobilized IL-7Rαγc agonist peptide of aspect 71, wherein the IL-7Rαγc agonist peptide comprises an amino acid sequence of any one of SEQ ID NO: 394-404.

Aspect 79. The immobilized IL-7Rαγc agonist peptide of any one of aspects 71 to 78, wherein the substrate comprises a bead.

Aspect 80. The immobilized IL-7Rαγc agonist peptide of any one of aspects 71 to 78, wherein the substrate comprises a bead, a matrix, or a plate.

Aspect 81. The immobilized IL-7Rαγc agonist peptide of any one of aspects 71 to 80, wherein the IL-7Rαγc agonist peptide is bound to the substrate through a substrate linker.

### EXAMPLES

The following examples describe in detail culture media comprising an IL-7Rαγ agonist peptide, methods of expanding a targeted immune cell population, and methods of manufacturing a targeted immune cell population.

It will be apparent to those skilled in the art that many modifications, both to materials and methods, may be practiced without departing from the scope of the invention.

### Example 1

### pSTAT5 Flow Cytometry

For resting conditions, frozen PBMCs were cultured 1 day. The cells were stained with a viability dye for 30 minutes at 37° C., followed by incubation with surface marker antibodies for 30 minutes on ice, and then treated with a serially diluted IL-7Rαγc agonist peptide (SEQ ID NO: 395) or a serially diluted IL-2Rβγc agonist peptide (SEQ ID NO: 98) ell medium for 30 minutes. After incubation, the cells were washed, fixed, permeabilized according to the manufacturer's instructions, and stained with pSTAT5 and intracellular antibodies for 50 minutes on ice. The antibody-stained cells were analyzed immediately by flow cytometry using a NovoCyte® Advanteon™ flow cytometer (Agilent), and the data were analyzed using FlowJo™ software (FlowJo LLC). Fluorescence minus one (FMO) controls were used to draw gates. For measuring pSTAT5 in activated PBMCs, the overnight rested cells were incubated with CD3/CD28 Dynabeads™ (ThermoFisher Scientific) at 1 bead to 1 cell ratio for 3 days and then rested for 2 days in the fresh medium without the Dynabeads™ before testing the compounds.

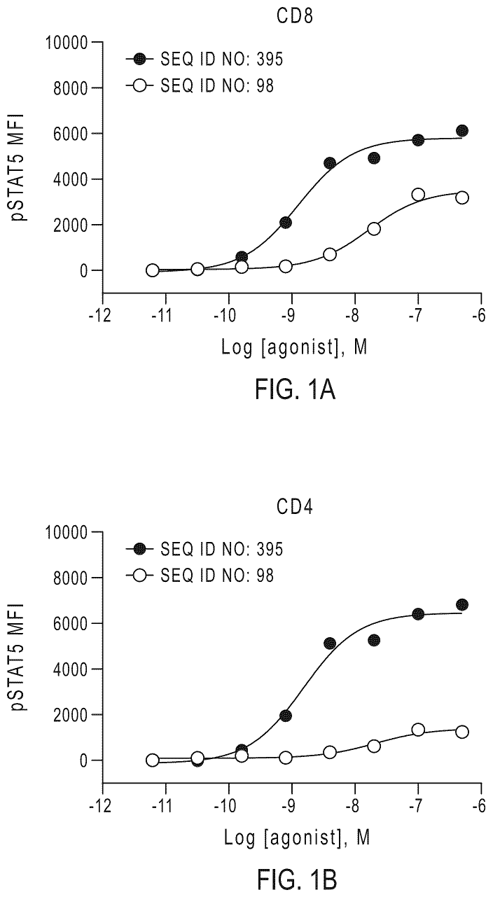

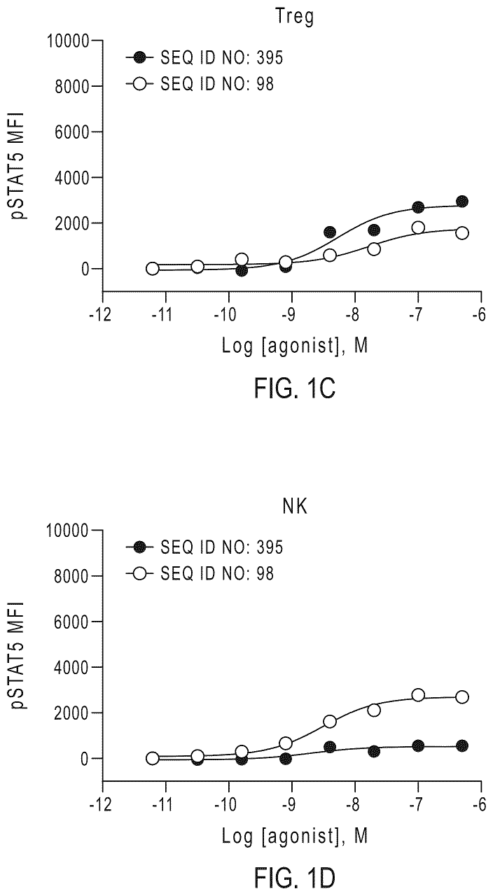

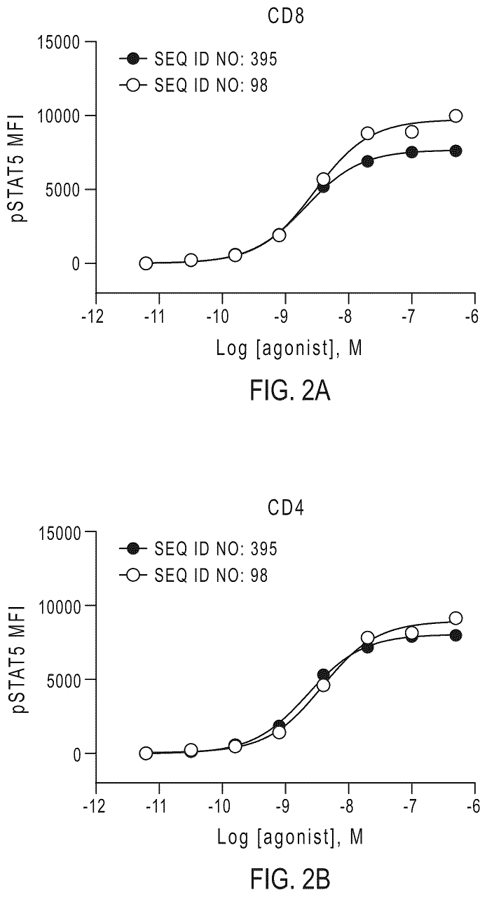

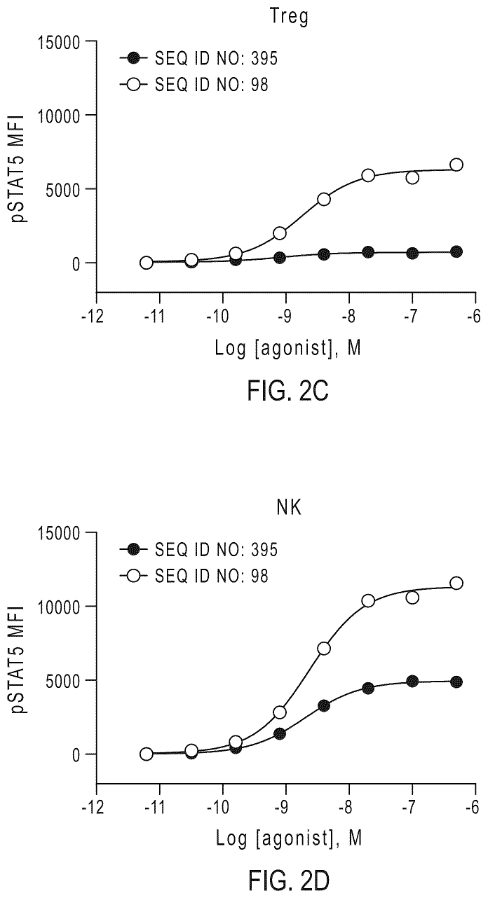

The results for resting PBMCs are shown in FIGS. 1A-ID and for PMBCs activated with anti-CD3 and anti-CD28 monoclonal antibodies are shown in FIGS. 2A-2D.

### Example 2

### Immune Cell Proliferation

Frozen human PBMCs from 5 healthy donors (Stem Cell Technologies, cat no. 70025.2) were thawed and rested overnight at 37° C., 5% CO₂ in CTS™ OpTmizer™ T Cell Expansion SFM (ThermoFisher Scientific No. A1048501). Four million cells were cultured in a plate coated with or without 10 ng/mL of CD3 antibody (Clone SP34-2, Fisher Scientific) and in the presence or absence of 250 nM of an IL-7Rαγc agonist peptide (SEQ ID NO: 395) or an IL-2Rβγc agonist peptide (SEQ ID NO: 99). Fresh media containing the agonist peptides were provided every 2-3 days. Cell aliquots were taken weekly, and cell surface staining was performed using antibodies, followed by fixable viability dye staining and analyzed using a NovoCyte® Advanteon™ flow cytometer (Agilent). The data were analyzed using FlowJo™ software (FloJo LLC). Fluorescent minus one (FMO) controls were used to draw gates.

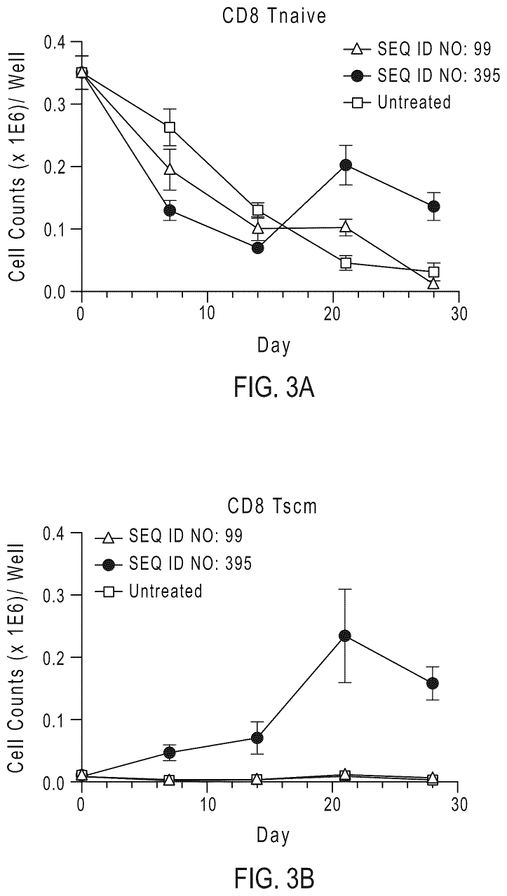

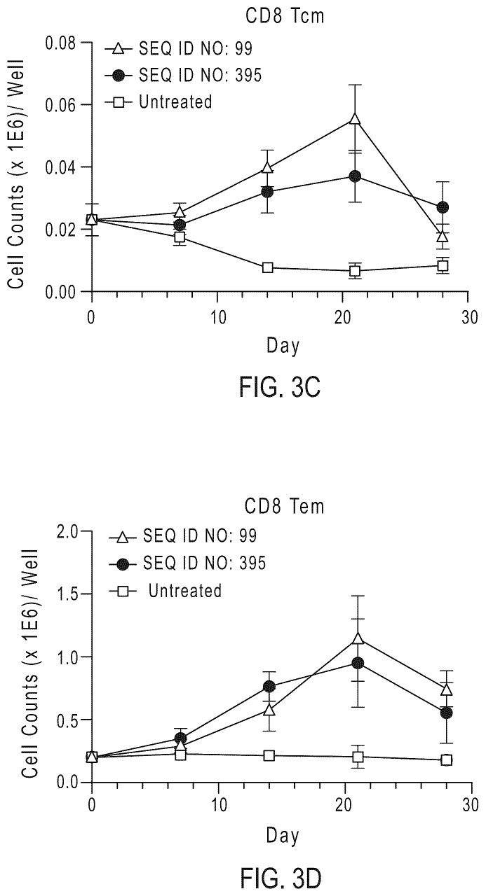

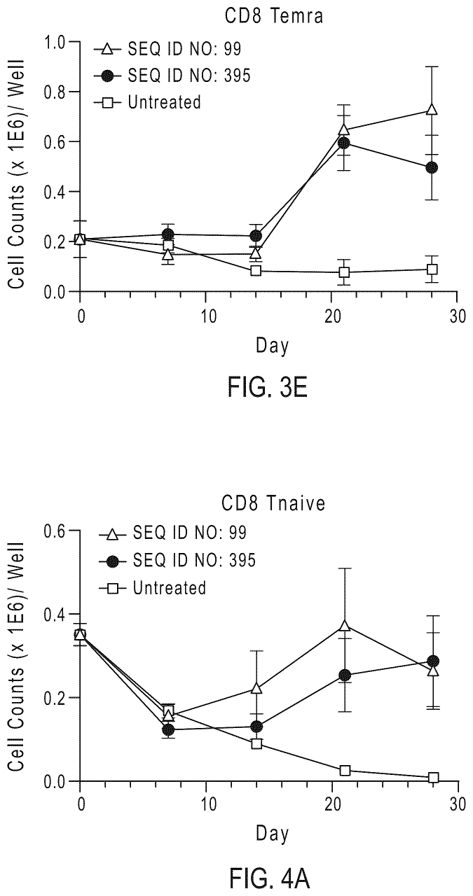

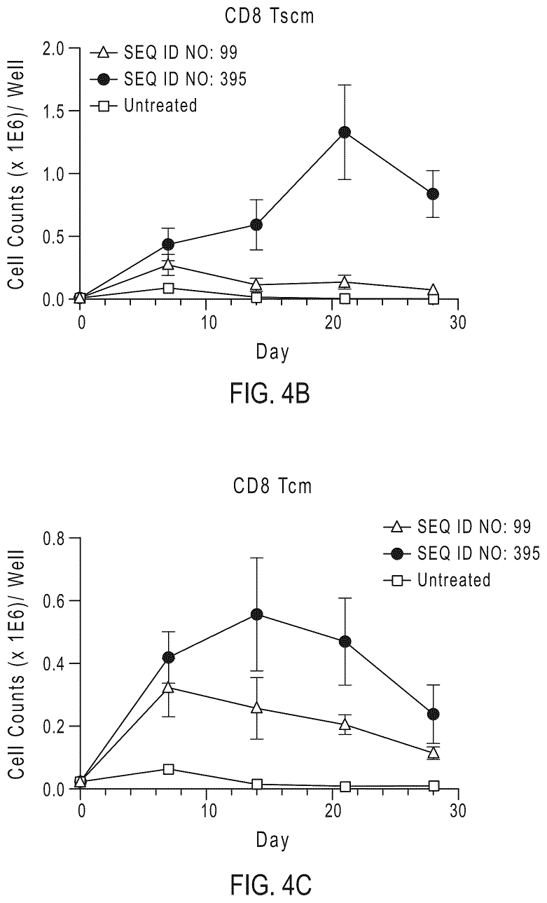

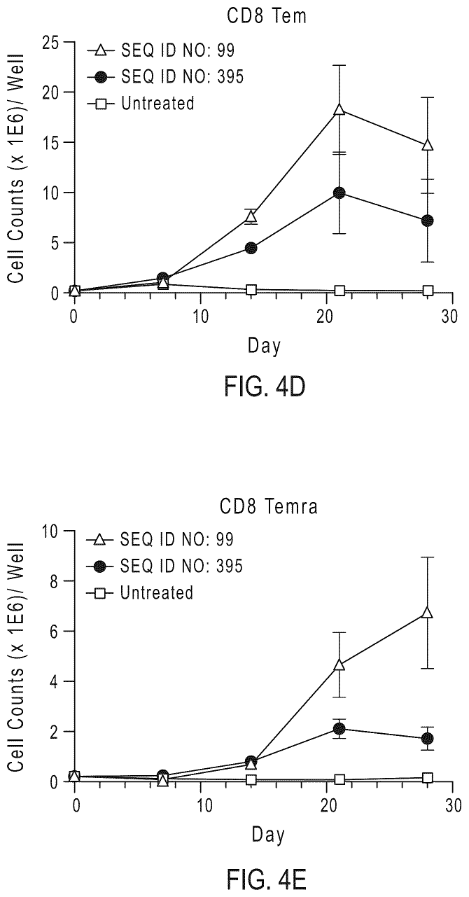

The cell counts for T cell subpopulations during incubation of resting PBMCs are shown in FIGS. 3A-3E and for PMBCs activated with 10 ng/mL anti-CD3 monoclonal antibody are shown in FIGS. 4A-4E.

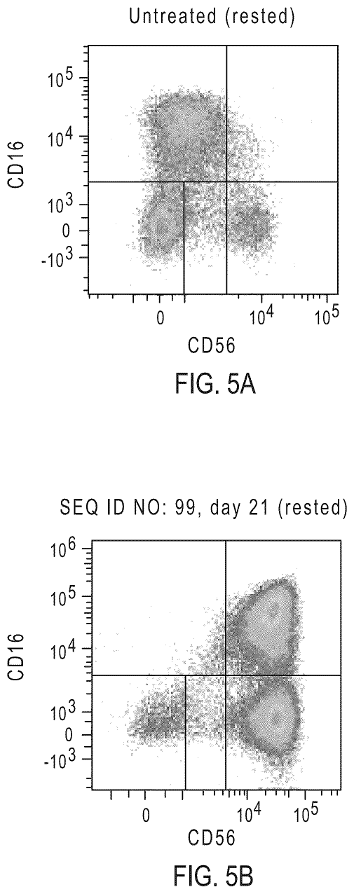

NK cell subpopulation profiles following incubation of resting and activated PBMCs with an IL-2Rβγc agonist peptide (SEQ ID NO: 99) for 21 days are shown in in FIGS. 5A and 5B, respectively.

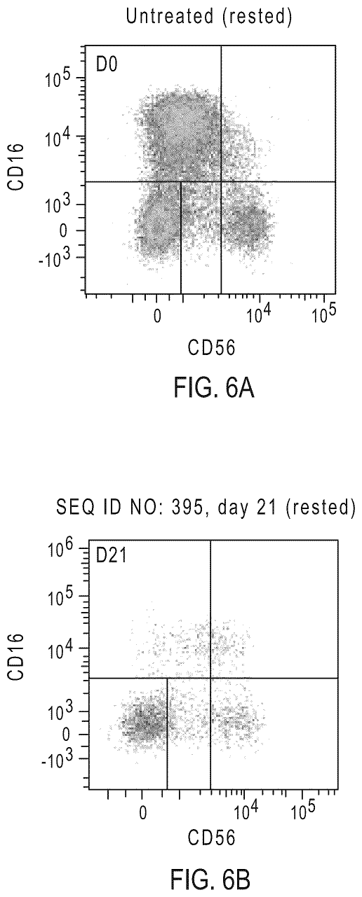

NK cell subpopulation profiles following incubation of resting and activated PBMCs with an IL-7Rαγc agonist peptide (SEQ ID NO: 395) for 21 days are shown in in FIGS. 6A and 6B, respectively.

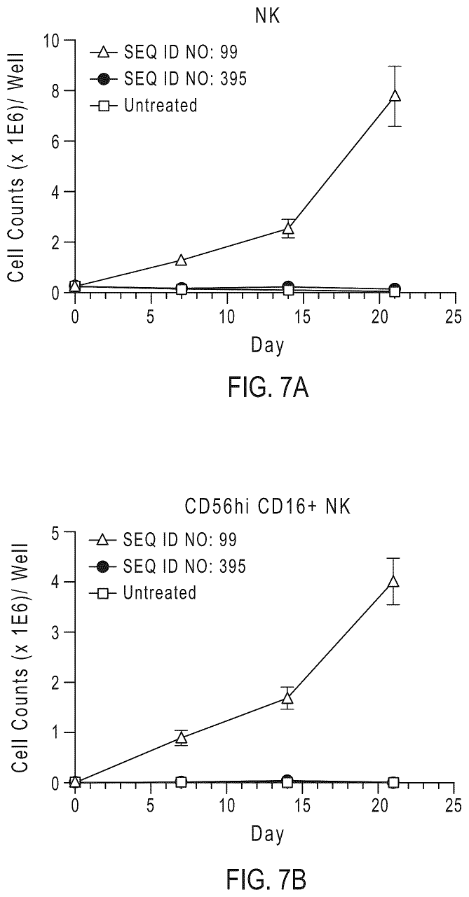

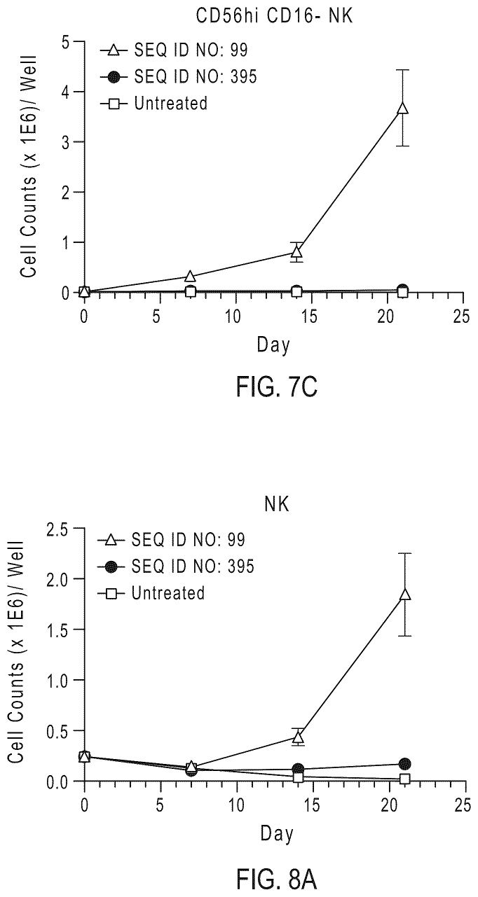

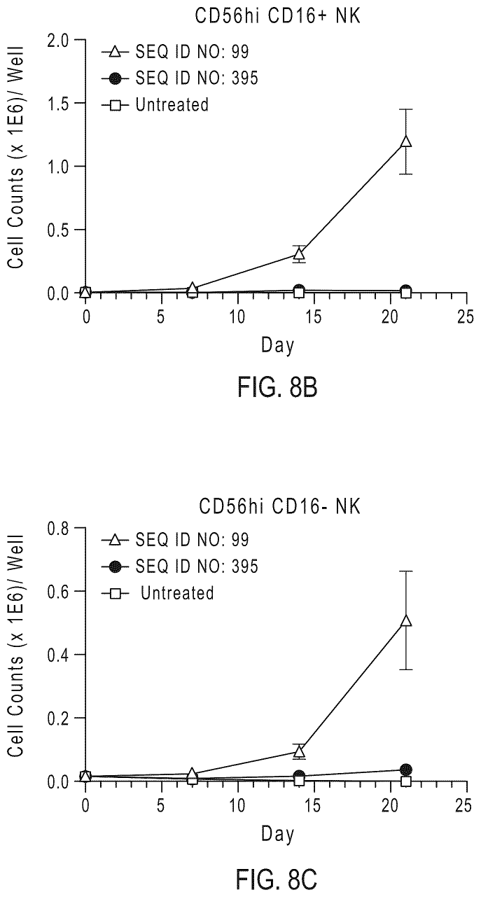

The cell counts for NK cell subpopulations during incubation of resting PBMCs without a agonist peptide, with an IL-2Rβγc agonist peptide (SEQ ID NO: 99), or with an IL-2Rβγc agonist peptide (SEQ ID NO: 99) are shown in FIGS. 7A-7C and for PMBCs activated with 10 ng/mL anti-CD3 monoclonal antibody are shown in FIGS. 8A-8C.

### Example 3

### Cell Counting

Cell cultures were initially plated at 4×10⁶ cells in 2 mL per well. At each assay time point, 50 μL/well of cells were taken from each culture and incubated with 200 μL of the viability dye solution. The Novocyte® Advanteon™ flow cytometer was set up to analyze 50 μL (12.5 μL original culture volume), which allowed the calculation of the absolute live cell counts. Because the culture volumes change over time due to splitting and removal, dilution factors were considered when calculating the total number of cells.

### Example 4

### Proliferation of NK-92 Cells with IL-2 and an IL-2Rγc Agonist Peptide

NK-92 cell proliferation in response to IL-2Rβγc agonist peptide (SEQ ID NO: 359 acylated) or IL-2 was measured using Ki67 staining. The nuclear protein Ki67 was present during all active phases of the cell cycle but was absent in resting cells. NK-92 cells were resuspended in starvation medium and plated at 2×10⁵ cells/well in a 96-well plate. Three-fold serial dilution of the test compound was then added to the cells for 48 h. Following the incubation period, cells were treated with Live/Dead® Fixable Aqua Dye (ThermoFisher No. L34957) for 30 min to stain for viable cells. The cells were then washed with PBS and then fixed and permeabilized for 1 h at 25° C. with Foxp3 Transcription Factor Fix/Perm® buffer (eBioscience No. 00-5523). Cells were washed and then stained with anti-Ki67 PE antibody (ThermoFisher No. 12-5698-82). After a final wash the cells were analyzed by flow cytometry on an LSR II instrument (Becton Dickinson). Data analysis was performed using FlowJo™ software. The median fluorescence intensity of Ki67+ cells was plotted as a function of test compound concentration.

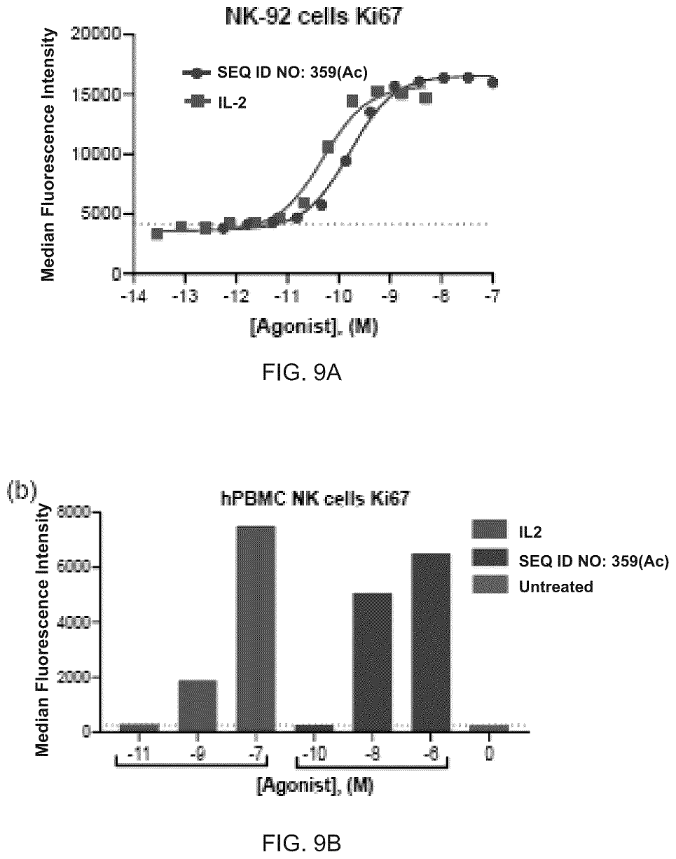

The results are presented in FIGS. 9A and 9B.

### Example 5

### Proliferation in NK Cells from Human PBMCs

Human PBMCs were isolated from a buffy coat by density gradient centrifugation (Lymphoprep®, Stemcell Technologies No. 07811) and cultured overnight in T-cell medium (CTS OpTmizer®, ThermoFisher #A1048501) at 3×10⁶ cells/mL in a T75 flask. The following day, cells were resuspended in fresh medium and plated at 5×10⁵ cells/well in a 96-well cell culture plate. Three-fold serial dilutions of either IL-2 or an IL-2Rβγc ligand (SEQ ID NO: 802 acylated) were added to the cells and incubated for 3 days at 37° C. After the treatment, cells were incubated in viability dye (Live/Dead® Fixable Aqua Cell Stain Kit, ThermoFisher #L349650) for 30 min at 37° C., after which surface antibody staining was then performed in PBS+2% FBS for 30 min on ice. Cells were fixed and permeabilized with Fixation/Permeabilization Buffer (eBioscience Foxp3/Transcription Staining Buffer Set, ThermoFisher #00-5523-00) for 30 min on ice. Intracellular (Ki-67) staining was performed in Permeabilization Buffer for 30 min on ice and the treated cells resuspended in PBS+2% FBS prior to FACS analysis. NK cells were identified as CD56+ and/or CD159a+ cells from CD3− and CD20− (non-T, non-B cell) populations. Antibody conjugates used for cell surface and intracellular staining are shown in Table 1.

TABLE 1
Antibody conjugates used for cell surface and intracellular staining.

| Marker | CD3 | Ki-67 | CD56 | CD20 | CD45RA | CD8 | CD159a | Live/Dead |
|---|---|---|---|---|---|---|---|---|
| Fluor | FITC | PE | PerCP- | PE-Cy7 | APC | BV421 | BV650 | Aqua |
| | | | eFluor710 | | | | | |
| Clone | UCHT1 | SolA15 | CMSSB | 2H7 | HI100 | SK1 | 13411 | — |
| Supplier | Invitrogen | Invitrogen | Invitrogen | BioLegend | BD | BioLegend | BD | Invitrogen |
| Cat. No. | CD300 | 12-2698-82 | 46-0567-42 | 302312 | 550855 | 344748 | 747920 | L349650 |

Finally, it should be noted that there are alternative ways of implementing the embodiments disclosed herein. Accordingly, the present embodiments are to be considered as illustrative and not restrictive, and the claims are not to be limited to the details given herein but may be modified within the scope and equivalents thereof.

What is claimed is:

1. A culture medium for expanding a target immune cell population comprising a first stimulant of proliferation for the target immune cell population proliferation, wherein the first stimulant comprises an IL-7Rαγc agonist peptide, wherein the IL-7Rαγc agonist peptide comprises:
(a) an IL-7Rα ligand, wherein the IL-7Rα ligand comprises:
an amino acid sequence of any one of SEQ ID NO: 43-46, 100-117, 118-193, 197-245 and 53-60;
a truncated amino acid sequence of any one of SEQ ID NO: 43-46, 100-117, 118-193, 197-245 and 53-60;
an amino acid sequence of any one of SEQ ID NO: 43-46, 100-117, 118-193, 197-245 and 53-606 having from 1 to 5 amino acid substitutions;
an amino acid sequence of any one of SEQ ID NO: 43-46, 100-117, 118-193, 197-245 and 53-60 having from 1 to 5 flanking glycines on the N-terminus, the C-terminus, or both the N-terminus and the C-terminus;
an amino acid sequence having greater than 60% sequence similarity to an amino acid sequence of any one of SEQ ID NO: 43-46, 100-117, 118-193, 197-245 and 53-60; or
a combination of any of the foregoing; and
(b) an Rγc ligand, wherein the Rγc ligand comprises:
an amino acid sequence of any one of SEQ ID NO: 246-327 and 47-52;
a truncated amino acid sequence of any one of SEQ ID NO: 246-327 and 47-52;
an amino acid sequence of any one of SEQ ID NO: 246-327 and 47-52 having from 1 to 5 amino acid substitutions;
an amino acid sequence of any one of SEQ ID NO: 246-327 and 47-52 having from 1 to 5 flanking glycines on the N-terminus, the C-terminus, or both the N-terminus and the C-terminus;
an amino acid sequence having greater than 60% sequence similarity to an amino acid sequence of any one of SEQ ID NO: 246-327 and 47-52; or
a combination of any of the foregoing.

2. The culture medium of claim 1, wherein the IL-7Rαγc agonist peptide comprises:
an IL-7Rα ligand comprising an amino acid sequence of any one of SEQ ID NO: 100-117, 118-193, 197-245 and 53-60; and
an Rγc ligand comprising an amino acid sequence of any one of SEQ ID NO: 246-327 and 47-52.

3. The culture medium of claim 1, wherein the IL-7Rα ligand comprises an amino acid sequence of any one of SEQ ID NO: 114 and 152.

4. The culture medium of claim 1, wherein the Rγc ligand comprises an amino acid sequence of any one of SEQ ID NO: 261 and 325-327.

5. The culture medium of claim 1, wherein the IL-7Rαγc agonist peptide comprises:
an amino acid sequence of any one of SEQ ID NO: 443-454 and 368-442;
a truncated amino acid sequence of any one of SEQ ID NO: 443-454 and 368-442;
an amino acid sequence of any one of SEQ ID NO: 443-454 and 368-442 having from 1 to 5 amino acid substitutions;
an amino acid sequence of any one of SEQ ID NO: 443-454 and 368-442 having from 1 to 5 flanking glycines on the N-terminus, the C-terminus, or both the N-terminus and the C-terminus;
an amino acid sequence having greater than 60% sequence similarity to an amino acid sequence of any one of SEQ ID NO: 443-454 and 368-442; or
a combination of any of the foregoing.

6. The culture medium of claim 1, wherein the IL-7Rαγc agonist peptide comprises an amino acid sequence of any one of SEQ ID NO: 394-404.

7. The culture medium any one of aspects claim 1, wherein the culture medium comprises a second stimulant of proliferation for the target immune cell population.

8. The culture medium of claim 7, wherein the second stimulant comprises an IL-2Rβγc agonist peptide.

9. The culture medium of claim 8, wherein the IL-2Rβγc agonist peptide comprises an amino acid sequence of any one of SEQ ID NO: 359-366, 61-76, and 77-99.

10. The culture medium of claim 9, wherein the IL-2Rβγc agonist peptide comprises an amino acid sequence of any one of SEQ ID NO: 91-99 and 359.

11. The culture medium of claim 7, wherein the second stimulant comprises an IL-15Rβγc agonist peptide.

12. The culture medium of claim 1, wherein the culture medium comprises an antibody directed to the target immune cell population.

13. A method of expanding a target immune cell population of an initial immune cell population, comprising incubating an initial immune cell population in the culture medium of claim 1 to provide an expanded target immune cell population.

14. A method of manufacturing an immune cell population, comprising activating a target immune cell population of a population of primary cells to provide an activated target immune cell population; and
expanding the activated target immune cell population, wherein expanding comprises incubating the activated target immune cell population in the presence of an IL-7Rαγc agonist peptide to provide an expanded target immune cell population, wherein the IL-7Rαγc agonist peptide comprises:
(a) an IL-7Rα ligand, wherein the IL-7Rα ligand comprises:
an amino acid sequence of any one of SEQ ID NO: 43-46, 100-117, 118-193, 197-245 and 53-60;
a truncated amino acid sequence of any one of SEQ ID NO: 43-46, 100-117, 118-193, 197-245 and 53-60;
an amino acid sequence of any one of SEQ ID NO: 43-46, 100-117, 118-193, 197-245 and 53-60 having from 1 to 5 amino acid substitutions;
an amino acid sequence of any one of SEQ ID NO: 43-46, 100-117, 118-193, 197-245 and 53-60 having from 1 to 5 flanking glycines on the N-terminus, the C-terminus, or both the N-terminus and the C-terminus;
an amino acid sequence having greater than 60% sequence similarity to an amino acid sequence of any one of SEQ ID NO: 43-46, 100-117, 118-193, 197-245 and 53-60; or
a combination of any of the foregoing; and
(b) an Rγc ligand, wherein the Rγc ligand comprises:
an amino acid sequence of any one of SEQ ID NO: 246-327 and 47-52;
a truncated amino acid sequence of any one of SEQ ID NO: 246-327 and 47-52;
an amino acid sequence of any one of SEQ ID NO: 246-327 and 47-52 having from 1 to 5 amino acid substitutions;
an amino acid sequence of any one of SEQ ID NO: 246-327 and 47-52 having from 1 to 5 flanking glycines on the N-terminus, the C-terminus, or both the N-terminus and the C-terminus;
an amino acid sequence having greater than 60% sequence similarity to an amino acid sequence of any one of SEQ ID NO: 246-327 and 47-52; or
a combination of any of the foregoing.

15. An enriched population of immune cells prepared using the culture medium of claim 1.

16. An enriched population of immune cells prepared using the method of claim 13.

17. An enriched population of immune cells prepared using the method of claim 14.

18. A pharmaceutical composition comprising the enriched population of claim 16.

19. A method of treating a disease in a patient comprising administering to a patient in need of such treatment a therapeutically effective amount of the pharmaceutical composition of claim 18.

20. A pharmaceutical composition comprising the enriched population of claim 17.

21. A method of treating a disease in a patient comprising administering to a patient in need of such treatment a therapeutically effective amount of the pharmaceutical composition of claim 20.

22. An immobilized IL-7Rαγc agonist peptide, comprising an IL-7Rαγc agonist peptide bound to a substrate, wherein the IL-7Rαγc agonist peptide comprises:
(a) an IL-7Rα ligand, wherein the IL-7Rα ligand comprises:
an amino acid sequence of any one of SEQ ID NO: 43-46, 100-117, 118-193, 197-245 and 53-60;
a truncated amino acid sequence of any one of SEQ ID NO: 43-46, 100-117, 118-193, 197-245 and 53-60;
an amino acid sequence of any one of SEQ ID NO: 43-46, 100-117, 118-193, 197-245 and 53-60 having from 1 to 5 amino acid substitutions;
an amino acid sequence of any one of SEQ ID NO: 43-46, 100-117, 118-193, 197-245 and 53-60 having from 1 to 5 flanking glycines on the N-terminus, the C-terminus, or both the N-terminus and the C-terminus;
an amino acid sequence having greater than 60% sequence similarity to an amino acid sequence of any one of SEQ ID NO: 43-46, 100-117, 118-193, 197-245 and 53-60; or
a combination of any of the foregoing; and
(b) an Rγc ligand, wherein the Rγc ligand comprises:
an amino acid sequence of any one of SEQ ID NO: 246-327 and 47-52;
a truncated amino acid sequence of any one of SEQ ID NO: 246-327 and 47-52;
an amino acid sequence of any one of SEQ ID NO: 246-327 and 47-52 having from 1 to 5 amino acid substitutions;
an amino acid sequence of any one of SEQ ID NO: 246-327 and 47-52 having from 1 to 5 flanking glycines on the N-terminus, the C-terminus, or both the N-terminus and the C-terminus;
an amino acid sequence having greater than 60% sequence similarity to an amino acid sequence of any one of SEQ ID NO: 246-327 and 47-52; or
a combination of any of the foregoing.


SEQUENCE LISTING (466 sequences)

SEQ ID NO: 1 - protein, 40 aa, synthetic construct
  VARIANT 1..40: note = This region may encompass 1-20 PA repeating units.
PAPAPAPAPAPAPAPAPAPAPAPAPAPAPAPAPAPAPAPA

SEQ ID NO: 2 - protein, 20 aa, synthetic construct
  VARIANT 1..20: note = This region may encompass 1-20 G repeating units, such as 1-15 G repeating units, 1-10 G repeating units, 1-5 G repeating units, 5-10 G repeating units, or 5-15 G repeating units.
GGGGGGGGGGGGGGGGGGGG

SEQ ID NO: 3 - protein, 40 aa, synthetic construct
  VARIANT 1..40: note = This region may encompass 1-20 GS repeating units, such as 1-15 GS repeating units, 1-10 GS repeating units, 1-5 GS repeating units, 5-10 GS repeating units, or 5-15 GS repeating units.
GSGSGSGSGSGSGSGSGSGSGSGSGSGSGSGSGSGSGSGS

SEQ ID NO: 4 - protein, 60 aa, synthetic construct
  VARIANT 1..60: note = This sequence may encompass 1-20 GGS repeating units, such as 1-15 GGS repeating units, 1-10 GGS repeating units, 1-5 GGGGS repeating units, 5-10 GGS repeating units, or 5-15 GGS repeating units.
GGSGGSGGSGGSGGSGGSGGSGGSGGSGGSGGSGGSGGSGGSGGSGGSGGSGGSGGSGGS

SEQ ID NO: 5 - protein, 80 aa, synthetic construct
  VARIANT 1..80: note = This region may encompass 1-20 GGGS repeating units, such as 1-15 GGGS repeating units, 1-10 GGGS repeating units, 1-5 GGGS repeating units, 5-10 GGGS repeating units, or 5-15 GGGS repeating units.
GGGSGGGSGGGSGGGSGGGSGGGSGGGSGGGSGGGSGGGSGGGSGGGSGGGSGGGSGGGSGGGSGGGSGGGSGGGSGGGS

SEQ ID NO: 6 - protein, 100 aa, synthetic construct
  VARIANT 1..100: note = This region may encompass 1-20 GGGGS repeating units, such as 1-15 GGGGS repeating units, 1-10 GGGGS repeating units, 1-5 GGGGS repeating units, 5-10 GGGGS repeating units, or 5-15 GGGGS repeating units.
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 7 - protein, 10 aa, synthetic construct
  VARIANT 1..10: note = This region may encompass 1-10 G repeating units, 1-8 G repeating units, 2-6 G repeating units, 2-4 G repeating units, or 2-3 G repeating units.
GGGGGGGGGG

SEQ ID NO: 8 - protein, 30 aa, synthetic construct
  VARIANT 1..30: note = This region may encompass 1-10 GS repeating units.
GSGSGSGSGSGSGSGSGSGSGSGSGSGSGS

SEQ ID NO: 9 - protein, 30 aa, synthetic construct
  VARIANT 9: note = This region may encompass 1-10 GGS repeating units.
GGSGGSGGSGGSGGSGGSGGSGGSGGSGGS

SEQ ID NO: 10 - protein, 40 aa, synthetic construct
  VARIANT 1..40: note = This region may encompass 1-10 GGGS repeating units.
GGGSGGGSGGGSGGGSGGGSGGGSGGGSGGGSGGGSGGGS

SEQ ID NO: 11 - protein, 100 aa, synthetic construct; identical to SEQ ID NO: 6
  VARIANT 1..100: note = This region may encompass 1-20 GGGGS repeating units.

SEQ ID NO: 12 - protein, 20 aa, synthetic construct
  VARIANT 1..20: note = This sequence may encompass 1-20 G repeating units.
GGGGGGGGGGGGGGGGGGGG

SEQ ID NO: 13 - protein, 40 aa, synthetic construct; identical to SEQ ID NO: 3
  VARIANT 1..40: note = This region may encompass 1-20 GS repeating units.

SEQ ID NO: 14 - protein, 60 aa, synthetic construct; identical to SEQ ID NO: 4
  VARIANT 1..60: note = This region may encompass 1-20 GGS repeating units.

SEQ ID NO: 15 - protein, 80 aa, synthetic construct; identical to SEQ ID NO: 5
  VARIANT 1..80: note = This region may encompass 1-20 GGGS repeating units.

SEQ ID NO: 16 - protein, 25 aa, synthetic construct
GGGGSGGGGSGGGGSGGGGSGGGGS

SEQ ID NO: 17 - protein, 5 aa, synthetic construct
GGGGS

SEQ ID NO: 18 - protein, 10 aa, synthetic construct
GGGGSGGGGS

SEQ ID NO: 19 - protein, 15 aa, synthetic construct
GGGGSGGGGSGGGGS

SEQ ID NO: 20 - protein, 20 aa, synthetic construct
GGGGSGGGGSGGGGSGGGGS

SEQ ID NO: 21-22 - skipped (2 entries)

SEQ ID NO: 23 - protein, 4 aa, synthetic construct
GGGG

SEQ ID NO: 24 - skipped

SEQ ID NO: 25 - protein, 4 aa, synthetic construct
GGGS

SEQ ID NO: 26 - protein, 7 aa, synthetic construct
GGGGSGG

SEQ ID NO: 27 - protein, 6 aa, synthetic construct
GGSGGS

SEQ ID NO: 28 - protein, 5 aa, synthetic construct
GGGGG

SEQ ID NO: 29 - protein, 20 aa, synthetic construct
GSGSGSGSGSGSGSGSGSGS

SEQ ID NO: 30 - protein, 20 aa, synthetic construct
  VARIANT 1..20: note = (P)n 1-20 This sequence may encompass from 1-20 P repeating units.
PPPPPPPPPPPPPPPPPPPP

SEQ ID NO: 31 - protein, 40 aa, synthetic construct; identical to SEQ ID NO: 1
  VARIANT 1..40: note = This region may encompass 1-20 PA repeating units.

SEQ ID NO: 32 - protein, 10 aa, synthetic construct
  VARIANT 1..10: note = This region may encompass 1-10 P repeating units.
PPPPPPPPPP

SEQ ID NO: 33 - protein, 20 aa, synthetic construct
PAPAPAPAPAPAPAPAPAPA

SEQ ID NO: 34 - protein, 5 aa, synthetic construct
  VARIANT 1..5: note = This region may encompass 1-5 P repeating units.
PPPPP

SEQ ID NO: 35 - protein, 10 aa, synthetic construct
  VARIANT 1..10: note = This region may encompass 1-5 PA repeating units.
PAPAPAPAPA

SEQ ID NO: 36 - protein, 10 aa, synthetic construct
PAPAPAPAPA

SEQ ID NO: 37 - protein, 14 aa, synthetic construct
PAPAPAPAPAPAPA

SEQ ID NO: 38 - protein, 20 aa, synthetic construct
PAPAPAPAPAPAPAPAPAPA

SEQ ID NO: 39 - protein, 40 aa, synthetic construct
PXPXPXPXPXPXPXPXPXPXPXPXPXPXPXPXPXPXPXPX

SEQ ID NO: 40 - protein, 12 aa, synthetic construct
PAPAPAPAPAPA

SEQ ID NO: 41 - protein, 16 aa, synthetic construct
PAPAPAPAPAPAPAPA

SEQ ID NO: 42 - protein, 18 aa, synthetic construct
PAPAPAPAPAPAPAPAPA

SEQ ID NO: 43-52 - skipped (10 entries)

SEQ ID NO: 53 - protein, 22 aa, synthetic construct
  VARIANT 1: note = This region may encompass one of the following amino acids: A, G, P, S, T, or V.
  VARIANT 2: note = This region may encompass one of the following amino acids: F, H, W, or Y.
  VARIANT 3: note = This region may encompass one of the following amino acids: A, G, H, K P, R, S, or T.
  VARIANT 13: note = This region may encompass one of the following amino acids: A, G, P, S, or T.
  VARIANT 20: note = This region may encompass one of the following amino acids: A, G, H, K, P, R, S, and T; or can be absent.
  VARIANT 21: note = This region may encompass an amino acid such as Q, G, or K; or can be absent.
  VARIANT 22: note = This region may encompass an amino acid such as G, H, or M; or can be absent.
XXXIPWCTLDPGXLQCAWLXXX

SEQ ID NO: 54 - protein, 21 aa, synthetic construct
  VARIANT 1: note = This region may encompass one of the following amino acids: F, H, W, or Y.
  VARIANT 2: note = This region may encompass one of the following amino acids: A, G, H, K P, R, S, or T.
  VARIANT 12: note = This region may encompass one of the following amino acids: A, G, P, S, or T.
  VARIANT 19: note = This region may encompass one of the following amino acids: A, G, H, K, P, R, S, and T; or can be absent.
  VARIANT 20: note = This region may encompass an amino acid such as Q, G, or K; or can be absent.
  VARIANT 21: note = This region may encompass an amino acid such as G, H, or M; or can be absent.
XXIPWCTLDPGXLQCAWLXXX

SEQ ID NO: 55 - protein, 20 aa, synthetic construct
  VARIANT 1: note = This region may encompass one of the following amino acids: A, G, H, K P, R, S, or T.
  VARIANT 11: note = This region may encompass one of the following amino acids: A, G, P, S, or T.
  VARIANT 18: note = This region may encompass one of the following amino acids: A, G, H, K, P, R, S, and T; or can be absent.
  VARIANT 19: note = This region may encompass an amino acid such as Q, G, or K; or can be absent.
  VARIANT 20: note = This region may encompass an amino acid such as G, H, or M; or can be absent.
XIPWCTLDPGXLQCAWLXXX

SEQ ID NO: 56 - protein, 19 aa, synthetic construct
  VARIANT 10: note = This region may encompass one of the following amino acids: A, G, P, S, or T.
  VARIANT 17: note = This region may encompass one of the following amino acids: A, G, H, K, P, R, S, and T; or can be a absent.
  VARIANT 19: note = This region may encompass an amino acid such as Q, G, or K; or can be absent.
  VARIANT 19: note = This region may encompass an amino acid such as G, H, or M; or can be absent.
IPWCTLDPGXLQCAWLXXX

SEQ ID NO: 57 - protein, 21 aa, synthetic construct
  VARIANT 20: note = This region may encompass one of the following amino acids: A, G, H, K, P, R, S, and T; or can be absent.
  VARIANT 21: note = This region may encompass an amino acid such as Q, G, or K; or can be absent.
  VARIANT 13: note = This region may encompass one of the following amino acids: A, G, P, S, or T.
  VARIANT 1: note = This region may encompass one of the following amino acids: A, G, P, S, T, or V.
  VARIANT 2: note = This region may encompass one of the following amino acids: F, H, W, or Y.
  VARIANT 3: note = This region may encompass one of the following amino acids: A, G, H, K P, R, S, or T.
XXXIPWCTLDPGXLQCAWLXX

SEQ ID NO: 58 - protein, 20 aa, synthetic construct
  VARIANT 13: note = This region may encompass one of the following amino acids: A, G, P, S, or T.
  VARIANT 20: note = This region may encompass one of the following amino acids: A, G, H, K, P, R, S, and T; or can be absent.
  VARIANT 1: note = This region may encompass one of the following amino acids: A, G, P, S, T, or V.
  VARIANT 2: note = This region may encompass one of the following amino acids: F, H, W, or Y.
  VARIANT 3: note = This region may encompass one of the following amino acids: A, G, H, K P, R, S, or T.
XXXIPWCTLDPGXLQCAWLX

SEQ ID NO: 59 - protein, 19 aa, synthetic construct
  VARIANT 1: note = This region may encompass one of the following amino acids: A, G, P, S, T, or V.
  VARIANT 2: note = This region may encompass one of the following amino acids: F, H, W, or Y.
  VARIANT 3: note = This region may encompass one of the following amino acids: A, G, H, K P, R, S, or T.
  VARIANT 13: note = This region may encompass one of the following amino acids: A, G, P, S, or T.
XXXIPWCTLDPGXLQCAWL

SEQ ID NO: 60 - protein, 18 aa, synthetic construct
  VARIANT 12: note = This region may encompass one of the following amino acids: A, G, P, S, or T.
  VARIANT 1: note = This region may encompass one of the following amino acids: A, G, P, S, T, or V.
  VARIANT 2: note = This region may encompass one of the following amino acids: F, H, W, or Y.
  VARIANT 3: note = This region may encompass one of the following amino acids: A, G, H, K P, R, S, or T.
XXXIPWCTLDPXLQCAWL

SEQ ID NO: 61 - protein, 47 aa, synthetic construct
GGWYPCWIARVGELCDLEEGPVNRGGGGSGGVVCQDWEGVELCWQGG

SEQ ID NO: 62 - protein, 46 aa, synthetic construct
GGAVEFYPCWLARIGELCDLVEPGGGGSGGVVCQDWEGVELCWQGG

SEQ ID NO: 63 - protein, 41 aa, synthetic construct
GGWYPCWIARVGELCDMEGGGGSGGVVCQDWEGVELCWQGG

SEQ ID NO: 64 - protein, 45 aa, synthetic construct
GGEWFHDCFLAKVGDLCDLFLWGGGGSGGVVCQDWEGVELCWQGG

SEQ ID NO: 65 - protein, 45 aa, synthetic construct
GGRYVHDCFIAQVGDLCDLFLHGGGGSGGVVCQDWEGVELCWQGG

SEQ ID NO: 66 - protein, 45 aa, synthetic construct
GGRSLVDCFLVKVGDLCDFFNWGGGGSGGVVCQDWEGVELCWQGG

SEQ ID NO: 67 - protein, 41 aa, synthetic construct
GGWYPCWIARVGELCDLEGGGGSGGVVCQDWEGVELCWQGG

SEQ ID NO: 68 - protein, 41 aa, synthetic construct
GGWYPCWLAQVGELCDLDGGGGSGGVVCQDWEGVELCWQGG

SEQ ID NO: 69 - protein, 52 aa, synthetic construct
GGWYPCWIARVGELCDLEEGPVNRGGGGSGGGGSGGVVCQDWEGVELCWQGG

SEQ ID NO: 70 - protein, 57 aa, synthetic construct
GGWYPCWIARVGELCDLEEGPVNRGGGGSGGGGSGGGGSGGVVCQDWEGVELCWQGG

SEQ ID NO: 71 - protein, 50 aa, synthetic construct
GGRYVHDCFIAQVGDLCDLFLHGGGGSGGGGSGGVVCQDWEGVELCWQGG

SEQ ID NO: 72 - protein, 55 aa, synthetic construct
GGRYVHDCFIAQVGDLCDLFLHGGGGSGGGGSGGGGSGGVVCQDWEGVELCWQGG

SEQ ID NO: 73 - protein, 60 aa, synthetic construct
GGRYVHDCFIAQVGDLCDLFLHGGGGSGGGGSGGGGSGGGGSGGVVCQDWEGVELCWQGG

SEQ ID NO: 74 - protein, 41 aa, synthetic construct
GGLVDCFKVKVGELCDLFGGGGSGGVVCQDWEGVELCWQGG

SEQ ID NO: 75 - protein, 45 aa, synthetic construct; identical to SEQ ID NO: 65

SEQ ID NO: 76 - protein, 41 aa, synthetic construct
GGWYSCWMAQLGELCDLDGGGGSGGVVCQDWEGVELCWQGG

SEQ ID NO: 77 - protein, 39 aa, synthetic construct
XWYPCWMAQLGELCDLDGGGGSGGVVCQDWEGVELCWQX

SEQ ID NO: 78 - protein, 39 aa, synthetic construct
XWYPCWMAQLGELCDLDGGGGSGGVVCQDWEGVALCWQX

SEQ ID NO: 79 - protein, 41 aa, synthetic construct
XWYPCWABUAQLGELCDLDGGGGSGGVVCQDWEGVELCWQX

SEQ ID NO: 80 - protein, 41 aa, synthetic construct
XWYPCWABUAQLGELCDLDGGGGSGGVVCQDWEGVALCWQX

SEQ ID NO: 81 - protein, 47 aa, synthetic construct
XGGELLVDCFKVKVGELCDLFFGGGGGSGGVVCQDWEGVELCWQGGX

SEQ ID NO: 82 - protein, 47 aa, synthetic construct
XGGRYVHDCFIAQVGDLCDLFLHGGGGSGGVVCQDWEGVELCWQGGX

SEQ ID NO: 83 - protein, 47 aa, synthetic construct
XGGKWVHDCFLAKVGDVCDLFVVGGGGSGGVVCQDWEGVELCWQGGX

SEQ ID NO: 84 - protein, 47 aa, synthetic construct
XGGRSLVDCFLVKVGDLCDFFNWGGGGSGGVVCQDWEGVELCWQGGX

SEQ ID NO: 85 - protein, 47 aa, synthetic construct
XGGEWFHDCFLAKVGDLCDLFLWGGGGSGGVVCQDWEGVELCWQGGX

SEQ ID NO: 86 - protein, 48 aa, synthetic construct
XGGGELLVDCFKVKVGELCDLFFGGGGGSGGVVCQDWEGVELCWQGGX

SEQ ID NO: 87 - protein, 48 aa, synthetic construct
XGGGRYVHDCFIAQVGDLCDLFLHGGGGSGGVVCQDWEGVELCWQGGX

SEQ ID NO: 88 - protein, 48 aa, synthetic construct
XGGGKWVHDCFLAKVGDVCDLFVVGGGGSGGVVCQDWEGVELCWQGGX

SEQ ID NO: 89 - protein, 49 aa, synthetic construct
ACGGGRSLVDCFLVKVGDLCDFFNWGGGGSGGVVCQDWEGVELCWQGGX

SEQ ID NO: 90 - protein, 49 aa, synthetic construct
ACGGGEWFHDCFLAKVGDLCDLFLWGGGGSGGVVCQDWEGVELCWQGGX

SEQ ID NO: 91 - protein, 56 aa, synthetic construct
GGGELLVDCFKVKVGELCDLFFGPAPAPAPAPAPAPAPAGVVCQDWEGVELCWQGG

SEQ ID NO: 92 - protein, 55 aa, synthetic construct
GGELLVDCFKVKVGELCDLFFGPAPAPAPAPAPAPAPAGVVCQDWEGVELCWQGG

SEQ ID NO: 93 - protein, 51 aa, synthetic construct
ELLVDCFKVKVGELCDLFFGPAPAPAPAPAPAPAPAGVVCQDWEGVELCWQ

SEQ ID NO: 94 - protein, 40 aa, synthetic construct
  VARIANT 23: note = This region may encompass one of the following sequences: (G)n where n is an integer from 1 to 5, (GS)n where n is an integer from 1 to 5, (GGS)n where n is an integer from 1 to 5 , (GGGS)n where n is an integer from 1 to 5 , (GGGGS)n where n is an integer from 1 to 5, (GGGGSGG)n where n is an integer from 1 to 5 or a combination of any of the foregoing.
GGELLVDCFKVKVGELCDLFFGXGVVCQDWEGVELCWQGG

SEQ ID NO: 95 - protein, 36 aa, synthetic construct
  VARIANT 21: note = This region may encompass one of the following sequences: (G)n where n is an integer from 1 to 5, (GS)n where n is an integer from 1 to 5, (GGS)n where n is an integer from 1 to 5 , (GGGS)n where n is an integer from 1 to 5 , (GGGGS)n where n is an integer from 1 to 5, (GGGGSGG)n where n is an integer from 1 to 5 or a combination of any of the foregoing.
ELLVDCFKVKVGELCDLFFGXGVVCQDWEGVELCWQ

SEQ ID NO: 96 - protein, 38 aa, synthetic construct
  VARIANT 22: note = This region may encompass one of the following sequences: (G)n where n is an integer from 1 to 5, (GS)n where n is an integer from 1 to 5, (GGS)n where n is an integer from 1 to 5 , (GGGS)n where n is an integer from 1 to 5 , (GGGGS)n where n is an integer from 1 to 5, (GGGGSGG)n where n is an integer from 1 to 5 or a combination of any of the foregoing.
  VARIANT 1: note = This region may encompass 1-5 G repeating units such as G, GG, GGG, GGGG, or GGGGG.
  VARIANT 38: note = This region may encompass 1-5 G repeating units such as G, GG, GGG, GGGG, or GGGGG.
XELLVDCFKVKVGELCDLFFGXGVVCQDWEGVELCWQX

SEQ ID NO: 97 - protein, 53 aa, synthetic construct
  VARIANT 1: note = This region may encompass 1-5 G repeating units such as G, GG, GGG, GGGG, or GGGGG.
  VARIANT 53: note = This region may encompass 1-5 G repeating units such as G, GG, GGG, GGGG, or GGGGG.
XELLVDCFKVKVGELCDLFFGPAPAPAPAPAPAPAPAGVVCQDWEGVELCWQX

SEQ ID NO: 98 - protein, 58 aa, synthetic construct
XGGGELLVDCFKVKVGELCDLFFGPAPAPAPAPAPAPAPAGVVCQDWEGVELCWQGGX

SEQ ID NO: 99 - protein, 57 aa, synthetic construct
XGGELLVDCFKVKVGELCDLFFGPAPAPAPAPAPAPAPAGVVCQDWEGVELCWQGGX

SEQ ID NO: 100 - protein, 20 aa, synthetic construct
HCLHWNIETLMSCVYGNFEE

SEQ ID NO: 101 - protein, 14 aa, synthetic construct
HCKHWDLESLLLCV

SEQ ID NO: 102 - protein, 22 aa, synthetic construct
HLGVPWCTLDPGSIQCAWLAKH

SEQ ID NO: 103 - protein, 22 aa, synthetic construct
IRSCLWQPGALHCTWWAEEEPV

SEQ ID NO: 104 - protein, 16 aa, synthetic construct
IPWCLLDPGGLQCVWL

SEQ ID NO: 105 - protein, 22 aa, synthetic construct
KAGSWFIPWCTLDPGSLQCAFL

SEQ ID NO: 106 - protein, 22 aa, synthetic construct
NPFRSVVPWCALDPGSLQCAWL

SEQ ID NO: 107 - protein, 14 aa, synthetic construct
QCIHWDIETLLSCV

SEQ ID NO: 108 - protein, 20 aa, synthetic construct
QCIHWDLESLLNCLRELKEP

SEQ ID NO: 109 - protein, 20 aa, synthetic construct
QCVHWDLDTLFGCIREQLEL

SEQ ID NO: 110 - protein, 22 aa, synthetic construct
RHFDDIIPWCTLDPGSLQCAYL

SEQ ID NO: 111 - protein, 20 aa, synthetic construct
SAKVLKQCLHWDLESLLSCL

SEQ ID NO: 112 - protein, 22 aa, synthetic construct
SLTVPWCTLDPGSMQCAWLQNR

SEQ ID NO: 113 - protein, 16 aa, synthetic construct
VPWCMLDPGSMQCAWL

SEQ ID NO: 114 - protein, 22 aa, synthetic construct
VHRIPWCTLDPGGLQCAWLRQM

SEQ ID NO: 115 - protein, 22 aa, synthetic construct
WVTIPWCILDPGSLQCEWQTKV

SEQ ID NO: 116 - protein, 22 aa, synthetic construct
GWGIPWCTLDPGSLQCAWLGKH

SEQ ID NO: 117 - protein, 22 aa, synthetic construct
YRSGHGIPWCMLDPGGLQCSWL

SEQ ID NO: 118 - protein, 19 aa, synthetic construct
IPWCTLDPGGLQCAWLRQM

SEQ ID NO: 119 - protein, 19 aa, synthetic construct
IPWCTLDPGGLQCAALRQM

SEQ ID NO: 120 - protein, 19 aa, synthetic construct
IPWCTLDPGGLQCAFLRQM

SEQ ID NO: 121 - protein, 19 aa, synthetic construct
IPWCTLDPGGLQCAYLRQM

SEQ ID NO: 122 - protein, 19 aa, synthetic construct
IPWCTLDPGGLQCAHLRQM

SEQ ID NO: 123 - protein, 19 aa, synthetic construct
IPWCTLDPGGLQCAWARQM

SEQ ID NO: 124 - protein, 19 aa, synthetic construct
IPWCTLDPGGLQCAWIRQM

SEQ ID NO: 125 - protein, 19 aa, synthetic construct
IPWCTLDPGGLQCAWVRQM

SEQ ID NO: 126 - protein, 19 aa, synthetic construct
IPWCTLDPGGLQCAWLAQM

SEQ ID NO: 127 - protein, 19 aa, synthetic construct
IPWCTLDPGGLQCAWLKQM

SEQ ID NO: 128 - protein, 19 aa, synthetic construct
IPWCTLDPGGLQCAWLHQM

SEQ ID NO: 129 - protein, 19 aa, synthetic construct
IPWCTLDPGGLQCAWLRAM

SEQ ID NO: 130 - protein, 19 aa, synthetic construct
IPWCTLDPGGLQCAWLRQA

SEQ ID NO: 131 - protein, 19 aa, synthetic construct
IPWCTLDPGGLQCAWLRAA

SEQ ID NO: 132 - protein, 19 aa, synthetic construct
IPWCTLDPGGLQCAWLAAA

SEQ ID NO: 133 - protein, 18 aa, synthetic construct
IPWCTLDPGGLQCAWLRQ

SEQ ID NO: 134 - protein, 17 aa, synthetic construct
IPWCTLDPGGLQCAWLR

SEQ ID NO: 135 - protein, 16 aa, synthetic construct
IPWCTLDPGGLQCAWL

SEQ ID NO: 136 - protein, 15 aa, synthetic construct
IPWCTLDPGGLQCAW

SEQ ID NO: 137 - protein, 21 aa, synthetic construct
IPWCTLDPGGLQCAWLRQMGG

SEQ ID NO: 138 - protein, 20 aa, synthetic construct
IPWCTLDPGGLQCAWLRQGG

SEQ ID NO: 139 - protein, 19 aa, synthetic construct
IPWCTLDPGGLQCAWLRGG

SEQ ID NO: 140 - protein, 18 aa, synthetic construct
IPWCTLDPGGLQCAWLGG

SEQ ID NO: 141 - protein, 17 aa, synthetic construct
IPWCTLDPGGLQCAWGG

SEQ ID NO: 142 - protein, 21 aa, synthetic construct
GGIPWCTLDPGGLQCAWLRQM

SEQ ID NO: 143 - protein, 20 aa, synthetic construct
GGIPWCTLDPGGLQCAWLRQ

SEQ ID NO: 144 - protein, 19 aa, synthetic construct
GGIPWCTLDPGGLQCAWLR

SEQ ID NO: 145 - protein, 18 aa, synthetic construct
GGIPWCTLDPGGLQCAWL

SEQ ID NO: 146 - protein, 17 aa, synthetic construct
GGIPWCTLDPGGLQCAW

SEQ ID NO: 147 - protein, 23 aa, synthetic construct
GGIPWCTLDPGGLQCAWLRQMGG

SEQ ID NO: 148 - protein, 22 aa, synthetic construct
GGIPWCTLDPGGLQCAWLRQGG

SEQ ID NO: 149 - protein, 21 aa, synthetic construct
GGIPWCTLDPGGLQCAWLRGG

SEQ ID NO: 150 - protein, 20 aa, synthetic construct
GGIPWCTLDPGGLQCAWLGG

SEQ ID NO: 151 - protein, 19 aa, synthetic construct
GGIPWCTLDPGGLQCAWGG

SEQ ID NO: 152 - protein, 24 aa, synthetic construct
VHRIPWCTLDPGGLQCAWLRQMGG

SEQ ID NO: 153 - protein, 24 aa, synthetic construct
GGVHRIPWCTLDPGGLQCAWLRQM

SEQ ID NO: 154 - protein, 26 aa, synthetic construct
GGVHRIPWCTLDPGGLQCAWLRQMGG

SEQ ID NO: 155 - protein, 21 aa, synthetic construct
VHRIPWCTLDPGGLQCAWLRQ

SEQ ID NO: 156 - protein, 20 aa, synthetic construct
VHRIPWCTLDPGGLQCAWLR

SEQ ID NO: 157 - protein, 21 aa, synthetic construct
VHRIPWCTLDPGGLQCAWLRM

SEQ ID NO: 158 - protein, 23 aa, synthetic construct
GGVHRIPWCTLDPGGLQCAWLRQ

SEQ ID NO: 159 - protein, 22 aa, synthetic construct
GGVHRIPWCTLDPGGLQCAWLR

SEQ ID NO: 160 - protein, 23 aa, synthetic construct
GGVHRIPWCTLDPGGLQCAWLRM

SEQ ID NO: 161 - protein, 22 aa, synthetic construct
VHRIPWCTLDPGGLQCAWARQM

SEQ ID NO: 162 - protein, 24 aa, synthetic construct
VHRIPWCTLDPGGLQCAWARQMGG

SEQ ID NO: 163 - protein, 24 aa, synthetic construct
GGVHRIPWCTLDPGGLQCAWARQM

SEQ ID NO: 164 - protein, 26 aa, synthetic construct
GGVHRIPWCTLDPGGLQCAWARQMGG

SEQ ID NO: 165 - protein, 22 aa, synthetic construct
VHRIPWCTLDPGGLQCAWVRQM

SEQ ID NO: 166 - protein, 24 aa, synthetic construct
VHRIPWCTLDPGGLQCAWVRQMGG

SEQ ID NO: 167 - protein, 24 aa, synthetic construct
GGVHRIPWCTLDPGGLQCAWVRQM

SEQ ID NO: 168 - protein, 26 aa, synthetic construct
GGVHRIPWCTLDPGGLQCAWVRQMGG

SEQ ID NO: 169 - protein, 22 aa, synthetic construct
VHRIPWCTLDPGGLQCAWIRQM

SEQ ID NO: 170 - protein, 24 aa, synthetic construct
VHRIPWCTLDPGGLQCAWIRQMGG

SEQ ID NO: 171 - protein, 24 aa, synthetic construct
GGVHRIPWCTLDPGGLQCAWIRQM

SEQ ID NO: 172 - protein, 26 aa, synthetic construct
GGVHRIPWCTLDPGGLQCAWIRQMGG

SEQ ID NO: 173 - protein, 20 aa, synthetic construct
IEGRGGQCIHWDIETLLSCV

SEQ ID NO: 174 - protein, 22 aa, synthetic construct
IEGRGGVPWCTLDPGSLQCAWF

SEQ ID NO: 175 - protein, 22 aa, synthetic construct
IEGRGGRYECADLPGGLHCEFR

SEQ ID NO: 176 - protein, 21 aa, synthetic construct
RSCLWQPGALHCTWWAEEEPV

SEQ ID NO: 177 - protein, 22 aa, synthetic construct
GGIEGRGGQCIHWDIETLLSCV

SEQ ID NO: 178 - protein, 24 aa, synthetic construct
GGIEGRGGVPWCTLDPGSLQCAWF

SEQ ID NO: 179 - protein, 24 aa, synthetic construct
GGIEGRGGRYECADLPGGLHCEFR

SEQ ID NO: 180 - protein, 23 aa, synthetic construct
GGRSCLWQPGALHCTWWAEEEPV

SEQ ID NO: 181 - protein, 22 aa, synthetic construct
QCVHWDLDTLFGCIREQLELGG

SEQ ID NO: 182 - protein, 22 aa, synthetic construct
GGQCVHWDLDTLFGCIREQLEL

SEQ ID NO: 183 - protein, 24 aa, synthetic construct
GGQCVHWDLDTLFGCIREQLELGG

SEQ ID NO: 184 - protein, 26 aa, synthetic construct
GGHLGVPWCTLDPGSIQCAWLAKHGG

SEQ ID NO: 185 - protein, 18 aa, synthetic construct
VHRIPWCTLDPGGLQCAW

SEQ ID NO: 186 - protein, 17 aa, synthetic construct
VHRIPWCTLDPGGLQCA

SEQ ID NO: 187 - protein, 16 aa, synthetic construct
VHRIPWCTLDPGGLQC

SEQ ID NO: 188 - protein, 23 aa, synthetic construct
HRIPWCTLDPGGLQCAWLRQMGG

SEQ ID NO: 189 - protein, 22 aa, synthetic construct
RIPWCTLDPGGLQCAWLRQMGG

SEQ ID NO: 190 - protein, 21 aa, synthetic construct
IPWCTLDPGGLQCAWLRQMGG

SEQ ID NO: 191 - protein, 20 aa, synthetic construct
PWCTLDPGGLQCAWLRQMGG

SEQ ID NO: 192 - protein, 19 aa, synthetic construct
WCTLDPGGLQCAWLRQMGG

SEQ ID NO: 193 - protein, 18 aa, synthetic construct
CTLDPGGLQCAWLRQMGG

SEQ ID NO: 194 - protein, 16 aa, synthetic construct
GQCIHWDIETLLSCVG

SEQ ID NO: 195 - protein, 18 aa, synthetic construct
GGQCIHWDIETLLSCVGG

SEQ ID NO: 196 - protein, 18 aa, synthetic construct
GGGQCIHWDIETLLSCVG

SEQ ID NO: 197 - protein, 18 aa, synthetic construct
IPWCTLDPGGLQCAWLRG

SEQ ID NO: 198 - protein, 19 aa, synthetic construct
IPWCTLDPGGLQCAWLRQG

SEQ ID NO: 199 - protein, 19 aa, synthetic construct
IPWCTLDPGGLQCAWLRGG

SEQ ID NO: 200 - protein, 17 aa, synthetic construct
IPWCTLDPGGLQCAWLG

SEQ ID NO: 201 - protein, 18 aa, synthetic construct
IPWCTLDPGGLQCAWLGK

SEQ ID NO: 202 - protein, 19 aa, synthetic construct
IPWCTLDPGGLQCAWLGKH

SEQ ID NO: 203 - protein, 19 aa, synthetic construct
VHRIPWCTLDPGGLQCAWL

SEQ ID NO: 204 - protein, 21 aa, synthetic construct
VHRIPWCTLDPGGLQCAWLRG

SEQ ID NO: 205 - protein, 22 aa, synthetic construct
VHRIPWCTLDPGGLQCAWLRQG

SEQ ID NO: 206 - protein, 22 aa, synthetic construct
VHRIPWCTLDPGGLQCAWLRGG

SEQ ID NO: 207 - protein, 20 aa, synthetic construct
VHRIPWCTLDPGGLQCAWLG

SEQ ID NO: 208 - protein, 21 aa, synthetic construct
VHRIPWCTLDPGGLQCAWLGK

SEQ ID NO: 209 - protein, 22 aa, synthetic construct
VHRIPWCTLDPGGLQCAWLGKH

SEQ ID NO: 210 - protein, 19 aa, synthetic construct
GWGIPWCTLDPGGLQCAWL

SEQ ID NO: 211 - protein, 20 aa, synthetic construct
GWGIPWCTLDPGGLQCAWLR

SEQ ID NO: 212 - protein, 21 aa, synthetic construct
GWGIPWCTLDPGGLQCAWLRQ

SEQ ID NO: 213 - protein, 21 aa, synthetic construct
GWGIPWCTLDPGGLQCAWLRG

SEQ ID NO: 214 - protein, 22 aa, synthetic construct
GWGIPWCTLDPGGLQCAWLRQG

SEQ ID NO: 215 - protein, 22 aa, synthetic construct
GWGIPWCTLDPGGLQCAWLRGG

SEQ ID NO: 216 - protein, 20 aa, synthetic construct
GWGIPWCTLDPGGLQCAWLG

SEQ ID NO: 217 - protein, 21 aa, synthetic construct
GWGIPWCTLDPGGLQCAWLGK

SEQ ID NO: 218 - protein, 22 aa, synthetic construct
GWGIPWCTLDPGGLQCAWLGKH

SEQ ID NO: 219 - protein, 16 aa, synthetic construct
IPWCTLDPGSLQCAWL

SEQ ID NO: 220 - protein, 17 aa, synthetic construct
IPWCTLDPGSLQCAWLR

SEQ ID NO: 221 - protein, 18 aa, synthetic construct
IPWCTLDPGSLQCAWLRQ

SEQ ID NO: 222 - protein, 18 aa, synthetic construct
IPWCTLDPGSLQCAWLRG

SEQ ID NO: 223 - protein, 19 aa, synthetic construct
IPWCTLDPGSLQCAWLRQG

SEQ ID NO: 224 - protein, 19 aa, synthetic construct
IPWCTLDPGSLQCAWLRGG

SEQ ID NO: 225 - protein, 17 aa, synthetic construct
IPWCTLDPGSLQCAWLG

SEQ ID NO: 226 - protein, 18 aa, synthetic construct
IPWCTLDPGSLQCAWLGK

SEQ ID NO: 227 - protein, 19 aa, synthetic construct
IPWCTLDPGSLQCAWLGKH

SEQ ID NO: 228 - protein, 19 aa, synthetic construct
VHRIPWCTLDPGSLQCAWL

SEQ ID NO: 229 - protein, 20 aa, synthetic construct
VHRIPWCTLDPGSLQCAWLR

SEQ ID NO: 230 - protein, 21 aa, synthetic construct
VHRIPWCTLDPGSLQCAWLRQ

SEQ ID NO: 231 - protein, 21 aa, synthetic construct
VHRIPWCTLDPGSLQCAWLRG

SEQ ID NO: 232 - protein, 22 aa, synthetic construct
VHRIPWCTLDPGSLQCAWLRQG

SEQ ID NO: 233 - protein, 22 aa, synthetic construct
VHRIPWCTLDPGSLQCAWLRGG

SEQ ID NO: 234 - protein, 20 aa, synthetic construct
VHRIPWCTLDPGSLQCAWLG

SEQ ID NO: 235 - protein, 21 aa, synthetic construct
VHRIPWCTLDPGSLQCAWLGK

SEQ ID NO: 236 - protein, 22 aa, synthetic construct
VHRIPWCTLDPGSLQCAWLGKH

SEQ ID NO: 237 - protein, 19 aa, synthetic construct
GWGIPWCTLDPGSLQCAWL

SEQ ID NO: 238 - protein, 20 aa, synthetic construct
GWGIPWCTLDPGSLQCAWLR

SEQ ID NO: 239 - protein, 21 aa, synthetic construct
GWGIPWCTLDPGSLQCAWLRQ

SEQ ID NO: 240 - protein, 21 aa, synthetic construct
GWGIPWCTLDPGSLQCAWLRG

SEQ ID NO: 241 - protein, 22 aa, synthetic construct
GWGIPWCTLDPGSLQCAWLRQG

SEQ ID NO: 242 - protein, 22 aa, synthetic construct
GWGIPWCTLDPGSLQCAWLRGG

SEQ ID NO: 243 - protein, 20 aa, synthetic construct
GWGIPWCTLDPGSLQCAWLG

SEQ ID NO: 244 - protein, 21 aa, synthetic construct
GWGIPWCTLDPGSLQCAWLGK

SEQ ID NO: 245 - protein, 22 aa, synthetic construct
GWGIPWCTLDPGSLQCAWLGKH

SEQ ID NO: 246 - protein, 14 aa, synthetic construct
IECDTSYGVYICWQ

SEQ ID NO: 247 - protein, 14 aa, synthetic construct
IECEEWRGVELCWQ

SEQ ID NO: 248 - protein, 20 aa, synthetic construct
PEGREVVVCRDWYGVELCWQ

SEQ ID NO: 249 - protein, 20 aa, synthetic construct
IWGRTVVECQDWEGVELCWQ

SEQ ID NO: 250 - protein, 20 aa, synthetic construct
LALRKEVVCQEYYGVELCWI

SEQ ID NO: 251 - protein, 20 aa, synthetic construct
HEAREVVVCQDWYGVELCWQ

SEQ ID NO: 252 - protein, 20 aa, synthetic construct
MVNREVVVCEDWYGVELCWQ

SEQ ID NO: 253 - protein, 20 aa, synthetic construct
TANQTVVECQVWGGVELCWQ

SEQ ID NO: 254 - protein, 14 aa, synthetic construct
VECQEWGGVELCWC

SEQ ID NO: 255 - protein, 15 aa, synthetic construct
DVECVDWGGVELCWH

SEQ ID NO: 256 - protein, 14 aa, synthetic construct
IVCEEWRGVELCWL

SEQ ID NO: 257 - protein, 20 aa, synthetic construct
DFERSYVVCQDWDGVELCWI

SEQ ID NO: 258 - protein, 20 aa, synthetic construct
AHSRQEVVCEEWYGVELCWI

SEQ ID NO: 259 - protein, 20 aa, synthetic construct
SAPERWVECEDWQGVELCWV

SEQ ID NO: 260 - protein, 20 aa, synthetic construct
YSRELYVQCEDWEGVELCWI

SEQ ID NO: 261 - protein, 14 aa, synthetic construct
VVCQDWEGVELCWQ

SEQ ID NO: 262 - protein, 15 aa, synthetic construct
DVVCQNWEGVDLCWH

SEQ ID NO: 263 - protein, 20 aa, synthetic construct
SAGRQEVVCQDWNGVELCWI

SEQ ID NO: 264 - protein, 20 aa, synthetic construct
GQGREVVVCHDWYGVELCWQ

SEQ ID NO: 265 - protein, 20 aa, synthetic construct
DWRRSVVECQDWYGVELCWQ

SEQ ID NO: 266 - protein, 15 aa, synthetic construct
DVVCQNWDGVDLCWH

SEQ ID NO: 267 - protein, 20 aa, synthetic construct
TLGRTVVECQDWGGVELCWQ

SEQ ID NO: 268 - protein, 20 aa, synthetic construct
RLLNSVVECLDWEGVELCWQ

SEQ ID NO: 269 - protein, 14 aa, synthetic construct
IVCEDWRGVELCWI

SEQ ID NO: 270 - protein, 14 aa, synthetic construct
VVCQEWEGVELCWC

SEQ ID NO: 271 - protein, 20 aa, synthetic construct
GDRPKEVVCEDWKGVELCWI

SEQ ID NO: 272 - protein, 20 aa, synthetic construct
ERPRSFIECQEWEGVELCWL

SEQ ID NO: 273 - protein, 20 aa, synthetic construct
EGSTTTIECEEWAGVELCWL

SEQ ID NO: 274 - protein, 20 aa, synthetic construct
ANQNTVVECQDWHGVELCWQ

SEQ ID NO: 275 - protein, 20 aa, synthetic construct
RSDDEVVVCQEWEGVELCWQ

SEQ ID NO: 276 - protein, 14 aa, synthetic construct
IECEEWAGVELCWL

SEQ ID NO: 277 - protein, 20 aa, synthetic construct
TWNMSELECQDWNGVEICWH

SEQ ID NO: 278 - protein, 20 aa, synthetic construct
GNDDSYIVCEEWKGVELCWI

SEQ ID NO: 279 - protein, 20 aa, synthetic construct
FAHHGVVECQEWYGVELCWQ

SEQ ID NO: 280 - protein, 20 aa, synthetic construct
LNRSVWIECEEYEGVELCWL

SEQ ID NO: 281 - protein, 20 aa, synthetic construct
WSKKAEVVCEEWGGVEFCWI

SEQ ID NO: 282 - protein, 20 aa, synthetic construct
RSNQTVVECQDWEGVELCWQ

SEQ ID NO: 283 - protein, 20 aa, synthetic construct
VVCQEWEGVELCWYAGECMQ

SEQ ID NO: 284 - protein, 20 aa, synthetic construct
ILCQEFEGVELCWLEESLAE

SEQ ID NO: 285 - protein, 20 aa, synthetic construct
KSQVECQDWEGVELCWVVSE

SEQ ID NO: 286 - protein, 20 aa, synthetic construct
KITVECQDWDGVELCWPTWI

SEQ ID NO: 287 - protein, 20 aa, synthetic construct
RPQIECQEWQGVELCWTREE

SEQ ID NO: 288 - protein, 20 aa, synthetic construct
VSCQEWDGVELCWVDGDLAA

SEQ ID NO: 289 - protein, 20 aa, synthetic construct
IMCQEWDGVELCWLERDKAN

SEQ ID NO: 290 - protein, 20 aa, synthetic construct
GLEIACEDWYGVELCWLRRA

SEQ ID NO: 291 - protein, 24 aa, synthetic construct
GYGVLCQEWQGVELCWPVQREAGV

SEQ ID NO: 292 - protein, 20 aa, synthetic construct
PYGVVCQDWAGVELCWVENR

SEQ ID NO: 293 - protein, 20 aa, synthetic construct
KLTVECQDWDGVELCWVGVE

SEQ ID NO: 294 - protein, 20 aa, synthetic construct
INCQTWNGVELCWVDEGLYQ

SEQ ID NO: 295 - protein, 20 aa, synthetic construct
VVCQEWEGVELCWVEPPLLP

SEQ ID NO: 296 - protein, 20 aa, synthetic construct
RVQVECEDWNGVELCWPVRV

SEQ ID NO: 297 - protein, 20 aa, synthetic construct
DRQVVCEEWDGVELCWIEES

SEQ ID NO: 298 - protein, 20 aa, synthetic construct
KTTVACQDWGGVELCWVERV

SEQ ID NO: 299 - protein, 20 aa, synthetic construct
RPEVVCQEWEGVELCWISPL

SEQ ID NO: 300 - protein, 20 aa, synthetic construct
RLGVECQEWEGVDLCWISAF

SEQ ID NO: 301 - protein, 20 aa, synthetic construct
KPVVVCEEWQGVELCWLEIQ

SEQ ID NO: 302 - protein, 20 aa, synthetic construct
VVCEVFQGVELCWCENEEFT

SEQ ID NO: 303 - protein, 20 aa, synthetic construct
TDEVSCQEWEGVELCWIERQ

SEQ ID NO: 304 - protein, 20 aa, synthetic construct
PVEVRCQEWEGVELCWVVGI

SEQ ID NO: 305 - protein, 20 aa, synthetic construct
GPEVVCEEFNRVELCWVEYN

SEQ ID NO: 306 - protein, 20 aa, synthetic construct
KYIVECQEWGGVELCWPEMV

SEQ ID NO: 307 - protein, 20 aa, synthetic construct
VTCQEYEGVELCWTVGCAYS

SEQ ID NO: 308 - protein, 21 aa, synthetic construct
VVCQEWEGVELCWQTGPGAHA

SEQ ID NO: 309 - protein, 20 aa, synthetic construct
IVCEEYNGVELCWVETSVKP

SEQ ID NO: 310 - protein, 20 aa, synthetic construct
EQQVVCQEWNGVELCWIEAG

SEQ ID NO: 311 - protein, 20 aa, synthetic construct
QLGVECQNWRGVELCWVSEI

SEQ ID NO: 312 - protein, 20 aa, synthetic construct
TAEVVCQEWDGVELCWIEVL

SEQ ID NO: 313 - protein, 20 aa, synthetic construct
SPSIVCEEWAGVELCWVDYS

SEQ ID NO: 314 - protein, 20 aa, synthetic construct
AVCQDWYGVELCWCMQDILD

SEQ ID NO: 315 - protein, 20 aa, synthetic construct
VECEEWGGVELCWLADEVMW

SEQ ID NO: 316 - protein, 20 aa, synthetic construct
HSTVICQDWDGVELCWIEND

SEQ ID NO: 317 - protein, 20 aa, synthetic construct
KKIVVCQDWGGVELCWTEDD

SEQ ID NO: 318 - protein, 20 aa, synthetic construct
SVEVVCEEWHGVELCWPVFI

SEQ ID NO: 319 - protein, 20 aa, synthetic construct
RWAVSCQDWQGIELCWPEWD

SEQ ID NO: 320 - protein, 20 aa, synthetic construct
RTGVECQDWHGVELCWPVWE

SEQ ID NO: 321 - protein, 20 aa, synthetic construct
GYGVVCEDFRGVELCWLERK

SEQ ID NO: 322 - protein, 17 aa, synthetic construct
RTEVECEDWEGVELCWL

SEQ ID NO: 323 - protein, 19 aa, synthetic construct
ILCEEWQGVELCWLEGGGS

SEQ ID NO: 324 - protein, 16 aa, synthetic construct
VGIECEEWAGVELCWL

SEQ ID NO: 325 - protein, 16 aa, synthetic construct
VVCQDWEGVELCWQGG

SEQ ID NO: 326 - protein, 16 aa, synthetic construct
GGVVCQDWEGVELCWQ

SEQ ID NO: 327 - protein, 18 aa, synthetic construct
GGVVCQDWEGVELCWQGG

SEQ ID NO: 328 - protein, 20 aa, synthetic construct
MGFYPCWTAQLGELCDLSVD

SEQ ID NO: 329 - protein, 19 aa, synthetic construct
GFYPCWTAQLGELCDLSVD

SEQ ID NO: 330 - protein, 18 aa, synthetic construct
FYPCWTAQLGELCDLSVD

SEQ ID NO: 331 - protein, 17 aa, synthetic construct
YPCWTAQLGELCDLSVD

SEQ ID NO: 332 - protein, 19 aa, synthetic construct
MGFYPCWTAQLGELCDLSV

SEQ ID NO: 333 - protein, 18 aa, synthetic construct
MGFYPCWTAQLGELCDLS

SEQ ID NO: 334 - protein, 17 aa, synthetic construct
MGFYPCWTAQLGELCDL

SEQ ID NO: 335 - protein, 18 aa, synthetic construct
GFYPCWTAQLGELCDLSV

SEQ ID NO: 336 - protein, 15 aa, synthetic construct
FYPCWTAQLGELCDL

SEQ ID NO: 337 - protein, 14 aa, synthetic construct
KYCGFAQLGELCVL

SEQ ID NO: 338 - protein, 15 aa, synthetic construct
GKYCGFAQLGELCVL

SEQ ID NO: 339 - protein, 16 aa, synthetic construct
GGKYCGFAQLGELCVL

SEQ ID NO: 340 - protein, 17 aa, synthetic construct
GGGKYCGFAQLGELCVL

SEQ ID NO: 341 - protein, 15 aa, synthetic construct
KYCGFAQLGELCVLG

SEQ ID NO: 342 - protein, 16 aa, synthetic construct
KYCGFAQLGELCVLGG

SEQ ID NO: 343 - protein, 17 aa, synthetic construct
KYCGFAQLGELCVLGGG

SEQ ID NO: 344 - protein, 16 aa, synthetic construct
GKYCGFAQLGELCVLG

SEQ ID NO: 345 - protein, 17 aa, synthetic construct
GGKYCGFAQLGELCVLG

SEQ ID NO: 346 - protein, 14 aa, synthetic construct
YWCWMAQVGELCDL

SEQ ID NO: 347 - protein, 14 aa, synthetic construct
YHCWMAQVGELCDL

SEQ ID NO: 348 - protein, 14 aa, synthetic construct
YHCWMGQVGELCDL

SEQ ID NO: 349 - protein, 14 aa, synthetic construct
YHCWMGQMGELCDL

SEQ ID NO: 350 - protein, 14 aa, synthetic construct
YHCWMGQMGELCEL

SEQ ID NO: 351 - protein, 14 aa, synthetic construct
YHCWMGQMGELCEM

SEQ ID NO: 352 - protein, 20 aa, synthetic construct
YPCWLARVGELCDLDSGDVH

SEQ ID NO: 353 - protein, 20 aa, synthetic construct
APCWLARVGELCDLDSGDVH

SEQ ID NO: 354 - protein, 20 aa, synthetic construct
APCALARVGELCDLDSGDVH

SEQ ID NO: 355 - protein, 20 aa, synthetic construct
APCALAAVGELCDLDSGDVH

SEQ ID NO: 356 - protein, 20 aa, synthetic construct
APCALAAVGALCDLDSGDVH

SEQ ID NO: 357 - protein, 20 aa, synthetic construct
APCALAAVGALCDLASGDVH

SEQ ID NO: 358 - protein, 20 aa, synthetic construct
APCALAAVGALCDLAAGDVH

SEQ ID NO: 359 - protein, 41 aa, synthetic construct
GGWYPCWMAQLGELCDLDGGGGSGGVVCQDWEGVELCWQGG

SEQ ID NO: 360 - protein, 37 aa, synthetic construct
WYPCWMAQLGELCDLDGGGGSGGVVCQDWEGVELCWQ

SEQ ID NO: 361 - protein, 35 aa, synthetic construct
WYPCWMAQLGELCDLDGGXGGVVCQDWEGVELCWQ

SEQ ID NO: 362 - protein, 31 aa, synthetic construct
WYPCWMAQLGELCDLDXVVCQDWEGVELCWQ

SEQ ID NO: 363 - protein, 39 aa, synthetic construct
GWYPCWMAQLGELCDLDGGGGSGGVVCQDWEGVELCWQG

SEQ ID NO: 364 - protein, 40 aa, synthetic construct
GGWYPCWMAQLGELCDLDGGGGSGGVVCQDWEGVELCWQG

SEQ ID NO: 365 - protein, 40 aa, synthetic construct
GWYPCWMAQLGELCDLDGGGGSGGVVCQDWEGVELCWQGG

SEQ ID NO: 366 - protein, 33 aa, synthetic construct
  VARIANT 18: note = This region may encompass from 1 to 20 amino acids or one of the following amino acid sequences: (G)n where n is an integer from 1 to 5, (GS)n where n is an integer from 1 to 5 , (GGS)n where n is an integer from 1 to 5, (GGGS)n where n is an integer from 1 to 5 , (GGGGS)n where n is an integer from 1 to 5, or a combination of any of the foregoing; such as GGGGG, GSGSGSGSGS, GGSGGSGGSGGSGGS, GGGSGGGSGGGSGGGSGGGS, or GGGGSGGGGSGGGGSGGGGSGGGGS.
  VARIANT 1: note = This region may encompass 1-5 G repeating units.
  VARIANT 33: note = This region may encompass 1-5 G repeating units.
XWYPCWMAQLGELCDLDXVVCQDWEGVELCWQX

SEQ ID NO: 367 - skipped

SEQ ID NO: 368 - protein, 39 aa, synthetic construct
  VARIANT 23: note = This region may encompass from 1 to 20 amino acids or one of the following amino acid sequences: (G)n where n is an integer from 1 to 5, (GS)n where n is an integer from 1 to 5 , (GGS)n where n is an integer from 1 to 5, (GGGS)n where n is an integer from 1 to 5 , (GGGGS)n where n is an integer from 1 to 5, or a combination of any of the foregoing; such as GGGGG, GSGSGSGSGS, GGSGGSGGSGGSGGS, GGGSGGGSGGGSGGGSGGGS, or GGGGSGGGGSGGGGSGGGGSGGGGS.
VHRIPWCTLDPGGLQCAWLRQMXGGVVCQDWEGVELCWQ

SEQ ID NO: 369 - protein, 37 aa, synthetic construct
  VARIANT 23: note = This region may encompass from 1 to 20 amino acids or one of the following amino acid sequences: (G)n where n is an integer from 1 to 5, (GS)n where n is an integer from 1 to 5 , (GGS)n where n is an integer from 1 to 5, (GGGS)n where n is an integer from 1 to 5 , (GGGGS)n where n is an integer from 1 to 5, or a combination of any of the foregoing; such as GGGGG, GSGSGSGSGS, GGSGGSGGSGGSGGS, GGGSGGGSGGGSGGGSGGGS, or GGGGSGGGGSGGGGSGGGGSGGGGS.
VHRIPWCTLDPGGLQCAWLRQMXVVCQDWEGVELCWQ

SEQ ID NO: 370 - protein, 41 aa, synthetic construct
IEGRGGQCIHWDIETLLSCVGGGGSGGVVCQDWEGVELCWQ

SEQ ID NO: 371 - protein, 43 aa, synthetic construct
IEGRGGVPWCTLDPGSLQCAWFGGGGSGGVVCQDWEGVELCWQ

SEQ ID NO: 372 - protein, 43 aa, synthetic construct
IEGRGGRYECADLPGGLHCEFRGGGGSGGVVCQDWEGVELCWQ

SEQ ID NO: 373 - protein, 43 aa, synthetic construct
RHFDDIIPWCTLDPGSLQCAYLGGGGSGGVVCQDWEGVELCWQ

SEQ ID NO: 374 - protein, 46 aa, synthetic construct
VHRIPWCTLDPGGLQCAWLRQMGGGGSGGVVCQDWEGVELCWQPPA

SEQ ID NO: 375 - protein, 45 aa, synthetic construct
VHRIPWCTLDPGGLQCAWLRQGGGGSGGVVCQDWEGVELCWQPPA

SEQ ID NO: 376 - protein, 44 aa, synthetic construct
VHRIPWCTLDPGGLQCAWLRGGGGSGGVVCQDWEGVELCWQPPA

SEQ ID NO: 377 - protein, 46 aa, synthetic construct
GWGIPWCTLDPGSLQCAWLGKHGGGGSGGVVCQDWEGVELCWQPPA

SEQ ID NO: 378 - protein, 46 aa, synthetic construct
VHRIPWCTLDPGGLQCAWLRQGGGGGSGGVVCQDWEGVELCWQPPA

SEQ ID NO: 379 - protein, 46 aa, synthetic construct
VHRIPWCTLDPGGLQCAWLRGGGGGGSGGVVCQDWEGVELCWQPPA

SEQ ID NO: 380 - protein, 44 aa, synthetic construct
VHRIPWCTLDPGGLQCAWLRQGGGGSGGVVCQDWEGVELCWQGG

SEQ ID NO: 381 - protein, 43 aa, synthetic construct
VHRIPWCTLDPGGLQCAWLRGGGGSGGVVCQDWEGVELCWQGG

SEQ ID NO: 382 - protein, 45 aa, synthetic construct
GWGIPWCTLDPGSLQCAWLGKHGGGGSGGVVCQDWEGVELCWQGG

SEQ ID NO: 383 - protein, 41 aa, synthetic construct
VHRIPWCTLDPGGLQCAWLRQMPAGVVCQDWEGVELCWQGG

SEQ ID NO: 384 - protein, 43 aa, synthetic construct
HLGVPWCTLDPGSIQCAWLAKHGGGGSGGVVCQDWEGVELCWQ

SEQ ID NO: 385 - protein, 43 aa, synthetic construct
VVCQDWEGVELCWQGGGGSGGRHFDDIIPWCTLDPGSLQCAYL

SEQ ID NO: 386 - protein, 43 aa, synthetic construct
VVCQDWEGVELCWQGGGGSGGHLGVPWCTLDPGSIQCAWLAKH

SEQ ID NO: 387 - protein, 35 aa, synthetic construct
HCKHWDLESLLLCVGGGGSGGVVCQDWEGVELCWQ

SEQ ID NO: 388 - protein, 41 aa, synthetic construct
QCVHWDLDTLFGCIREQLELGGGGSGGVVCQDWEGVELCWQ

SEQ ID NO: 389 - protein, 41 aa, synthetic construct
VVCQDWEGVELCWQGGGGSGGQCVHWDLDTLFGCIREQLEL

SEQ ID NO: 390 - protein, 43 aa, synthetic construct
IRSCLWQPGALHCTWWAEEEPVGGGGSGGVVCQDWEGVELCWQ

SEQ ID NO: 391 - protein, 43 aa, synthetic construct
VVCQDWEGVELCWQGGGGSGGIRSCLWQPGALHCTWWAEEEPV

SEQ ID NO: 392 - protein, 37 aa, synthetic construct
IPWCLLDPGGLQCVWLGGGGSGGVVCQDWEGVELCWQ

SEQ ID NO: 393 - protein, 37 aa, synthetic construct
VVCQDWEGVELCWQGGGGSGGIPWCLLDPGGLQCVWL

SEQ ID NO: 394 - protein, 44 aa, synthetic construct
VHRIPWCTLDPGGLQCAWLRQMGGGGSGGVVCQDWEGVELCWQG

SEQ ID NO: 395 - protein, 45 aa, synthetic construct
VHRIPWCTLDPGGLQCAWLRQMGGGGSGGVVCQDWEGVELCWQGG

SEQ ID NO: 396 - protein, 46 aa, synthetic construct
GVHRIPWCTLDPGGLQCAWLRQMGGGGSGGVVCQDWEGVELCWQGG

SEQ ID NO: 397 - protein, 47 aa, synthetic construct
GGVHRIPWCTLDPGGLQCAWLRQMGGGGSGGVVCQDWEGVELCWQGG

SEQ ID NO: 398 - protein, 41 aa, synthetic construct
VHRIPWCTLDPGGLQCAWLRGGGGSGGVVCQDWEGVELCWQ

SEQ ID NO: 399 - protein, 42 aa, synthetic construct
WGIPWCTLDPGSLQCAWLGKHGGGGSGGVVCQDWEGVELCWQ

SEQ ID NO: 400 - protein, 42 aa, synthetic construct
VHRIPWCTLDPGGLQCAWLRQGGGGSGGVVCQDWEGVELCWQ

SEQ ID NO: 401 - protein, 42 aa, synthetic construct
VHRIPWCTLDPGGLQCAWLRMGGGGSGGVVCQDWEGVELCWQ

SEQ ID NO: 402 - protein, 43 aa, synthetic construct
VHRIPWCTLDPGGLQCAWIRQMGGGGSGGVVCQDWEGVELCWQ

SEQ ID NO: 403 - protein, 43 aa, synthetic construct
VHRIPWCTLDPGGLQCAWVRQMGGGGSGGVVCQDWEGVELCWQ

SEQ ID NO: 404 - protein, 43 aa, synthetic construct
VHRIPWCTLDPGGLQCAWARQMGGGGSGGVVCQDWEGVELCWQ

SEQ ID NO: 405 - protein, 45 aa, synthetic construct
GVHRIPWCTLDPGGLQCAWLRQMGGGGSGGVVCQDWEGVELCWQG

SEQ ID NO: 406 - protein, 42 aa, synthetic construct; identical to SEQ ID NO: 400

SEQ ID NO: 407 - protein, 41 aa, synthetic construct; identical to SEQ ID NO: 398

SEQ ID NO: 408 - protein, 42 aa, synthetic construct
VHRIPWCTLDPGGLQCAWLGKHGGGGSGGVVCQWEGVELCWQ

SEQ ID NO: 409 - protein, 42 aa, synthetic construct; identical to SEQ ID NO: 401

SEQ ID NO: 410 - protein, 43 aa, synthetic construct
GWGIPWCTLDPGGLQCAWLRQMGGGGSGGVVCQDWEGVELCWQ

SEQ ID NO: 411 - protein, 42 aa, synthetic construct
GWGIPWCTLDPGGLQCAWLRQGGGGSGGVVCQDWEGVELCWQ

SEQ ID NO: 412 - protein, 41 aa, synthetic construct
GWGIPWCTLDPGGLQCAWLRGGGGSGGVVCQDWEGVELCWQ

SEQ ID NO: 413 - protein, 43 aa, synthetic construct
GWGIPWCTLDPGGLQCAWLGKHGGGGSGGVVCQDWEGVELCWQ

SEQ ID NO: 414 - protein, 42 aa, synthetic construct
GWGIPWCTLDPGGLQCAWLRMGGGGSGGVVCQDWEGVELCWQ

SEQ ID NO: 415 - protein, 40 aa, synthetic construct
IPWCTLDPGGLQCAWLRQMGGGGSGGVVCQDWEGVELCWQ

SEQ ID NO: 416 - protein, 39 aa, synthetic construct
IPWCTLDPGGLQCAWLRQGGGGSGGVVCQDWEGVELCWQ

SEQ ID NO: 417 - protein, 38 aa, synthetic construct
IPWCTLDPGGLQCAWLRGGGGSGGVVCQDWEGVELCWQ

SEQ ID NO: 418 - protein, 40 aa, synthetic construct
IPWCTLDPGGLQCAWLGKHGGGGSGGVVCQDWEGVELCWQ

SEQ ID NO: 419 - protein, 39 aa, synthetic construct
IPWCTLDPGGLQCAWLRMGGGGSGGVVCQDWEGVELCWQ

SEQ ID NO: 420 - protein, 37 aa, synthetic construct; identical to SEQ ID NO: 369
  VARIANT 23: note = This region may encompass one of the following sequences: (G)n where n is an integer from 1 to 5, (GS)n where n is an integer from 1 to 5, (GGS)n where n is an integer from 1 to 5 , (GGGS)n where n is an integer from 1 to 5 , (GGGGS)n where n is an integer from 1 to 5, or a combination of any of the foregoing; such as GGGGG, GSGSGSGSGS, GGSGGSGGSGGSGGS, GGGSGGGSGGGSGGGSGGGS, or GGGGSGGGGSGGGGSGGGGSGGGGS

SEQ ID NO: 421 - protein, 36 aa, synthetic construct
  VARIANT 22: note = This region may encompass one of the following sequences: (G)n where n is an integer from 1 to 5, (GS)n where n is an integer from 1 to 5, (GGS)n where n is an integer from 1 to 5 , (GGGS)n where n is an integer from 1 to 5 , (GGGGS)n where n is an integer from 1 to 5, or a combination of any of the foregoing; such as GGGGG, GSGSGSGSGS, GGSGGSGGSGGSGGS, GGGSGGGSGGGSGGGSGGGS, or GGGGSGGGGSGGGGSGGGGSGGGGS
VHRIPWCTLDPGGLQCAWLRQXVVCQDWEGVELCWQ

SEQ ID NO: 422 - protein, 35 aa, synthetic construct
  VARIANT 21: note = This region may encompass one of the following sequences: (G)n where n is an integer from 1 to 5, (GS)n where n is an integer from 1 to 5, (GGS)n where n is an integer from 1 to 5 , (GGGS)n where n is an integer from 1 to 5 , (GGGGS)n where n is an integer from 1 to 5, or a combination of any of the foregoing; such as GGGGG, GSGSGSGSGS, GGSGGSGGSGGSGGS, GGGSGGGSGGGSGGGSGGGS, or GGGGSGGGGSGGGGSGGGGSGGGGS
VHRIPWCTLDPGGLQCAWLRXVVCQDWEGVELCWQ

SEQ ID NO: 423 - protein, 37 aa, synthetic construct
  VARIANT 23: note = This region may encompass one of the following sequences: (G)n where n is an integer from 1 to 5, (GS)n where n is an integer from 1 to 5, (GGS)n where n is an integer from 1 to 5 , (GGGS)n where n is an integer from 1 to 5 , (GGGGS)n where n is an integer from 1 to 5, or a combination of any of the foregoing; such as GGGGG, GSGSGSGSGS, GGSGGSGGSGGSGGS, GGGSGGGSGGGSGGGSGGGS, or GGGGSGGGGSGGGGSGGGGSGGGGS
VHRIPWCTLDPGGLQCAWLGKHXVVCQDWEGVELCWQ

SEQ ID NO: 424 - protein, 36 aa, synthetic construct
  VARIANT 22: note = This region may encompass one of the following sequences: (G)n where n is an integer from 1 to 5, (GS)n where n is an integer from 1 to 5, (GGS)n where n is an integer from 1 to 5 , (GGGS)n where n is an integer from 1 to 5 , (GGGGS)n where n is an integer from 1 to 5, or a combination of any of the foregoing; such as GGGGG, GSGSGSGSGS, GGSGGSGGSGGSGGS, GGGSGGGSGGGSGGGSGGGS, or GGGGSGGGGSGGGGSGGGGSGGGGS
VHRIPWCTLDPGGLQCAWLRMXVVCQDWEGVELCWQ

SEQ ID NO: 425 - protein, 37 aa, synthetic construct
  VARIANT 23: note = This region may encompass one of the following sequences: (G)n where n is an integer from 1 to 5, (GS)n where n is an integer from 1 to 5, (GGS)n where n is an integer from 1 to 5 , (GGGS)n where n is an integer from 1 to 5 , (GGGGS)n where n is an integer from 1 to 5, or a combination of any of the foregoing; such as GGGGG, GSGSGSGSGS, GGSGGSGGSGGSGGS, GGGSGGGSGGGSGGGSGGGS, or GGGGSGGGGSGGGGSGGGGSGGGGS
GWGIPWCTLDPGGLQCAWLRQMXVVCQDWEGVELCWQ

SEQ ID NO: 426 - protein, 36 aa, synthetic construct
  VARIANT 22: note = This region may encompass one of the following sequences: (G)n where n is an integer from 1 to 5, (GS)n where n is an integer from 1 to 5, (GGS)n where n is an integer from 1 to 5 , (GGGS)n where n is an integer from 1 to 5 , (GGGGS)n where n is an integer from 1 to 5, or a combination of any of the foregoing; such as GGGGG, GSGSGSGSGS, GGSGGSGGSGGSGGS, GGGSGGGSGGGSGGGSGGGS, or GGGGSGGGGSGGGGSGGGGSGGGGS
GWGIPWCTLDPGGLQCAWLRQXVVCQDWEGVELCWQ

SEQ ID NO: 427 - protein, 35 aa, synthetic construct
  VARIANT 21: note = This region may encompass one of the following sequences: (G)n where n is an integer from 1 to 5, (GS)n where n is an integer from 1 to 5, (GGS)n where n is an integer from 1 to 5 , (GGGS)n where n is an integer from 1 to 5 , (GGGGS)n where n is an integer from 1 to 5, or a combination of any of the foregoing; such as GGGGG, GSGSGSGSGS, GGSGGSGGSGGSGGS, GGGSGGGSGGGSGGGSGGGS, or GGGGSGGGGSGGGGSGGGGSGGGGS
GWGIPWCTLDPGGLQCAWLRXVVCQDWEGVELCWQ

SEQ ID NO: 428 - protein, 37 aa, synthetic construct
  VARIANT 23: note = This region may encompass one of the following sequences: (G)n where n is an integer from 1 to 5, (GS)n where n is an integer from 1 to 5, (GGS)n where n is an integer from 1 to 5 , (GGGS)n where n is an integer from 1 to 5 , (GGGGS)n where n is an integer from 1 to 5, or a combination of any of the foregoing; such as GGGGG, GSGSGSGSGS, GGSGGSGGSGGSGGS, GGGSGGGSGGGSGGGSGGGS, or GGGGSGGGGSGGGGSGGGGSGGGGS
GWGIPWCTLDPGGLQCAWLGKHXVVCQDWEGVELCWQ

SEQ ID NO: 429 - protein, 36 aa, synthetic construct
  VARIANT 22: note = This region may encompass one of the following sequences: (G)n where n is an integer from 1 to 5, (GS)n where n is an integer from 1 to 5, (GGS)n where n is an integer from 1 to 5 , (GGGS)n where n is an integer from 1 to 5 , (GGGGS)n where n is an integer from 1 to 5, or a combination of any of the foregoing; such as GGGGG, GSGSGSGSGS, GGSGGSGGSGGSGGS, GGGSGGGSGGGSGGGSGGGS, or GGGGSGGGGSGGGGSGGGGSGGGGS
GWGIPWCTLDPGGLQCAWLRMXVVCQDWEGVELCWQ

SEQ ID NO: 430 - protein, 34 aa, synthetic construct
  VARIANT 20: note = This region may encompass one of the following sequences: (G)n where n is an integer from 1 to 5, (GS)n where n is an integer from 1 to 5, (GGS)n where n is an integer from 1 to 5 , (GGGS)n where n is an integer from 1 to 5 , (GGGGS)n where n is an integer from 1 to 5, or a combination of any of the foregoing; such as GGGGG, GSGSGSGSGS, GGSGGSGGSGGSGGS, GGGSGGGSGGGSGGGSGGGS, or GGGGSGGGGSGGGGSGGGGSGGGGS
IPWCTLDPGGLQCAWLRQMXVVCQDWEGVELCWQ

SEQ ID NO: 431 - protein, 33 aa, synthetic construct
  VARIANT 19: note = This region may encompass one of the following sequences: (G)n where n is an integer from 1 to 5, (GS)n where n is an integer from 1 to 5, (GGS)n where n is an integer from 1 to 5 , (GGGS)n where n is an integer from 1 to 5 , (GGGGS)n where n is an integer from 1 to 5, or a combination of any of the foregoing; such as GGGGG, GSGSGSGSGS, GGSGGSGGSGGSGGS, GGGSGGGSGGGSGGGSGGGS, or GGGGSGGGGSGGGGSGGGGSGGGGS
IPWCTLDPGGLQCAWLRQXVVCQDWEGVELCWQ

SEQ ID NO: 432 - protein, 32 aa, synthetic construct
  VARIANT 18: note = This region may encompass one of the following sequences: (G)n where n is an integer from 1 to 5, (GS)n where n is an integer from 1 to 5, (GGS)n where n is an integer from 1 to 5 , (GGGS)n where n is an integer from 1 to 5 , (GGGGS)n where n is an integer from 1 to 5, or a combination of any of the foregoing; such as GGGGG, GSGSGSGSGS, GGSGGSGGSGGSGGS, GGGSGGGSGGGSGGGSGGGS, or GGGGSGGGGSGGGGSGGGGSGGGGS
IPWCTLDPGGLQCAWLRXVVCQDWEGVELCWQ

SEQ ID NO: 433 - protein, 34 aa, synthetic construct
  VARIANT 20: note = This region may encompass one of the following sequences: (G)n where n is an integer from 1 to 5, (GS)n where n is an integer from 1 to 5, (GGS)n where n is an integer from 1 to 5 , (GGGS)n where n is an integer from 1 to 5 , (GGGGS)n where n is an integer from 1 to 5, or a combination of any of the foregoing; such as GGGGG, GSGSGSGSGS, GGSGGSGGSGGSGGS, GGGSGGGSGGGSGGGSGGGS, or GGGGSGGGGSGGGGSGGGGSGGGGS
IPWCTLDPGGLQCAWLGKHXVVCQDWEGVELCWQ

SEQ ID NO: 434 - protein, 33 aa, synthetic construct
  VARIANT 19: note = This region may encompass one of the following sequences: (G)n where n is an integer from 1 to 5, (GS)n where n is an integer from 1 to 5, (GGS)n where n is an integer from 1 to 5 , (GGGS)n where n is an integer from 1 to 5 , (GGGGS)n where n is an integer from 1 to 5, or a combination of any of the foregoing; such as GGGGG, GSGSGSGSGS, GGSGGSGGSGGSGGS, GGGSGGGSGGGSGGGSGGGS, or GGGGSGGGGSGGGGSGGGGSGGGGS
IPWCTLDPGGLQCAWLRMXVVCQDWEGVELCWQ

SEQ ID NO: 435 - protein, 37 aa, synthetic construct
  VARIANT 1: note = This region may encompass one of the following amino acids: A, G, P, S, T, and V.
  VARIANT 2: note = This region may encompass one of the following amino acids: F, H, W, or Y.
  VARIANT 3: note = This region may encompass one of the following amino acids:A, G, H, K P, R, S, or T.
  VARIANT 20: note = This region may encompass one of the following amino acids: A, G, H, K, P, R, S, or T.
  VARIANT 13: note = This region may encompass one of the following amino acids: A, G, P, S, or T.
  VARIANT 21: note = This region may encompass an amino acid or a single bond. This region can encompass Q, G, and K.
  VARIANT 22: note = This region may encompass an amino acid or a single bond. This region can encompass Q, G, and M.
  VARIANT 23: note = This region may encompass one of the following sequences: from (G)n where n is an integer from 1 to 5 (SEQ ID NO: 9390), (GS)n where n is an integer from 1 to 5 (SEQ ID NO: 9391), (GGS)n where n is an integer from 1 to 5 (SEQ ID NO: 9392), (GGGS)n where n is an integer from 1 to 5 (SEQ ID NO: 9393), (GGGGS)n where n is an integer from 1 to 5 (SEQ ID NO: 9394), (GGGGSGG) where n is an integer from 1 to 5 (SEQ ID NO: 9404), or a combination of any of the foregoing; GGGGG, GSGSGSGSGS, GGSGGSGGSGGSGGS, GGGSGGGSGGGSGGGSGGGS, GGGGSGGGGSGGGGSGGGGSGGGGS, or GGGGSGGGGGGSGGGGGGSGGGGGGSGGGGGGSGG
XXXIPWCTLDPGXLQCAWLXXXXVVCQDWEGVELCWQ

SEQ ID NO: 436 - protein, 36 aa, synthetic construct
  VARIANT 22: note = This region may encompass one of the following sequences: from (G)n where n is an integer from 1 to 5 (SEQ ID NO: 9390), (GS)n where n is an integer from 1 to 5 (SEQ ID NO: 9391), (GGS)n where n is an integer from 1 to 5 (SEQ ID NO: 9392), (GGGS)n where n is an integer from 1 to 5 (SEQ ID NO: 9393), (GGGGS)n where n is an integer from 1 to 5 (SEQ ID NO: 9394), (GGGGSGG) where n is an integer from 1 to 5 (SEQ ID NO: 9404), or a combination of any of the foregoing; GGGGG, GSGSGSGSGS, GGSGGSGGSGGSGGS, GGGSGGGSGGGSGGGSGGGS, GGGGSGGGGSGGGGSGGGGSGGGGS, or GGGGSGGGGGGSGGGGGGSGGGGGGSGGGGGGSGG
  VARIANT 1: note = This region may encompass one of the following amino acids: F, H, W, or Y.
  VARIANT 2: note = This region may encompass one of the following amino acids:A, G, H, K P, R, S, or T.
  VARIANT 12: note = This region may encompass one of the following amino acids: A, G, P, S, or T.
  VARIANT 19: note = This region may encompass one of the following amino acids: A, G, H, K, P, R, S, or T.
  VARIANT 20: note = This region may encompass an amino acid.
  VARIANT 21: note = This region may encompass an amino acid.
XXIPWCTLDPGXLQCAWLXXXXVVCQDWEGVELCWQ

SEQ ID NO: 437 - protein, 35 aa, synthetic construct
  VARIANT 1: note = This region may encompass one of the following amino acids:A, G, H, K P, R, S, or T.
  VARIANT 11: note = This region may encompass one of the following amino acids:A, G, H, K P, R, S, or T.
  VARIANT 18: note = This region may encompass one of the following amino acids: A, G, H, K, P, R, S, or T.
  VARIANT 19: note = This region may encompass an amino acid and a single bond. This region can encompass can encompass Q, G, and K.
  VARIANT 20: note = This region may encompass an amino acid and a single bond. This region can encompass can encompass Q, G, and K.
  VARIANT 21: note = This region may encompass one of the following sequences: from (G)n where n is an integer from 1 to 5 (SEQ ID NO: 9390), (GS)n where n is an integer from 1 to 5 (SEQ ID NO: 9391), (GGS)n where n is an integer from 1 to 5 (SEQ ID NO: 9392), (GGGS)n where n is an integer from 1 to 5 (SEQ ID NO: 9393), (GGGGS)n where n is an integer from 1 to 5 (SEQ ID NO: 9394), (GGGGSGG) where n is an integer from 1 to 5 (SEQ ID NO: 9404), or a combination of any of the foregoing; GGGGG, GSGSGSGSGS, GGSGGSGGSGGSGGS, GGGSGGGSGGGSGGGSGGGS, GGGGSGGGGSGGGGSGGGGSGGGGS, or GGGGSGGGGGGSGGGGGGSGGGGGGSGGGGGGSGG.
XIPWCTLDPGXLQCAWLXXXXVVCQDWEGVELCWQ

SEQ ID NO: 438 - protein, 34 aa, synthetic construct
  VARIANT 20: note = This region may encompass one of the following sequences: from (G)n where n is an integer from 1 to 5 (SEQ ID NO: 9390), (GS)n where n is an integer from 1 to 5 (SEQ ID NO: 9391), (GGS)n where n is an integer from 1 to 5 (SEQ ID NO: 9392), (GGGS)n where n is an integer from 1 to 5 (SEQ ID NO: 9393), (GGGGS)n where n is an integer from 1 to 5 (SEQ ID NO: 9394), (GGGGSGG) where n is an integer from 1 to 5 (SEQ ID NO: 9404), or a combination of any of the foregoing; GGGGG, GSGSGSGSGS, GGSGGSGGSGGSGGS, GGGSGGGSGGGSGGGSGGGS, GGGGSGGGGSGGGGSGGGGSGGGGS, or GGGGSGGGGGGSGGGGGGSGGGGGGSGGGGGGSGG.
  VARIANT 19: note = This region may encompass an amino acid or can be absent. This region can encompass Q, G, and M.
  VARIANT 10: note = This region may encompass one of the following amino acids: A, G, P, S, or T.
  VARIANT 17: note = This region may encompass one of the following amino acids: A, G, P, S, or T.
  VARIANT 18: note = This region may encompass an amino acid or can be absent. This region can encompass Q, G, or K.
IPWCTLDPGXLQCAWLXXXXVVCQDWEGVELCWQ

SEQ ID NO: 439 - protein, 36 aa, synthetic construct
  VARIANT 1: note = This region may encompass one of the following amino acids: A, G, S, T, or V.
  VARIANT 2: note = This region may encompass one of the following amino acids: F, H, W, or Y.
  VARIANT 3: note = This region may encompass one of the following amino acids:A, G, H, K P, R, S, or T.
  VARIANT 13: note = This region may encompass one of the following amino acids: A, G, P, S, or T.
  VARIANT 20: note = This region may encompass one of the following amino acids: A, G, H, K, P, R, S, or T.
  VARIANT 21: note = This region may encompass an amino acid.
  VARIANT 22: note = This region may encompass one of the following sequences: from (G)n where n is an integer from 1 to 5 (SEQ ID NO: 9390), (GS)n where n is an integer from 1 to 5 (SEQ ID NO: 9391), (GGS)n where n is an integer from 1 to 5 (SEQ ID NO: 9392), (GGGS)n where n is an integer from 1 to 5 (SEQ ID NO: 9393), (GGGGS)n where n is an integer from 1 to 5 (SEQ ID NO: 9394), (GGGGSGG) where n is an integer from 1 to 5 (SEQ ID NO: 9404), or a combination of any of the foregoing; GGGGG, GSGSGSGSGS, GGSGGSGGSGGSGGS, GGGSGGGSGGGSGGGSGGGS, GGGGSGGGGSGGGGSGGGGSGGGGS, or GGGGSGGGGGGSGGGGGGSGGGGGGSGGGGGGSGG.
XXXIPWCTLDPGXLQCAWLXXXVVCQDWEGVELCWQ

SEQ ID NO: 440 - protein, 35 aa, synthetic construct
  VARIANT 21: note = This region may encompass one of the following sequences: from (G)n where n is an integer from 1 to 5 (SEQ ID NO: 9390), (GS)n where n is an integer from 1 to 5 (SEQ ID NO: 9391), (GGS)n where n is an integer from 1 to 5 (SEQ ID NO: 9392), (GGGS)n where n is an integer from 1 to 5 (SEQ ID NO: 9393), (GGGGS)n where n is an integer from 1 to 5 (SEQ ID NO: 9394), (GGGGSGG) where n is an integer from 1 to 5 (SEQ ID NO: 9404), or a combination of any of the foregoing; GGGGG, GSGSGSGSGS, GGSGGSGGSGGSGGS, GGGSGGGSGGGSGGGSGGGS, GGGGSGGGGSGGGGSGGGGSGGGGS, or GGGGSGGGGGGSGGGGGGSGGGGGGSGGGGGGSGG.
  VARIANT 20: note = This region may encompass one of the following amino acids: A, G, H, K, P, R, S, or T.
  VARIANT 1: note = This region may encompass one of the following amino acids: A, G, S, T, or V.
  VARIANT 2: note = This region may encompass one of the following amino acids: F, H, W, or Y.
  VARIANT 3: note = This region may encompass one of the following amino acids:A, G, H, K P, R, S, or T.
  VARIANT 13: note = This region may encompass one of the following amino acids: A, G, P, S, or T.
XXXIPWCTLDPGXLQCAWLXXVVCQDWEGVELCWQ

SEQ ID NO: 441 - protein, 34 aa, synthetic construct
  VARIANT 13: note = This region may encompass one of the following amino acids: A, G, P, S, or T.
  VARIANT 20: note = This region may encompass one of the following sequences: from (G)n where n is an integer from 1 to 5 (SEQ ID NO: 9390), (GS)n where n is an integer from 1 to 5 (SEQ ID NO: 9391), (GGS)n where n is an integer from 1 to 5 (SEQ ID NO: 9392), (GGGS)n where n is an integer from 1 to 5 (SEQ ID NO: 9393), (GGGGS)n where n is an integer from 1 to 5 (SEQ ID NO: 9394), (GGGGSGG) where n is an integer from 1 to 5 (SEQ ID NO: 9404), or a combination of any of the foregoing; GGGGG, GSGSGSGSGS, GGSGGSGGSGGSGGS, GGGSGGGSGGGSGGGSGGGS, GGGGSGGGGSGGGGSGGGGSGGGGS, or GGGGSGGGGGGSGGGGGGSGGGGGGSGGGGGGSGG.
  VARIANT 1: note = This region may encompass one of the following amino acids: A, G, S, T, or V.
  VARIANT 2: note = This region may encompass one of the following amino acids: F, H, W, or Y.
  VARIANT 3: note = This region may encompass one of the following amino acids:A, G, H, K P, R, S, or T.
XXXIPWCTLDPGXLQCAWLXVVCQDWEGVELCWQ

SEQ ID NO: 442 - protein, 33 aa, synthetic construct
  VARIANT 12: note = This region may encompass one of the following amino acids: A, G, P, S, or T.
  VARIANT 1: note = This region may encompass one of the following amino acids: A, G, S, T, or V.
  VARIANT 2: note = This region may encompass one of the following amino acids: F, H, W, or Y.
  VARIANT 3: note = This region may encompass one of the following amino acids: A, G, H, K P, R, S, or T.
  VARIANT 19: note = This region may encompass one of the following sequences: from (G)n where n is an integer from 1 to 5 (SEQ ID NO: 9390), (GS)n where n is an integer from 1 to 5 (SEQ ID NO: 9391), (GGS)n where n is an integer from 1 to 5 (SEQ ID NO: 9392), (GGGS)n where n is an integer from 1 to 5 (SEQ ID NO: 9393), (GGGGS)n where n is an integer from 1 to 5 (SEQ ID NO: 9394), (GGGGSGG) where n is an integer from 1 to 5 (SEQ ID NO: 9404), or a combination of any of the foregoing; GGGGG, GSGSGSGSGS, GGSGGSGGSGGSGGS, GGGSGGGSGGGSGGGSGGGS, GGGGSGGGGSGGGGSGGGGSGGGGS, or GGGGSGGGGGGSGGGGGGSGGGGGGSGGGGGGSGG.
XXXIPWCTLDPXLQCAWLXVVCQDWEGVELCWQ

SEQ ID NO: 443 - protein, 43 aa, synthetic construct
VHRIPWCTLDPGGLQCAWLRQMGGGGSGGVVCQDWEGVELCWQ

SEQ ID NO: 444 - protein, 42 aa, synthetic construct; identical to SEQ ID NO: 400

SEQ ID NO: 445 - protein, 41 aa, synthetic construct; identical to SEQ ID NO: 398

SEQ ID NO: 446 - protein, 42 aa, synthetic construct
HRIPWCTLDPGGLQCAWLRQMGGGGSGGVVCQDWEGVELCWQ

SEQ ID NO: 447 - protein, 41 aa, synthetic construct
HRIPWCTLDPGGLQCAWLRQGGGGSGGVVCQDWEGVELCWQ

SEQ ID NO: 448 - protein, 40 aa, synthetic construct
HRIPWCTLDPGGLQCAWLRGGGGSGGVVCQDWEGVELCWQ

SEQ ID NO: 449 - protein, 41 aa, synthetic construct
RIPWCTLDPGGLQCAWLRQMGGGGSGGVVCQDWEGVELCWQ

SEQ ID NO: 450 - protein, 40 aa, synthetic construct
RIPWCTLDPGGLQCAWLRQGGGGSGGVVCQDWEGVELCWQ

SEQ ID NO: 451 - protein, 39 aa, synthetic construct
RIPWCTLDPGGLQCAWLRGGGGSGGVVCQDWEGVELCWQ

SEQ ID NO: 452 - protein, 40 aa, synthetic construct; identical to SEQ ID NO: 415

SEQ ID NO: 453 - protein, 39 aa, synthetic construct; identical to SEQ ID NO: 416

SEQ ID NO: 454 - protein, 38 aa, synthetic construct; identical to SEQ ID NO: 417

SEQ ID NO: 455 - protein, 14 aa, synthetic construct
QCIHWDIETLLSCV

SEQ ID NO: 456 - protein, 14 aa, synthetic construct
VYCAEIGEYRVCRQ

SEQ ID NO: 457 - protein, 14 aa, synthetic construct
YMACSSGLSLCRLS

SEQ ID NO: 458 - protein, 20 aa, synthetic construct
QCVHWDLDTLFGCIREQLEL

SEQ ID NO: 459 - protein, 18 aa, synthetic construct
GGVPWCTLDPGSLQCAWF

SEQ ID NO: 460 - protein, 14 aa, synthetic construct
YDCRIAQVGELCDL

SEQ ID NO: 461 - protein, 14 aa, synthetic construct
KVCEMWGGVLLCWN

SEQ ID NO: 462 - protein, 14 aa, synthetic construct
RTCTEWENVVLCWV

SEQ ID NO: 463 - protein, 14 aa, synthetic construct
MCWLEWGEWVGSCL

SEQ ID NO: 464 - protein, 14 aa, synthetic construct
CPSMLQGPERTWVC

SEQ ID NO: 465 - protein, 14 aa, synthetic construct
CPSMLQGPERTWVC

SEQ ID NO: 466 - protein, 14 aa, synthetic construct
CPGELRGPERAWVC